(19) United States
(12) Patent Application Publication
(10) Pub. No.: US 2025/0297320 A1
(43) Pub. Date: Sep. 25, 2025

(54) METHYLATION SIGNATURES IN CELL-FREE DNA FOR TUMOR CLASSIFICATION AND EARLY DETECTION

(71) Applicant: H. LEE MOFFITT CANCER CENTER AND RESEARCH INSTITUTE, INC., Tampa, FL (US)

(72) Inventors: Liang WANG, Tampa, FL (US); Brandon J. MANLEY, Tampa, FL (US); Anders E. BERGLUND, Tampa, FL (US); Xuefeng WANG, Tampa, FL (US); Krupal B. PATEL, Tampa, FL (US); Jinyong HUANG, Guangdong (CN)

(21) Appl. No.: 18/709,722

(22) PCT Filed: Nov. 11, 2022
(86) PCT No.: PCT/US2022/079735

§ 371 Date: May 13, 2024

Related U.S. Application Data
(60) Provisional application No. 63/278,921, filed on Nov. 12, 2021.

(51) Int. Cl. C12Q 1/6886

(52) U.S. Cl. CPC C12Q 1/6886; C12Q 2600/112; C12Q 2600/154

(57) ABSTRACT

Disclosed are methods of using cell-free DNA (cfDNA) methylation for the detection, typing, and grading of cancer.

This application claims the benefit of U.S. Application No. 63/278,921, filed on Nov. 12, 2021, which is incorporated herein by reference in its entirety.

This invention was made with government support under Grant No. CA212097 and CA250018 awarded by the National Institutes of Health. The government has certain rights in the invention.

### I. BACKGROUND

Lung and colorectal cancer are among the most common causes of cancer-related deaths in the US, while pancreatic cancer is the deadliest form of solid malignancy with an alarming 10% five-year survival rate. The dismal mortality rates seen in patients with these malignancies are associated with advanced stage at the time of diagnosis. To improve the outcomes of this patient population, many technologies and assays that enable cancer detection at its early stage have been investigated. What are needed are new proven methods for accurate and early detection of cancer.

### II. SUMMARY

Disclosed are methods related to the use of methyl-CpG-binding domain sequencing (MBD-seq) to detect, type, grade, and/or treat a cancer.

In one aspect, disclosed herein are methods of detecting, diagnosing, and/or grading a cancer and/or metastasis (such as, for example, colorectal, lung, and/or pancreatic cancer), said method comprising a) obtaining a fluid biological sample (such as, for example, plasma, serum, and/or cerebrospinal fluid); b) extracting cfDNA; c) generating methylated filler DNA; d) ligating an adapter to the cfDNA and combining with filler DNA thereby creating methylation cfDNA library; e) enriching for methylated cfDNA; f) amplifying and sequencing enriched methylated cfDNA library; and g) assaying CpG islands for hypermethylation relative to a normal control (autologous noncancerous tissue from the subject or a negative/normal control standard); wherein the presence of CpG hypermethylation at a CpG islands chr4:174427892-174428192, chr7:27265159-27265493, chr7:65037625-65037864, chr8:124172801-124173541, chr12:54408427-54408713, chr13:28549840-28550246, chr1:50798668-50799536, chr5:92939796-92940216, or chr12:114881650-114881937, and/or at CpG island associated with CLIP4 (chr2:29337984-29338909), LONRF2 (chr2:100937780-100939059), RNF217 (chr6:125283125-125284389), MEIS1 (chr2:66672432-66673636), ZNF638 (chr2:71503548-71504233), WNT6 (chr2:219736133-219736592), MGST2 (chr4:140655963-140657135), PTGER4 (chr5:40679503-40682081), C9orf129 (chr9:96108467-96108992), B4GALNT1 (chr12:58021295-58022037), HOXB8 (chr17:46691521-46692097), TBX4 (chr17:59539363-59539834), SOX9 (chr17:70112825-70114271), RNF220 (chr1:44883137-44884272), CELF2 (chr10:11059443-11060524), and/or DBX1 (chr11:20177609-20178824) indicates the presence of a cancer; and wherein presence of hypermethylation indicates the severity (i.e, grade of the cancer). In one aspect, the DNA is not denatured. In one aspect the sample is subject to two centrifuge cycles. In some aspects the cfDNA is collected in streck tubes.

In one aspect disclosed herein are methods of typing a cancer and/or metastasis, said method comprising a) obtaining a fluid biological sample (such as, for example, plasma, serum, and/or cerebrospinal fluid); b) extracting cfDNA; c) generating methylated filler DNA; d) ligating an adapter to the cfDNA and combining with filler DNA thereby creating methylation cfDNA libaray; e) enriching for methylated cfDNA; f) amplifying and sequencing enriched methylated cfDNA library; and g) assaying CpG islands for hypermethylation relative to normal controls (autologous noncancerous tissue from the subject or a negative/normal control standard); wherein the presence of CpG hypermethylation at a CpG islands associated with CLIP4 (chr2:29337984-29338909), LONRF2 (chr2:100937780-100939059), and/or RNF217 (chr6:125283125-125284389) indicate colorectal cancer; wherein the presence of CpG hypermethylation at a CpG islands at chr4:174427892-174428192, chr7:27265159-27265493, chr7:65037625-65037864, chr8:124172801-124173541, and/or chr12:54408427-54408713, and/or at CpG islands associated with MEIS1 (chr2:66672432-66673636), ZNF638 (chr2:71503548-71504233), WNT6 (chr2:219736133-219736592), MGST2 (chr4:140655963-140657135), PTGER4 (chr5:40679503-40682081), C9orf129 (chr9:96108467-96108992), B4GALNT1 (chr12:58021295-58022037), HOXB8 (chr17:46691521-46692097), TBX4 (chr17:59539363-59539834), and/or SOX9 (chr17:70112825-70114271) indicate lung cancer; and wherein the presence of CpG hypermethylation at a CpG islands at chr13:28549840-28550246, chr1:50798668-50799536, chr5:92939796-92940216, and/or chr12:114881650-114881937, and/or at CpG island associated with RNF220 (chr1:44883137-44884272), CELF2 (chr10:11059443-11060524), and/or DBX1 (chr11:20177609-20178824) indicates the presence of a pancreatic cancer. In one aspect, the DNA is not denatured. In one aspect the sample is subject to two centrifuge cycles. In some aspects the cfDNA is collected in streck tubes.

Also disclosed herein are methods of detecting, diagnosing, typing, and/or grading a cancer and/or metastasis of any preceding aspect, wherein the methylated filler DNA is generated by treating amplicons of Enterobacteria phage λ DNA with CpG methyltransferase.

In one aspect, disclosed herein are methods of treating, inhibiting, decreasing, reducing, ameliorating, and/or preventing a cancer (such as, for example, pancreatic, colorectal, and/or lung cancer), said method comprising a) obtaining a fluid biological sample; b) extracting cfDNA; c) generating methylated filler DNA; d) ligating an adapter to the cfDNA and combining with filler DNA thereby creating methylation cfDNA library; e) enriching for methylated cfDNA; f) amplifying and sequencing enriched methylated cfDNA library; g) assaying CpG islands for hypermethylation relative to a normal control (autologous noncancerous tissue from the subject or a negative/normal control standard); wherein the presence of CpG hypermethylation at a CpG islands chr4:174427892-174428192, chr7:27265159-27265493, chr7:65037625-65037864, chr8:124172801-124173541, chr12:54408427-54408713, chr13:28549840-28550246, chr1:50798668-50799536, chr5:92939796-92940216, or chr12:114881650-114881937, and/or at CpG island associated with CLIP4 (chr2:29337984-29338909), LONRF2 (chr2:100937780-100939059), RNF217 (chr6:125283125-125284389), MEIS1 (chr2:66672432-66673636), ZNF638 (chr2:71503548-71504233), WNT6 (chr2:219736133-219736592), MGST2 (chr4:140655963-140657135), PTGER4 (chr5:40679503-40682081), C9orf129 (chr9:96108467-96108992), B4GALNT1 (chr12:58021295-58022037), HOXB8 (chr17:46691521-46692097), TBX4 (chr17:59539363-59539834), SOX9 (chr17:70112825-70114271), RNF220 (chr1:44883137-44884272), CELF2 (chr10:11059443-11060524), and/or DBX1 (chr11:20177609-20178824) indicates the presence of a cancer; and h) treating the cancer with an effective amount of a therapeutic agent. In one aspect, the DNA is not denatured. In one aspect the sample is subject to two centrifuge cycles. In some aspects the cfDNA is collected in streck tubes.

Also disclosed herein are methods of treating, inhibiting, decreasing, reducing, ameliorating, and/or preventing a cancer of any preceding aspect, wherein the methylated filler DNA is generated by treating amplicons of Enterobacteria phage λ DNA with CpG methyltransferase.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows workflow chart of data generation and analysis. BH-FDR, Benjamini-Hochberg false discovery rate; DMRs, differentially methylated regions; DMCGIs, differentially methylated CpG islands; LASSO, least absolute shrinkage and selection operator.

FIG. 2A-D show quality controls of cfMBD-seq. FIG. 2A shows cfDNA concentration (ng cfDNA per ml plasma) from colorectal cancer (N=13), lung cancer (N=12), pancreatic cancer (N=12) patients, and non-cancer controls (N=16). FIG. 2B shows total sequence reads and high-quality sequence reads across different groups. FIG. 2C shows the percentage of transcripts per million (TPM) normalized reads on CpG islands across different groups. FIG. 2D shows the percentage of TPM normalized reads on CpG islands/shores/shelves across different groups. For all box plots, the extremes of the boxes represent the upper and lower quartiles and the center lines define the median. Whiskers indicate 1.5× interquartile range.

FIGS. 3A-3F show quality controls of cfMBD-seq methylation capture and library construction. FIG. 3A shows the specificity of MBD methylation capture reaction across different groups (i.e., Healthy, non-cancer individuals; Colorectal, colorectal cancer patients; Lung, lung cancer patients; Pancreas, pancreatic cancer patients) calculated using qPCR Ct value of methylated and unmethylated spiked-in A. thaliana DNA. FIG. 3B shows the percentage of sequence reads that doesn't contain any CpG tandem across different groups. FIG. 3C shows the ratio of average non-CpG coverage to average CpG coverage across different groups. Non-CpG coverage is defined as the average coverage of fragments without any CpG tandem. CpG coverage is defined as the average coverage of fragments with no less than one CpG tandem. FIG. 3D shows the CpG density at peak across different groups. CpG density is defined as number of CpG tandems per fragment. Peak is defined as fragments with highest coverage. FIG. 3E shows the percentage of sequencing coverage across different CpG annotation features (i.e., CpG islands, CpG shores, CpG shelves, and inter CpG regions) for all samples. FIG. 3F shows the percentage of different CpG annotation features in base pair size in hg19 human genome. For all box plots, the extremes of the boxes represent the upper and lower quartiles, and the center lines define the median. Whiskers indicate 1.5× interquartile range.

FIGS. 4A-4D show differentially methylated regions between cases and controls detected by cfMBD-seq. FIG. 4A shows Volcano plots of differentially methylated regions (DMRs) at extended CpG islands (CGI) (i.e., CpG islands, CpG shores, and CpG shelves) between lung cancer patients (N=12) and non-cancer controls (N=16). Black dots indicate non-significant regions. Blue and red dots indicate statistical significance at Benjamini-Hochberg false discovery rate (FDR)<0.1 (negative binomial model, Wald test). Red dots also indicate regions with absolute fold change (FC)>2. FIG. 4B shows Volcano plots of DMRs at CpG islands between lung cancer patients and non-cancer controls. FIG. 4C shows unsupervised hierarchical clustering (z scores normalization of DESeq2 normalized counts, Euclidean distance, and Ward Clustering) of the top 100 differentially hypermethylated CpG islands between lung cancer patients and non-cancer controls. FIG. 4D shows principal component (PC) analysis using DESeq2 normalized counts of the top 1,000 differentially hypermethylated CpG islands between lung cancer patients and non-cancer controls.

FIGS. 5A-F show DMRs between cases and controls detected by cfMBD-seq. FIGS. 5A and 5B show Volcano plots of DMRs at CpG islands/shores/shelves between colorectal cancer (5a)/pancreatic cancer (5b) patients and non-cancer controls. Black dots indicate non-significant regions. Blue and red dots indicate regions significant at Benjamini-Hochberg false discovery rate (BH-FDR)<0.1 (negative binomial model, Wald test). Red dots also indicate regions with absolute fold change >2. FIGS. 5C and 5D show Volcano plots of DMRs at CpG islands between colorectal cancer (5c)/pancreatic cancer (5d) patients and non-cancer controls. FIGS. 5E and 5F show unsupervised hierarchical clustering (z score normalization of DESeq2 normalized counts, Euclidean distance, and Ward Clustering) of the top 100 differentially hypermethylated CpG islands between colorectal cancer (5e)/pancreatic cancer (5f) patients and non-cancer controls. Dendrogram shows separation by sample type (case or control).

FIGS. 6A-E show DMRs between cases and controls detected by cfMBD-seq. FIGS. 6A and 6B show the principal component analysis using DESeq2 normalized counts of top 1,000 differentially hypermethylated CpG islands between colorectal cancer (6a)/pancreatic cancer (6b) patients and non-cancer controls. The 95% confidence ellipses for the case and control are displayed. FIGS. 6C, 6D, and 6E show the proportion of variance explained by each principal component.

FIGS. 7A-F show HM450K DMCs between primary tumors and adjacent normal tissues/normal blood cells. FIG. 7A shows pathology stage (according to the AJCC/UICC 7th Edition) in the HM450K cohort including N=66 paired primary tumors and adjacent normal tissues, and N=61 non-cancer peripheral blood mononuclear cells (PBMCs). Early-stage consists of stage I and II. Late-stage consists of stage III and IV. FIGS. 7B and 7C show Volcano plots of DMCs between primary tumors and adjacent normal tissues for COAD (N=35) (7b) or PAAD (N=10) (7c) from HM450K data. FIGS. 7D, 7E, and 7F show Volcano plots of DMCs between primary tumors and PBMCs for COAD (7d), LUAD (N=21) (7e), or PAAD (7f). For all volcano plots, black dots indicate non-significant regions. Blue and red dots indicate regions significant at Benjamini-Hochberg false discovery rate (BH-FDR)<0.1 (F-test). Red dots also indicate regions with mean of Abeta value >0.2.

FIGS. 8A-8D show differentially methylated CpG islands are mainly driven by tumor-specific DNA methylation patterns. FIG. 8A shows Volcano plots of differentially methylated CpG sites between lung adenocarcinoma (LUAD) primary tumors and matched adjacent normal tissues from 21 patients from Infinium HumanMethylation450 BeadChip (HM450K) data. Black dots indicate non-significant regions. Blue and red dots indicate regions significant at Benjamini-Hochberg false discovery rate (FDR)<0.1 (F-test). Red dots also indicate regions with mean of Δ beta value (DBV)>0.2. FIG. 8B shows Venn diagram showing the number of overlapping regions between plasma-derived differentially methylated CpG islands (DMCGIs) from cfMBD-seq and tissues-derived DMCGIs from HM450K in three cancer types (i.e., C, colorectal cancer; L, lung cancer; P, pancreatic cancer). FIG. 8C shows predictive modeling using LASSO regularized logistic regression case-versus-control models on cfMeDIP-seq cohort including lung cancer patients (N=80) and non-cancer controls (N=86). ROC curve for 20% of held-out testing set is shown. AUC values represent median and interquartile range for 100 repeats of the model. FIG. 8D shows t-distributed stochastic neighbor embedding (t-sne) plot using 3 classifiers identified from training set for plasma samples of the entire cfMeDIP-seq cohort (N=166).

FIG. 9A-9C show performance of overlapping DMCGIs in cfMeDIP seq cohort. FIG. 9a shows pathology stage (according to the AJCC/UICC 7th Edition) in the cfMeDIP-seq cohort. Early-stage consists of stage I and II. Late-stage consists of stage III and IV. FIG. 9B shows t-sne plot using all 939 lung DMCGIs that are overlapped between cfMBD-seq and HM450K data for the entire cfMeDIP-seq plasma samples (N=166). FIG. 9C shows Log transformed transcripts per kilobase million (TPM) of the 3 classifiers from the cfMeDIP-seq training set. The extremes of the boxes define the upper and lower quartiles, and the center lines define the median. Whiskers indicate 1.5× interquartile range.

FIGS. 10A-10D show performance of differentially methylated CpG islands in cancer classification. FIG. 10A shows a Venn diagram showing the number of tissue specific DMCGIs for each cancer type and the number of DMCGIs that are common in all three cancer types. FIG. 10A shows predictive modeling using LASSO regularized logistic regression one-versus-all-others models on the HM450K cohort including 210 colon adenocarcinoma (COAD) samples, 385 lung adenocarcinoma (LUAD) samples, and 162 pancreatic adenocarcinoma (PAAD) samples. Area under the curve (AUC) values are calculated from 20% of held-out testing set. Boxplots represent median and interquartile range for 100 repeats of the models. FIGS. 10C and 10D show t-sne plot using tissue specific classifiers identified from training set for the entire cfMBD-seq plasma cohort (N=53) (10c) and HM450K tissue cohort (N=757 primary tumor and N=61 non-cancer PBMCs) (10d).

FIGS. 11A and B show performance of cancer type specific DMCGIs in independent HM450K cohort. FIG. 11A shows pathology stage (according to the AJCC/UICC 7th Edition) in the TCGA HM450K cohort of different tumor (N=757). Early-stage consists of stage I and II. Late-stage consists of stage III and IV. FIG. 11B shows Beta value of cancer type specific classifiers (Colorectal cancer specific: chr2:29337984-29338909; Lung cancer specific: chr7:27265159-27265493; Pancreatic cancer specific: chr10:11059443-11060524) across COAD, LUAD, PAAD, and PBMC samples. The extremes of the boxes define the upper and lower quartiles, and the center lines define the median. Whiskers indicate 1.5× interquartile range.

FIGS. 12A-12D show reduced MethylCap protein improves low-input methylation enrichment. FIGS. 12A and 12B show the total normalized CpG islands coverage and CpG islands/shores/shelves coverage across different amounts of MethylCap protein and magnetic beads. (N=4 for the first condition, N=3 for other conditions. Mean with the standard error of the mean (SEM).) FIG. 12C shows coverage by CpG density plot across different amounts of MethylCap protein and magnetic beads. Coverage is defined as the average number of fragments covering CpGs. The CpG density is the number of CpGs per fragment. FIG. 12D shows CpG density at peak and noise under different MethylCap proteins and magnetic beads. The CpG density at the peak is the CpG density at the point of highest coverage on the ‘coverage by CpG density plot’ (left y-axis). Noise is the ratio of average non-CpG coverage to average CpG coverage (right y-axis).

FIG. 13A-13D show Methylated filler DNA is needed to compensate for low-input methylation enrichment. FIGS. 13A and 13B show total normalized CpG islands coverage and CpG islands/shores/shelves coverage across different methylation states of filler DNA. (N=4 for the first condition, N=3 for other conditions. Mean with SEM.) FIG. 13C shows coverage by CpG density plots across different methylation states of filler DNA. FIG. 13D show the CpG density at peak (left y-axis) and noise (right y-axis) at different methylation states of filler DNA.

FIGS. 14A-14D show different input DNA amounts in cfMBD-seq. FIG. 14A shows genome-wide Pearson correlations of normalized read counts between cfMBD-seq signal for 1-1000 ng of input HCT116 DNA (2 technical replicates per concentration). The input control is from an input library of a ChIP-seq study (ENCODE: ENCFF280GWX). Log transformed counts were used in the scatter plots. FIGS. 14B and 14C show total normalized CpG islands coverage and CpG islands/shores/shelves coverage across different mixtures of cfDNA and filler DNA. (N=4 for the first condition, N=2 for other conditions. Mean with SEM.) FIG. 14D shows CpG density at peak (left y-axis) and noise (right y-axis) of different mixtures of cfDNA and filler DNA.

FIGS. 15A-15D shows a comparison of cfMBD-seq with low input MBD-seq and cfMeDIP-seq. FIG. 15A shows receiver operating characteristic curve and corresponding area under the ROC curve for methylation status of CpG islands from Infinium HM450K data predicted by cfMBD-seq normalized read counts. FIGS. 15B and 15C show total normalized CpG annotations coverage and CpG islands/shores/shelves coverage of cfMBD-seq (N=8), cfMeDIP-seq (N=24), and low-input MBD-seq (N=4). (Mean with SEM.) FIG. 15D shows coverage by CpG density plot of cfMBD-seq, cfMeDIP-seq, and low-input MBD-seq.

FIG. 16 shows cfMBD-seq recapitulates methylation profiles from other technologies. Genome Browser snapshot of HCT116 cfMBD-seq signal across chr8:145,095,942-145,116,942, at different starting DNA inputs (1 to 100 ng), compared with cfMeDIP-seq (Gene Expression Omnibus (GEO): GSE79838), RRBS (ENCODE: ENCSROOODFS), and WGBS (GEO: GSM1465024) data. For cfMBD-seq and cfMeDIP-seq, the y-axis indicates RPKMs normalized reads; for RRBS, red and green blocks represent hypermethylated and hypomethylated CpGs, respectively. For the WGBS track, peak heights indicate methylation levels.

FIGS. 17A and 17B show a schematic diagram of cfMBD-seq and CpG annotations FIG. 17A shows schematic workflow of cfMBD-seq protocol. From cfDNA extraction to generation of methylation profile. FIG. 17B shows schematic diagram of CpG annotations. Numbers on the left (in brackets) represent the percentage of the CpG features in the human genome. For example, CpG islands account for only 0.7% of the human genome. Numbers on the right represent total number of features. For example, there are 28,691 CpG islands in the hg19 reference genome.

FIGS. 18A-18D show a library yield and enrichment specificity are important pre-sequencing quality controls FIGS. 18a and 18B show library concentration (ng/μl) measured by Qubit assay across different conditions. FIGS. 18C and 18D show the specificity of methylation enrichment measured by qPCR, using methylated and unmethylated spiked-in A. thaliana DNA control.

FIG. 19 shows saturation analysis of cfMBD-seq data. Saturation analysis from the MEDIPS package analyzing different HCT116 DNA input. The saturation analysis determines if the given set of mapped reads is sufficient to generate a saturated and reproducible coverage profile of the reference genome.

FIGS. 20A and 20B show additional wash does not improve methylation enrichment. FIG. 20A shows the total normalized CpG annotations coverage across different wash conditions. (N=4 for each condition. Mean with SEM.) FIG. 20B shows CpG density at peak (left y-axis) and noise (right y-axis) of different wash conditions.

FIG. 21A-21E show the effect of elution buffer in cfMBD capture. FIG. 21A shows coverage by CpG density plot across elution buffers with different salt concentration. FIG. 21B shows CpG density at peak (left y-axis) and noise (right y-axis) of different elution buffers. FIG. 21C shows the total normalized CpG annotations coverage across different wash conditions. (Mean with SEM.) FIG. 21D shows a genome Browser snapshot of cfMBD-seq signal at the CpG island of MGAT3, which is used as an example in the manual of MethylCap kit. Data were processed by MEDIPS package for RPKMs normalization and were exported as wiggle files for visualization. FIG. 21E shows the coverage by CpG density plot across multiple fractions of elution conditions.

FIG. 22 shows cfMBD-seq shares similar methylation profile with cfMeDIP-seq. Genome Browser snapshot of different input of HCT116 DNA signal by cfMBD-seq and cfMeDIP-seq across a region with consecutive CpG islands (chr8:86,703,816-86,880,439). Data were processed by MEDIPS package for RPKMs normalization and were exported as wiggle files for visualization.

FIGS. 23A and 23B show an overview of the proposed DMR analysis on OCSCC plasma samples. FIG. 23A shows the experimental design and overall analytical workflow for cfDNA methylation profiling on pre- and post-treatment OCSCC patient samples. FIG. 23B shows Pie charts showing the distribution of methylation status and genomic locations in the top detected DMRs.

FIG. 24 shows the normalized methylation levels of top DMRs across the matched plasma samples from 8 patients.

FIGS. 25A, 25B, 25C, 25D, and 25E show prioritizing cfDNA DMRs based on the four-patient subgroup. FIG. 25A shows methylation levels in top regions that were identified based on the TCGA-concordant patient subgroup (P1, P4, P7 and P8). FIGS. 25B, 25C, 25D, and 25E show Kaplan-Meier plots validating the prognostic significance of four genes that include detected DMRs (based on the gene expression and survival data from the TCGA-HNSC data).

FIGS. 26A and 26B show clustering analysis of targeted plasma cfDNA methylation regions. We tested the performance of top DMRs (as well as the model saturation in terms of the number of biomarkers included) in discriminating pre- and post-treatment plasma samples. The two heatmaps in FIG. 26 illustrate the unsupervised clustering results generated based on top 30 DMRs (26A) and top 200 DMRs (26B)(from the DMR test using all samples but P5), respectively. It shows that the top 30 regions (most of them are hypermethylated in pre-treatment samples) are already sufficient to separate pre- and post-treatment plasma samples except for the P5 pre-treatment sample. This was expected, because the global PCA analysis also indicated that this sample could be a potential outlier. But when top 200 regions were included, this sample, together with all other samples, can be correctly separated.

FIGS. 27A and 27B show genome-wide PCA of cfDNA methylation profiles (promoter regions only) using (27A) all pre- and post-treatment plasma samples; (27B) all patients but Patient 5.

FIG. 28 shows a comparison of methylation enriched read counts in MPNST group compared to NF1 group. Heat map showing intensity based on normalized read counts in both groups (n=6 in each group) for A, 73 CpG islands and B, 16 significant CpG islands (one-tailed unpaired t-test, q<0.1). C, Violin plot showing comparison of methylation enriched read counts for 73 CpG islands. Mann-Whitney test (****P<0.0001). tSNE plot showing methylation pattern between both groups based on D, 73 probes and E, 16 probes.

FIG. 29 shows a comparison of methylation enriched read counts in MPNST group compared to NF1 group. Heat map showing intensity based on normalized read counts in both groups (n=6 in each group) for A, 73 CpG islands and B, 16 significant CpG islands (one-tailed unpaired t-test, q<0.1). C, Violin plot showing comparison of methylation enriched read counts for 73 CpG islands. Mann-Whitney test (****P<0.0001). tSNE plot showing methylation pattern between both groups based on D, 73 probes and E, 16 probes.

FIG. 30 shows a comparison of methylation enriched read counts in MPNST group compared to NF1 group. Heat map showing intensity based on normalized read counts in both groups (n=6 in each group) for A, 73 CpG islands and B, 16 significant CpG islands (one-tailed unpaired t-test, q<0.1). C, Violin plot showing comparison of methylation enriched read counts for 73 CpG islands. Mann-Whitney test (****P<0.0001). tSNE plot showing methylation pattern between both groups based on D, 73 probes and E, 16 probes.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Method of Detecting, Typing, Grading, and Treating Cancer

Among new technologies for detection of cancer, the use of liquid biopsies is rapidly gaining prominence for minimally invasive cancer detection and management. Specifically, the detection of tumor-specific circulating cell-free DNA (cfDNA) methylation aberrations holds great promise as a blood-based test for cancer diagnosis for several reasons: First, aberrant DNA methylation occurs early during tumorigenesis and is abundantly present in the entire cancer process. Second, in contrast to the highly heterogeneous nature of gene mutations, tumors of the same histological type tend to exhibit similar DNA methylation changes among different individuals. Third, circulating components are shed from multiple body sites, while the methylation patterns of cfDNA are consistent with the tissues where they originated from. In this context, systemic analysis of cfDNA methylation profiles is under development for early cancer detection, minimal residual disease monitoring, treatment response and prognosis assessment, and to determine the tissue of origin.

DNA methylation is one of the best-studied epigenetic modifications, occurring frequently at cytosine in a 5′-C-phosphate-G-3′ (CpG) dinucleotide context. In the mammalian genome, the majority of CpGs are methylated, except for unmethylated CpG-rich regions called CpG islands. In contrast, the cancer methylome is characterized by global hypomethylation and CpG islands-specific hypermethylation. Hypermethylation of CpG island can affect the cell cycle, DNA repair, metabolism, cell-to-cell interaction, apoptosis, and angiogenesis, all of which are involved in tumorigenesis and cancer progression. CpG island hypermethylation has been described in almost every tumor type. One of the most well-studied DNA methylation signatures is the methylation of SEPT9 promoter, which is an FDA-approved biomarker for colorectal cancer (CRC) detection. A blood-based test for methylated SEPT9 (Epi proColon) has been applied to plasma cfDNA in patients undergoing CRC screening, however this test has low sensitivity for early-stage CRC detection. Nonetheless, CpG island hypermethylation has demonstrated its great versatility and potential for the detection and management of cancer.

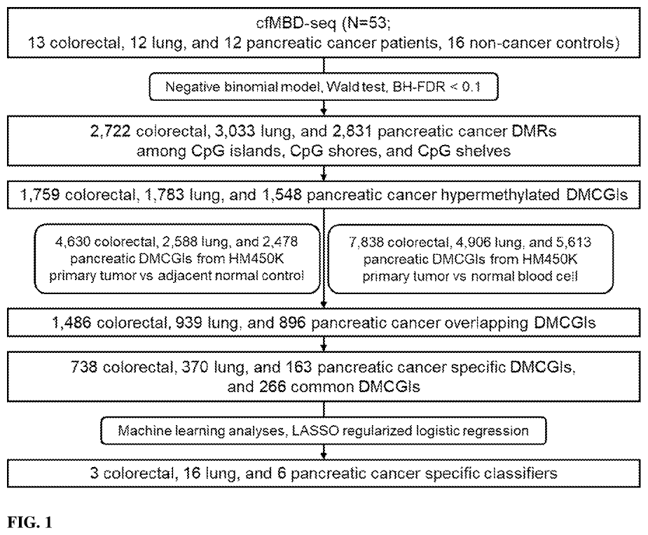

Enrichment-based methylation profiling methods such as methyl-CpG-binding domain sequencing (MBD-seq) and methylated DNA immunoprecipitation sequencing (MeDIP-seq) have shown similar sensitivity and specificity for the detection of differentially methylated regions (DMRs) when compared to bisulfite conversion-based methods. Nonetheless, such technologies are restricted to tumor tissue application due to the need of high amounts of DNA input. To address this issue, Shen et al. optimized the MeDIP-seq protocol to allow methylome analysis of small quantities of cfDNA, termed cfMeDIP-seq. cfMeDIP-seq has shown high accuracy in the classification of a wide variety of cancer types and characterization of renal cell carcinoma patients across all stages. To expand the use of enrichment-based methods in cfDNA, we optimized the MBD-seq protocol for low input cfDNA methylation profiling, termed cfMBD-seq. cfMBD-seq provides higher sequencing data quality with more sequenced reads passing filter and a lower duplicate rate than cfMeDIP-seq. In contrast to cfMeDIP-seq, cfMBD-seq does not require DNA to be denatured. cfMBD-seq also outperforms cfMeDIP-seq in the enrichment of high CpG density regions (i.e., CpG islands). However, the clinical feasibility of cfMBD-seq is unknown. Based on our findings, we hypothesized that cfMBD-seq can identify hypermethylated CpG islands as biomarkers for cancer detection and classification. In this study, we applied cfMBD-seq to the plasma samples of patients with advanced lung, colorectal, and pancreatic cancer and cancer-free individuals to determine whether cfMBD-seq can reliably identify differentially methylated regions (DMRs) between cases and controls. We also investigated whether these DMRs enable accurate discrimination between different cancer types (FIG. 1).

In one aspect, disclosed herein are methods of detecting, diagnosing, and/or grading a cancer and/or metastasis (such as, for example, colorectal, lung, and/or pancreatic cancer), said method comprising a) obtaining a fluid biological sample (such as, for example, whole blood, plasma, serum, and/or cerebrospinal fluid); b) extracting cfDNA; c) generating methylated filler DNA; d) ligating an adapter to the cfDNA and combining with filler DNA thereby creating methylation cfDNA library; e) enriching for methylated cfDNA; f) amplifying and sequencing enriched methylated cfDNA library; and g) assaying CpG islands for hypermethylation relative to a normal control (autologous noncancerous tissue from the subject or a negative/normal control standard); wherein the presence of CpG hypermethylation at a CpG islands chr4:174427892-174428192, chr7:27265159-27265493, chr7:65037625-65037864, chr8:124172801-124173541, chr12:54408427-54408713, chr13:28549840-28550246, chr1:50798668-50799536, chr5:92939796-92940216, or chr12:114881650-114881937, and/or at CpG island associated with CLIP4 (chr2:29337984-29338909), LONRF2 (chr2:100937780-100939059), RNF217 (chr6:125283125-125284389), MEIS1 (chr2:66672432-66673636), ZNF638 (chr2:71503548-71504233), WNT6 (chr2:219736133-219736592), MGST2 (chr4:140655963-140657135), PTGER4 (chr5:40679503-40682081), C9orf129 (chr9:96108467-96108992), B4GALNT1 (chr12:58021295-58022037), HOXB8 (chr17:46691521-46692097), TBX4 (chr17:59539363-59539834), SOX9 (chr17:70112825-70114271), RNF220 (chr1:44883137-44884272), CELF2 (chr10:11059443-11060524), and/or DBX1 (chr11:20177609-20178824) indicates the presence of a cancer; and wherein hypermethylation indicates the severity (i.e., grade of the cancer). In one aspect the sample is subject to two centrifuge cycles. In some aspects the cfDNA is collected in streck tubes.

In one aspect disclosed herein are methods of typing a cancer and/or metastasis, said method comprising a) obtaining a fluid biological sample (such as, for example, whole blood, plasma, serum, and/or cerebrospinal fluid); b) extracting cfDNA; c) generating methylated filler DNA; d) ligating an adapter to the cfDNA and combining with filler DNA thereby creating methylation cfDNA library; e) enriching for methylated cfDNA; f) amplifying and sequencing enriched methylated cfDNA library; and g) assaying CpG islands for hypermethylation relative to normal controls (autologous noncancerous tissue from the subject or a negative/normal control standard); wherein the presence of CpG hypermethylation at a CpG islands associated with CLIP4 (chr2:29337984-29338909), LONRF2 (chr2:100937780-100939059), and/or RNF217 (chr6:125283125-125284389) indicate colorectal cancer; wherein the presence of CpG hypermethylation at a CpG islands at chr4:174427892-174428192, chr7:27265159-27265493, chr7:65037625-65037864, chr8:124172801-124173541, and/or chr12:54408427-54408713, and/or at CpG islands associated with MEIS1 (chr2:66672432-66673636), ZNF638 (chr2:71503548-71504233), WNT6 (chr2:219736133-219736592), MGST2 (chr4:140655963-140657135), PTGER4 (chr5:40679503-40682081), C9orf129 (chr9:96108467-96108992), B4GALNT1 (chr12:58021295-58022037), HOXB8 (chr17:46691521-46692097), TBX4 (chr17:59539363-59539834), and/or SOX9 (chr17:70112825-70114271) indicate lung cancer; and wherein the presence of CpG hypermethylation at a CpG islands at chr13:28549840-28550246, chr1:50798668-50799536, chr5:92939796-92940216, and/or chr12:114881650-114881937, and/or at CpG island associated with RNF220 (chr1:44883137-44884272), CELF2 (chr10:11059443-11060524), and/or DBX1 (chr11:20177609-20178824) indicates the presence of a pancreatic cancer. In one aspect the sample is subject to two centrifuge cycles. In some aspects the cfDNA is collected in streck tubes.

Also disclosed herein are methods of detecting, diagnosing, typing, and/or grading a cancer and/or metastasis, wherein the methylated filler DNA is generated by treating amplicons of Enterobacteria phage λ DNA with CpG methyltransferase.

It is understood and herein contemplated that the ability to utilize cell free DNA and liquid biopsies affords the ability for earlier detection, typing, and grading of cancer than is otherwise able to be accomplished using traditional techniques. By detecting the presence of a cancer and knowing the type of cancer earlier means that treatment can be initiated sooner and/or more aggressively thereby increasing the potential for a successful treatment outcome. Thus, in one aspect, the disclosed methods of detecting, diagnosing, typing, and/or grading a cancer and/or metastasis disclosed herein can comprise the further step of treating the subject for the cancer. In one aspect, disclosed herein are methods of treating, inhibiting, decreasing, reducing, ameliorating, and/or preventing a cancer (such as, for example, pancreatic, colorectal, and/or lung cancer), said method comprising a) obtaining a fluid biological sample; b) extracting cfDNA; c) generating methylated filler DNA; d) ligating an adapter to the cfDNA and combining with filler DNA thereby creating methylation cfDNA library; e) enriching for methylated cfDNA; f) amplifying and sequencing enriched methylated cfDNA library; g) assaying CpG islands for hypermethylation relative to a normal control (autologous noncancerous tissue from the subject or a negative/normal control standard); wherein the presence of CpG hypermethylation at a CpG islands chr4:174427892-174428192, chr7:27265159-27265493, chr7:65037625-65037864, chr8:124172801-124173541, chr12:54408427-54408713, chr13:28549840-28550246, chr1:50798668-50799536, chr5:92939796-92940216, or chr12:114881650-114881937, and/or at CpG island associated with CLIP4 (chr2:29337984-29338909), LONRF2 (chr2:100937780-100939059), RNF217 (chr6:125283125-125284389), MEIS1 (chr2:66672432-66673636), ZNF638 (chr2:71503548-71504233), WNT6 (chr2:219736133-219736592), MGST2 (chr4:140655963-140657135), PTGER4 (chr5:40679503-40682081), C9orf129 (chr9:96108467-96108992), B4GALNT1 (chr12:58021295-58022037), HOXB8 (chr17:46691521-46692097), TBX4 (chr17:59539363-59539834), SOX9 (chr17:70112825-70114271), RNF220 (chr1:44883137-44884272), CELF2 (chr10:11059443-11060524), and/or DBX1 (chr11:20177609-20178824) indicates the presence of a cancer; and h) treating the cancer with an effective amount of a therapeutic agent. In one aspect the sample is subject to two centrifuge cycles. In some aspects the cfDNA is collected in streck tubes.

The present methods are significant as diagnosis of sarcoma, kidney cancer, and head and neck cancer was not possible, but can be detected using the present methods assaying CPG islands.

The disclosed treatments methods can also include the administration any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Treatment methods can include or further include checkpoint inhibitors including, but not limited to, antibodies that block PD-1 (Pembrolizumab, Nivolumab (BMS-936558 or MDX1106), CT-011, MK-3475), PD-L1 (MDX-1105 (BMS-936559), MPDL3280A, or MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

Also disclosed herein are methods of treating, inhibiting, decreasing, reducing, ameliorating, and/or preventing a cancer as disclosed herein, wherein the methylated filler DNA is generated by treating amplicons of Enterobacteria phage λ DNA with CpG methyltransferase.

### C. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed is a kit for assessing a subject's risk for acquiring pancreatic, lung, and/or colorectal cancer, comprising filler Enterobacteria phage λ DNA, primers for amplification of said Enterobacteria phage λ DNA, and a control standard.

### D. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Cancer Detection and Classification by CpG Island Hypermethylation Signatures in Plasma Cell-Free DNA

### a) Materials and Methods

### (1) Sample Acquisition and Clinical Cohort

The study subjects were recruited at Moffitt Cancer Center following Total Cancer Care protocol (https://moffitt.org/research-science/total-cancer-care/). A total of 53 subjects including colorectal (N=13), lung (N=12), pancreatic (N=12) cancer patients, and non-cancer controls (N=16) were used in this study (Clinical demographic characteristics in Table 2). All cancer patients had metastatic disease at the time of sample collection. Most cancer patients had adenocarcinoma histology: 11 of 13 were colorectal adenocarcinoma; 9 of 12 were lung adenocarcinoma; and 10 of 12 were pancreatic adenocarcinoma. Subjects in the non-cancer cohort were specifically negative for any form of cancer. Samples were randomized and blinded during cfDNA extraction, library preparation, and sequencing. Samples were unblinded during data analysis. All patients provided written informed consent. The study was approved by Institutional Review Boards (IRB #00000971) of H. Lee Moffitt Cancer Center & Research Institute (MCC 20563).

TABLE 2

| | | | Colorectal | Lung | Pancreatic |
|---|---|---|---|---|---|
| Type | | Non-cancer | cancer | cancer | cancer |
| Age | Mean | 49.38 | 67.88 | 58.33 | 61.25 |
| | Median | 52.5 | 72.5 | 57.5 | 62.5 |
| | Range | 42.5-52.5 | 47.5-82.5 | 42.5-67.5 | 42.5-67.5 |
| Gender | Male | 5 | 11 | 9 | 8 |
| | Female | 11 | 2 | 3 | 4 |
| Race | Caucasian | 15 | 11 | 12 | 11 |
| | African American | 0 | 2 | 0 | 1 |
| | Asian | 1 | 0 | 0 | 0 |
| Pathological | I-II | \ | 0 | 0 | 0 |
| Stage | III-IV | \ | 13 | 12 | 12 |
| Histology | Adenocarcinoma | \ | 11 | 9 | 10 |
| | Others | \ | 2 | 3 | 2 |

### (2) Plasma Sample Collection

Moffitt Cancer Center Total Cancer Care followed standard operating procedure for blood sampling: Whole blood specimens were obtained by routine venous phlebotomy and collected in Purple top EDTA blood tubes. Plasma was isolated from whole blood at the time of subject enrollment. Centrifugation of whole blood was performed at 1300×g for 10 min at room temperature. Plasma layer was transferred into 1.5 ml cryovials and stored as three 1 mL aliquots. Plasma samples were frozen immediately at −80° C. after isolation.

(3) cfDNA Extraction

Plasma samples were thawed and centrifuged at 3,000 g for 15 mins to ensure complete depletion of cell debris. cfDNA was extracted using QIAamp Circulating Nucleic Acid Kit (Qiagen; Hilden, Germany) following the manufacturer's protocol, except for the addition of carrier RNA in Buffer AVE. All cfDNA eluates were quantified by Qubit Fluorometer with iQuant™ NGS-HS dsDNA Assay Kit (Genecopoeia; Rockville, MD, USA) and then submitted to Moffitt Cancer Center Molecular Genomics Core for D1000 ScreenTape Assay (Agilent; Santa Clara, CA, USA) to ensure the absence of high molecular weight DNA contamination from white blood cell lysis.

### (4) Filler DNA Generation

To generate filler DNA, Enterobacteria phage λ DNA was polymerase chain reaction (PCR) amplified with GoTaq Master Mix (Promega; Madison, WI, USA). Primers sequences are as follows: Forward primer 5′-CGATGGGTTAATTCGCTCGTTGTGG-3′ (SEQ ID NO: 1), reverse primer 5′-GCACAACGGAAAGAGCACTG-3′(SEQ ID NO: 2). The 274 bp amplicons were treated with CpG methyltransferase (M.SssI; Thermo Fisher Scientific) to methylate amplicons. Methylated amplicons were purified by DNA Clean & Concentrator-5 Kit (ZYMO Research; Irvine, CA, USA) and quantified by Qubit Fluorometer. CpG methylation-sensitive restriction enzyme HpyCH4IV (New England BioLabs; Ipswitch, MA, USA) digestion followed by agarose gel electrophoresis was performed to ensure complete methylation of filler DNA.

### (5) Library Preparation

cfDNA was subjected to end repair/A-tailing and adapter ligation using KAPA Hyper Prep Kit (Kapa Biosystems; Wilmington, MA, USA) with the sequencing adapter from NEBNext Multiplex Oligos for Illumina (New England BioLabs). The amount of adapter was adjusted to an adapter: insert molar ratio of 200:1. Adapter ligated DNA were purified with 0.8×SPRI Beads (Beckman Coulter; Pasadena, CA, USA) and digested with USER enzyme (New England BioLabs) followed by purification with DNA Clean & Concentrator-5 Kit. Adapter ligated DNA was first combined with methylated filler DNA to ensure that the total amount of input for methylation enrichment was 100 ng, which was further mixed with 0.2 ng of methylated and 0.2 ng of unmethylated spike-in A. thaliana DNA from DNA Methylation control package (Diagenode, Seraing, Belgium).

(6) cfMBD Methylation Capture

The DNA mixture was subjected to methylation enrichment using MethylCap Kit (Diagenode) following the manufacture's protocol with some modifications. Total volume brought up by Buffer B was reduced from 141.8 μl to 136 μl to minimize DNA waste. The amount of MethylCap protein and magnetic beads were decreased proportionally according to the recommended input DNA to protein and beads ratio (0.2 μg protein and 3 μl beads per 100 ng DNA input). MethylCap protein was 10-fold diluted to 0.2 μg/μl using Buffer B. Single fraction elution with High Elution Buffer was applied. The eluted fraction was purified by DNA Clean & Concentrator-5 Kit. The purified DNA was divided into two parts, one for qPCR (PowerUp™ SYBR™ Green Master Mix, Thermo Fisher) amplification of spiked-in DNA for methylation enrichment quality control, another for library amplification. Recovery of the spiked-in methylated and unmethylated controls can be calculated based on cycle threshold (Ct) value of the enriched and unenriched samples. Specificity of the capture reaction can be calculated by (1−[recovery of unmethylated control DNA over recovery of methylated control DNA])×100). The specificity of the reaction should be ≥99% before proceeding to the next step.

### (7) DNA Sequencing and Alignment

Methylation-enriched DNA libraries were amplified as follows: 95° C. for 3 min, followed by 12 cycles of 98° C. for 20 s, 65° C. for 15 s and 72° C. for 30 s and a final extension of 72° C. for 1 min. During the amplification, unique indexes from primer (NEBNext Multiplex Oligos for Illumina) were added to sequencing adapter of each sample. The amplified libraries were purified using 1×SPRI Beads followed by a dual size selection (0.6× followed by 1.2×) to remove any adapter dimers. All final libraries were first quantified using the Qubit assay and NEBNext® Library Quant Kit for Illumina® (New England BioLabs) and then submitted to Moffitt Cancer Center Molecular Genomics Core for D1000 ScreenTape Assay for measurement of fragment size. Libraries were sequenced on the NextSeq 550 platform (Illumina; San Diego, CA, USA), high-output 75 bp single-end read, multiplexed as 12 samples per run. After sequencing, quality control for raw sequence reads was performed using fastp (Version 0.20.1) with the default settings. The sequence reads were then aligned to the human genome (hg19) using Bowtie-2 (Version 2.4.2) with default settings. After the alignment, the generated sam files were converted to bam files, followed by sorting, indexing, removal of duplicate reads, and extraction of read count on chr1-chr22 using SAMtools (Version 1.11) ‘view’, ‘sort’, ‘index’, and ‘markdup’ command lines.

### (8) Quality Control of Methylation Enrichment

R (Version 4.0.3 or greater) package RaMWAS (Version 1.12.0) with default parameters was used for quality control of overall mapping quality and calculation of non-CpG reads percentage, average non-CpG/CpG coverage (noise), and CpG density at peak. CpG annotation reference was obtained from R package annotatr (Version 1.16.0): annots=‘hg19_cpgs’. BEDtools (Version 2.28.0) ‘coverage’ command line was used to call the number of sequenced reads on each CpG feature. CpG feature coverage of each sample was combined as a count matrix. Transcripts per kilobase million (TPM) normalization was performed before comparing the percentage of CpG feature coverage between different groups.

(9) Differential Methylation Analysis of cfMBD-Seq Data

Rows with inter CpG regions and 0 read count among all samples were filtered out from CpG feature raw count matrix. Filtered matrix were further subset for single cancer type and non-cancer control and fit a negative binomial model to call DMRs at BH-FDR<0.1 (Wald test) using R package DESeq2 (Version 1.32.0). Since most DMRs were hypermethylated CpG islands, count matrix with only CpG islands were used for identification of DMRs. R package EnhancedVolcano (Version 1.10.0) was used for visualization of fold change and BH-FDR (q value) for all CpG islands and extended CpG islands. Unsupervised hierarchical clustering was performed on Partek genomics suite (Version 7.0) for visualization of DMCGIs, using log transformed DESeq2 normalized values, z scores, Euclidean distance, and Ward Clustering. R package pcaExplorer (Version 2.18.0) was used for principal component analysis of DESeq2 normalized values of top 1,000 hypermethylated CpG islands selected by highest row variance. The 95% confidence ellipses for the case and control were displayed. DMCGIs with fold change >2 were used for intersection with tissue derived DMCs.

### (10) Methylation Analyses for Tumor Tissue Specific DMCGIs

HM450K data of primary tumors and adjacent normal tissues from patients with colon adenocarcinoma (COAD) (35 pairs), lung adenocarcinoma (LUAD) (21 pairs), and pancreatic adenocarcinoma (PAAD) (10 pairs) were acquired from TCGA. HM450K data of non-cancer individuals' PBMCs (N=61) from GEO (non-smoker controls in GSE53045) were also used to deconvolute clonal hematopoiesis effect. R package minfi (Version 1.36.0) was used to call DMCs (Mean of A beta value >0.2 and BH-FDR<0.1) between primary tumor and normal tissue/non-cancer PBMCs. R package EnhancedVolcano was used for visualization of A beta value and q value for all HM450K CpG sites. To make tissue derived DMCs comparable with plasma derived DMRs, all DMCs were annotated to hg19 HM450K annotation file and their corresponding CpG islands were identified for intersection. Tissue-derived DMCGIs were identified by intersecting plasma case vs control, primary tumor vs. normal tissue, and primary tumor vs. PBMCs DMCGIs. Tissue-specific DMCGIs were identified by intersecting colorectal, lung, and pancreas-derived DMCGIs. Venn diagrams were used for visualization of intersection.

### (11) Machine Learning Analyses

Two independent cohorts were used for machine learning analyses: cfMeDIP-seq cohort and HM450K cohort. cfMeDIP-seq data of lung cancer patients (N=80) and non-cancer individuals (N=86) were used for evaluation of early cancer detection in plasma cfDNA. An independent HM450K cohort including primary tumors from TCGA (N=210 for COAD, N=385 for LUAD, and N=162 for PAAD) was used for evaluation of cancer classification performance. HM450K data were converted to a CpG islands beta value matrix by calculating the mean beta values of CpG sites annotated to the same CpG island. R package Caret (Version 6.0-88) was used to partition the discovery cohort data into 100 class-balanced independent training and testing sets in an 80-20% manner. Top overlapping DMCGIs between cfMBD-seq and HM450K datasets were selected for predictive modeling analyses. R package glmnet (Version 4.1-2) was used to preform regularized logistic regression model on the training sets. LASSO regularization method (alpha=1) with 10-fold cross validation was applied to determine minimum lambda penalty value. The process was repeated 100 times to prevent training-set biases. DMCGIs with non-zero coefficient across all repeats were determined as binomial classifiers. Classification performance of predictive models was evaluated on the held-out testing set using ROC statistics. R package Rtsne (Version 0.15) was used for t-sne plot to visualize cancer classification in cfMBD-seq, cfMeDIP-seq, and HM450K data sets.

### b) Results

(1) Significant Enrichment of Methylated CpG Islands in cfDNA

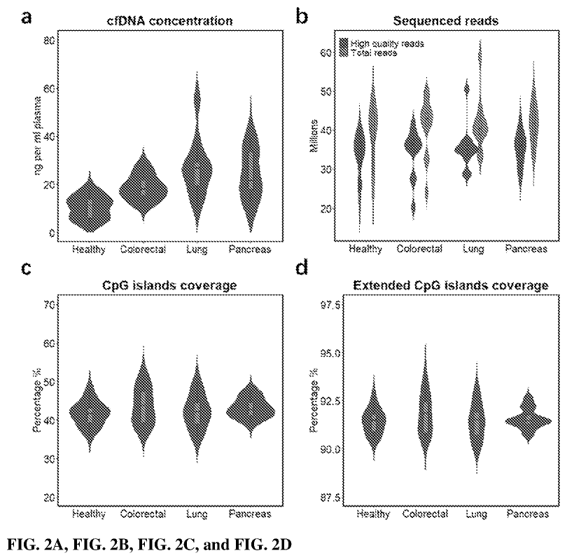

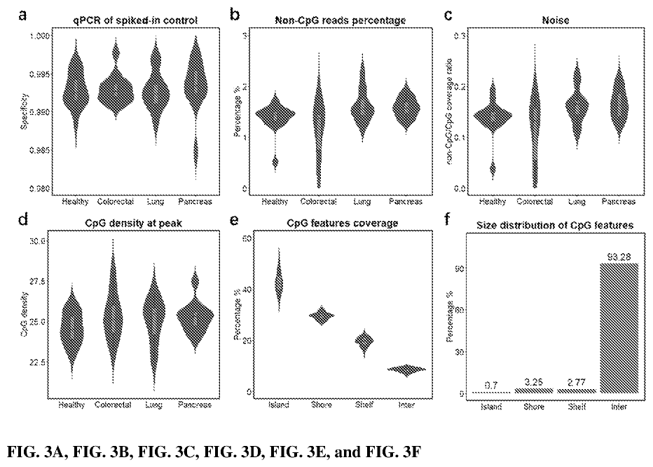

To study the clinical feasibility of cfMBD-seq, we retrospectively profiled cfDNA methylome of 53 blood samples from patients with metastatic carcinoma of the colon/rectum (N=13), lung (N=12), and pancreas (N=12), and from cancer-free individuals (N=16). We quantified cfDNA concentration from plasma samples and showed that cancer patients had higher cfDNA yield than non-cancer controls (FIG. 2a, Table 3). To investigate methylation capture efficiency of cfMBD-seq, we compared spiked-in controls between methylated and unmethylated A. thaliana DNA in the capture reaction and observed a median specificity at 99.3% [99.16% (Q1)-99.43% (Q3)] across all samples (FIG. 3a). From sequencing data, we filtered out duplicate reads and reads with low alignment scores from total sequence reads (41.62 [38.75-44.43] million) and obtained 35.33 [32.77-37.37] million high-quality reads (FIG. 2b). We then investigated genome-wide methylation enrichment and found that the number of captured fragments without any CpG tandem accounted for only 1.47% [1.33%-1.59%] of high-quality reads (FIG. 3b). The average coverage ratio of fragments without any CpG tandem to fragments with at least one CpG, known as noise, was 0.15 [0.13-0.17](FIG. 3c). The median CpG density of fragments with the highest read coverage was 25.2 [24.2-25.7](FIG. 3d), corresponding to high-density CpG islands. Intrigued by the high sequencing coverage on CpG islands, we further studied the distribution of sequence reads by calculating the percentage of normalized reads on different CpG annotation features (i.e., CpG islands, CpG shores, CpG shelves, and inter CpG regions). We found a median of 42.16% [39.47-45.15] of reads mapped to CpG islands, when CpG islands only account for 0.7% of the hg19 reference genome (FIG. 3e&f, FIG. 2c). Since methylation alterations may occur at a short distance away from the CpG islands, we also calculated the sum of reads mapped to extended CpG islands (i.e., CpG islands, CpG shores, and CpG shelves). A median of 91.46% [90.89%-92.13%] of reads were mapped to the extended CpG islands, which accounts for only 6.72% of the reference genome (FIG. 3e&f, FIG. 2d). These results demonstrate that most of the sequence reads captured by cfMBD-seq were significantly enriched on CpG island-centered regions, illustrating successful cfMBD-seq methylation enrichment and library construction across all samples.

TABLE 3

| | | cfDNA | | | | | | |
|---|---|---|---|---|---|---|---|---|
| | | Yield ng | qPCR | | Reads | RA % of | Reads after | RAF % of |
| Sample ID | Type | per m [symbol] | specifici [symbol] | Total rea [symbol] | aligned | total | filter | aligne [symbol] |
| COLORECTAL_01 | Colorectal | 13.57 | 0.992 | 40,897,509 | 40,897,509 | 100.00% | 35,662,464 | 87.20% |
| COLORECTAL_02 | Colorectal | 18.19 | 0.993 | 41,045,370 | 41,045,370 | 100.00% | 35,940,353 | 87.60% |
| COLORECTAL_03 | Colorectal | 12.61 | 0.991 | 24,076,673 | 24,076,673 | 100.00% | 20,684,202 | 85.90% |
| COLORECTAL_04 | Colorectal | 19.31 | 0.993 | 49,314,698 | 49,314,698 | 100.00% | 42,702,838 | 86.60% |
| COLORECTAL_05 | Colorectal | 16.78 | 0.998 | 44,294,734 | 44,294,734 | 100.00% | 38,106,791 | 86.00% |
| COLORECTAL_06 | Colorectal | 15.90 | 0.994 | 46,474,939 | 46,474,939 | 100.00% | 40,703,952 | 87.60% |
| COLORECTAL_07 | Colorectal | << | 0.993 | 49,353,137 | 49,353,137 | 100.00% | 41,096,622 | 83.30% |
| COLORECTAL_08 | Colorectal | 11.94 | 0.992 | 42,896,411 | 42,896,411 | 100.00% | 37,377,700 | 87.10% |
| COLORECTAL_09 | Colorectal | 28.20 | 0.992 | 44,432,802 | 44,432,802 | 100.00% | 39,258,780 | 88.40% |
| COLORECTAL_10 | Colorectal | 21.24 | 0.991 | 42,608,477 | 42,608,477 | 100.00% | 37,077,964 | 87.00% |
| COLORECTAL_11 | Colorectal | 17.64 | 0.991 | 44,295,305 | 44,295,305 | 100.00% | 38,428,187 | 86.80% |
| COLORECTAL_12 | Colorectal | 25.80 | 0.995 | 33,238,455 | 33,238,455 | 100.00% | 28,650,884 | 86.20% |
| COLORECTAL_13 | Colorectal | 23.28 | 0.994 | 32,080,627 | 32,080,627 | 100.00% | 27,610,074 | 86.10% |
| CONTROL_01 | Healthy | 12.32 | 0.994 | 39,350,577 | 39,350,577 | 100.00% | 34,348,229 | 87.30% |
| CONTROL_02 | Healthy | 14.08 | 0.994 | 40,392,841 | 40,392,841 | 100.00% | 35,267,940 | 87.30% |
| CONTROL_03 | Healthy | 4.18 | 0.991 | 24,038,559 | 24,038,559 | 100.00% | 20,906,758 | 87.00% |
| CONTROL_04 | Healthy | 5.78 | 0.992 | 30,210,339 | 30,210,339 | 100.00% | 26,439,973 | 87.50% |
| CONTROL_05 | Healthy | 5.61 | 0.991 | 41,318,931 | 41,318,931 | 100.00% | 35,595,600 | 86.10% |
| CONTROL_06 | Healthy | 11.44 | 0.995 | 45,958,890 | 45,958,890 | 100.00% | 39,841,435 | 86.70% |
| CONTROL_07 | Healthy | 3.19 | 0.996 | 44,601,028 | 44,601,028 | 100.00% | 38,060,082 | 85.30% |
| CONTROL_08 | Healthy | 6.05 | 0.994 | 38,751,974 | 38,751,974 | 100.00% | 33,269,200 | 85.90% |
| CONTROL_09 | Healthy | 13.14 | 0.996 | 43,493,542 | 43,493,542 | 100.00% | 38,138,496 | 87.70% |
| CONTROL_10 | Healthy | 8.70 | 0.996 | 44,275,035 | 44,275,035 | 100.00% | 38,180,984 | 86.20% |
| CONTROL_11 | Healthy | 7.26 | 0.991 | 42,010,014 | 42,010,014 | 100.00% | 36,611,422 | 87.10% |
| CONTROL_12 | Healthy | 15.06 | 0.993 | 47,287,823 | 47,287,823 | 100.00% | 41,356,521 | 87.50% |
| CONTROL_13 | Healthy | 17.40 | 0.988 | 47,921,685 | 47,921,685 | 100.00% | 41,736,492 | 87.10% |
| CONTROL_14 | Healthy | 13.36 | 0.992 | 41,110,126 | 41,110,126 | 100.00% | 36,155,556 | 87.90% |
| CONTROL_15 | Healthy | 10.86 | 0.992 | 32,401,696 | 32,401,696 | 100.00% | 28,578,428 | 88.20% |
| CONTROL_16 | Healthy | 18.00 | 0.992 | 28,199,208 | 28,199,208 | 100.00% | 24,887,711 | 88.30% |
| LUNG_01 | Lung | 26.68 | 0.993 | 40,903,906 | 40,903,906 | 100.00% | 36,177,090 | 88.40% |
| LUNG_02 | Lung | 27.39 | 0.993 | 58,765,761 | 58,765,761 | 100.00% | 51,861,044 | 88.30% |
| LUNG_03 | Lung | 10.41 | 0.991 | 41,776,541 | 41,776,541 | 100.00% | 36,531,690 | 87.40% |
| LUNG_04 | Lung | 37.03 | 0.997 | 43,058,523 | 43,058,523 | 100.00% | 37,582,842 | 87.30% |
| LUNG_05 | Lung | 27.50 | 0.994 | 39,017,889 | 39,017,889 | 100.00% | 33,461,245 | 85.80% |
| LUNG_06 | Lung | 30.95 | 0.997 | 42,183,509 | 42,183,509 | 100.00% | 36,011,070 | 85.40% |
| LUNG_07 | Lung | 23.00 | 0.992 | 39,574,961 | 39,574,961 | 100.00% | 34,775,835 | 87.90% |
| LUNG_08 | Lung | 1904.00 | 0.989 | 38,556,909 | 38,556,909 | 100.00% | 35,054,115 | 90.90% |
| LUNG_09 | Lung | 55.20 | 0.991 | 39,783,583 | 39,783,583 | 100.00% | 35,124,161 | 88.30% |
| LUNG_10 | Lung | 22.56 | 0.991 | 45,689,570 | 45,689,570 | 100.00% | 39,914,703 | 87.40% |
| LUNG_11 | Lung | 16.50 | 0.992 | 33,941,247 | 33,941,247 | 100.00% | 29,794,589 | 87.80% |
| LUNG_12 | Lung | 16.38 | 0.992 | 33,045,912 | 33,045,912 | 100.00% | 29,050,283 | 87.90% |
| PANCREAS_01 | Pancreas | 148.50 | 0.991 | 45,008,944 | 45,008,944 | 100.00% | 39,489,141 | 87.70% |
| PANCREAS_02 | Pancreas | 34.65 | 0.993 | 49,927,723 | 49,927,723 | 100.00% | 43,141,931 | 86.40% |
| PANCREAS_03 | Pancreas | 11.39 | 0.996 | 45,330,853 | 45,330,853 | 100.00% | 38,983,692 | 86.00% |
| PANCREAS_04 | Pancreas | 11.50 | 0.997 | 47,222,912 | 47,222,912 | 100.00% | 39,707,164 | 84.10% |
| PANCREAS_05 | Pancreas | 30.30 | 0.993 | 41,618,614 | 41,618,614 | 100.00% | 36,104,103 | 86.70% |
| PANCREAS_06 | Pancreas | 19.26 | 0.997 | 37,313,527 | 37,313,527 | 100.00% | 32,166,630 | 86.20% |
| PANCREAS_07 | Pancreas | 17.46 | 0.992 | 39,187,919 | 39,187,919 | 100.00% | 34,206,464 | 87.30% |
| PANCREAS_08 | Pancreas | 20.28 | 0.992 | 43,138,907 | 43,138,907 | 100.00% | 37,306,406 | 86.50% |
| PANCREAS_09 | Pancreas | 750.00 | 0.995 | 41,778,689 | 41,778,689 | 100.00% | 37,239,492 | 89.10% |
| PANCREAS_10 | Pancreas | 36.30 | 0.985 | 39,757,428 | 39,757,428 | 100.00% | 35,155,375 | 88.40% |
| PANCREAS_11 | Pancreas | 39.30 | 0.993 | 34,488,911 | 34,488,911 | 100.00% | 30,196,957 | 87.60% |
| PANCREAS_12 | Pancreas | 22.62 | 0.994 | 33,537,103 | 33,537,103 | 100.00% | 29,173,812 | 87.00% |

| | Reads | | Reads | | | Avg | |
|---|---|---|---|---|---|---|---|
| | removed as | RRAD % of | used for | RUFC % of | Non-CpG | non-CpG | Avg CpG |
| Sample ID | duplicat [symbol] | aligne [symbol] | coverag [symbol] | aligne [symbol] | reads ( [symbol] | covera [symbol] | covera [symbol] |
| COLORECTAL_01 | 951,450 | 2.30% | 34,711,014 | 84.90% | 1.32% | 0.92 | 6.96 |
| COLORECTAL_02 | 789,207 | 1.90% | 35,151,146 | 85.60% | 1.36% | 0.98 | 6.95 |
| COLORECTAL_03 | 390,646 | 1.60% | 20,293,556 | 84.30% | 1.41% | 0.57 | 4.49 |
| COLORECTAL_04 | 967,658 | 2.00% | 41,735,180 | 84.60% | 1.80% | 1.56 | 8.37 |
| COLORECTAL_05 | 988,889 | 2.20% | 37,117,902 | 83.80% | 1.79% | 1.36 | 7.69 |
| COLORECTAL_06 | 730,007 | 1.60% | 39,973,945 | 86.00% | 1.45% | 1.13 | 7.19 |
| COLORECTAL_07 | 5,763,326 | 11.70% | 35,333,296 | 71.60% | 0.15% | 0.1 | 11.47 |
| COLORECTAL_08 | 894,598 | 2.10% | 36,483,102 | 85.00% | 1.37% | 1.02 | 6.86 |
| COLORECTAL_09 | 693,587 | 1.60% | 38,565,193 | 86.80% | 1.49% | 1.18 | 7.3 |
| COLORECTAL_10 | 753,005 | 1.80% | 36,324,959 | 85.30% | 1.23% | 0.87 | 7.23 |
| COLORECTAL_11 | 956,483 | 2.20% | 37,471,704 | 84.60% | 0.70% | 0.45 | 8.23 |
| COLORECTAL_12 | 555,503 | 1.70% | 28,095,381 | 84.50% | 0.74% | 0.38 | 6.85 |
| COLORECTAL_13 | 608,265 | 1.90% | 27,001,809 | 84.20% | 0.27% | 0.12 | 7.52 |
| CONTROL_01 | 916,636 | 2.30% | 33,431,593 | 85.00% | 1.23% | 0.83 | 6.75 |
| CONTROL_02 | 930,780 | 2.30% | 34,337,160 | 85.00% | 1.33% | 0.93 | 6.64 |
| CONTROL_03 | 365,976 | 1.50% | 20,540,782 | 85.40% | 1.47% | 0.62 | 4.37 |
| CONTROL_04 | 389,419 | 1.30% | 26,050,554 | 86.20% | 1.59% | 0.86 | 5.43 |
| CONTROL_05 | 697,114 | 1.70% | 34,898,486 | 84.50% | 1.35% | 0.96 | 7.49 |
| CONTROL_06 | 1,005,682 | 2.20% | 38,835,753 | 84.50% | 1.43% | 1.15 | 7.91 |
| CONTROL_07 | 1,068,391 | 2.40% | 36,991,691 | 82.90% | 1.48% | 1.13 | 8.66 |
| CONTROL_08 | 718,889 | 1.90% | 32,550,311 | 84.00% | 1.50% | 0.99 | 7.04 |
| CONTROL_09 | 719,071 | 1.70% | 37,419,425 | 86.00% | 1.72% | 1.32 | 6.83 |
| CONTROL_10 | 1,672,965 | 3.80% | 36,508,019 | 82.50% | 1.49% | 1.09 | 7.35 |
| CONTROL_11 | 1,131,014 | 2.70% | 35,480,408 | 84.50% | 1.28% | 0.92 | 6.66 |
| CONTROL_12 | 1,125,075 | 2.40% | 40,231,446 | 85.10% | 1.44% | 1.19 | 7.77 |
| CONTROL_13 | 2,378,326 | 5.00% | 39,358,166 | 82.10% | 0.51% | 0.33 | 8.84 |
| CONTROL_14 | 830,926 | 2.00% | 35,324,630 | 85.90% | 1.58% | 1.14 | 6.45 |
| CONTROL_15 | 461,034 | 1.40% | 28,117,394 | 86.80% | 1.26% | 0.72 | 5.8 |
| CONTROL_16 | 350,319 | 1.20% | 24,537,392 | 87.00% | 1.44% | 0.72 | 4.95 |
| LUNG_01 | 632,727 | 1.50% | 35,544,363 | 86.90% | 1.55% | 1.16 | 6.68 |
| LUNG_02 | 1,377,651 | 2.30% | 50,483,393 | 85.90% | 1.52% | 1.59 | 9.65 |
| LUNG_03 | 769,756 | 1.80% | 35,761,934 | 85.60% | 1.67% | 1.23 | 7.27 |
| LUNG_04 | 614,861 | 1.40% | 36,967,981 | 85.90% | 2.00% | 1.53 | 7.35 |
| LUNG_05 | 689,928 | 1.80% | 32,771,317 | 84.00% | 1.25% | 0.79 | 7.21 |
| LUNG_06 | 1,176,028 | 2.80% | 34,835,042 | 82.60% | 1.59% | 1.15 | 8.05 |
| LUNG_07 | 512,911 | 1.30% | 34,262,924 | 86.60% | 1.92% | 1.37 | 6.12 |
| LUNG_08 | 292,709 | 0.80% | 34,761,406 | 90.20% | 2.29% | 1.67 | 5.4 |
| LUNG_09 | 486,894 | 1.20% | 34,637,267 | 87.10% | 1.55% | 1.1 | 6.63 |
| LUNG_10 | 2,548,272 | 5.60% | 37,366,431 | 81.80% | 1.41% | 1.06 | 6.96 |
| LUNG_11 | 652,790 | 1.90% | 29,141,799 | 85.90% | 1.25% | 0.73 | 5.95 |
| LUNG_12 | 644,359 | 1.90% | 28,405,924 | 86.00% | 1.44% | 0.84 | 5.71 |
| PANCREAS_01 | 713,965 | 1.60% | 38,775,176 | 86.10% | 1.58% | 1.25 | 7.74 |
| PANCREAS_02 | 975,001 | 2.00% | 42,166,930 | 84.50% | 1.89% | 1.68 | 8.24 |
| PANCREAS_03 | 840,483 | 1.90% | 38,143,209 | 84.10% | 1.65% | 1.27 | 7.85 |
| PANCREAS_04 | 1,464,595 | 3.10% | 38,242,569 | 81.00% | 1.51% | 1.18 | 8.75 |
| PANCREAS_05 | 692,780 | 1.70% | 35,411,323 | 85.10% | 1.55% | 1.09 | 6.64 |
| PANCREAS_06 | 747,655 | 2.00% | 31,418,975 | 84.20% | 1.77% | 1.15 | 5.92 |
| PANCREAS_07 | 801,372 | 2.00% | 33,405,092 | 85.20% | 1.81% | 1.27 | 5.95 |
| PANCREAS_08 | 838,574 | 1.90% | 36,467,832 | 84.50% | 1.36% | 1.02 | 7.15 |
| PANCREAS_09 | 460,565 | 1.10% | 36,778,927 | 88.00% | 1.67% | 1.24 | 6.74 |
| PANCREAS_10 | 734,287 | 1.80% | 34,421,088 | 86.60% | 1.52% | 1.06 | 6.28 |
| PANCREAS_11 | 527,667 | 1.50% | 29,669,290 | 86.00% | 1.36% | 0.82 | 6.24 |
| PANCREAS_12 | 815,884 | 2.40% | 28,357,928 | 84.60% | 1.33% | 0.77 | 5.78 |

| | Non- | | | | | | | | |
|---|---|---|---|---|---|---|---|---|---|
| | Cpg/CpG | | | | CpG | CpG | CpG | Inter | |
| | coverage | Peak | Peak CpG | Peak | Island | Shore | Shelf | CpG | Extended |
| Sample ID | ratio | SQ [symbol] | densit [symbol] | covera [symbol] | (%) | (%) | (%) | (%) | CGI (%) |
| COLORECTAL_01 | 0.1315 | 4.82 | 23.23 | 19.50 | 38.14 | 31.45 | 21.19 | 9.22 | 90.78 |
| COLORECTAL_02 | 0.1405 | 4.87 | 23.72 | 19.66 | 38.04 | 31.69 | 21.09 | 9.17 | 90.83 |
| COLORECTAL_03 | 0.1275 | 5.06 | 25.60 | 15.16 | 46.4 | 28.39 | 17.63 | 7.58 | 92.42 |
| COLORECTAL_04 | 0.1861 | 4.99 | 24.90 | 26.13 | 43.86 | 29.11 | 18.86 | 8.16 | 91.84 |
| COLORECTAL_05 | 0.1771 | 5.1 | 26.01 | 26.28 | 47.3 | 27.73 | 17.4 | 7.56 | 92.44 |
| COLORECTAL_06 | 0.157 | 4.92 | 24.21 | 19.16 | 38.41 | 30.58 | 21.57 | 9.44 | 90.56 |
| COLORECTAL_07 | 0.0089 | 5.16 | 26.63 | 46.63 | 51.74 | 27.2 | 14.86 | 6.21 | 93.79 |
| COLORECTAL_08 | 0.1485 | 4.93 | 24.30 | 18.38 | 39.37 | 30.36 | 21.05 | 9.22 | 90.78 |
| COLORECTAL_09 | 0.1619 | 5.01 | 25.10 | 19.72 | 40.73 | 29.85 | 20.5 | 8.92 | 91.08 |
| COLORECTAL_10 | 0.1207 | 5 | 25.00 | 20.99 | 41.91 | 29.63 | 19.86 | 8.6 | 91.4 |
| COLORECTAL_11 | 0.055 | 5.05 | 25.50 | 23.70 | 43.66 | 29.31 | 18.89 | 8.14 | 91.86 |
| COLORECTAL_12 | 0.0549 | 5.3 | 28.09 | 22.86 | 49.4 | 27.12 | 16.43 | 7.06 | 92.94 |
| COLORECTAL_13 | 0.016 | 5.23 | 27.35 | 26.54 | 48.99 | 27.59 | 16.42 | 6.99 | 93.01 |
| CONTROL_01 | 0.1223 | 4.85 | 23.52 | 19.12 | 38.31 | 31.45 | 21.13 | 9.11 | 90.89 |
| CONTROL_02 | 0.1403 | 4.79 | 22.94 | 17.73 | 35.98 | 32.23 | 22.2 | 9.59 | 90.41 |
| CONTROL_03 | 0.1415 | 4.93 | 24.30 | 13.87 | 43.02 | 29.77 | 19.04 | 8.17 | 91.83 |
| CONTROL_04 | 0.1585 | 4.95 | 24.50 | 16.93 | 42.98 | 29.67 | 19.1 | 8.24 | 91.76 |
| CONTROL_05 | 0.1285 | 4.89 | 23.91 | 22.99 | 41.16 | 30.48 | 19.85 | 8.52 | 91.48 |
| CONTROL_06 | 0.1455 | 4.83 | 23.33 | 22.91 | 38.66 | 31.31 | 20.92 | 9.11 | 90.89 |
| CONTROL_07 | 0.1309 | 5.08 | 25.81 | 31.39 | 48.33 | 27.62 | 16.86 | 7.19 | 92.81 |
| CONTROL_08 | 0.1411 | 5.02 | 25.20 | 23.76 | 45.15 | 28.94 | 18.13 | 7.78 | 92.22 |
| CONTROL_09 | 0.1934 | 5.02 | 25.20 | 19.57 | 41.73 | 29.38 | 20.09 | 8.79 | 91.21 |
| CONTROL_10 | 0.1479 | 5.09 | 25.91 | 23.22 | 45.91 | 28.11 | 18.13 | 7.85 | 92.15 |
| CONTROL_11 | 0.1386 | 4.88 | 23.81 | 17.57 | 37.61 | 31.34 | 21.6 | 9.45 | 90.55 |
| CONTROL_12 | 0.1528 | 4.98 | 24.80 | 22.30 | 40.97 | 30.02 | 20.2 | 8.81 | 91.19 |
| CONTROL_13 | 0.0377 | 4.95 | 24.50 | 25.81 | 41.39 | 30.19 | 19.86 | 8.55 | 91.45 |
| CONTROL_14 | 0.1765 | 4.92 | 24.21 | 17.28 | 39.47 | 30.13 | 21.11 | 9.29 | 90.71 |
| CONTROL_15 | 0.1233 | 5.07 | 25.70 | 17.21 | 42.93 | 29.44 | 19.29 | 8.34 | 91.66 |
| CONTROL_16 | 0.1456 | 5.08 | 25.81 | 14.46 | 43.23 | 29.16 | 19.26 | 8.36 | 91.64 |
| LUNG_01 | 0.1732 | 4.76 | 22.66 | 17.69 | 35.56 | 32.15 | 22.52 | 9.77 | 90.23 |
| LUNG_02 | 0.1651 | 4.8 | 23.04 | 26.35 | 36.37 | 32.25 | 21.9 | 9.49 | 90.51 |
| LUNG_03 | 0.1696 | 4.92 | 24.21 | 22.15 | 42.16 | 29.81 | 19.55 | 8.48 | 91.52 |
| LUNG_04 | 0.2075 | 5.17 | 26.73 | 24.98 | 47.72 | 27.38 | 17.38 | 7.52 | 92.48 |
| LUNG_05 | 0.1094 | 5.06 | 25.60 | 23.61 | 46.49 | 28.43 | 17.56 | 7.52 | 92.48 |
| LUNG_06 | 0.1428 | 5.16 | 26.63 | 30.53 | 50.04 | 26.91 | 16.14 | 6.91 | 93.09 |
| LUNG_07 | 0.223 | 5.02 | 25.20 | 17.41 | 41.14 | 29.72 | 20.27 | 8.87 | 91.13 |
| LUNG_08 | 0.3089 | 5.07 | 25.70 | 12.21 | 39.11 | 29.41 | 21.66 | 9.82 | 90.18 |
| LUNG_09 | 0.1657 | 5.08 | 25.81 | 18.84 | 43.95 | 28.76 | 19 | 8.29 | 91.71 |
| LUNG_10 | 0.1528 | 4.92 | 24.21 | 18.46 | 39.02 | 30.67 | 21.09 | 9.21 | 90.79 |
| LUNG_11 | 0.1225 | 5.03 | 25.30 | 17.39 | 42.04 | 29.68 | 19.72 | 8.55 | 91.45 |
| LUNG_12 | 0.1465 | 5.08 | 25.81 | 16.88 | 43.13 | 29.33 | 19.2 | 8.34 | 91.66 |
| PANCREAS_01 | 0.1617 | 4.89 | 23.91 | 22.15 | 40.98 | 30.17 | 20.14 | 8.71 | 91.29 |
| PANCREAS_02 | 0.2037 | 4.95 | 24.50 | 25.14 | 41.98 | 29.78 | 19.7 | 8.54 | 91.46 |
| PANCREAS_03 | 0.1614 | 5.02 | 25.20 | 25.86 | 45.8 | 28.17 | 18.16 | 7.87 | 92.13 |
| PANCREAS_04 | 0.1348 | 5.05 | 25.50 | 31.85 | 47.53 | 28.02 | 17.09 | 7.36 | 92.64 |
| PANCREAS_05 | 0.1636 | 5.04 | 25.40 | 19.14 | 42.22 | 29.45 | 19.71 | 8.62 | 91.38 |
| PANCREAS_06 | 0.1946 | 5.05 | 25.50 | 17.90 | 43.77 | 28.6 | 19.26 | 8.37 | 91.63 |
| PANCREAS_07 | 0.2136 | 4.99 | 24.90 | 16.21 | 40.73 | 29.74 | 20.51 | 9.02 | 90.98 |
| PANCREAS_08 | 0.1429 | 4.98 | 24.80 | 20.71 | 41.29 | 30 | 20.03 | 8.68 | 91.32 |
| PANCREAS_09 | 0.1837 | 5.1 | 26.01 | 18.14 | 44.1 | 27.98 | 19.37 | 8.55 | 91.45 |
| PANCREAS_10 | 0.1689 | 4.93 | 24.30 | 16.58 | 39.31 | 30.27 | 21.14 | 9.27 | 90.73 |
| PANCREAS_11 | 0.1306 | 5.24 | 27.46 | 18.75 | 46.56 | 27.82 | 17.88 | 7.75 | 92.25 |
| PANCREAS_12 | 0.1337 | 5.06 | 25.60 | 17.11 | 43.16 | 29.21 | 19.23 | 8.4 | 91.6 |

[symbol]

indicates data missing or illegible when filed

### (2) Differential Methylation Analyses Between Cancer Patients and Non-Cancer Controls

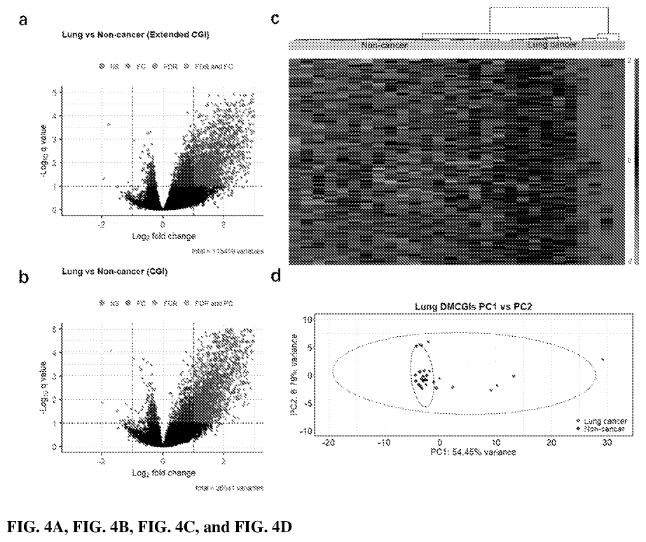

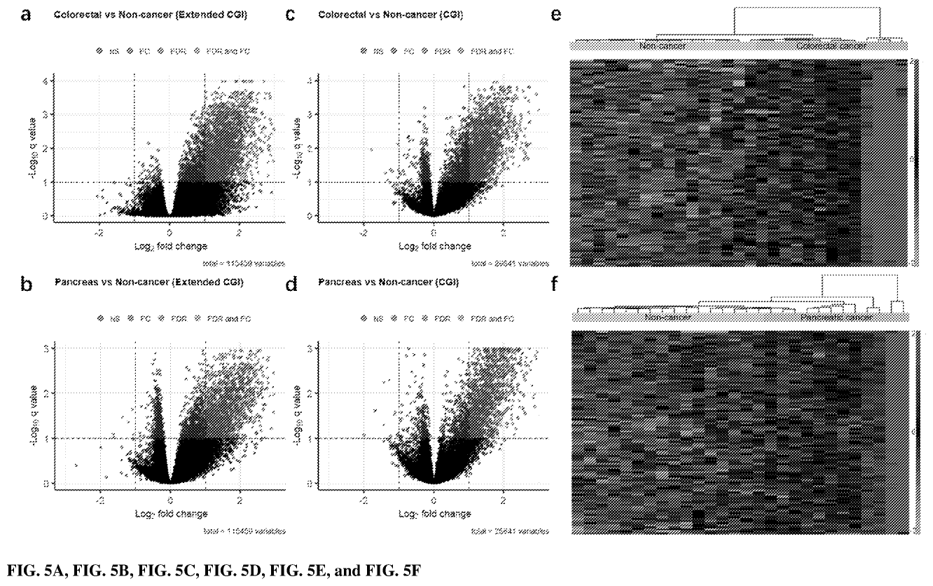

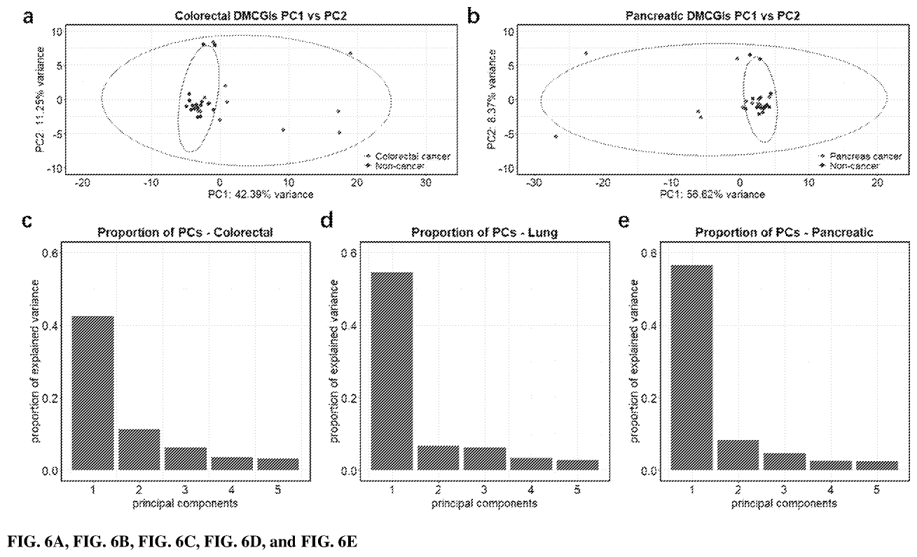

To identify differences in methylation patterns between cases and controls, we generated a read count matrix for each cancer type versus non-cancer control. In this matrix, each row represents a different CpG feature, and each column represents a unique individual sample. We then removed rows annotated as inter CpG and rows with 0 read count across all samples and obtained 115,459 genomic regions. Next, we performed differential methylation analysis based on a negative binomial model of feature counts at a significance level of 0.1 using Benjamini-Hochberg false discovery rate (BH-FDR) and identified 2,722, 3,033, and 2,831 DMRs for colorectal, lung, and pancreatic cancer, respectively (FIG. 4a, FIG. 5a&b). We further filtered these DMRs using a more stringent criteria: absolute fold change >2, which resulted in 2,009 DMRs (2,007 hypermethylated and 2 hypomethylated) in colorectal cancer, 1,818 DMRs (1,814 hypermethylated and 4 hypomethylated) in lung cancer, and 1,488 DMRs (1,482 hypermethylated and 6 hypomethylated) in pancreatic cancer. As the majority of the remaining DMRs were hypermethylated, and most of them were CpG islands (97%, 85%, and 93% in colorectal, lung and pancreatic cancer patients, respectively), to enhance computational efficiency, we reduced our dataset to 26,441 CpG islands and applied the same criteria for differential methylation analysis (BH-FDR<0.1 and fold change >2). This optimized analysis identified 1,759, 1,783, and 1,548 differentially methylated CpG islands (DMCGIs) in colorectal, lung, and pancreatic cancer, respectively (FIG. 3b, FIG. 5c&d). Unsupervised hierarchical clustering of the top 100 hypermethylated CpG islands ranked by p-value well distinguished cancer patients from non-cancer individuals by dividing these groups into two clusters (FIG. 4c, FIG. 5e&f). Principal component analysis (PCA) using the top 1,000 DMCGIs revealed partitioning of cancer patients from the non-cancer controls (FIG. 4d, FIG. 6). In the PCA plots, non-cancer samples clustered tightly together, while cancer samples were not clustered, which can be attributed to tumor heterogeneity. These combined findings indicate that cfMBD-seq can identify DMCGIs in plasma cfDNA of cancer patients and non-cancer controls.

(3) Significant Overlap Between Tumor Tissue-Derived and cfDNA-Derived Differentially Methylated CpG Islands

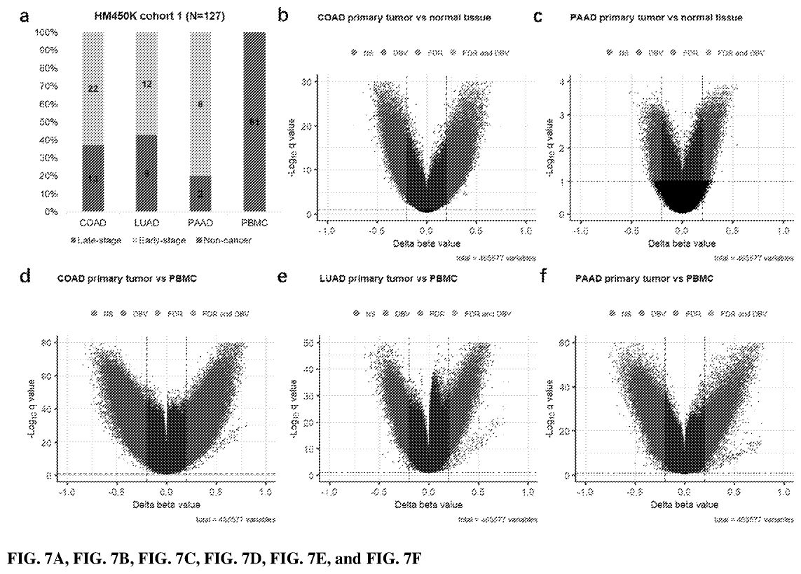

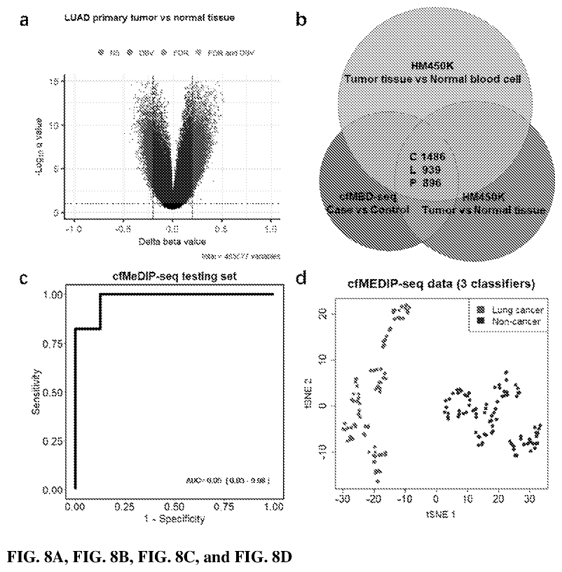

To explore whether DMCGIs detected by cfMBD-seq were originated from tumor tissues, we acquired Infinium HumanMethylation450 BeadChip (HM450K) data of primary tumors and matched adjacent normal tissues from the same patients, including colon adenocarcinoma (COAD, 35 pairs), lung adenocarcinoma (LUAD, 21 pairs), and pancreatic adenocarcinoma (PAAD, 10 pairs) from The Cancer Genome Atlas (TCGA) (FIG. 7a). We identified 21,274, 7,635, and 7,458 hypermethylated differentially methylated CpG sites (DMCs) (Mean of Δ beta value >0.2, BH-FDR<0.1, F-test) between primary tumors and matched normal tissues of COAD, LUAD, and PADD, respectively (FIG. 8a, FIG. 7b&c). To make HM450K results comparable to cfMBD-seq, we excluded the DMCs that were not annotated to CpG islands and kept the remaining 94.05%, 84.44%, and 90.73% of DMCs in the three cancer types. After further removal of duplicated CpG islands, we obtained 4,630, 2,588, 2,478 unique DMCGIs for COAD, LUAD, and PAAD, respectively. As non-tumor-derived cfDNA is mostly released from peripheral blood mononuclear cells (PBMCs), we conducted an analysis to determine whether the DMCGIs identified by cfMBD-seq were not derived from clonal hematopoiesis differences between cases and controls. For this purpose, we performed similar differential methylation analyses between HM450K data of primary tumors and cancer-free individuals' PBMCs (N=61 from Gene Expression Omnibus (GEO), non-smoker controls in GSE53045) and identified a set of DMCs for each cancer type (FIG. 7d-f). After annotation and exclusion of DMCs, we obtained 7,838, 4,906, and 5,613 unique DMCGIs for COAD, LUAD, and PAAD, respectively. Intersection analyses of three sets of DMCGIs showed that 84.5% of colorectal (1,486/1,759), 52.7% of lung (939/1,783), and 57.9% of pancreatic (896/1,548) cancer DMCGIs detected by cfMBD-seq overlapped with not only DMCGIs between primary tumor and adjacent normal tissue, but also DMCGIs between primary tumor and PBMCs (FIG. 8b). These findings indicate that plasma derived DMCGIs detected by cfMBD-seq were mainly driven by tumor-specific DNA methylation patterns rather than by background noise of cell composition in the tumor microenvironment.

### (4) Differentially Methylated CpG Islands for Early Lung Cancer Detection

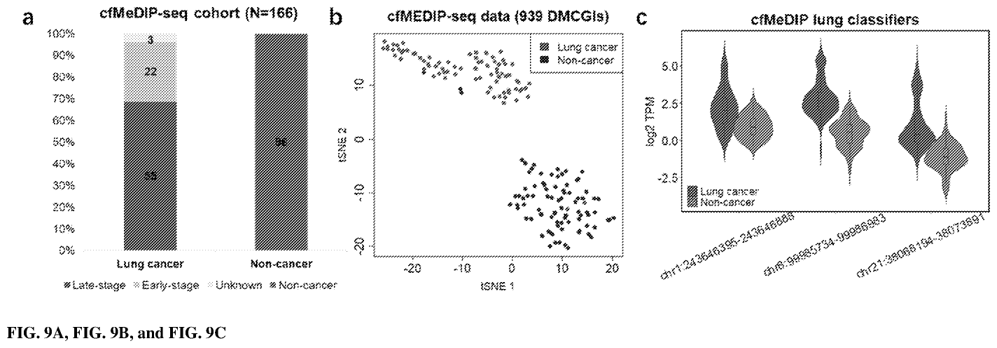

Since most HM450K data are originated from early-stage cancer tumor tissue samples, we hypothesized that the identified overlapping DMCGIs can be used for the early cancer detection. To test this hypothesis, we acquired an additional cohort of 166 plasma samples including 80 lung cancer patients (N=22 with early-stage disease) and 86 non-cancer individuals from a cfMeDIP-seq study (FIG. 9a). t-distributed stochastic neighbor embedding (t-sne) plot using the 939 overlapping lung cancer DMCGIs identified a clear separation between lung cancer and non-cancer individuals in the cfMeDIP-seq cohort, and only 5 individuals were misclassified (FIG. 9b). To rigorously evaluate the utility of these overlapping DMCGIs for cancer detection, we selected the top 300 lung cancer DMCGIs based on their rank on fold change in the cfMBD-seq results and carried out a set of machine learning analyses on the cfMeDIP-seq cohort. We randomly split these samples into balanced training (80%) and testing (20%) sets. To select the most discriminating markers, we trained a series of case-versus-control binomial generalized linear models (logistic regression) using these features on the training sets. The training procedure consisted of least absolute shrinkage and selection operator (LASSO) regularization method with 10-fold cross-validation. The process was repeated 100 times to prevent training-set biases. Eventually, we identified 3 DMCGIs that had non-zero coefficients across all repeats and selected those as cancer classifiers (FIG. 9c). To evaluate the performance of these classifiers, we fit the predictive model on the testing dataset and used receive operating characteristic (ROC) statistics to calculate area under the ROC curve (AUC) for evaluation. The results showed that the model can predict lung cancer in the testing set with high accuracy (AUC=0.949 [0.929-0.982]) (FIG. 8c). Using only the 3 classifiers for t-sne plot, all samples were correctly classified (FIG. 8d). These results indicate that early cancer detection is possible when using tissue-specific DMCGIs identified by cfMBD-seq.

### (5) Differentially Methylated CpG Islands for Cancer Classification

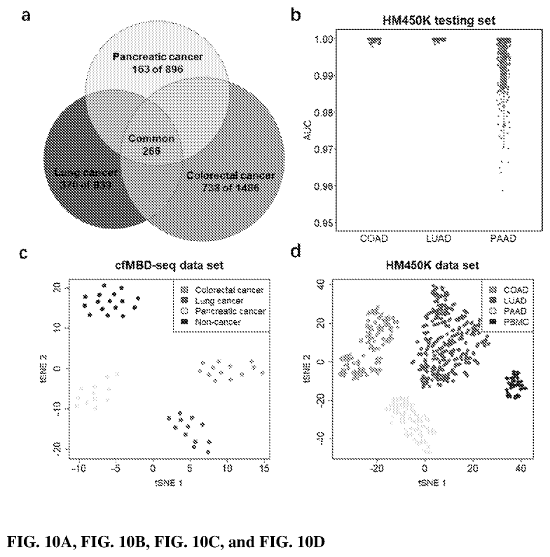

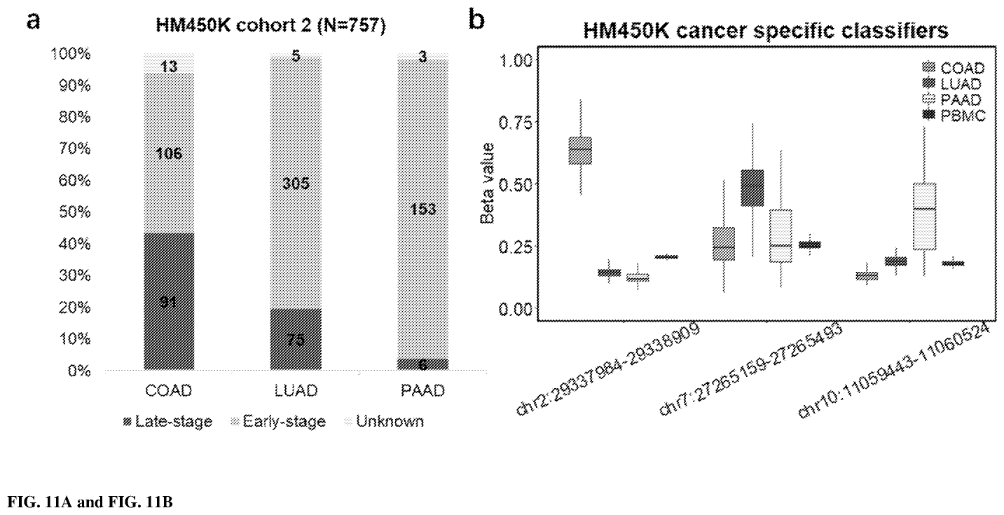

To further investigate the candidate DMCGIs shared between cfDNA and tumor tissue, we intersected the three sets of selected DMCGIs for colorectal (N=1,486), lung (N=939), and pancreatic (N=896) cancer. We identified a total of 1,271 cancer type specific DMCGIs, including 738 for colorectal cancer, 370 for lung cancer, and 163 for pancreatic cancer. Also, a total of 266 DMCGIs were shared by these three cancer types (FIG. 10a). To rigorously evaluate the performance of these cancer-type specific DMCGIs in cancer classification, we acquired an additional independent TCGA HM450K tumor tissue data cohort including primary tumors for COAD (N=210), LUAD (N=385), and PAAD (N=162) (FIG. 11a). To convert HM450K data to CpG islands-based beta value, we filtered out CpG sites that weren't annotated to CpG islands from 485,577 HM450K locus and used the remaining 309,465 CpG sites for subsequent analysis. Given the methylation level between neighboring CpG sites are positively correlated, we calculated the mean beta values of CpG sites annotated to the same CpG island and generated a beta value matrix for all CpG islands. We then performed similar machine learning analyses on the HM450K cohort using the top 100 cancer type specific DMCGIs. The analyses consisted of 4:1 sample partition, LASSO regularization with 10-fold cross validation, and logistic regression modeling. Rather than a case-versus-control model, here we built a one-versus-all-others model for each cancer type. After 100 repeats of the training process, we identified 3 colorectal, 16 lung, 6 pancreatic specific DMCGIs (non-zero coefficients) as classifiers. Again, we fit the predictive model on the held-out testing set and applied ROC statistics for evaluation. The results showed great performance in the prediction of cancer type (median AUC=1 for COAD, 1 for LUAD, and 0.989 for PAAD) (FIG. 10b). To better visualize the classification performance, we generated t-sne plot using these classifiers and observed clear separation by tumor type in the cfMBD-seq plasma cohort (FIG. 10c). This separation was notably reproduced in the HM450k cohort of 757 cancer tissue and 61 blood cell samples (FIG. 10d). These results indicate the robust ability of cfMBD-seq to recover tumor tissue-derived methylation profiles in cfDNA across a range of cancer types and enable cancer type classification.

### (6) Gene Annotation of Differentially Methylated CpG Islands

To gain an understanding of the biological process behind tissue specific DMCGIs, we linked these DMCGIs to their associated genes (Table 1). We found that some genes with promoter CpG island hypermethylation are implicated in immune response, which is generally downregulated in cancer. For example, the protein encoded by PTGER4 is a member of the G-protein coupled receptor family that can activate T-cell factor signaling. We not only identified DMCGIs in this gene promotor regions, but also found DMCGIs in gene bodies and intergenic regions. (Table 1). In contrast to promoter CpG islands hypermethylation that prevents gene expression, hypermethylation in gene body CpG islands can enhance gene expression levels. Consistent with our findings, genes with gene body CpG islands hypermethylation were associated with the regulation of developmental processes. For example, the protein encoded by WNT6 and HOXB8 has been implicated in oncogenesis and in several developmental processes such as embryogenesis. Overexpression of both WNT6 and HOXB8 plays key roles in carcinogenesis. These results indicate that cfMBD-seq can capture tumor relevant biological signals in the plasma cfDNA methylome. Taken together, our results indicate that DMCGIs in cfDNA are useful in cancer detection and classification, indicating that tumor-derived epigenomic signals are retained in the cfDNA methylome profiled by cfMBD-seq.

TABLE 1
Annotation of cancer type specific classifiers

| CpG islands | Size | Coefficients | Gene | Location |
|---|---|---|---|---|
| Colorectal cancer | | | | |
| chr2: 29337984-29338909 | 926 | −9.83 | CLIP4 | Promoter |
| chr2: 100937780-100939059 | 1280 | −29.19 | LONRF2 | Promoter |
| chr6: 125283125-125284389 | 1265 | 7.04 | RNF217 | Promoter |
| Lung cancer | | | | |
| chr2: 66672432-66673636 | 1205 | −9.04 | MEIS1 | Gene body |
| chr2: 71503548-71504233 | 686 | −5.54 | ZNF638 | Promoter |
| chr2: 219736133-219736592 | 460 | 5.80 | WNT6 | Gene body |
| chr4: 140655963-140657135 | 1173 | 13.48 | MGST2 | Gene body |
| chr4: 174427892-174428192 | 301 | 7.82 | \ | Intergenic |
| chr5: 40679503-40682081 | 2579 | −43.06 | PTGER4 | Promoter |
| chr7: 27265159-27265493 | 335 | −7.47 | \ | Intergenic |
| chr7: 65037625-65037864 | 240 | −14.04 | \ | Intergenic |
| chr8: 124172801-124173541 | 741 | −14.47 | \ | Intergenic |
| chr9: 96108467-96108992 | 526 | 12.82 | C9orf129 | Promoter |
| chr12: 54408427-54408713 | 287 | −5.11 | \ | Intergenic |
| chr12: 58021295-58022037 | 743 | 15.82 | B4GALNT1 | Gene body |
| chr13: 28549840-28550246 | 407 | 5.60 | \ | Intergenic |
| chr17: 46691521-46692097 | 577 | −4.75 | HOXB8 | Gene body |
| chr17: 59539363-59539834 | 472 | −12.18 | TBX4 | Gene body |
| chr17: 70112825-70114271 | 1447 | 9.87 | SOX9 | Promoter |
| Pancreatic cancer | | | | |
| chr1: 44883137-44884272 | 1136 | −10.54 | RNF220 | Gene body |
| chr1: 50798668-50799536 | 869 | 8.22 | \ | Intergenic |
| chr5: 92939796-92940216 | 421 | 7.85 | \ | Intergenic |
| chr10: 11059443-11060524 | 1082 | 10.34 | CELF2 | Promoter |
| chr11: 20177609-20178824 | 1216 | 6.63 | DBX1 | Gene body |
| chr12: 114881650-114881937 | 288 | −27.52 | \ | Intergenic |

### c) Discussion

Blood-based assays that can identify the tissue of origin associated with cfDNA fragments could be instrumental in detecting and classifying malignancies based on histological subtypes. Currently, cfDNA-based approaches that focus on the detection of cancer-associated single-nucleotide variants (SNVs) and somatic copy number variants (CNVs) have been used in clinical settings. However, SNV assays have limitations associated with confounding signals from blood cells due to clonal hematopoiesis. Similarly, CNV assays are limited by minor differences between cases and controls resulting in a need for increased sequencing depths, which translates into higher costs. More importantly, these genetic variations have not yet demonstrated robust tissue of origin classification across a broad range of tumor types. In contrast, given the inherited ability of tracing tissue of origin, cfDNA methylation is a promising biomarker in liquid biopsies. Therefore, detection of tumor-specific cfDNA methylation signatures is believed to be a more robust approach. In this study, we highlight the potential of hypermethylated CpG islands in cancer detection and classification.

Currently, most cfDNA methylation profiling technologies are based on chemical treatment using sodium bisulfite. Although whole-genome bisulfite sequencing of cfDNA has been attempted, this approach is not feasible for clinical applications because of high cost and limited information recovery due to the low abundance of CpG in the human genome. To address this issue, highly sensitive targeted assays such as targeted bisulfite sequencing and digital methylation-specific PCR have been developed. Targeted bisulfite sequencing of cfDNA has demonstrated high accuracy for detection of hepatocellular carcinoma and CRC in large cohort of cancer patients and non-cancer controls. However, the target methylation markers of these studies were selected from HM450K data. It is known that methylation array has poor genome-wide coverage of all CpG sites, which may result in omission of important targets. Alternatively, enrichment-based approaches such as cfMeDIP-seq and cfMBD-seq have also shown great potential in profiling the cfDNA methylome. These discovery assays enable the identification of novel blood-based methylation signatures, expanding on the existing biomarkers selected from tumor tissue. Our study focused on the feasibility of cfMBD-seq in identifying hypermethylated CpG islands in plasma cfDNA that can facilitate the development of blood-based molecular diagnostic tests.

Generally, sequencing data from methylation enrichment-based methods are analyzed by comparing the relative abundance of captured fragments. The genome is divided into non-overlapping adjacent genomic windows of a specified width and the number of sequence read counts is called for each window. Taking 300 bp window as an example, there are more than 10 million genomic regions which requires a significant amount of computing memory. In this study, instead of genomic windows, we called read counts according to CpG annotation features. This is because MBD methylation enrichment has bias toward hypermethylation on high CpG density regions. We found that 42.16% of the sequence reads in this study were mapped to CpG islands, and that 91.46% of the reads were mapped to the extended CpG islands, which account for only a small fraction of the human genome (FIG. 2e&f). Therefore, by excluding the large fraction of low value inter CpG regions, the computational efficiency was significantly enhanced. Additionally, well established RNA-seq data analysis packages such as DESeq2 can be directly applied to the CpG features read count matrix. Together, this CpG island-centered strategy is a preferred data analysis method for cfMBD-seq.

Differential methylation analysis based on a negative binomial model of CpG island read counts identified overwhelming differentially hypermethylated CpG islands (DMCGIs) (FIG. 3b). This is consistent with the fact that the tumor methylome is characterized by DNA methylation alterations with CpG islands-specific hypermethylation. However, confounding factors such as age and gender were not well matched between the cases and controls, which can result in false positive DMCGIs. To assess whether the DMCGIs identified by cfMBD-seq represented tumor-derived DNA methylation changes, we compared our findings against the HM450K tumor tissue data. We first identified a set of DMCGIs between paired primary tumor tissues and adjacent normal tissues. Since non-tumor-derived cfDNA released from blood cells can also lead to false positive results, we then identified a set of DMCGIs between primary tumor tissues and non-cancer PBMCs to deconvolute the clonal hematopoiesis effect. In our intersection analysis, the majority of the DMCGIs identified in plasma using cfMBD-seq were consistent with tumor-derived DMCGIs across all analyzed cancer types (FIG. 4b).

The main limitation of this study is the small sample size which prevented us from building prediction models using cfMBD-seq dataset. Instead, we selected to use the cfMeDIP-seq and HM450K datasets for predictive modeling. In the LASSO regularized logistic regression analysis using overlapping lung cancer DMCGIs in cfMeDIP-seq dataset, the model was able to discriminate lung cancer patients and non-cancer controls in the testing set with high accuracy (FIG. 4c). However, when we tried fitting the model to our cfMBD-seq dataset for validation purpose, the prediction performance was relatively poor. Although the methylation capture principle and data analysis pipeline of these two technologies are similar, the capture efficiency on fragments with different CpG density is different. cfMeDIP-seq preferentially enriches methylated regions with a modest CpG density, while cfMBD-seq captures a broad range of CpG densities and identifies a larger proportion of CpG islands. These differences can explain the impaired performance of these classifiers in our study cohort. Additionally, HM450K and cfMBD-seq are completely different technological platforms. Unlike bisulfite conversion-based methods, cfMBD-seq is an enrichment-based method that cannot provide the absolute methylation level at each CpG site. Taking advantage of the fact that the methylation level between neighboring CpG sites is positively correlated, we transformed the CpG sites beta value matrix into a CpG islands beta value matrix. This transformation not only mitigates the disadvantage that HM450K has poor coverage of all CpG sites, but also makes HM450K data comparable with cfMBD-seq DMCGIs.

In summary, in this proof of principle study we provide important insights into the possible future clinical applications of cfMBD-seq. Highlights of the study include: 1) cfMBD-seq enables the identification of cancer-associated DMCGIs from plasma cfDNA in cancer patients; 2) the identified DMCGIs are mainly driven by tumor-specific DNA methylation patterns and demonstrate promise for future studies using this technology for cancer detection and classification; 3) the most discriminating DMCGIs selected by our prediction models are associated with important biological processes that are contribute to carcinogenesis.

### 2. Example 2: Cell-Free DNA Methylome Profiling by MBD-Seq with Ultra-Low Input

### a) Results

(1) Characterization of cfMBD-Seq Technology

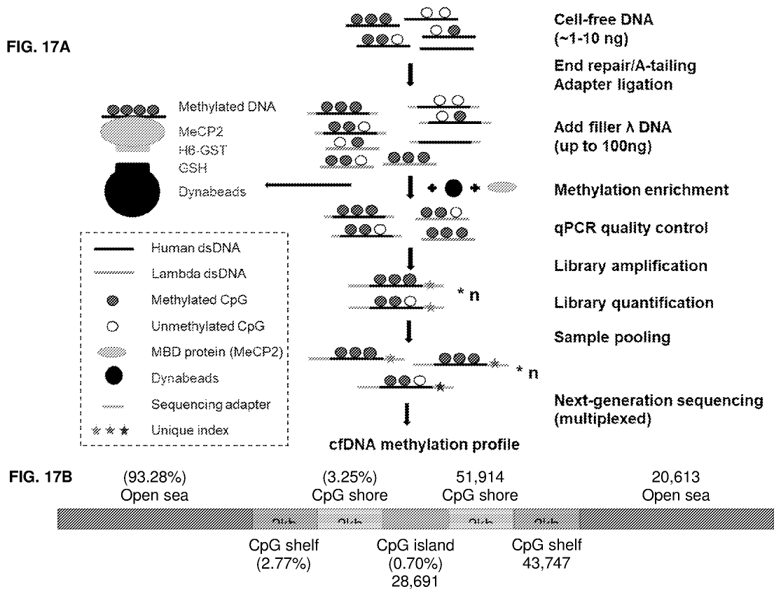

The standard protocol for methylation enrichment requires a minimum of 1000 ng DNA as input. Since the yield of cfDNA is extremely low at 2-10 ng per ml plasma, the current protocol is not suitable for cfDNA methylation analysis. To ensure amplification of methylation-enriched cfDNA, we added sequencing adapters to cfDNA by end repair/A-tailing and ligation before methylation enrichment and library amplification. This pre-enrichment adapter ligation preserves the methylation status of cfDNA because newly synthesized DNA are not methylated. To meet the high input requirement for methylation enrichment, we added exogenous Enterobacteria phage λ DNA (filler DNA) to the adapter-ligated cfDNA to increase the final DNA input to 100 ng. The filler DNA ensures a constant MethylCap protein/DNA ratio and helps maintain a similar methylation enrichment efficiency across different samples with different cfDNA yields while minimizing non-specific binding and DNA loss. Since filler DNA is not amplified during library amplification and is not aligned with the human genome, it will not interfere with the analysis of sequencing data. Unlike genome-wide sequencing, cfMBD-seq captures only a fraction of the genome (methylated DNA) and thus allows adequate sequencing coverage with fewer total reads. Therefore, it enables pooling of multiple uniquely indexed samples for a single run while retaining high sensitivity. This makes cfMBD-seq a cost-effective method for methylome-wide association analysis in a large-scale study (for details, see Methods and FIG. 17a).

### (2) Reduced MethylCap Protein Improves Methylation Enrichment

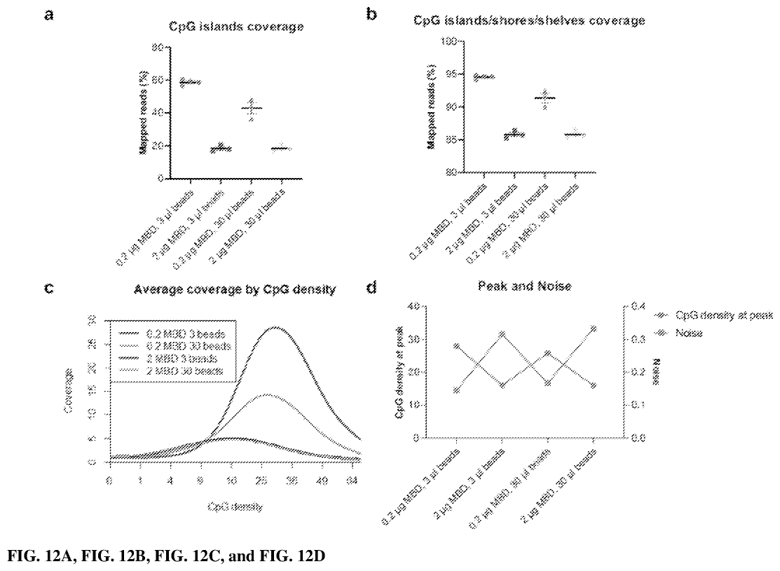

Based on the use of filler DNA, we performed extensive benchmarking to identify an optimal methylation enrichment condition. One of the key adaptations for this purpose is to determine the appropriate amount of MethylCap protein to bind the input DNA mixture. If the amount of protein is too high, non-specific binding will occur due to extra binding sites on the protein. If too low, a portion of methylated fragments will not be captured. We thus tested across different ratios of MethylCap protein and magnetic beads to input DNA. When MethylCap protein/DNA ratio is kept the same as recommended by the manufacturer, where 2 μg MethylCap protein is used for 1 μg DNA (2:1 ratio), the captured CpG islands reached up to 58.65% of all mapped reads (FIG. 12a). Since methylation differences sometimes occur at a short distance away from the CpG islands, we also calculated the sum of captured reads from CpG islands/shores/shelves regions. Under the recommended ratio, 94.56% of reads fell into the extended regions while these regions only account for 6.72% of the entire genome (FIG. 12b), 17b). We then plotted the genome-wide coverage (average number of fragments covering CpGs) against CpG density (number of CpGs per fragment). The curve shows that the coverage is relatively low in CpG-poor regions and ultra-dense regions, while peaks in regions have moderate CpG density. As the peak represents CpG-rich regions such as CpG islands, the higher coverage at the peak indicates the better methylation enrichment (FIG. 12c). To better characterize these distributions, we termed the CpG density at the point of the highest coverage as ‘peak’. We also used the term ‘noise’ to illustrate the ratio of average non-CpG coverage to average CpG coverage. Consistently, the 2:1 ratio gives the highest peak and the lowest noise (FIG. 12d). Unlike the MethylCap protein, the volume of magnetic beads had less impact on the performance of methylation enrichment. Given that redundant beads can increase the risk of nonspecific binding, we determined the best enrichment conditions as 0.2 μg protein and 3 μl beads with a total input DNA of 100 ng.

### (3) Methylated Filler DNA is Needed to Increase Enrichment Efficiency and Reduce Background Noise

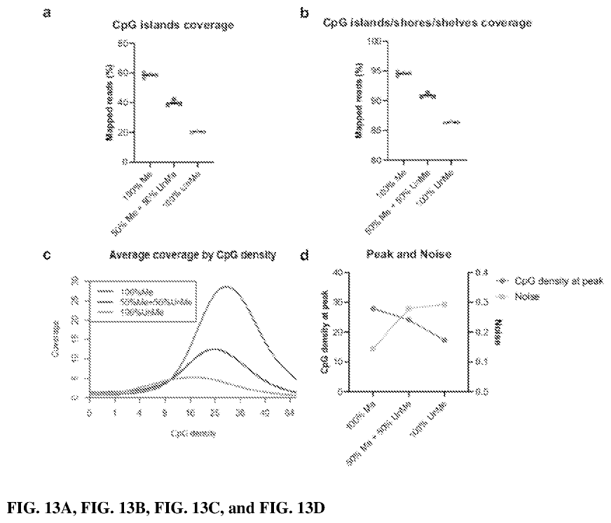

In MBD-based enrichment, the typical yields of methylated DNA are 3-20% of the input DNA mass. Since cfDNA only accounts for a small fraction (<10%) in the mixture of cfDNA and filler DNA, the methylated fragments in cfDNA are able to fill all binding sites in the MethylCap protein. If the filler DNA is not methylated, the risk of unspecific binding is increased. To test the potential impact of filler DNA methylation status on enrichment efficiency, we treated the filler DNA with CpG methyltransferase and used the mixture of the treated and untreated filler DNA as input. When filler DNA is methylated, we observe preferential enrichment in both the CpG islands and CpG islands/shores/shelves regions. The coverages of enriched target regions decreased with reduced methylation level of filler DNA (FIG. 13a, b). For example, CpG islands coverage was 58.65%, 40.05%, and 20.53% when methylation level of filler DNA was 100%, 50%, and 0%, respectively. The extended regions show the same trend. The coverage by CpG density plot (FIG. 13c) and peak/noise trend plot (FIG. 13d) further confirmed the importance of methylated filler DNA. Since the methylated filler DNA can block the extra binding sites on the protein, it is not difficult to explain why the specificity of the reaction was enhanced.

### (4) Library Yield and Spike-In Control are Used for Pre-Sequencing Quality Controls

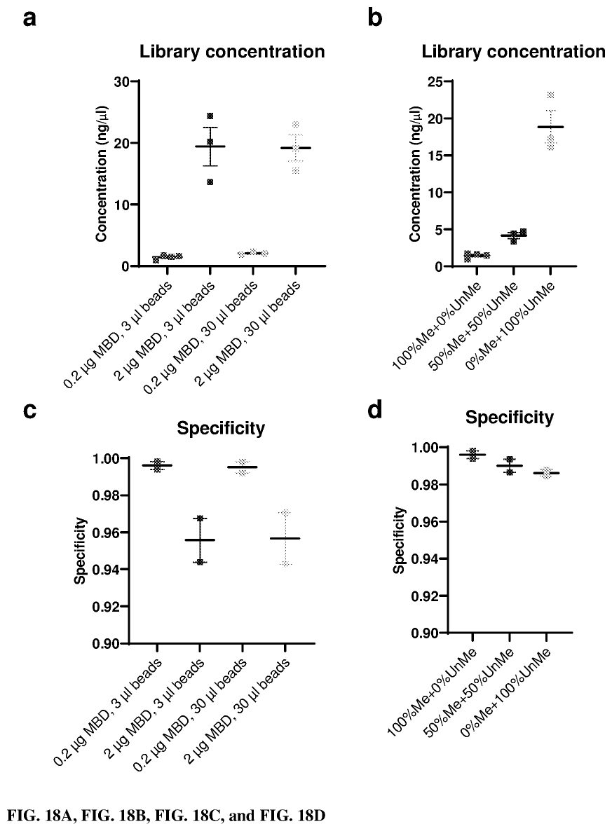

We empirically find that the library yield for different conditions is different and hypothesize that a non-specific capture increases the final library yield. To test this, we examined the final library concentration and the quality of methylation enrichment. We find that optimal condition tended to have a lower library concentration while suboptimal conditions generate more final library DNA under the same amplification cycles (FIG. 18a, 18b). Besides library concentration, real-time PCR (qPCR) often provides a more accurate pre-sequencing quality control. Since cfDNA is highly fragmented, the use of large amplicon (such as 170 bp of methylated control TSH2B) is not recommended. In fact, it is very hard to detect unmethylated control GAPDH in a successful enrichment due to low input. Therefore, instead of the TSH2B and GAPDH control pair, we used A. thaliana DNA as spike-in control to estimate the enrichment efficiency. We observed a significant enrichment of methylated DNAs when compared spike-in controls before and after capture reaction. Under the optimal condition, the specificity of capturing methylated control DNA was ≥99%, with the recovery rate of spiked-in methylated control should be ˜50%-90% and the recovery of spiked-in unmethylated control should be <1% (FIG. 18c, 18d).

### (5) 1 ng Input Achieved High-Quality Results Similar to 1000 ng Input DNA

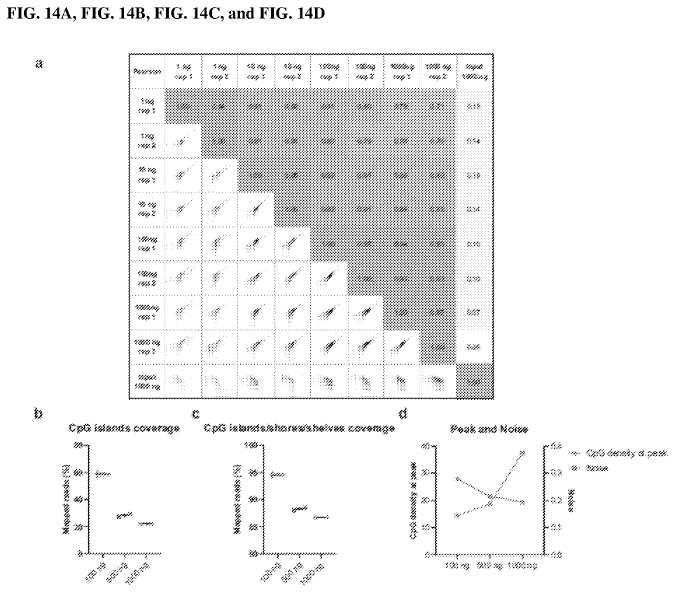

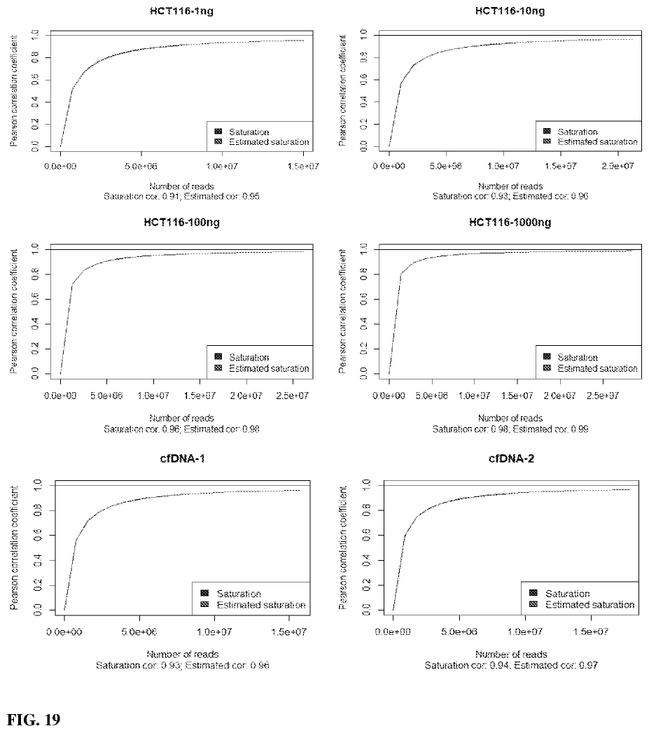

To compare the low-input cfMBD-seq with standard MBD-seq (>1000 ng input), we sheared colorectal cancer HCT116 DNA into small fragments with a peak of ˜167 bp to mimic cfDNA and tested different DNA inputs for methylation enrichment (1, 10, 100, and 1000 ng). For 1 ng and 10 ng DNA, we used methylated filler DNA to increase the final DNA input to 100 ng. For 1000 ng input, standard MBD-seq was used for library preparation. The results show robust genome-wide inter-replicate Pearson correlation (FIG. 14a). More importantly, saturation analysis showed a high saturation correlation of 0.91 with only 1 ng DNA as input (FIG. 19), indicating that the methylome profile from 1 ng DNA is sufficient to generate a saturated and reproducible coverage profile of the reference genome. The saturation correlation of 3 ng cfDNA input is consistent with low genomic DNA (gDNA) input (FIG. 19). Together, these results indicate cfMBD-seq can generate high-quality methylome profiles similar to standard MBD-seq while allowing ultra-low DNA input. As the 1000 ng input has a high genome-wide inter-replicate correlation, we further investigated if increased amount of filler DNA can enhance the performance of the reaction. We thus increased the DNA input by adding more filler DNA, with the quantity of cfDNA unchanged (in total 100, 500, and 1000 ng). However, we did not observe an improved methylation enrichment even when the amounts of MethylCap protein and beads were adjusted accordingly. The higher filler DNA reduced the performance of target region enrichment (FIG. 14b, c) and increased background noise (FIG. 14d), indicating that the increased amount of methylated filler DNA overshadowed the trace amount of methylated cfDNA. Thus, we determined 100 ng as an optimized DNA input, due to the robust recovery of high CpG density regions with low noise.

### (6) Additional Wash and Elution Buffers Did not Significantly Affect Methylation Enrichment

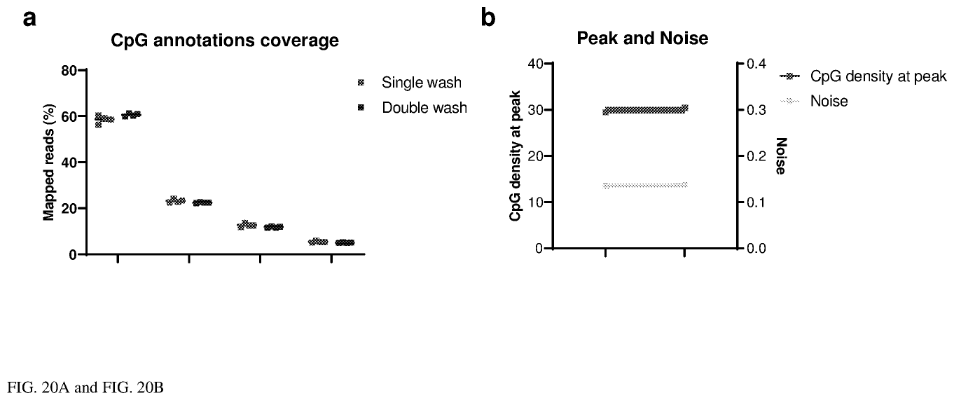

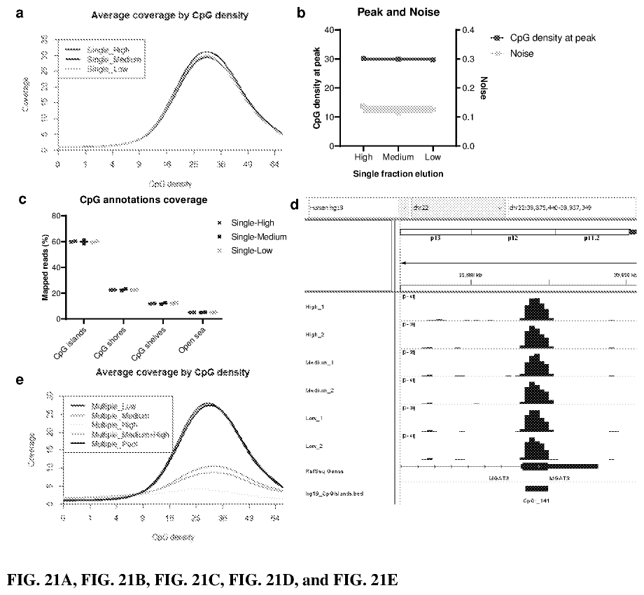

Given the confirmed MethylCap protein-to-DNA ratio and amount of methylated filler DNA, we evaluated other experimental conditions to see if methylation enrichment can be further improved. First, we examined the effect of a more stringent washing condition on non-specific binding. Compared to single wash, the double wash did not significantly increase coverage of CpG islands. The additional wash also did not decrease the coverage of the open sea regions, where non-specific bindings are most likely to occur (FIG. 20a). Likewise, there was no significant difference in noise between the standard wash and double wash (FIG. 20b). Second, we examined the effect of the elution buffer salt concentration on methylation enrichment. We performed single fraction elution for three different elution buffers provided in the MethylCap kit. Theoretically, an increased salt concentration preferentially enriches regions with a higher CpG density. However, we did not observe a notable shift in the coverage by density plot nor coverage difference in each CpG annotations (FIG. 21a-c). For example, the coverage signals at the CpG island MGAT3 showed no difference among three elution buffers (FIG. 21d). The finding that MethylCap protein (MeCP2) is not sensitive to the salt concentration of elution buffer is consistent with the findings herein. We also tested multiple fractions elution, that is, sequential elution with low, medium, and high salt elution buffer from one capture reaction. The coverage by density plots illustrated robust methylation enrichment in both the first fraction (low salt) and the pool of three fractions (FIG. 21e). However, the second fraction (medium salt), the third fraction (high salt), and the pool of both fractions had very low coverage due to the intrinsic limitation of low input (FIG. 21e). In summary, our results indicate an optimal condition for low input MBD methylation enrichment includes 0.2 μg MethylCap protein and 3 μl beads for 100 ng DNA mixture (cfDNA+methylated filler DNA), standard wash, and single fraction elution.

(7) Comparison of cfMBD-Seq with Other Technologies

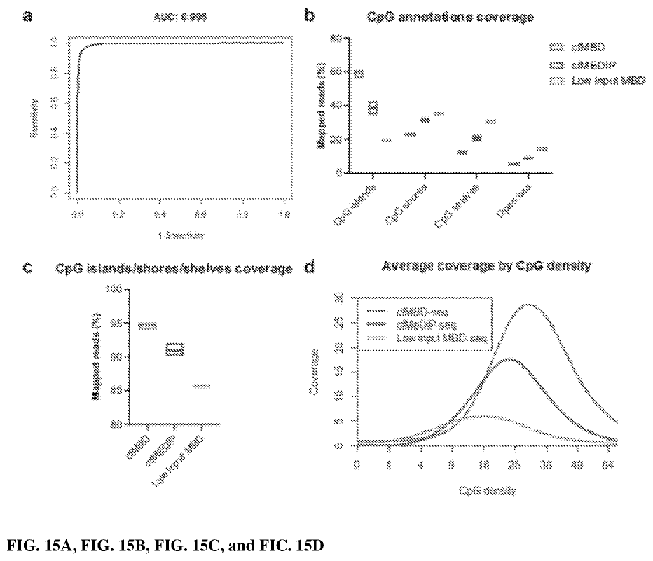

To evaluate the methylation capture accuracy of cfMBD-seq, we calculated its sensitivity (proportion of methylated CpG islands detected) and specificity (proportion of non-methylated CpG islands detected). We used Infinium HM450K data (Gene Expression Omnibus (GEO): GSE55491, peripheral blood mononuclear cell (PBMC) from N=5 healthy controls) as standard to determine whether a CpG island was methylated or non-methylated. It is known that the methylation level between neighbouring CpG sites is positively correlated. Therefore, to obtain a comparable measurement between cfMBD-seq and methylation array, we averaged beta-values of adjacent CpG sites within each CpG island and defined the methylation status of that CpG island. We then built a logistic regression model for all CpG islands in the microarray using normalized read counts from cfMBD-seq and methylation status from the microarray (AUC=0.995, FIG. 15a). At the cut-off of 13.25, derived from the intersection of the specificity and sensitivity curves translated to normalized read counts, the sensitivity of cfMBD-seq is 0.94 and the specificity is 0.98. Namely, at this threshold, cfMBD-seq detected 94% of the methylated CpG islands that were reliably detected by Infinium methylation array while correctly classifying 98% of the non-methylated sites.

To determine the performance of cfMBD-seq over existing methylation enrichment assays, we compared cfMBD-seq with a low-input MBD-seq protocol (N=4 from GEO: GSM2593327-GSM2593330) that did not use filler DNA. This protocol uses MBD2, another MBD family member that is sensitive to the salt concentration of the elution buffer, for methylation capture. In order to balance the methylome-wide coverage, this protocol uses a low-salt buffer for elution, which results in a very low recovery rate (median 19.95% [(Q1) 19.25%-(Q3) 20.11%]) of the high CpG density regions (CpG islands) and a relatively high recovery rate (14.30% [14.24%-14.49%]) of the open sea regions (FIGS. 15b and c). Worst of all, the overall coverage is low, which makes it difficult to discriminate methylated fragments from non-specific fragments and reduces the statistical power of differentially methylated analyses (FIG. 15d). We next compared cfMBD-seq with cfMeDIP-seq (N=24 from published dataset) which showed adequate performance in capturing tumour-specific methylation in cfDNA. According to the summary QC from the RaMWAS package, we observed a higher percentage of reads that passed the filter in cfMBD-seq (83.15% [82.93%-83.68%]) than in cfMeDIP-seq (74.90% [74.53%-75.45%]) and a lower duplicate rate (3.45% [3.40%-3.90%] vs. 12.00% [9.00%-19.23%]). Taken together, cfMBD-seq generated higher quality of sequencing data and provided more informative sequences than cfMeDIP-seq given the same amount of aligned reads (79.60% [79.15%-80.43%] vs. 62.65% [55.60%-66.65%]) (Table 4). From CpG annotation-based coverage report, cfMBD-seq showed a significantly higher recovery rate at CpG islands (60.13% [58.78%-60.81%] vs. 38.16% [37.21%-41.28%], FIG. 15(b)) and a slightly higher recovery rate at combined CpG islands/shores/shelves (94.81% [94.61%-94.98%] vs. 90.90% [90.91%-91.55%], FIG. 15(c)), indicating that cfMBD-seq preferentially enriches CpG islands while cfMeDIP-seq has more signal on CpG shores and CpG shelves. This finding is consistent with the coverage by the CpG density plot, where cfMBD-seq peaks at higher CpG density than cfMeDIP-seq (29.98 [29.54-30.33] vs. 22.88 [22.37-23.50], FIG. 15(d)). The comparison between cfMBD-seq, low input MBD-seq, and cfMeDIP-seq is summarized in Table 4.

TABLE 4
Feature comparision among cfMBD-seq,
low input MBD-seq, and cfMeDIP-seq.

| | | Low input | |
|---|---|---|---|
| | cfMBD-seq | MBD-seq | cfMeDIP-seq |
| | (N = 8) | (N = 4) | (N = 24) |
| Experiment | | | |
| Filler DNA | Methylated | No filler | Mixture of |
| | DNA only | | methylated and |
| | | | unmethylated |
| | | | DNA |
| DNA | Not required | Not required | Required |
| Denaturation | | | |
| Capture | MeCP2 | MBD2 | Anti-5mc |
| protein | | | |
| Capture time | 5 | 5 hours | 23 hours |
| hours | (including | (including | (including |
| | 3 hours | 3 hours | 17 hours |
| | incubation) | incubation) | overnight |
| | | | incubation) |
| Quality Control | | | |
| Reads passed | 83.15% | 85.40% | 74.90% |
| filter | [82.93%- | [85.03%- | [74.53%- |
| | 83.68%] | 85.70%] | 75.45%] |
| Duplicate rate | 3.45% | 2.65% | 12.00% |
| | [3.40%- | [2.60%- | [9.00%- |
| | 3.90%] | 2.78%] | 19.23%] |
| Used reads | 79.60% | 82.75% | 62.65% |
| | [79.15%- | [82.25%- | [55.60%- |
| | 80.43%] | 83.10%] | 66.65%] |
| Methylation | | | |
| Enrichment | | | |
| Reads on CpG | 60.13% | 19.95% | 38.16% |
| islands | [58.78%- | [19.25%- | [37.21%- |
| | 60.81%] | 20.11%] | 41.28%] |
| Reads on CpG | 94.81% | 85.70% | 90.90% |
| islands/ | [94.61%- | [85.51%- | [90.91%- |
| shores/ | 94.98%] | 85.76%] | 91.55%] |
| shelves | | | |
| CpG density | 29.98 | 15.76 | 22.88 |
| at peak | [29.54- | [15.41- | [22.37- |
| | 30.33] | 15.88] | 23.50] |
| Noise | 0.12 | 0.13 | 0.02 |
| | [0.12- | [0.10- | [0.02- |
| | 0.15] | 0.16] | 0.02] |

Median along with [first quartile (Q1) − third quartile (Q3)] are shown. See supplementary table for detail including numbers of raw reads, duplicate reads, uniquely mapping reads, peak, and noise per sample.

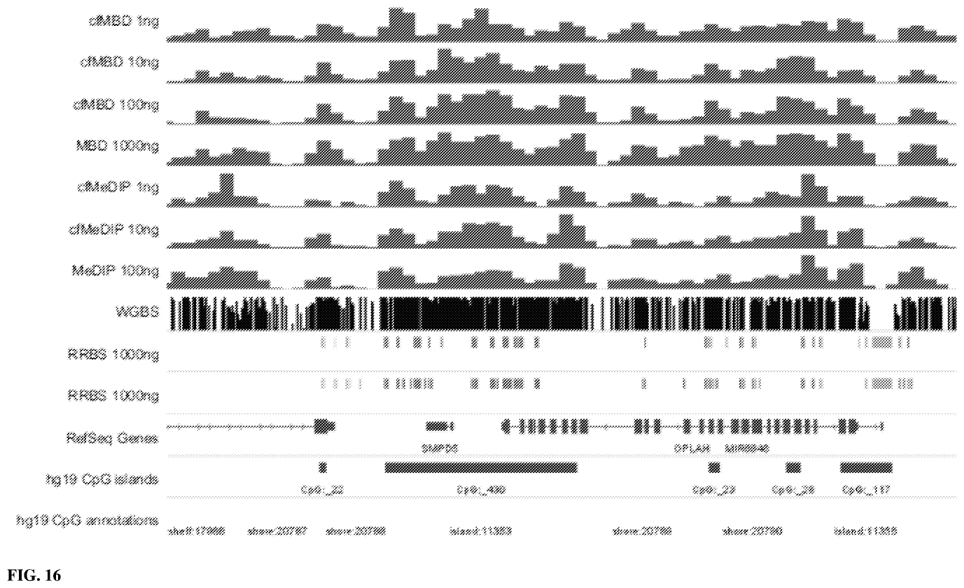

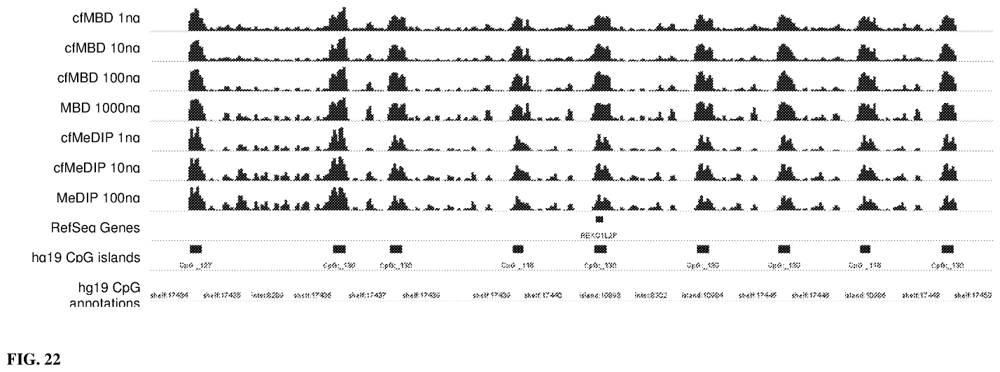

To better demonstrate the reproducibility of cfMBD-seq, we show a snapshot of a genomic region with consecutive CpG islands (chr8:86,703,816-86,880,439). We observed peaks with high similarity among cfMBD-seq (1 to 100 ng input DNA), standard MBD-seq (1000 ng), cfMeDIP-seq (1 to 10 ng), and standard MeDIP-seq (100 ng) (FIG. 22). We then compare the signal peaks among different methylation profiling technologies. We show that cfMBD-seq also recapitulated methylation profiles from reduced representation bisulphite sequencing (RRBS, 1000 ng) and WGBS (2000 ng) (FIG. 16). All these findings indicate that cfMBD-seq, allowing for ultra-low amounts of starting material, can extend the methylome-wide investigations that can be conducted with MBD-seq.

### b) Discussion

In this study, we further optimized the MBD-seq protocol to enable methylation enrichment from ultra-low DNA input. Our data show that cfMBD-seq achieves high genome-wide inter-replicate Pearson correlation with the standard MBD-seq (>1000 ng input) even when the input DNA is as little as 1 ng. cfMBD-seq also performs better than a low input MBD-seq protocol without using filler DNA in methylation enrichment of CpG islands/shores/shelves regions. Moreover, cfMBD-seq outperforms cfMeDIP-seq in the enrichment of fragments with higher CpG density such as CpG islands. This finding is consistent with comparisons of the standard MBD-seq with the standard MEDIP-seq: MeDIP commonly enriches methylated regions with a low CpG density while MBD captures a broad range of CpG densities and identifies the greatest proportion of CpG islands. It is known that CpG-rich fragments do not undergo complete denaturation into single stranded DNA, which is required for an efficient MeDIP capture and can explain why MeDIP-seq is less sensitive towards fragments with high CpG density. In contrast, MBD capture does not require DNA denaturation because the MethylCap protein is sensitive towards double stranded DNA. Therefore, temperature control of DNA-protein mixture during MBD capture is less strict than that of MeDIP capture. In addition, MBD enrichment in cfMBD-seq can be finished within 5 hours (including 3 hours of incubation) while cfMeDIP enrichment requires overnight incubation. Thus, the reaction to MBD enrichment is less time-consuming. cfMBD-seq showed a slightly higher noise than cfMeDIP-seq in the summary QC of RaMWAS package. Noise is defined as the ratio of the average coverage of fragments that do not contain a CpG tandem to the average coverage of fragments that contain a CpG tandem in this package. As cfMBD-seq preferentially enriches methylated fragments with high CpG density, the coverage of fragments with low CpG density is expected to be low. However, low CpG density fragments are widely distributed in the human genome (open sea, FIG. 17b), resulting in a relatively low average CpG coverage of cfMBD-seq. The average non-CpG coverage of cfMBD-seq and cfMeDIP-seq is less than 1, indicating high specificity of both assays. Overall, cfMBD-seq is a method of choice for interrogating regulation of gene expression (methylation changes in CpG islands). On the other hand, cfMeDIP-seq can be preferable in investigating transcriptional regulation of non-coding RNAs (methylation changes in gene bodies and CpG shores).

There are a few caveats to ensure successful cfMBD-seq. First, the quality of the MethylCap protein is very important. We notice that the use of the MethylCap protein, which has experienced multiple freeze-thaw cycles negatively impacts the data quality. Because the MethylCap protein is used with 10-fold dilution before adding to the reaction, it can be used for more reactions than standard MBD capture. Therefore, we recommend splitting the MethylCap protein into multiple aliquots to minimize the freeze-thaw cycles and using fresh diluted protein for each batch. Second, the success of the methylation enrichment reaction must be validated by qPCR to detect recovery of spiked-in control. The specificity of the reaction should be ≥99% before proceeding to the next step. Third, accurate library quantification is critical. Since methylated filler DNA is used in the methylation enrichment, qPCR-based library quantification is recommended because of its ability to quantify amounts of amplifiable DNA. Finally, adequate sequencing depth is crucial for high-quality data. Based on the saturation analysis, at least 30 million mapped reads are required to generate a saturated and reproducible coverage profile. The cost of cfMBD-seq from cfDNA extraction through the generation of sequencing data (single-end and pooling 12-15 indexed libraries) using the Illumina NextSeq 550 platform is less than $300 per sample. This cost-effective feature allows large-scale methylome-wide association analysis that is crucial for the establishment of a diagnostic model with high accuracy.

It is worth mentioning that the current study also has some limitations. First, it is well known that methylation status is different between individuals. The differences observed among cfMBD-seq, low-input MBD-seq, and cfMeDIP-seq can be partly attributed to differences in the samples that were used. Thus, this approach requires further validation. Second, the main application of cfMBD-seq is to identify cancer biomarkers in cfDNA. However, current study was limited to technology development and optimization. Further study in patient's samples is warranted to test the feasibility of cfMBD-seq in clinical settings, in particular to elaborate how well this technology can differentiate the tumour-derived cfDNA (ctDNA) methylation from high background overall cfDNA.

Our study demonstrates the potential benefits of using cfMBD-seq to profile the methylome of cfDNA with ultra-low DNA input. Current results provide justification for further validation using case and control plasma samples from different malignancies to perform differential methylation analyses. Since enrichment-based methods are analysed by comparing the relative abundance of sequenced fragments, cfMBD-seq shares similar analysis workflows with cfMeDIP-seq for identification of DMRs and other downstream machine learning analyses. Another potential for cfMBD-seq is its use in other methylome-wide investigations that are limited by DNA yield. We confidently believe that cfMBD-seq, being non-invasive and cost-effective, has great potential in identifying biomarkers for cancer detection and classification.

### c) Methods

(1) cfDNA and HCT116 DNA Extraction

Pooled human plasma (IPLAWBK3E50ML) was purchased from Innovative Research (Novi, MI, USA). Whole blood (K3 EDTA tube) was collected from donors in an FDA-approved collection centre. Plasma was frozen immediately after isolation. After thawing, additional centrifugation of 3000 rpm for 10 min was performed to ensure complete depletion of cell debris. cfDNA was extracted using QIAamp Circulating Nucleic Acid Kit (Qiagen; Hilden, Germany) and quantified using Qubit Fluorometer with iQuant™ NGS-HS dsDNA Assay Kit (Genecopoeia; Rockville, MD, USA). The average cfDNA yield from 1 ml plasma was ˜7.5 ng. The colorectal carcinoma cell line HCT116 was purchased from ATCC (CCL-247™) and cultured according to the recommended cell culture method. HCT116 DNA was extracted using QIAamp DNA Blood Mini Kit (Qiagen) and quantified using Nanodrop (NanoDrop Technologies; Wilmington, Delaware, USA). gDNA was sheared to 160 bp using Covaris ME220 Focused Ultrasonicator to mimic the fragment size of cfDNA. HCT116 was chosen because of the availability of public DNA methylation data.

### (2) Library Preparation and Filler DNA Generation

DNA was subjected to end repair/A-tailing and adapter ligation using KAPA Hyper Prep Kit (Kapa Biosystems; Wilmington, MA, USA) with the sequencing adapter from NEBNext Multiplex Oligos for Illumina (New England BioLabs; Ipswitch, MA, USA). The number of adapters used in the reaction was adjusted according to an adapter:insert molar ratio of 200:1. Adapter ligated DNA was purified with SPRI Beads (Beckman Coulter; Pasadena, CA, USA) and digested with USER enzyme (New England BioLabs) followed by purification with DNA Clean & Concentrator-5 Kit (ZYMO Research; Irvine, CA, USA). Meanwhile, filler DNA was generated via polymerase chain reaction (PCR) with GoTaq Master Mix (Promega; Madison, WI, USA), using Enterobacteria phage λ DNA as template. Amplicons were treated with CpG methyltransferase (M.SssI; Thermo Fisher Scientific; Waltham, MA, USA) for CpG methylation. The CpG methylation-sensitive restriction enzyme HpyCH4IV (New England BioLabs) digestion followed by agarose gel electrophoresis was used to ensure complete methylation of filler DNA.

(3) cfMBD-Seq

Adapter ligated DNA was first combined with methylated filler DNA to ensure that the total amount of input for methylation enrichment was 100 ng, which was further mixed with 0.2 ng of methylated and 0.2 ng of unmethylated A. thaliana DNA from DNA Methylation control package (Diagenode, Seraing, Belgium). The DNA mixture was then subjected to methylation enrichment using MethylCap Kit (Diagenode) following the manufacturer's protocol with some modifications. The total volume brought up by Buffer B was reduced to 140 μl to minimize DNA waste. The amounts of MethylCap protein and magnetic beads were decreased proportionally according to the recommended DNA to protein and beads ratio (0.2 μg protein and 3 μl beads per 100 ng DNA input). Single fraction elution with High Elution Buffer was applied. The eluted fraction was purified by DNA Clean & Concentrator-5 Kit. The purified DNA was divided into two parts, one for qPCR (PowerUp™ SYBR™ Green Master Mix, Thermo Fisher) quality control and another for library amplification. The recovery of spiked-in methylated and unmethylated control can be calculated based on the cycle threshold (Ct) value of the enriched sample and input control. The specificity can be calculated by (1−[recovery of unmethylated control DNA over recovery of methylated control DNA])×100. The methylation-enriched DNA libraries were amplified as follows: 95° C. for 3 min, followed by 12 cycles of 98° C. for 20 s, 65° C. for 15 s, and 72° C. for 30 s and a final extension of 72° C. for 1 min. During amplification, a unique index from the primer was added to the sequencing adapter for each sample. The amplified libraries were purified using SPRI Beads followed by a dual size selection (0.6× followed by 1.2×) to remove any adapter dimers. All final libraries were first quantified using Qubit Assay and KAPA Library Quantification Kits (Kapa Biosystems) and then submitted to Moffitt Cancer Center Molecular Genomics Core for D1000 ScreenTape Assay (Agilent; Santa Clara, CA, USA). Libraries were sequenced on the NextSeq 550 platform (Illumina; San Diego, CA, USA), high-output 75 bp single-end read, multiplexed as ˜12-15 samples per run.

### (4) Data Processing

After sequencing, pre-alignment quality control was performed for the raw sequenced reads using fastp (Version 0.20.1) with the default settings. The sequenced reads were then aligned to the human genome (hg19) using Bowtie-2 (Version 2.4.2) with the default settings. After the alignment, the generated sam files were converted to bam files, followed by sorting and indexing duplicate read removal, and read count extractions on chr1-chr22 using SAMtools (Version 1.11) ‘view’, ‘sort’, ‘index’, and ‘markdup’ command lines. R (Version 4.0.3 or greater) package RaMWAS (Version 1.12.0) was used for quality control of the overall mapping quality and calculation of average non-CpG/CpG coverage and coverage by CpG density. To ensure the comparability between different conditions, bam files of the same experimental condition were merged and 30 million sequenced reads were randomly extracted from each condition for plotting of coverage by CpG density plot. R package MEDIPS (Version 1.40.0) was then applied for saturation analysis and calculation of correlations of genome-wide short read coverage profiles between samples based on counts per 1000 bp non-overlapping windows. Normalized data were exported as wiggle files for visualization on the Integrative Genomics Viewer.

CpG annotations reference was obtained from R package annotatr (Version 1.16.0). BEDtools (Version 2.28.0) ‘coverage’ command line was used to call the coverage according to the CpG annotations reference. TPM (Transcripts Per Kilobase Million) normalization was performed before comparing the CpG annotations coverage between different samples. Data from low-input MBD-seq and cfMeDIP-seq were reprocessed from raw data (fastq level) using the same workflow. R package minfi (Version 1.36.0) was used to call and annotate (hg19) methylation signal from Infinium HM450K data. The average beta-values of each CpG site among different samples were first calculated. Methylation status of CpG islands was then determined by the average beta-values of adjacent CpG sites within the same CpG island (<0.5 as unmethylated and >0.5 as methylated). Logistic regression model was built using normalized read counts from cfMBD-seq and methylation status (methylated as 1 and unmethylated as 0) from microarray. R package ROCR (Version 1.0-11) was used to generate the receiver operating characteristic curve. All data and R images were imported into GraphPad Prism 8 for preparation of figures. A detailed bioinformatics analysis pipeline was coded in git bash and is available in GitHub (see availability of materials and data).

### 3. Example 3: Plasma Cell-Free DNA Methylome Profiling in Pre- and Post-Surgery Oral Cavity Squamous Cell Carcinoma

Head and neck squamous cell carcinoma cancer (HNSCC) is a highly heterogeneous disease that involves multiple anatomic sites including oral cavity, larynx, and pharynx. Outcomes of patients with HNSCC have not improved significantly over the past decade, with an overall five-year survival rate around 50%. There is a pressing need in the area to develop reliable prognostic and diagnostic biomarkers to enable better patient management, from early detection of disease to efficient monitoring of cancer recurrence following treatment. Despite their established role in cancer development, epigenetic biomarkers and DNA methylation (DNAme) remain understudied in HNSCC research. Currently, TCGA-HNSC is the only publicly available resource for mining DNAme patterns in head and neck cancer. Cell free DNA (cfDNA) includes both genetic and epigenetic information and offers several advantages including monitoring tumor burden, and novel discovery of biomarkers for diagnosis and prognosis. cfDNA is thought to potentially incorporate metastatic sites thus addressing tumor heterogeneity. Aberrant DNA methylation changes are thought to occur early during tumorigenesis and enables tumor progression and thus may be a more specific and sensitive approach to identify minimal residual disease and prognosis. While genetic analysis of cfDNA can be challenging due to its low yield and being highly fragmented, plasma cfDNA next generation assays are starting to be utilized in routine clinical use for solid malignancies such as lung and colon cancers to make treatment decisions.

cfDNA has been reported to decrease to background level following surgery. Therefore, we hypothesized that comparing methylation profiles in pre- and post-surgery plasma samples can help validate HNSCC-specific prognostic and diagnostic biomarkers, and provides an opportunity for novel biomarker discovery. Here, we focus on single anatomic site and assess the feasibility of detecting cfDNA methylome in patients with locoregional oral cavity squamous cell carcinomas (OCSCC) and the methylome dynamics in post-operative setting. A high-sensitive cfDNA methylome profiling technique called cfMBD-seq was applied on collected plasma samples. Different from bisulfite conversion-based sequencing methods, cfMBD-seq capture and quantify methylated DNA by methyl-CpG binding protein (MBD). cfMBD-seq is able to generate high-quality sequencing read with ultra-low amount of input DNA (2-10 ng per ml), and has demonstrated better performance in terms of enrichment of CpG islands compared to similar protocols such as cfMeDIP-seq. To facilitate cfDNA methylation biomarker prioritization with limited sample size, we first conducted a bioinformatics analysis to detect differentially methylated regions (DMRs) based on the matched tumor-normal tissue data collected from the TCGA-HNSC project. We hypothesized that the top cancer-specific DMRs detected in the TCGA-HNSC cohort will also exhibit differential methylation patterns between before- and after-surgery plasma samples. As an alternative strategy, we performed a genome-wide search of top DMRs based on the matched cfDNA methylation profiles. DMR analyses were conducted on different patient subgroups as a sensitivity analysis considering the presence of patient and sample heterogeneity. Once we identified top DMRs, we also examined their prognostic relevance in the TCGA patient data, as well as their performance in discriminating pre- and post-treatment plasma samples.

### a) Material and Methods

### (1) Patient Sample Collection

To detect HNSC-specific cfDNA biomarkers, we studied the plasma samples collected from a cohort of head and neck cancer patients treated at Moffitt Cancer Center (Tampa, USA). In this pilot study, a total of 16 matched plasma samples were collected from 8 patients before and at least 4 weeks after surgery. The basic clinical characteristics of these patients are displayed in Table 5. The study was approved by Institutional Review Board at Moffitt. All patients were consented to the protocol and all samples are de-identified during the methylation profiling process and in the downstream analysis.

TABLE 5
Baseline clinical characteristics and treatment of patients 481 included in the plasma cfMBD-seq analysis.

| | | Age | | | | | Tumor | DOI | | | LN | | Previous |
|---|---|---|---|---|---|---|---|---|---|---|---|---|---|
| Patient | Sex | range | Site | pT | pN | M | size (cm) | (mm) | PNI | LVI | deposit (cm) | ENE | treatment |
| P1 | Male | 56-60 | Mandible | 4a | 3b | 0 | 5.3 | 30 | Y | N | 2.0 | Y | Y |
| P2 | Male | 51-55 | Mandible | 4a | 3b | 0 | 4 | 28 | N | N | 2.5 | Y | N |
| P3 | Male | 56-60 | Tongue | 4a | 3b | 0 | 6.5 | 35 | Y | Y | 1.9 | Y | Y |
| P4 | Female | 65-70 | Maxilla | 1 | X | 0 | 0.3 | 1 | N | N | N/A | N/A | N |
| P5 | Male | 76-80 | Tongue | 3 | 1 | 0 | 3.6 | 14 | Y | N | 0.9 | N | N |
| P6 | Female | 41-45 | Tongue | 2 | 0 | 0 | 1.2 | 7 | Y | Y | N/A | N/A | N |
| P7 | Male | 76-80 | Tongue | 4a | 2a | 0 | 5.2 | 41 | Y | Y | 1.7 | Y | Y |
| P8 | Female | 76-80 | Buccal | 1 | 1 | 0 | 2 | 2 | N | N | 0.6 | N | N |
| | | | Mucosa | | | | | | | | | | |

**pN—pathologic nodal classification;
pT—pathologic tumor classification;
M—Metastasis;
PNI—Perineural invasion;
LVI—Lymphovascular invasion;
LN—Lymph Node;
ENE—Extranodal extension

### (2) TCGA-HNSC Analysis

To facilitate cfDNA methylation biomarker discovery, we first conducted a bioinformatics analysis to detect differentially methylated regions (DMRs) based on the tumor tissue methylation data collected from the TCGA-HNSC project. A total of 580 samples were profiled by the Illumina Infinium HumanMethylation450 BeadChip (450K array) in this cohort. Because the goal is to identify cancer-specific regions, our DMR analysis focuses on the 100 paired tumor and normal tissue methylation data collected from 50 patients. We downloaded level 3 DNA methylation data (beta value) from Broad Firehose web portal and all clinical data from GDC data portal. DMR analysis was performed using the bumphunter function implemented in the R package “minfi”, with the effect size cutoff set at 0.3 and the resampling number at 1000. We restricted downstream analyses on regions >5 bp in length, in which all regions contain at least two probes (L≥2). The detected regions were annotated against genome build UCSC hg19 by using the annotateDMRInfo function implemented in the “methyAnalysis” package.

(3) Plasma cfDNA Methylome Profiling by cfMBD-Seq

Cell-free DNA in plasma were profiled by cfMBD-seq, which is an enrichment-based ultra-low input cfDNA methylation profiling method recently developed by Moffitt. Briefly, Maxwell RSC ccf DNA Plasma kit was used to extract the cfDNA from 1 ml of plasma. If one sample contains less than 5 ng DNA, we extract DNA from another 1 ml plasma sample. We then combined the cfDNA from the first and second extraction (if needed for a patient sample) for the methylation enrichment and sequencing library preparation. Methylated DNA fragments were enriched and captured by using a MethylCap Kit. cfMBD libraries were prepared and quantified following the steps as described in the cfMBD-seq protocol, and sequenced by Illumina NextSeq 500/550 High Output Kit (75 cycles).

After fastq file merging and adapter trimming steps, sequence reads were aligned to hg19 assembly using BWA MEM (v0.7.10). Mapped reads were further sorted and filtered to remove low-quality and duplicated reads using samtools (v1.9) and picard tools (v1.82). R package “MEDIPS” was used to conduct coverage saturation analysis and downstream QC analysis. The “qsea” R package was used to calculate normalized methylation (beta) levels both at a genome-wide level and in targeted ROI regions (such as promoter regions). We applied fitNBglm function in “qsea” to perform the differential coverage analysis. The function fits a negative-binomial model for each genomic widow similar to the differential expression analysis function implemented in the package “edgeR”. To maximize the statistical power, the design matrix in the DMR analysis was formed in a paired DMR setting, in which an additive model formula is formed to include both treatment effect and subject effect. For statistical testing, a reduced model was fit by the function without the treatment term and the p-value is generated by comparing the likelihood ratio of the models against a Chi-square distribution. To remove potential noise regions with low coverage, we only consider regions (1 kb window) with a minimum of 50 reads in all the DMR tests.

(4) Prioritizing Candidate cfDNA Methylation Biomarkers

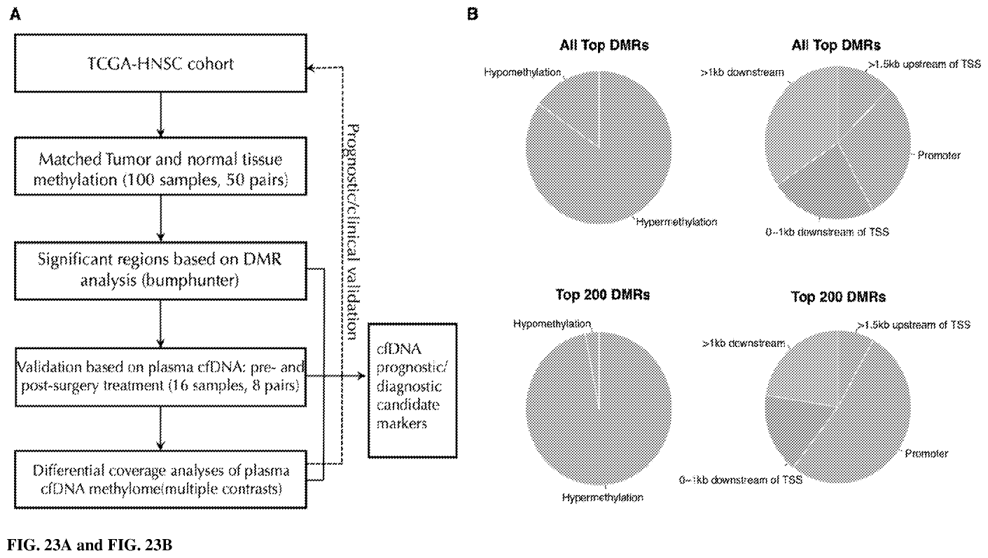

Because the current study has limited sample size and HNSC samples exhibit high heterogeneity in nature, we propose two schemes to efficiently prioritize the most robust cfDNA biomarker panels while minimizing the false discovery markers or ones with weak clinical significance. As illustrated in the FIG. 23A, the first biomarker discovery scheme only investigates DMRs that have been detected based on the analysis using the TCGA data. We hypothesize that the top cancer-specific DMRs detected based on the matched tumor-normal tissues will also exhibit differential methylation patterns between before- and after-surgery plasma samples. The stringent genome-wide multiple-testing correction is not required in this setting because it becomes a targeted biomarker validation analysis. In the second scheme, we perform genome-wide differential coverage analysis on cfDNA methylation data by only considering regions that are located in or nearby the promoter of known genes (defined as 5 kb upstream and 2 kb downstream of the TSS regions). Furthermore, as is explained more in the next section, we considered different patient subgroups for the pre- and post-treatment DMR analysis. In each test, regions with an adjusted p-value less than 0.1 or unadjusted p-values less than 1×10⁻⁶ are reported. Similar to the TCGA analysis, the detected regions were annotated using the “methyAnalysis” package. In summary, we reason that both schemes are useful in identifying promising targets that can be further tested as diagnostic and prognostic markers in managing HNSC patients.

### b) Results

### (1) Candidate Regions Based on TCGA-HSNC Analysis

The DMR analysis by comparing the TCGA-HNSC matched tumor and normal methylation profiles identified a total of 1468 significant regions (effect size cutoff of 0.3 and FWER adjusted p value<0.01) (Table 8). As shown in FIG. 23B, the majority (84.7%) of these top regions are hypermethylated DMRs; and more than half of regions are located in promoter (29.8%) or nearly (0-1 kb) downstream regions of TSS (23.5%). When we narrow the list to the top 200 DMRs only, the proportion of hypermethylated DMRs and DMRs in promoter region further increased to 97% and 52.5%, respectively. The average length of top 200 DMRs is 461 bp. The top five DMRs in the promoter region are located in genes MARCHF11, ZNF154, ELMO1, ADCYAPI1 and PIEZ02. A summary of top DMRs and their associated genes is provided in Table 6. Interestingly, we observed that many zinc-finger genes were enriched in the top DMR list, to only list those in the top 100 list: ZNF154, ZNF582, ZNF135, ZNF136, ZNF577, ZNF781, ZNF529, NF132, ZNF85, ZNF583, ZNF471, and ZNF665.

TABLE 6
Table of top genes containing significant DMRs based on TCGA-
HNSC matched tumor and normal tissue methylation profiles

| Rank Methods | Top genes* |
|---|---|
| Genes contain top 100 DMRs | MARCHF11, ZNF154, ELMO1, ADCYAP1, PIEZO2, CCDC181, |
| (promoter regions) | KCNA3, OTX2-AS1, PCDHGC4, DPP10-AS1, MIR129-2, ZNF582, |
| | WT1, DRD5, CBLN4, DPP6, SORCS3, ZNF135, GALR1, BOLL, |
| | ZSCAN18, BARHL2, ZNF577, CLVS2, ABCC9, INSC, GRM6, |
| | ZNF781, ZNF529, TBXT, ITGA8, GCSAML, CDO1, ZNF132, IRF8, |
| | NKX2-6, ZC4H2, ZSCAN1, NKAPL, NPY, PENK, ZNF85, |
| | ADAMTS16, EVX2, NETO1, ZNF583, VAX1, HOXD9, KLHL34, |
| | CFTR, PCSK1, ZNF471, ZIK1, SHISA3, SIX6, ZNF665 |
| Genes contain top 100 DMRs | HOXD9, TRH, GRIA4, ZNF542P, PAX6, GSC, NTM, PPFIA3, |
| (1 kb downstream of TSS) | PABPC5, SFTA3, NID2, HOXA11-AS, ASCL1, MAGI2-AS3, |
| | LOC100289656 |
| Genes contain top 100 DMRs | EN1, SOX17, LHX1, PANTR1, NXPH1, ZNF136, LINC00461, |
| (other regions) | PAX2, SEPTIN9, VAX1, ZIC4, HOTAIR, UBD, GBX2, ZNF559- |
| | ZNF177, NKX2-2, SOX1, SOX11, GSC, LINC01304, LINC01623, |
| | PITX2, PCDHA13, RMCX5-GPRASP2 |
| Top genes ranked by number of | ZIC4 (11), ZIC1 (9), EN1(8), SOX1(8), TBX5-AS1(8), SOX17(7), |
| DMRs | BARHL2(6), ZBED9(6), EVX2(5), HOXB3(5), HOXD10(5), |
| (>3 DMRs; No. of DMRs | LINC00461(5), OTX2(5), PAX2(5), SOX3(5), VAX1(5), HOTAIR(4), |
| indicated in parentheses; Genes | HOXA3(4), HOXC4(4), HOXC6(4), LHX8(4), MIR124-2HG(4), |
| containing top 100 DMRs are | MKI67(4), NXPH1(4), ONECUT2(4), PAX6(6), PAX6-AS1(4), |
| highlighted in bold) | PDX1(4), PITX2(4), PTF1A(4), PTPRN2(4), TBX15(4), TLX1(4), |
| | TLX3(4), UBD(4) |

*Full list of DMRs and their locations are listed in Supplementary Table 8.

TABLE 8
Significant DMRs identified in the TCGA HNSC data (matched tumor and normal tissues)

| chr | start | end | value | area | cluster | width | EntrezID | GeneSymbol | distance 2TSS | nearestTx | PROMOTER |
|---|---|---|---|---|---|---|---|---|---|---|---|
| chr2 | 119614489 | 119617128 | 0.391010998 | 4.692131982 | 110890 | 2640 | 2019 | EN1 | −8730 | uc002tlm.3 | FALSE |
| chr5 | 16179873 | 16180266 | 0.446688581 | 4.466885806 | 151021 | 394 | 441061 | MARCHF11 | 0 | uc003jfo.2 | TRUE |
| chr19 | 58220295 | 58220837 | 0.433658989 | 4.336589889 | 102591 | 543 | 7710 | ZNF154 | 0 | uc010euf.3 | TRUE |
| chr2 | 176987918 | 176989485 | 0.427516449 | 4.275164495 | 114073 | 1568 | 3235 | HOXD9 | 505 | uc010zex.2 | FALSE |
| chr7 | 37488162 | 37488671 | 0.371416939 | 3.714169394 | 176083 | 510 | 9844 | ELMO1 | 0 | uc003tfk.2 | TRUE |
| chr18 | 904523 | 905156 | 0.410016476 | 3.69014828 | 91207 | 634 | 116 | ADCYAP1 | 0 | uc010dkg.3 | TRUE |
| chr18 | 11148769 | 11149763 | 0.398565239 | 3.587087153 | 91500 | 995 | 63895 | PIEZO2 | −8 | uc002kos.2 | TRUE |
| chr1 | 169396635 | 169396868 | 0.433984767 | 3.471878137 | 14588 | 234 | 57821 | CCDC181 | 0 | uc001ggc.1 | TRUE |
| chr3 | 129693370 | 129694156 | 0.424105124 | 3.392840993 | 136029 | 787 | 7200 | TRH | 134 | uc003enc.4 | FALSE |
| chr1 | 111217194 | 111217785 | 0.423458348 | 3.387666785 | 11100 | 592 | 3738 | KCNA3 | 0 | uc001dzv.1 | TRUE |
| chr14 | 57278084 | 57279496 | 0.421513453 | 3.372107623 | 60073 | 1413 | 100309464 | OTX2-AS1 | 0 | uc021rtn.1 | TRUE |
| chr5 | 140864020 | 140864834 | 0.367914621 | 3.311231593 | 156947 | 815 | 56098 | PCDHGC4 | 0 | uc003lky.2 | TRUE |
| chr11 | 105480979 | 105481509 | 0.366929308 | 3.302363773 | 39697 | 531 | 2893 | GRIA4 | 179 | uc009yxk.1 | FALSE |
| chr19 | 56879554 | 56879933 | 0.460284617 | 3.221992321 | 102402 | 380 | 147947 | ZNF542P | 64 | uc002qmr.3 | FALSE |
| chr8 | 55379034 | 55380185 | 0.399099169 | 3.192793355 | 189191 | 1152 | 64321 | SOX17 | 8539 | uc003xsb.4 | FALSE |
| chr2 | 115918745 | 115920221 | 0.398426896 | 3.187415171 | 110777 | 1477 | 389023 | DPP10-AS1 | 0 | uc002tld.3 | TRUE |
| chr17 | 35299270 | 35300874 | 0.384863095 | 3.07890476 | 84711 | 1605 | 3975 | LHX1 | 4498 | uc010cux.1 | FALSE |
| chr11 | 43602845 | 43602965 | 0.438984362 | 3.072890532 | 34315 | 121 | 406918 | MIR129-2 | 0 | uc001mxo.1 | TRUE |
| chr19 | 56904901 | 56905032 | 0.430902544 | 3.016317809 | 102405 | 132 | 147948 | ZNF582 | −7 | uc002qmy.3 | TRUE |
| chr11 | 32456910 | 32457257 | 0.37141416 | 2.971313281 | 33812 | 348 | 7490 | WT1 | 0 | uc009yjs.2 | TRUE |
| chr4 | 9783192 | 9783398 | 0.423150669 | 2.962054684 | 142057 | 207 | 1816 | DRD5 | 0 | uc003gmb.4 | TRUE |
| chr20 | 54578898 | 54580677 | 0.36932061 | 2.954564881 | 121752 | 1780 | 140689 | CBLN4 | 0 | uc002xxa.4 | TRUE |
| chr7 | 153584416 | 153584873 | 0.419044131 | 2.933308915 | 183949 | 458 | 1804 | DPP6 | 0 | uc003wli.3 | TRUE |
| chr2 | 105459164 | 105461096 | 0.418428267 | 2.928997869 | 110027 | 1933 | 100506421 | PANTR1 | 6838 | uc002tck.2 | FALSE |
| chr7 | 8480865 | 8482107 | 0.416542113 | 2.915794791 | 174366 | 1243 | 30010 | NXPH1 | 7280 | uc011jxh.2 | FALSE |
| chr10 | 106400667 | 106401479 | 0.416378263 | 2.914647843 | 27988 | 813 | 22986 | SORCS3 | 0 | uc001kyi.1 | TRUE |
| chr19 | 58570419 | 58570790 | 0.412109429 | 2.884766006 | 102660 | 372 | 7694 | ZNF135 | 0 | uc021vcu.1 | TRUE |
| chr19 | 12305553 | 12306497 | 0.407451491 | 2.852160436 | 96492 | 945 | 7695 | ZNF136 | 31681 | uc010xmh.2 | FALSE |
| chr18 | 74961966 | 74962794 | 0.393523197 | 2.754662381 | 93064 | 829 | 2587 | GALR1 | 0 | uc002lms.4 | TRUE |
| chr2 | 119609557 | 119610526 | 0.449628675 | 2.697772052 | 110888 | 970 | 2019 | EN1 | −3798 | uc002tlm.3 | FALSE |
| chr2 | 198650880 | 198651498 | 0.447823549 | 2.686941291 | 114990 | 619 | 66037 | BOLL | 0 | uc010zha.1 | TRUE |
| chr5 | 87974369 | 87974547 | 0.383012043 | 2.681084302 | 154019 | 179 | 645323 | LINC00461 | 6073 | uc011cub.2 | FALSE |
| chr19 | 58609361 | 58609770 | 0.381286235 | 2.669003644 | 102669 | 410 | 65982 | ZSCAN18 | 0 | uc002qrl.2 | TRUE |
| chr1 | 91182989 | 91184413 | 0.379542331 | 2.65679632 | 10123 | 1425 | 343472 | BARHL2 | −195 | uc001dns.3 | TRUE |
| chr19 | 52391078 | 52391304 | 0.374458042 | 2.621206291 | 101551 | 227 | 84765 | ZNF577 | 0 | uc010yde.2 | TRUE |
| chr11 | 31827084 | 31827920 | 0.374227702 | 2.619593914 | 33763 | 837 | 5080 | PAX6 | 90 | uc021qfn.1 | FALSE |
| chr6 | 123317408 | 123317875 | 0.372020394 | 2.604142756 | 168809 | 468 | 134829 | CLVS2 | 0 | uc003pzi.1 | TRUE |
| chr10 | 102419209 | 102419617 | 0.424969949 | 2.549819696 | 27376 | 409 | 5076 | PAX2 | −85851 | uc001krk.4 | FALSE |
| chr12 | 22094563 | 22095330 | 0.362376906 | 2.536638341 | 44379 | 768 | 10060 | ABCC9 | −227 | uc001rfk.3 | TRUE |
| chr17 | 75368902 | 75369224 | 0.420519012 | 2.523114071 | 89464 | 323 | 10801 | SEPTIN9 | 53305 | uc002jtv.3 | FALSE |
| chr11 | 15136150 | 15136505 | 0.357569919 | 2.502989432 | 32947 | 356 | 387755 | INSC | 0 | uc001mlz.4 | TRUE |
| chr14 | 95234658 | 95235127 | 0.356762516 | 2.497337615 | 62828 | 470 | 145258 | GSC | 1372 | uc001ydu.3 | FALSE |
| chr5 | 178421711 | 178422260 | 0.415566924 | 2.493401545 | 159874 | 550 | 2916 | GRM6 | 0 | uc003mjr.3 | TRUE |
| chr10 | 118892211 | 118893174 | 0.414221199 | 2.485327196 | 28617 | 964 | 11023 | VAX1 | 4638 | uc009xyx.3 | FALSE |
| chr19 | 38183055 | 38183262 | 0.410305457 | 2.461832743 | 98899 | 208 | 163115 | ZNF781 | 0 | uc002ogz.2 | TRUE |
| chr19 | 37096010 | 37096487 | 0.409598332 | 2.457589991 | 98791 | 478 | 57711 | ZNF529 | 0 | uc002oej.3 | TRUE |
| chr11 | 31825756 | 31826574 | 0.409490914 | 2.456945482 | 33762 | 819 | 5080 | PAX6 | 1436 | uc031pzk.1 | FALSE |
| chr3 | 147106010 | 147106890 | 0.408612204 | 2.451673227 | 136996 | 881 | 84107 | ZIC4 | 3327 | uc021xfe.1 | FALSE |
| chr6 | 166582188 | 166582393 | 0.406016551 | 2.436099305 | 171569 | 206 | 6862 | TBXT | −31 | uc003quu.2 | TRUE |
| chr12 | 54354590 | 54355528 | 0.405265016 | 2.431590095 | 46501 | 939 | 100124700 | HOTAIR | 7012 | uc010soq.2 | FALSE |
| chr10 | 15761854 | 15762091 | 0.392961783 | 2.357770698 | 21893 | 238 | 8516 | ITGA8 | −84 | uc001ioc.1 | TRUE |
| chr11 | 131780329 | 131780492 | 0.391256328 | 2.347537967 | 42115 | 164 | 50863 | NTM | 829 | uc001qgo.3 | FALSE |
| chr6 | 29521598 | 29521695 | 0.384184047 | 2.305104282 | 163140 | 98 | 10537 | UBD | 6007 | uc003nmo.3 | FALSE |
| chr1 | 247712110 | 247712383 | −0.378761162 | 2.272566975 | 20169 | 274 | 148823 | GCSAML | 0 | uc001idf.3 | TRUE |
| chr5 | 115152420 | 115152785 | 0.377428393 | 2.264570357 | 155076 | 366 | 1036 | CDO1 | −15 | uc003krg.3 | TRUE |
| chr2 | 237078223 | 237078733 | 0.449499772 | 2.247498859 | 117855 | 511 | 2637 | GBX2 | −1571 | uc002vvw.1 | TRUE |
| chr19 | 58951599 | 58951885 | 0.370409292 | 2.222455751 | 102743 | 287 | 7691 | ZNF132 | −10 | uc002qst.4 | TRUE |
| chr16 | 85932214 | 85932853 | 0.370226774 | 2.221360642 | 79233 | 640 | 3394 | IRF8 | 0 | uc002fjh.3 | TRUE |
| chr19 | 49646093 | 49646246 | 0.444111398 | 2.220556988 | 100960 | 154 | 8541 | PPFIA3 | 32 | uc002pmt.3 | FALSE |
| chr8 | 23564025 | 23564591 | 0.36955702 | 2.217342122 | 187319 | 567 | 137814 | NKX2-6 | 0 | uc011kzy.3 | TRUE |
| chr19 | 9473598 | 9473691 | 0.442263368 | 2.211316842 | 95963 | 94 | 100529215 | ZNF559-ZNF177 | 38227 | uc002mlk.3 | FALSE |
| chrX | 90689603 | 90690118 | 0.440433294 | 2.202166471 | 202824 | 516 | 140886 | PABPC5 | 6 | uc004efg.3 | FALSE |
| chr20 | 21502728 | 21503898 | 0.366916762 | 2.201500574 | 119992 | 1171 | 4821 | NKX2-2 | −8064 | uc002wsi.3 | FALSE |
| chr13 | 112759355 | 112760088 | 0.439606829 | 2.198034143 | 57403 | 734 | 6656 | SOX1 | 37442 | uc001vsb.1 | FALSE |
| chr2 | 5836231 | 5837002 | 0.431408175 | 2.157040876 | 103482 | 772 | 6664 | SOX11 | 3432 | uc002qyj.3 | FALSE |
| chr14 | 36986363 | 36987408 | 0.43135415 | 2.156770752 | 59159 | 1046 | 253970 | SFTA3 | 1409 | uc001wts.3 | FALSE |
| chrX | 64254409 | 64255215 | 0.359077408 | 2.154464446 | 202298 | 807 | 55906 | ZC4H2 | 0 | uc022byd.1 | TRUE |
| chr19 | 58545122 | 58545285 | 0.430049128 | 2.150245641 | 102650 | 164 | 284312 | ZSCAN1 | −149 | uc002qra.1 | TRUE |
| chr6 | 28227068 | 28227127 | 0.35783996 | 2.147039762 | 162921 | 60 | 222698 | NKAPL | 0 | uc003nkt.4 | TRUE |
| chr7 | 24323764 | 24323939 | 0.356378754 | 2.138272526 | 174999 | 176 | 4852 | NPY | 0 | uc003sww.2 | TRUE |
| chr8 | 57358240 | 57358713 | 0.353489984 | 2.120939905 | 189306 | 474 | 5179 | PENK | 0 | uc003xsz.2 | TRUE |
| chr19 | 21106002 | 21106053 | 0.351352215 | 2.108113289 | 97948 | 52 | 7639 | ZNF85 | −6 | uc002npf.4 | TRUE |
| chr5 | 5139853 | 5140001 | 0.351321971 | 2.107931825 | 150397 | 149 | 170690 | ADAMTS16 | −442 | uc003jdj.1 | TRUE |
| chr2 | 176948693 | 176948759 | 0.421244028 | 2.106220142 | 114055 | 67 | 344191 | EVX2 | −3 | uc010zeu.2 | TRUE |
| chr14 | 95239381 | 95240143 | 0.343807988 | 2.062847926 | 62830 | 763 | 145258 | GSC | −2882 | uc001ydu.3 | FALSE |
| chr14 | 52534596 | 52535758 | 0.409092054 | 2.045460269 | 59679 | 1163 | 22795 | NID2 | 188 | uc001wzp.3 | FALSE |
| chr18 | 70534298 | 70535406 | 0.335995455 | 2.01597273 | 92879 | 1109 | 81832 | NETO1 | 0 | uc002lky.2 | TRUE |
| chr19 | 56915650 | 56915732 | 0.400626174 | 2.003130872 | 102408 | 83 | 147949 | ZNF583 | 0 | uc010ygm.1 | TRUE |
| chr10 | 118899024 | 118899788 | 0.400499723 | 2.002498616 | 28619 | 765 | 11023 | VAX1 | −1212 | uc001ldb.1 | TRUE |
| chr2 | 119599744 | 119600348 | 0.398492514 | 1.992462569 | 110885 | 605 | 2019 | EN1 | 5411 | uc002tlm.3 | FALSE |
| chr7 | 27225143 | 27225448 | 0.331599566 | 1.989597393 | 175244 | 306 | 221883 | HOXA11-AS | 116 | uc003syz.1 | FALSE |
| chr2 | 4050695 | 4050845 | −0.397485457 | 1.987427286 | 103428 | 151 | 100505964 | LINC01304 | −29073 | uc002qyg.4 | FALSE |
| chr12 | 103352235 | 103352454 | 0.393846179 | 1.969230896 | 49167 | 220 | 429 | ASCL1 | 783 | uc001tjr.4 | FALSE |
| chr2 | 176987383 | 176987465 | 0.39336292 | 1.966814599 | 114073 | 83 | 3235 | HOXD9 | 0 | uc010zex.2 | TRUE |
| chrX | 21676344 | 21676605 | 0.39311591 | 1.965579548 | 201125 | 262 | 257240 | KLHL34 | 0 | uc004czz.1 | TRUE |
| chr7 | 117119393 | 117119637 | 0.391657072 | 1.958285362 | 181225 | 245 | 1080 | CFTR | −380 | uc003vjd.3 | TRUE |
| chr7 | 79083408 | 79083509 | 0.391262665 | 1.956313326 | 178817 | 102 | 100505881 | MAGI2-AS3 | 132 | uc022ags.1 | FALSE |
| chr5 | 95768915 | 95769025 | 0.391057443 | 1.955287217 | 154369 | 111 | 5122 | PCSK1 | 0 | uc003kls.2 | TRUE |
| chr19 | 57019005 | 57019279 | 0.390082372 | 1.950411859 | 102414 | 275 | 57573 | ZNF471 | 0 | uc002qnh.3 | TRUE |
| chr15 | 29033881 | 29033901 | 0.389257493 | 1.946287466 | 65306 | 21 | 100289656 | LOC100289656 | 492 | uc021sgs.1 | FALSE |
| chr6 | 28778091 | 28778382 | 0.387095835 | 1.935479173 | 163032 | 292 | 401242 | LINC01623 | 53072 | uc003nlq.2 | FALSE |
| chr20 | 25062777 | 25062860 | 0.386872955 | 1.934364777 | 120136 | 84 | 30813 | VSX1 | 155 | uc002wug.2 | FALSE |
| chr19 | 58095581 | 58095659 | 0.482390181 | 1.929560722 | 102564 | 79 | 284307 | ZIK1 | 0 | uc002qpj.3 | TRUE |
| chr4 | 111550040 | 111550830 | 0.385309326 | 1.926546632 | 146011 | 791 | 5308 | PITX2 | 12449 | uc003iac.3 | FALSE |
| chr6 | 29521751 | 29521788 | 0.384803015 | 1.924015076 | 163140 | 38 | 10537 | UBD | 5914 | uc003nmo.3 | FALSE |
| chr5 | 140306181 | 140306231 | 0.384078957 | 1.920394785 | 156791 | 51 | 56136 | PCDHA13 | 44327 | uc003lig.1 | FALSE |
| chr4 | 42399792 | 42399858 | 0.383717073 | 1.918585363 | 143362 | 67 | 152573 | SHISA3 | 0 | uc003gwp.3 | TRUE |
| chrX | 101906035 | 101906181 | 0.383497459 | 1.917487296 | 202988 | 147 | 100528062 | ARMCX5-GPRASP2 | 51759 | uc004eji.4 | FALSE |
| chr14 | 60975846 | 60976285 | 0.377982711 | 1.889913555 | 60297 | 440 | 4990 | SIX6 | 0 | uc001xfa.4 | TRUE |
| chr19 | 53696569 | 53696649 | 0.471943641 | 1.887774564 | 101730 | 81 | 79788 | ZNF665 | 0 | uc010eqm.1 | TRUE |
| chr17 | 46655289 | 46655561 | 0.377294977 | 1.886474886 | 86619 | 273 | 3214 | HOXB4 | 182 | uc002inp.3 | FALSE |
| chr21 | 34398199 | 34398265 | 0.377220545 | 1.886102726 | 123279 | 67 | 10215 | OLIG2 | 0 | uc002yqx.2 | TRUE |
| chr2 | 98963582 | 98964436 | 0.377021075 | 1.885105375 | 109546 | 855 | 1261 | CNGA3 | 964 | uc002syu.3 | FALSE |
| chr2 | 119606695 | 119607395 | 0.376991664 | 1.884958318 | 110886 | 701 | 2019 | EN1 | −936 | uc002tlm.3 | TRUE |
| chr7 | 97361241 | 97361408 | 0.470893558 | 1.883574233 | 179579 | 168 | 6863 | TAC1 | 0 | uc003uos.4 | TRUE |
| chr8 | 65290109 | 65291287 | 0.374666747 | 1.873333734 | 189646 | 1179 | 100130155 | MIR124-2HG | 4334 | uc003xvf.3 | FALSE |
| chr8 | 101118083 | 101118548 | 0.372171072 | 1.86085536 | 191280 | 466 | 26166 | RGS22 | 0 | uc010mbo.1 | TRUE |
| chr10 | 129534613 | 129535378 | 0.370722354 | 1.85361177 | 29603 | 766 | 399823 | FOXI2 | −160 | uc009yas.2 | TRUE |
| chr1 | 91194674 | 91195336 | 0.370123197 | 1.850615987 | 10127 | 663 | 343472 | BARHL2 | −11880 | uc001dns.3 | FALSE |
| chr8 | 72917147 | 72917695 | 0.369227411 | 1.846137053 | 190029 | 549 | 100132891 | MSC-AS1 | 160796 | uc003xza.3 | FALSE |
| chr6 | 62995876 | 62996130 | 0.368909748 | 1.844548739 | 166531 | 255 | 202559 | KHDRBS2 | 0 | uc003peg.2 | TRUE |
| chr13 | 84456127 | 84456486 | 0.366045707 | 1.830228534 | 55976 | 360 | 114798 | SLITRK1 | 42 | uc001vlk.3 | FALSE |
| chr4 | 11428822 | 11429531 | 0.363869736 | 1.819348682 | 142157 | 710 | 9957 | HS3ST1 | 1006 | uc003gmq.3 | FALSE |
| chr6 | 100911687 | 100911727 | 0.454009208 | 1.816036833 | 167735 | 41 | 6492 | SIM1 | 84 | uc021zdg.1 | FALSE |
| chr4 | 8395941 | 8396410 | 0.360382095 | 1.801910477 | 141930 | 470 | 8310 | ACOX3 | 33798 | uc010idk.3 | FALSE |
| chr7 | 27145972 | 27146445 | 0.359423964 | 1.797119822 | 175204 | 474 | 3200 | HOXA3 | 12769 | uc003syh.3 | FALSE |
| chr19 | 53635967 | 53636229 | 0.358241027 | 1.791205136 | 101718 | 263 | 55786 | ZNF415 | 0 | uc002qba.3 | TRUE |
| chr13 | 112760898 | 112761184 | 0.356731818 | 1.783659092 | 57403 | 287 | 6656 | SOX1 | 38985 | uc001vsb.1 | FALSE |
| chr1 | 63785321 | 63785946 | 0.44505007 | 1.780200282 | 8891 | 626 | 199899 | LINC00466 | −2420 | uc001daw.2 | FALSE |
| chr16 | 55690378 | 55690564 | 0.444850813 | 1.779403253 | 76551 | 187 | 6530 | SLC6A2 | 26 | uc021tio.1 | FALSE |
| chr3 | 27764810 | 27765409 | 0.355804419 | 1.779022093 | 129844 | 600 | 8320 | EOMES | −604 | uc003cdx.4 | TRUE |
| chr7 | 149389669 | 149389941 | 0.444730605 | 1.778922421 | 183370 | 273 | 84626 | KRBA1 | −22161 | uc010lpj.3 | FALSE |
| chr20 | 11898478 | 11898851 | 0.438986534 | 1.755946137 | 119731 | 374 | 22903 | BTBD3 | 0 | uc002wnz.3 | TRUE |
| chr16 | 22825769 | 22826243 | 0.350225321 | 1.751126607 | 74680 | 475 | 9956 | HS3ST2 | 0 | uc002dli.3 | TRUE |
| chr6 | 105400884 | 105401186 | 0.437663354 | 1.750653414 | 167815 | 303 | 389421 | LIN28B | −3737 | uc003pqv.2 | FALSE |
| chr8 | 65291682 | 65292217 | 0.349585174 | 1.747925868 | 189646 | 536 | 406908 | MIR124-2 | 0 | uc003xvf.3 | TRUE |
| chr2 | 79220029 | 79220510 | 0.436195047 | 1.744780186 | 108588 | 482 | 130120 | REG3G | −32302 | uc002snw.4 | FALSE |
| chrX | 139587304 | 139587372 | 0.435969217 | 1.743876866 | 204028 | 69 | 6658 | SOX3 | −79 | uc004fbd.1 | TRUE |
| chr8 | 133573739 | 133574073 | −0.348387564 | 1.741937819 | 192971 | 335 | 93668 | HPYR1 | −13 | uc011liz.1 | TRUE |
| chr19 | 20348926 | 20349275 | 0.435319051 | 1.741276205 | 97935 | 350 | 90649 | ZNF486 | 70903 | uc002nou.3 | FALSE |
| chr2 | 176980837 | 176981328 | 0.434593837 | 1.738375348 | 114070 | 492 | 3236 | HOXD10 | −164 | uc002ukj.3 | TRUE |
| chr2 | 124782253 | 124782713 | 0.347291419 | 1.736457095 | 111256 | 461 | 129684 | CNTNAP5 | −151 | uc002tno.3 | TRUE |
| chr6 | 24358236 | 24358327 | 0.347164437 | 1.735822185 | 162389 | 92 | 51473 | DCDC2 | 0 | uc003ndx.3 | TRUE |
| chr19 | 13135527 | 13136168 | 0.433587911 | 1.734351645 | 96689 | 642 | 4784 | NFIX | 132 | uc002mwg.2 | FALSE |
| chr2 | 228736253 | 228736357 | 0.432855595 | 1.731422379 | 117023 | 105 | 164781 | DAW1 | 0 | uc010zlw.1 | TRUE |
| chr7 | 145813417 | 145813439 | 0.345629856 | 1.72814928 | 183163 | 23 | 26047 | CNTNAP2 | −14 | uc003weu.2 | TRUE |
| chr7 | 29605808 | 29606349 | 0.34515619 | 1.725780948 | 175474 | 542 | 222171 | PRR15 | 2381 | uc003tac.1 | FALSE |
| chr19 | 15580400 | 15580721 | 0.430117546 | 1.720470184 | 97088 | 322 | 114770 | PGLYRP2 | 9594 | uc002nbe.2 | FALSE |
| chr11 | 20690628 | 20690930 | 0.3431303 | 1.7156515 | 33430 | 303 | 4745 | NELL1 | −187 | uc001mqe.3 | TRUE |
| chr5 | 140787504 | 140787864 | 0.428835716 | 1.715342864 | 156920 | 361 | 56100 | PCDHGB6 | 0 | uc003lkj.2 | TRUE |
| chr13 | 96204870 | 96205295 | 0.342417 | 1.712085001 | 56212 | 426 | 9071 | CLDN10 | 0 | uc001vmh.2 | TRUE |
| chr19 | 22034418 | 22034799 | 0.420597637 | 1.682390547 | 98000 | 382 | 7594 | ZNF43 | 71 | uc031rkb.1 | FALSE |
| chr14 | 57261256 | 57262177 | 0.335258606 | 1.676293029 | 60065 | 922 | 5015 | OTX2 | 10204 | uc031qor.1 | FALSE |
| chr6 | 29521138 | 29521162 | 0.334519851 | 1.672599257 | 163140 | 25 | 10537 | UBD | 6540 | uc003nmo.3 | FALSE |
| chr18 | 10588980 | 10589357 | −0.416519295 | 1.66607718 | 91486 | 378 | 8774 | NAPG | 49859 | uc002kop.3 | FALSE |
| chr7 | 27192056 | 27192339 | 0.416390218 | 1.665560873 | 175224 | 284 | 100133311 | HOXA-AS3 | 5274 | uc003sys.3 | FALSE |
| chr6 | 28602543 | 28602626 | 0.333078967 | 1.665394836 | 163001 | 84 | 114821 | ZBED9 | −47431 | uc003nlo.3 | FALSE |
| chr3 | 179754529 | 179754615 | 0.330207379 | 1.651036894 | 138557 | 87 | 51555 | PEX5L | 226 | uc011bqh.2 | FALSE |
| chr19 | 15121591 | 15122142 | 0.326187175 | 1.630935875 | 96983 | 552 | 126402 | CCDC105 | 52 | uc002nae.2 | FALSE |
| chr13 | 53422691 | 53422808 | 0.407628262 | 1.630513047 | 55247 | 118 | 5100 | PCDH8 | 0 | uc001vhj.3 | TRUE |
| chr11 | 40314978 | 40315404 | 0.407447015 | 1.629788059 | 34233 | 427 | 57689 | LRRC4C | 1165782 | uc001mxb.2 | FALSE |
| chr4 | 122686432 | 122686493 | 0.407370104 | 1.629480418 | 146422 | 62 | 100192379 | PP12613 | 692 | uc003idx.1 | FALSE |
| chr19 | 13616871 | 13617094 | 0.405436846 | 1.621747385 | 96757 | 224 | 773 | CACNA1A | 180 | uc021upt.1 | FALSE |
| chr19 | 40314862 | 40315011 | 0.403860335 | 1.61544134 | 99243 | 150 | 9149 | DYRK1B | 9830 | uc002oml.3 | FALSE |
| chr6 | 28956226 | 28956268 | 0.403435502 | 1.613742007 | 163077 | 43 | 282890 | ZNF311 | 15723 | uc011dlk.1 | FALSE |
| chr19 | 53496440 | 53496738 | 0.401991763 | 1.607967051 | 101702 | 299 | 79986 | ZNF702P | 46 | uc002qan.4 | FALSE |
| chr1 | 197888469 | 197888894 | 0.396862527 | 1.587450106 | 15925 | 426 | 56956 | LHX9 | 1952 | uc001guk.1 | FALSE |
| chr2 | 47797133 | 47797590 | 0.396376308 | 1.585505232 | 106680 | 458 | 56660 | KCNK12 | 0 | uc002rwb.3 | TRUE |
| chr1 | 34642396 | 34642609 | 0.396286449 | 1.585145797 | 6125 | 214 | 84970 | C1orf94 | 0 | uc001bxt.3 | TRUE |
| chr19 | 53757910 | 53758289 | 0.396068176 | 1.584272703 | 101734 | 380 | 342926 | ZNF677 | 0 | uc002qbh.3 | TRUE |
| chr12 | 128751460 | 128752058 | 0.395288531 | 1.581154125 | 51729 | 599 | 92293 | TMEM132C | 0 | uc021rgn.1 | TRUE |
| chr2 | 80530255 | 80530770 | 0.393968456 | 1.575873824 | 108656 | 516 | 347730 | LRRTM1 | 717 | uc002sok.1 | FALSE |
| chr19 | 53073309 | 53073404 | 0.392879603 | 1.571518414 | 101637 | 96 | 55762 | ZNF701 | −122 | uc002pzs.2 | TRUE |
| chr14 | 52536066 | 52536436 | 0.391108835 | 1.564435341 | 59679 | 371 | 22795 | NID2 | −120 | uc001wzo.3 | TRUE |
| chr8 | 55370579 | 55371369 | 0.390361682 | 1.561446729 | 189187 | 791 | 64321 | SOX17 | 84 | uc003xsb.4 | FALSE |
| chr6 | 133562470 | 133562492 | 0.312027516 | 1.560137579 | 169283 | 23 | 2070 | EYA4 | 734 | uc011ecr.2 | FALSE |
| chr7 | 121956643 | 121956890 | 0.390007657 | 1.560030627 | 181377 | 248 | 93664 | CADPS2 | 304881 | uc003vkc.3 | FALSE |
| chr17 | 3438857 | 3439042 | 0.389595586 | 1.558382343 | 81343 | 186 | 162514 | TRPV3 | 0 | uc002fvs.2 | TRUE |
| chr6 | 27235822 | 27235891 | 0.519317456 | 1.557952369 | 162762 | 70 | 10279 | PRSS16 | 16367 | uc003njd.3 | FALSE |
| chr12 | 43945680 | 43946284 | 0.387630852 | 1.550523408 | 45284 | 605 | 80070 | ADAMTS20 | 0 | uc010skx.2 | TRUE |
| chr7 | 27197555 | 27197879 | 0.387604737 | 1.550418946 | 175227 | 325 | 3204 | HOXA7 | −1259 | uc003sys.3 | TRUE |
| chr3 | 187387999 | 187388225 | 0.387217243 | 1.548868973 | 139208 | 227 | 6750 | SST | 0 | uc003frn.3 | TRUE |
| chr6 | 28956332 | 28956426 | 0.386316562 | 1.545266246 | 163077 | 95 | 282890 | ZNF311 | 15565 | uc011dlk.1 | FALSE |
| chr7 | 15726821 | 15727525 | 0.385938191 | 1.543752763 | 174517 | 705 | 4223 | MEOX2 | −513 | uc003stc.3 | TRUE |
| chr18 | 55019708 | 55020099 | 0.384250371 | 1.537001482 | 92574 | 392 | 51046 | ST8SIA3 | 0 | uc002lgn.3 | TRUE |
| chr12 | 113913695 | 113914222 | 0.382924665 | 1.531698659 | 50193 | 528 | 64211 | LHX5 | −3818 | uc001tvj.1 | FALSE |
| chr16 | 51184355 | 51184562 | 0.381662022 | 1.526648089 | 76312 | 208 | 6299 | SALL1 | 0 | uc021tid.1 | TRUE |
| chr14 | 36973204 | 36973691 | 0.381244278 | 1.524977114 | 59152 | 488 | 253970 | SFTA3 | 9299 | uc001wtr.3 | FALSE |
| chr10 | 130339527 | 130339677 | 0.381086111 | 1.524344445 | 29687 | 151 | 4288 | MKI67 | −415059 | uc001lke.3 | FALSE |
| chr10 | 25464418 | 25464719 | 0.380721366 | 1.522885464 | 22364 | 302 | 57512 | GPR158 | 128 | uc001isj.3 | FALSE |
| chr19 | 20278013 | 20278059 | 0.380068096 | 1.520272383 | 97934 | 47 | 90649 | ZNF486 | 0 | uc002nou.3 | TRUE |
| chr10 | 729204 | 729479 | −0.380012473 | 1.520049891 | 20479 | 276 | 22982 | DIP2C | 6129 | uc001ifp.3 | FALSE |
| chr7 | 8473279 | 8473462 | 0.379368475 | 1.5174739 | 174362 | 184 | 30010 | NXPH1 | −123 | uc003srv.3 | TRUE |
| chr6 | 30130819 | 30131001 | −0.504939314 | 1.514817941 | 163337 | 183 | 89870 | TRIM15 | 0 | uc010jrx.3 | TRUE |
| chr19 | 39754974 | 39755901 | 0.378516335 | 1.514065338 | 99147 | 928 | 282616 | IFNL2 | −3256 | uc002oku.1 | FALSE |
| chr14 | 29235123 | 29235376 | 0.377162523 | 1.508650091 | 58843 | 254 | 2290 | FOXG1 | −902 | uc001wqe.4 | TRUE |
| chr11 | 69634296 | 69634372 | 0.376745405 | 1.50698162 | 37434 | 77 | 2248 | FGF3 | −104 | uc001oph.3 | TRUE |
| chr7 | 19184555 | 19185139 | 0.376278475 | 1.505113902 | 174680 | 585 | 222894 | FERD3L | 0 | uc003suo.1 | TRUE |
| chr3 | 147127012 | 147127143 | 0.376117756 | 1.504471023 | 137005 | 132 | 7545 | ZIC1 | −38 | uc003ewe.3 | TRUE |
| chr8 | 39172097 | 39172120 | 0.374718287 | 1.498873148 | 188372 | 24 | 255926 | ADAM5 | −62 | uc003xmw.3 | TRUE |
| chr8 | 65283649 | 65284473 | 0.37356569 | 1.49426276 | 189643 | 825 | 100130155 | MIR124-2HG | −1302 | uc022avc.2 | TRUE |
| chr8 | 54789207 | 54789978 | 0.373509717 | 1.494038869 | 189147 | 772 | 8601 | RGS20 | 24839 | uc003xrr.2 | FALSE |
| chr12 | 75601465 | 75601928 | 0.372455008 | 1.489820034 | 47938 | 464 | 3747 | KCNC2 | 1600 | uc031qif.1 | FALSE |
| chr8 | 72754469 | 72755162 | 0.371568011 | 1.486272045 | 190018 | 694 | 9242 | MSC | 1569 | uc011lff.2 | FALSE |
| chr19 | 54485494 | 54486058 | 0.370858317 | 1.48343327 | 101899 | 565 | 100126325 | MIR935 | 0 | uc021vbd.1 | TRUE |
| chr7 | 1272515 | 1272559 | 0.370212054 | 1.480848215 | 172932 | 45 | 340260 | UNCX | −95 | uc011jvw.2 | TRUE |
| chr8 | 132052779 | 132052887 | 0.369469119 | 1.477876476 | 192901 | 109 | 114 | ADCY8 | 0 | uc010mds.3 | TRUE |
| chr2 | 105471960 | 105472193 | 0.366605328 | 1.466421313 | 110034 | 234 | 5455 | POU3F3 | 0 | uc010ywg.2 | TRUE |
| chr1 | 25257931 | 25258146 | 0.366347521 | 1.465390083 | 4856 | 216 | 864 | RUNX3 | 33329 | uc001bjq.3 | FALSE |
| chr3 | 127634188 | 127634449 | 0.365767067 | 1.46306827 | 135710 | 262 | 166348 | KBTBD12 | 0 | uc010hsq.3 | TRUE |
| chr13 | 100641409 | 100641867 | 0.365680941 | 1.462723763 | 56561 | 459 | 7546 | ZIC2 | 7383 | uc001von.3 | FALSE |
| chr11 | 121970768 | 121971232 | 0.364772987 | 1.459091949 | 41068 | 465 | 399959 | MIR100HG | 0 | uc001pya.3 | TRUE |
| chr19 | 36909413 | 36909831 | 0.363916974 | 1.455667895 | 98773 | 419 | 284406 | ZFP82 | 0 | uc002ody.1 | TRUE |
| chr16 | 49311934 | 49312543 | 0.363616035 | 1.454464138 | 76099 | 610 | 869 | CBLN1 | 3199 | uc002efq.3 | FALSE |
| chr19 | 58238850 | 58239012 | 0.363499857 | 1.453999428 | 102595 | 163 | 79891 | ZNF671 | 0 | uc010yhf.2 | TRUE |
| chr6 | 100056787 | 100057160 | 0.483763136 | 1.451289409 | 167687 | 374 | 59336 | PRDM13 | 2137 | uc003pqg.1 | FALSE |
| chr13 | 79170146 | 79170303 | 0.483537825 | 1.450613476 | 55867 | 158 | 100874222 | OBI1-AS1 | 541156 | uc001vkv.3 | FALSE |
| chr5 | 2755820 | 2756113 | 0.361449017 | 1.445796069 | 150233 | 294 | 153571 | C5orf38 | 3558 | uc011cmj.3 | FALSE |
| chr8 | 35092687 | 35092876 | 0.361122822 | 1.444491286 | 188063 | 190 | 137970 | UNC5D | −99 | uc003xjr.2 | TRUE |
| chr19 | 58715577 | 58716032 | 0.360987157 | 1.443948628 | 102685 | 456 | 10782 | ZNF274 | 21181 | uc010eum.1 | FALSE |
| chr1 | 200009830 | 200010283 | 0.360748164 | 1.442992655 | 16003 | 454 | 2494 | NR5A2 | 1172 | uc009wzh.3 | FALSE |
| chr8 | 86350568 | 86350889 | 0.360510044 | 1.442040177 | 190519 | 322 | 377677 | CA13 | 217723 | uc003ydj.3 | FALSE |
| chr19 | 21657626 | 21658001 | 0.360200434 | 1.440801737 | 97971 | 376 | 400680 | LINC00664 | −8175 | uc002nqa.3 | FALSE |
| chr19 | 46974567 | 46974908 | 0.359766235 | 1.439064942 | 100379 | 342 | 55228 | PNMA8A | 0 | uc002per.4 | TRUE |
| chr4 | 111535334 | 111536024 | 0.358888921 | 1.435555682 | 146002 | 691 | 5308 | PITX2 | 8230 | uc003iag.1 | FALSE |
| chr12 | 130388748 | 130389138 | 0.358812189 | 1.435248756 | 51867 | 391 | 121256 | TMEM132D | −536 | uc009zyl.1 | TRUE |
| chr20 | 61560373 | 61560630 | 0.358646277 | 1.434585108 | 122392 | 258 | 11083 | DIDO1 | 8674 | uc002ydr.2 | FALSE |
| chr5 | 33936362 | 33936520 | 0.358471095 | 1.433884379 | 151497 | 159 | 51289 | RXFP3 | 0 | uc003jic.2 | TRUE |
| chr3 | 147113700 | 147113918 | 0.477834688 | 1.433504065 | 137000 | 219 | 84107 | ZIC4 | 1055 | uc003ewc.2 | FALSE |
| chr20 | 22558834 | 22559346 | 0.357837534 | 1.431350136 | 120019 | 513 | 140828 | LINC00261 | 0 | uc010zsp.2 | TRUE |
| chr17 | 36103953 | 36104585 | 0.356858935 | 1.427435742 | 84788 | 633 | 6928 | HNF1B | 511 | uc021tvw.1 | FALSE |
| chr11 | 14995167 | 14995233 | 0.356856777 | 1.427427106 | 32931 | 67 | 796 | CALCA | −1335 | uc001mlt.2 | TRUE |
| chr6 | 31696333 | 31696482 | 0.356541698 | 1.426166793 | 163688 | 150 | 23564 | DDAH2 | 1087 | uc003nwp.3 | FALSE |
| chr2 | 176947786 | 176948343 | 0.355717577 | 1.422870307 | 114055 | 558 | 344191 | EVX2 | 347 | uc010zeu.2 | FALSE |
| chr2 | 119916017 | 119916510 | 0.355645882 | 1.422583528 | 110909 | 494 | 165257 | C1QL2 | 0 | uc002tlo.2 | TRUE |
| chr15 | 27215997 | 27216083 | 0.355209878 | 1.420839511 | 65184 | 87 | 2567 | GABRG3 | −346 | uc001zbf.4 | TRUE |
| chr5 | 178368183 | 178368253 | 0.35335291 | 1.413411641 | 159860 | 71 | 285676 | ZNF454 | 0 | uc021yjc.1 | TRUE |
| chr2 | 119607603 | 119607885 | 0.470336647 | 1.41100994 | 110886 | 283 | 2019 | EN1 | −1844 | uc002tlm.3 | TRUE |
| chr6 | 110678956 | 110679566 | 0.351100506 | 1.404402026 | 168232 | 611 | 728464 | METTL24 | 0 | uc010kdu.1 | TRUE |
| chr15 | 89949617 | 89950204 | 0.350808949 | 1.403235795 | 70551 | 588 | 254559 | MIR9-3HG | 28344 | uc002bnw.2 | FALSE |
| chr10 | 100993583 | 100993597 | 0.349134743 | 1.396538971 | 27210 | 15 | 60495 | HPSE2 | 2035 | uc009xwd.2 | FALSE |
| chr12 | 54321346 | 54321653 | 0.464276733 | 1.392830198 | 46488 | 308 | 100874366 | HOXC13-AS | 11774 | uc001sei.3 | FALSE |
| chr6 | 28584155 | 28584288 | 0.348205329 | 1.392821316 | 162997 | 134 | 114821 | ZBED9 | −29043 | uc003nlo.3 | FALSE |
| chr16 | 71264545 | 71264678 | 0.34803761 | 1.392150441 | 77866 | 134 | 54768 | HYDIN | 0 | uc002ezw.4 | TRUE |
| chr12 | 54070891 | 54071111 | 0.347909344 | 1.391637374 | 46470 | 221 | 517 | ATP5MC2 | −379 | uc001sed.3 | TRUE |
| chr2 | 54087189 | 54087343 | 0.346982729 | 1.387930917 | 106896 | 155 | 51130 | ASB3 | −19 | uc002rxi.4 | TRUE |
| chr6 | 100442105 | 100442151 | 0.346855906 | 1.387423626 | 167698 | 47 | 84539 | MCHR2 | 0 | uc003pqi.1 | TRUE |
| chr1 | 119543057 | 119543336 | 0.346759973 | 1.38703989 | 11740 | 280 | 6913 | TBX15 | −10878 | uc001ehl.1 | FALSE |
| chr1 | 200011684 | 200011858 | 0.34623399 | 1.384935962 | 16003 | 175 | 2494 | NR5A2 | 3026 | uc010pph.3 | FALSE |
| chr22 | 29875947 | 29876363 | 0.346181382 | 1.384725528 | 125892 | 417 | 4744 | NEFH | 0 | uc003afo.3 | TRUE |
| chr1 | 79472343 | 79472452 | 0.346039768 | 1.384159074 | 9595 | 110 | 64123 | ADGRL4 | 43 | uc001diq.4 | FALSE |
| chr22 | 40296407 | 40297048 | −0.345566926 | 1.382267705 | 126853 | 642 | 9402 | GRAP2 | −38 | uc003ayh.2 | TRUE |
| chr17 | 46719761 | 46720050 | 0.345184929 | 1.380739715 | 86661 | 290 | 406972 | MIR196A1 | −9840 | uc010wln.1 | FALSE |
| chr4 | 113441608 | 113441944 | 0.343449489 | 1.373797957 | 146076 | 337 | 63973 | NEUROG2 | −4280 | uc003ias.3 | FALSE |
| chr12 | 4919081 | 4919230 | 0.45780736 | 1.373422081 | 43043 | 150 | 3742 | KCNA6 | 739 | uc001qng.3 | FALSE |
| chr3 | 62860802 | 62861142 | 0.342995892 | 1.371983569 | 132725 | 341 | 8618 | CADPS | 0 | uc021wzv.1 | TRUE |
| chr7 | 96626151 | 96626788 | 0.342468514 | 1.369874056 | 179551 | 638 | 285987 | DLX6-AS1 | 6822 | uc003uok.3 | FALSE |
| chr10 | 57387702 | 57387978 | 0.455219733 | 1.365659198 | 24139 | 277 | 65217 | PCDH15 | 0 | uc001jjv.1 | TRUE |
| chr6 | 28733661 | 28733950 | 0.341204357 | 1.364817428 | 163022 | 290 | 401242 | LINC01623 | 97504 | uc003nlq.2 | FALSE |
| chr5 | 122620929 | 122621240 | 0.341103923 | 1.364415691 | 155379 | 312 | 153241 | CEP120 | 138046 | uc011cwq.2 | FALSE |
| chr10 | 102890974 | 102891019 | 0.339401671 | 1.357606686 | 27494 | 46 | 3195 | TLX1 | −42 | uc001ksw.3 | TRUE |
| chr5 | 113698140 | 113698506 | 0.338837436 | 1.355349746 | 154993 | 367 | 3781 | KCNN2 | 124 | uc031skt.1 | FALSE |
| chr12 | 54359712 | 54360131 | 0.338142026 | 1.352568105 | 46503 | 420 | 100124700 | HOTAIR | 2409 | uc009zne.4 | FALSE |
| chr2 | 468179 | 468413 | 0.450655561 | 1.351966684 | 102866 | 235 | 285016 | ALKAL2 | −179871 | uc002qwi.4 | FALSE |
| chr1 | 235814009 | 235814163 | 0.33740266 | 1.349610642 | 19343 | 155 | 2786 | GNG4 | 0 | uc009xfz.3 | TRUE |
| chrX | 102000694 | 102000758 | 0.337153529 | 1.348614117 | 202999 | 65 | 80823 | BHLHB9 | 25052 | uc011mru.2 | FALSE |
| chr1 | 50513766 | 50513927 | 0.449532705 | 1.348598114 | 7864 | 162 | 1996 | ELAVL4 | 80 | uc001cry.3 | FALSE |
| chr6 | 99271924 | 99272559 | 0.337120501 | 1.348482004 | 167621 | 636 | 5454 | POU3F2 | −10021 | uc003ppe.3 | FALSE |
| chr22 | 48971959 | 48972536 | 0.337020441 | 1.348081764 | 127719 | 578 | 25817 | TAFA5 | 0 | uc003bio.4 | TRUE |
| chr6 | 33048502 | 33048558 | 0.336839814 | 1.347359258 | 164072 | 57 | 3115 | HLA-DPB1 | 53 | uc021ywh.1 | FALSE |
| chr17 | 7832852 | 7832943 | 0.33611106 | 1.34444424 | 82120 | 92 | 9196 | KCNAB3 | −99 | uc002gjm.2 | TRUE |
| chr10 | 108924366 | 108924560 | 0.448108211 | 1.344324633 | 28034 | 195 | 114815 | SORCS1 | 0 | uc001kyo.3 | TRUE |
| chr6 | 27647713 | 27647896 | 0.44669602 | 1.340088059 | 162830 | 184 | 100507173 | LINC01012 | −13918 | uc021yrb.1 | FALSE |
| chr2 | 118981391 | 118982056 | 0.334584302 | 1.338337208 | 110846 | 666 | 51141 | NSIG2 | 120618 | uc002tll.3 | FALSE |
| chrX | 125300078 | 125300111 | 0.334325903 | 1.337303614 | 203598 | 34 | 340578 | DCAF12L2 | 0 | uc004euk.2 | TRUE |
| chr6 | 28304075 | 28304161 | 0.333923122 | 1.335692487 | 162933 | 87 | 64288 | ZSCAN31 | 0 | uc021yrt.1 | TRUE |
| chr8 | 72468820 | 72469507 | 0.332120333 | 1.328481331 | 190002 | 688 | 2138 | EYA1 | −194353 | uc003xyu.3 | FALSE |
| chr7 | 27140942 | 27141139 | 0.330897513 | 1.323590052 | 175203 | 198 | 3199 | HOXA2 | 1255 | uc003syh.3 | FALSE |
| chr7 | 12151221 | 12151638 | 0.330725705 | 1.322902819 | 174430 | 418 | 54664 | TMEM106B | −99210 | uc003ssh.3 | FALSE |
| chr7 | 158937494 | 158938051 | 0.330686373 | 1.322745491 | 184979 | 558 | 7434 | VIPR2 | 0 | uc010lqy.3 | TRUE |
| chr13 | 28491540 | 28491615 | 0.440392626 | 1.321177878 | 53540 | 76 | 3651 | PDX1 | −2553 | uc001urt.2 | FALSE |
| chr11 | 20181725 | 20181911 | 0.440368534 | 1.321105602 | 33399 | 187 | 120237 | DBX1 | 0 | uc021qez.1 | TRUE |
| chr7 | 27225485 | 27225528 | 0.439549529 | 1.318648588 | 175244 | 44 | 221883 | HOXA11-AS | 458 | uc003syz.1 | FALSE |
| chr20 | 61638518 | 61638588 | 0.438242143 | 1.314726428 | 122420 | 71 | 128408 | BHLHE23 | −131 | uc002yeb.2 | TRUE |
| chrX | 136656287 | 136656581 | 0.43647515 | 1.30942545 | 203969 | 295 | 7547 | ZIC3 | 7941 | uc004fak.3 | FALSE |
| chr17 | 48545950 | 48546258 | 0.327131923 | 1.308527693 | 87010 | 309 | 80221 | ACSF2 | 260 | uc010dbs.3 | FALSE |
| chr3 | 147087225 | 147087653 | 0.435441409 | 1.306324227 | 136990 | 429 | 84107 | ZIC4 | 22564 | uc021xfe.1 | FALSE |
| chr2 | 45169548 | 45169681 | 0.434316363 | 1.302949088 | 106363 | 134 | 6496 | SIX3 | 511 | uc002run.2 | FALSE |
| chr4 | 41882163 | 41882580 | 0.433091756 | 1.299275268 | 143327 | 418 | 55161 | TMEM33 | −54557 | uc003gwi.2 | FALSE |
| chr10 | 101875028 | 101875138 | 0.432273572 | 1.296820716 | 27305 | 111 | 1369 | CPN1 | −33386 | uc001kql.2 | FALSE |
| chr3 | 147111660 | 147112096 | 0.431819483 | 1.295458449 | 136999 | 437 | 84107 | ZIC4 | 2877 | uc021xfc.1 | FALSE |
| chr19 | 57078765 | 57078794 | 0.323465073 | 1.29386029 | 102421 | 30 | 388566 | ZNF470 | −96 | uc002qnl.4 | TRUE |
| chr12 | 54653364 | 54653427 | 0.431104621 | 1.293313863 | 46588 | 64 | 23468 | CBX5 | 0 | uc001sfj.4 | TRUE |
| chr1 | 62660624 | 62660861 | 0.431089742 | 1.293269226 | 8838 | 238 | 54596 | L1TD1 | 150 | uc021ooc.1 | FALSE |
| chr1 | 1475209 | 1475737 | 0.430932569 | 1.292797708 | 398 | 529 | 339453 | TMEM240 | 3 | uc009vkf.3 | FALSE |
| chr11 | 31846819 | 31846849 | 0.430539071 | 1.291617214 | 33775 | 31 | 440034 | PAX6-AS1 | 8705 | uc009yjr.3 | FALSE |
| chr8 | 105478683 | 105479058 | 0.430510754 | 1.291532263 | 191630 | 376 | 1807 | DPYS | 219 | uc003yly.4 | FALSE |
| chr5 | 1446165 | 1446217 | 0.429689649 | 1.289068947 | 149953 | 53 | 6531 | SLC6A3 | −622 | uc003jck.3 | TRUE |
| chr13 | 37004812 | 37004990 | 0.320878725 | 1.283514899 | 54130 | 179 | 8900 | CCNA1 | −667 | uc010teo.2 | TRUE |
| chr12 | 52626889 | 52627047 | 0.425828255 | 1.277484766 | 46173 | 159 | 3855 | KRT7 | 0 | uc001saa.1 | TRUE |
| chr2 | 176936397 | 176936581 | 0.421858677 | 1.265576031 | 114051 | 185 | 344191 | EVX2 | 12109 | uc010zeu.2 | FALSE |
| chr12 | 114852091 | 114852359 | 0.42146607 | 1.264398209 | 50279 | 269 | 255480 | TBX5-AS1 | 5532 | uc001tvs.2 | FALSE |
| chr7 | 54612325 | 54612475 | 0.421465945 | 1.264397834 | 177225 | 151 | 222008 | VSTM2A | 2306 | uc010kzf.3 | FALSE |
| chr6 | 28603230 | 28603292 | 0.421222359 | 1.263667077 | 163001 | 63 | 114821 | ZBED9 | −48118 | uc003nlo.3 | FALSE |
| chr6 | 28557846 | 28558006 | 0.315870401 | 1.263481603 | 162994 | 161 | 114821 | ZBED9 | −2734 | uc003nlo.3 | FALSE |
| chr22 | 22862802 | 22863219 | 0.315280581 | 1.261122323 | 125401 | 418 | 140883 | ZNF280B | 286 | uc002zwc.1 | FALSE |
| chr6 | 137814728 | 137814971 | 0.420007913 | 1.260023738 | 169601 | 244 | 167826 | OLIG3 | 560 | uc003qhp.1 | FALSE |
| chr12 | 25055676 | 25056083 | 0.418289432 | 1.254868295 | 44487 | 408 | 586 | BCAT1 | 0 | uc010six.2 | TRUE |
| chr4 | 74864165 | 74864313 | 0.417738313 | 1.253214938 | 144402 | 149 | 6374 | CXCL5 | 133 | uc003hhk.4 | FALSE |
| chr2 | 120281719 | 120281999 | 0.417690014 | 1.253070041 | 110957 | 281 | 6344 | SCTR | 29 | uc002tma.3 | FALSE |
| chr12 | 127940086 | 127940654 | 0.415628261 | 1.246884782 | 51692 | 569 | 400087 | LINC02393 | −425508 | uc001uhp.2 | FALSE |
| chr5 | 178004012 | 178004204 | 0.414642092 | 1.243926275 | 159817 | 193 | 91522 | COL23A1 | 13352 | uc021yiz.1 | FALSE |
| chr2 | 175208588 | 175208605 | 0.414550602 | 1.243651806 | 113942 | 18 | 9541 | CIR1 | 51838 | uc010zem.1 | FALSE |
| chr1 | 2425860 | 2426035 | −0.413835552 | 1.241506657 | 842 | 176 | 9651 | PLCH2 | 14237 | uc001ajl.1 | FALSE |
| chr14 | 60952097 | 60952405 | 0.413491751 | 1.240475254 | 60292 | 309 | 317761 | C14orf39 | 359 | uc010apo.3 | FALSE |
| chrX | 136632433 | 136633004 | 0.413057764 | 1.239173292 | 203960 | 572 | 7547 | ZIC3 | −15342 | uc004fak.3 | FALSE |
| chr8 | 97171827 | 97172012 | 0.411887161 | 1.235661483 | 191027 | 186 | 392255 | GDF6 | 1008 | uc003yhp.3 | FALSE |
| chr1 | 44873064 | 44873592 | 0.410376535 | 1.231129604 | 7399 | 529 | 55182 | RNF220 | 2014 | uc001clw.1 | FALSE |
| chr19 | 21646329 | 21646470 | 0.409931683 | 1.229795048 | 97968 | 142 | 400680 | LINC00664 | −19706 | uc002nqa.3 | FALSE |
| chr8 | 61194072 | 61194099 | 0.409620276 | 1.228860829 | 189463 | 28 | 767 | CA8 | −118 | uc003xtz.1 | TRUE |
| chr11 | 32458656 | 32458769 | 0.408332105 | 1.224996314 | 33813 | 114 | 51352 | WT1-AS | 1071 | uc010red.2 | FALSE |
| chr3 | 157821407 | 157821919 | 0.406981023 | 1.22094307 | 137635 | 513 | 6474 | SHOX2 | 2033 | uc010hvw.3 | FALSE |
| chr19 | 54926431 | 54926514 | 0.406186931 | 1.218560793 | 101990 | 84 | 57348 | TTYH1 | −91 | uc010yey.2 | TRUE |
| chr19 | 21769190 | 21769430 | 0.406019717 | 1.218059152 | 97982 | 241 | 353088 | ZNF429 | 80753 | uc010ecu.2 | FALSE |
| chr14 | 38724648 | 38724945 | 0.405849162 | 1.217547485 | 59248 | 298 | 161198 | CLEC14A | 630 | uc001wum.2 | FALSE |
| chr12 | 62585031 | 62585467 | 0.405152464 | 1.215457391 | 47266 | 437 | 338811 | TAFA2 | 1153 | uc001sqw.3 | FALSE |
| chr13 | 88326244 | 88326752 | 0.404898631 | 1.214695892 | 56011 | 509 | 26050 | SLITRK5 | 1374 | uc001vln.3 | FALSE |
| chr17 | 46802871 | 46803008 | 0.404243689 | 1.212731066 | 86670 | 138 | 10481 | HOXB13 | 3103 | uc021tzl.1 | FALSE |
| chr11 | 47359017 | 47359223 | −0.403653165 | 1.210959496 | 34833 | 207 | 4607 | MYBPC3 | 5473 | uc010rhl.2 | FALSE |
| chr3 | 44626453 | 44626538 | 0.40345002 | 1.210350061 | 130896 | 86 | 285349 | ZNF660 | 0 | uc003cnl.1 | TRUE |
| chr6 | 28603033 | 28603059 | 0.402937269 | 1.208811806 | 163001 | 27 | 114821 | ZBED9 | −47921 | uc003nlo.3 | FALSE |
| chr8 | 55370423 | 55370434 | 0.402905684 | 1.208717053 | 189187 | 12 | 64321 | SOX17 | −61 | uc003xsb.4 | TRUE |
| chr1 | 91301651 | 91301962 | 0.402741193 | 1.20822358 | 10132 | 312 | 84146 | ZNF644 | 185103 | uc001dns.3 | FALSE |
| chr5 | 178367827 | 178368123 | 0.402506782 | 1.207520347 | 159860 | 297 | 285676 | ZNF454 | −71 | uc003mjp.3 | TRUE |
| chr1 | 2374849 | 2375420 | 0.401978873 | 1.20593662 | 808 | 572 | 5192 | PEX10 | −30839 | uc001ajg.3 | FALSE |
| chr6 | 31696223 | 31696240 | 0.401417667 | 1.204253 | 163688 | 18 | 23564 | DDAH2 | 1329 | uc003nwp.3 | FALSE |
| chr10 | 23463094 | 23463377 | 0.398757708 | 1.196273125 | 22258 | 284 | 256297 | PTF1A | −18083 | uc001irp.3 | FALSE |
| chr17 | 38347710 | 38347968 | 0.398567081 | 1.195701243 | 85169 | 259 | 51195 | RAPGEFL1 | 2920 | uc010wfd.1 | FALSE |
| chr16 | 56672387 | 56672525 | 0.398546024 | 1.195638072 | 76645 | 139 | 4489 | MT1A | 29909 | uc002ejq.3 | FALSE |
| chr2 | 119591097 | 119591627 | 0.398207289 | 1.194621868 | 110879 | 531 | 2019 | EN1 | 14132 | uc002tlm.3 | FALSE |
| chr14 | 57275967 | 57276257 | 0.398153924 | 1.194461772 | 60073 | 291 | 5015 | OTX2 | 937 | uc031qot.1 | FALSE |
| chr8 | 50653870 | 50653939 | −0.397230702 | 1.191692105 | 188987 | 70 | 54212 | SNTG1 | −168410 | uc010lxy.1 | FALSE |
| chr15 | 26107658 | 26108263 | 0.397058076 | 1.191174227 | 65101 | 606 | 57194 | ATP10A | 86 | uc010ayu.3 | FALSE |
| chr13 | 112758491 | 112758625 | 0.397038697 | 1.191116092 | 57402 | 135 | 6656 | SOX1 | 36578 | uc001vsb.1 | FALSE |
| chr17 | 46799640 | 46799755 | 0.396924555 | 1.190773665 | 86670 | 116 | 84366 | PRAC1 | 127 | uc002iny.3 | FALSE |
| chr17 | 5000803 | 5001047 | 0.396004941 | 1.188014822 | 81668 | 245 | 124961 | ZFP3 | 19049 | uc010vsv.1 | FALSE |
| chr8 | 23563859 | 23563970 | 0.395934383 | 1.187803149 | 187319 | 112 | 137814 | NKX2-6 | 141 | uc011kzy.3 | FALSE |
| chr20 | 26188639 | 26188919 | 0.395784769 | 1.187354306 | 120205 | 281 | 724033 | MIR663A | 0 | uc021wbn.1 | TRUE |
| chr11 | 70917159 | 70917533 | 0.39556039 | 1.18668117 | 37642 | 375 | 22941 | SHANK2 | 18309 | uc001oqc.3 | FALSE |
| chr10 | 50604499 | 50604569 | 0.395050565 | 1.185151695 | 23883 | 71 | 644168 | DRGX | −437 | uc021pqd.2 | TRUE |
| chr1 | 50513645 | 50513661 | 0.393219735 | 1.179659204 | 7864 | 17 | 1996 | ELAVL4 | −25 | uc001cry.3 | TRUE |
| chr16 | 22926538 | 22926625 | −0.392777702 | 1.178333105 | 74689 | 88 | 9956 | HS3ST2 | 100678 | uc002dli.3 | FALSE |
| chr7 | 150786044 | 150786082 | 0.392627728 | 1.177883184 | 183641 | 39 | 116988 | AGAP3 | 2218 | uc003wjg.1 | FALSE |
| chr20 | 25062254 | 25062754 | 0.392377837 | 1.177133512 | 120136 | 501 | 30813 | VSX1 | 261 | uc002wug.2 | FALSE |
| chr2 | 176981422 | 176981654 | 0.391655137 | 1.17496541 | 114070 | 233 | 3236 | HOXD10 | 0 | uc002ukj.3 | TRUE |
| chr2 | 229046325 | 229046515 | 0.391075611 | 1.173226832 | 117031 | 191 | 80309 | SPHKAP | 0 | uc010zlx.1 | TRUE |
| chr1 | 91300215 | 91300288 | 0.390444171 | 1.171332512 | 10131 | 74 | 84146 | ZNF644 | 186777 | uc001dns.3 | FALSE |
| chr18 | 31739202 | 31739423 | 0.390212075 | 1.170636224 | 92033 | 222 | 8715 | NOL4 | 63011 | uc010xbt.2 | FALSE |
| chr16 | 3220475 | 3220915 | 0.389975464 | 1.169926393 | 73015 | 441 | 7760 | ZNF213 | 33308 | uc010uwt.3 | FALSE |
| chr5 | 170742118 | 170742608 | 0.38976382 | 1.169291459 | 158821 | 491 | 30012 | TLX3 | 5830 | uc003mbf.3 | FALSE |
| chr19 | 53038972 | 53039444 | 0.389439269 | 1.168317808 | 101633 | 473 | 388558 | ZNF808 | 8063 | uc002pzq.2 | FALSE |
| chr19 | 54445377 | 54445888 | 0.389077754 | 1.167233263 | 101889 | 512 | 59284 | CACNG7 | 29386 | uc002qcs.2 | FALSE |
| chr11 | 31821298 | 31821388 | 0.388651387 | 1.165954161 | 33759 | 91 | 5080 | PAX6 | 4206 | uc031pzl.1 | FALSE |
| chr8 | 55370283 | 55370336 | 0.387386116 | 1.162158349 | 189187 | 54 | 64321 | SOX17 | −159 | uc003xsb.4 | TRUE |
| chr7 | 24324570 | 24325079 | 0.38673964 | 1.160218921 | 174999 | 510 | 4852 | NPY | 763 | uc003sww.2 | FALSE |
| chr4 | 174450016 | 174450722 | 0.386139853 | 1.15841956 | 148425 | 707 | 9464 | HAND2 | 0 | uc003itg.1 | TRUE |
| chr2 | 176976802 | 176977285 | 0.385515705 | 1.156547116 | 114068 | 484 | 3237 | HOXD11 | 4718 | uc002ukj.3 | FALSE |
| chr14 | 57274677 | 57274763 | 0.385498643 | 1.156495928 | 60072 | 87 | 5015 | OTX2 | 2431 | uc001xcp.4 | FALSE |
| chr10 | 103043991 | 103044194 | 0.384881756 | 1.154645269 | 27531 | 204 | 399806 | LBX1-AS1 | 54640 | uc010qpy.2 | FALSE |
| chr5 | 45696455 | 45696491 | 0.384854968 | 1.154564905 | 152083 | 37 | 348980 | HCN1 | −235 | uc003jok.3 | TRUE |
| chr4 | 13536910 | 13537089 | 0.38396916 | 1.15190748 | 142213 | 180 | 285547 | LINC01097 | −3869 | uc010idp.1 | FALSE |
| chr10 | 129535669 | 129535893 | 0.383942726 | 1.151828177 | 29603 | 225 | 399823 | FOXI2 | 131 | uc009yas.2 | FALSE |
| chr3 | 141516232 | 141516291 | 0.383028678 | 1.149086033 | 136815 | 60 | 131890 | GRK7 | 19189 | uc011bnd.2 | FALSE |
| chr2 | 5831213 | 5831722 | 0.382879986 | 1.148639959 | 103478 | 510 | 6664 | SOX11 | −1077 | uc002qyj.3 | TRUE |
| chr10 | 118897836 | 118897857 | 0.382864972 | 1.148594917 | 28618 | 22 | 11023 | VAX1 | −24 | uc001ldb.1 | TRUE |
| chr12 | 81471867 | 81471884 | 0.381978515 | 1.145935546 | 48259 | 18 | 79611 | ACSS3 | 31 | uc001szm.1 | FALSE |
| chr19 | 53561386 | 53561563 | 0.380710736 | 1.142132209 | 101708 | 178 | 100271846 | ERVV-2 | 13395 | uc021uzd.1 | FALSE |
| chr8 | 98290148 | 98290310 | 0.380099444 | 1.140298333 | 191105 | 163 | 85453 | TSPYL5 | 0 | uc003yhy.3 | TRUE |
| chr1 | 47900256 | 47900320 | 0.378662928 | 1.135988784 | 7759 | 65 | 84793 | FOXD2-AS1 | 0 | uc001crl.3 | TRUE |
| chr17 | 36103066 | 36103289 | 0.377847052 | 1.133541157 | 84787 | 224 | 6928 | HNF1B | 1807 | uc021tvw.1 | FALSE |
| chr1 | 58715532 | 58715553 | 0.377801457 | 1.13340437 | 8606 | 22 | 1600 | DAB1 | 658 | uc001cys.1 | FALSE |
| chr2 | 172953032 | 172953270 | 0.377289459 | 1.131868378 | 113730 | 239 | 1745 | DLX1 | 2824 | uc002uhm.3 | FALSE |
| chr1 | 66258687 | 66259084 | 0.376899189 | 1.130697566 | 9111 | 398 | 5142 | PDE4B | 0 | uc001dco.3 | TRUE |
| chr18 | 56887194 | 56887279 | 0.376717404 | 1.130152211 | 92671 | 86 | 2922 | GRP | −121 | uc002lhu.3 | TRUE |
| chr15 | 29410127 | 29410245 | −0.375924675 | 1.127774026 | 65347 | 119 | 321 | APBA2 | 196284 | uc001zcm.1 | FALSE |
| chr2 | 172958009 | 172958337 | 0.375834606 | 1.127503817 | 113732 | 329 | 1745 | DLX1 | 7801 | uc002uhm.3 | FALSE |
| chr11 | 31848488 | 31848649 | 0.375395714 | 1.126187141 | 33775 | 162 | 440034 | PAX6-AS1 | 10374 | uc009yjr.3 | FALSE |
| chr4 | 147561775 | 147562073 | 0.375172125 | 1.125516374 | 147360 | 299 | 5458 | POU4F2 | 1730 | uc003ikv.3 | FALSE |
| chr11 | 2890647 | 2890705 | 0.37509218 | 1.125276539 | 31682 | 59 | 55539 | KCNQ1DN | −558 | uc009ydq.3 | TRUE |
| chr14 | 57264919 | 57265560 | 0.374816769 | 1.124450308 | 60067 | 642 | 5015 | OTX2 | 6821 | uc031qor.1 | FALSE |
| chr9 | 79633350 | 79633737 | 0.374809351 | 1.124428052 | 196099 | 388 | 442425 | FOXB2 | −834 | uc004ako.1 | TRUE |
| chr8 | 23083353 | 23083578 | −0.37473435 | 1.124203049 | 187247 | 226 | 389641 | LOC389641 | 619 | uc003xdb.1 | FALSE |
| chr12 | 95941988 | 95942761 | 0.374702047 | 1.124106141 | 48776 | 774 | 84101 | USP44 | 0 | uc001teg.3 | TRUE |
| chr19 | 44324747 | 44324898 | 0.374552161 | 1.123656484 | 99913 | 152 | 284348 | LYPD5 | 0 | uc002oxn.4 | TRUE |
| chr5 | 76932016 | 76932062 | 0.374109433 | 1.1223283 | 153513 | 47 | 23440 | OTP | 2460 | uc003kfg.3 | FALSE |
| chr19 | 9609397 | 9609422 | 0.374013747 | 1.122041241 | 95975 | 26 | 147741 | ZNF560 | −118 | uc002mlp.1 | TRUE |
| chr7 | 158936538 | 158936739 | 0.373989897 | 1.121969692 | 184979 | 202 | 7434 | VIPR2 | 910 | uc010lqy.3 | FALSE |
| chr13 | 112927190 | 112927331 | −0.373645216 | 1.120935648 | 57453 | 142 | 122258 | SPACA7 | −103320 | uc001vsd.2 | FALSE |
| chr7 | 27205217 | 27205230 | 0.373611274 | 1.120833822 | 175231 | 14 | 100534589 | HOXA10-HOXA9 | 14650 | uc003syt.3 | FALSE |
| chr1 | 66258022 | 66258046 | 0.37344656 | 1.120339679 | 9111 | 25 | 5142 | PDE4B | −147 | uc001dcn.3 | TRUE |
| chr12 | 72667386 | 72667493 | 0.373435757 | 1.120307271 | 47888 | 108 | 29953 | TRHDE | 857 | uc010stv.2 | FALSE |
| chrX | 108868271 | 108868465 | 0.373421693 | 1.120265079 | 203239 | 195 | 23630 | KCNE5 | 0 | uc004eoh.3 | TRUE |
| chr5 | 11904110 | 11904127 | 0.372332877 | 1.116998632 | 150779 | 18 | 1501 | CTNND2 | 0 | uc011cmz.1 | TRUE |
| chr14 | 29243234 | 29243504 | 0.372070603 | 1.11621181 | 58847 | 271 | 387978 | LINC01551 | 1324 | uc001wqf.3 | FALSE |
| chr19 | 56988813 | 56989543 | 0.37183717 | 1.11551151 | 102413 | 731 | 100128252 | ZNF667-AS1 | 0 | uc021vcf.1 | TRUE |
| chr12 | 54409485 | 54409525 | 0.371727846 | 1.115183539 | 46529 | 41 | 3223 | HOXC6 | −1117 | uc001ses.3 | TRUE |
| chr6 | 32116933 | 32116994 | 0.371673458 | 1.115020375 | 163826 | 62 | 80863 | PRRT1 | 2706 | uc003nzs.3 | FALSE |
| chr4 | 37246688 | 37246827 | 0.37160966 | 1.114828981 | 142971 | 140 | 57495 | NWD2 | 0 | uc011bxz.2 | TRUE |
| chr2 | 182544307 | 182544413 | 0.37160153 | 1.114804591 | 114384 | 107 | 375298 | CERKL | 979 | uc002uof.4 | FALSE |
| chr3 | 181438122 | 181438328 | 0.371405473 | 1.114216419 | 138629 | 207 | 347689 | SOX2-OT | 110000 | uc003fkx.3 | FALSE |
| chr19 | 11784731 | 11784774 | 0.370273949 | 1.110821847 | 96415 | 44 | 401898 | ZNF833P | 34140 | uc021upi.1 | FALSE |
| chr1 | 158390649 | 158391119 | −0.370163298 | 1.110489893 | 13639 | 471 | 391107 | OR10K2 | 0 | uc010pii.2 | TRUE |
| chr15 | 27112197 | 27112902 | 0.369476011 | 1.108428032 | 65167 | 706 | 2558 | GABRA5 | 0 | uc021sgi.1 | TRUE |
| chr21 | 36421857 | 36421955 | −0.369463481 | 1.108390444 | 123419 | 99 | 861 | RUNX1 | 935092 | uc002yuk.4 | FALSE |
| chr8 | 133072269 | 133072873 | 0.369317211 | 1.107951632 | 192947 | 605 | 729330 | OC90 | −642 | uc011lix.1 | TRUE |
| chr13 | 112712475 | 112712795 | 0.369163285 | 1.107489856 | 57385 | 321 | 6656 | SOX1 | −9118 | uc001vsb.1 | FALSE |
| chr8 | 121137879 | 121138232 | 0.368556738 | 1.105670215 | 192179 | 354 | 7373 | COL14A1 | 532 | uc003yox.4 | FALSE |
| chr15 | 86233214 | 86233236 | 0.368384292 | 1.105152876 | 70295 | 23 | 11214 | AKAP13 | 12946 | uc002blx.2 | FALSE |
| chr6 | 100912906 | 100912946 | 0.368221765 | 1.104665295 | 167736 | 41 | 6492 | SIM1 | −101 | uc010kcu.3 | TRUE |
| chr2 | 108602937 | 108603005 | 0.368046359 | 1.104139076 | 110253 | 69 | 60482 | SLC5A7 | 0 | uc010ywn.2 | TRUE |
| chr7 | 153583318 | 153583867 | 0.368038639 | 1.104115916 | 183948 | 550 | 1804 | DPP6 | −552 | uc003wli.3 | TRUE |
| chr6 | 118228650 | 118228871 | 0.368020813 | 1.104062439 | 168652 | 222 | 222553 | SLC35F1 | 0 | uc003pxx.4 | TRUE |
| chr2 | 176964137 | 176964456 | 0.368008687 | 1.104026061 | 114061 | 320 | 3238 | HOXD12 | −74 | uc010zev.1 | TRUE |
| chr9 | 138660958 | 138660998 | −0.551680645 | 1.10336129 | 199804 | 41 | 57582 | KCNT1 | 15361 | uc004cgo.1 | FALSE |
| chr7 | 38350921 | 38351094 | 0.367146083 | 1.101438249 | 176139 | 174 | 445347 | TARP | 5615 | uc003tgf.1 | FALSE |
| chr3 | 138679347 | 138679512 | 0.366460154 | 1.099380463 | 136621 | 166 | 401089 | FOXL2NB | 13271 | uc003esx.1 | FALSE |
| chr20 | 23015908 | 23015936 | 0.366254943 | 1.098764828 | 120032 | 29 | 6754 | SSTR4 | −121 | uc002wsr.2 | TRUE |
| chr14 | 52735026 | 52735485 | 0.366212638 | 1.098637914 | 59690 | 460 | 5729 | PTGDR | 595 | uc001wzq.3 | FALSE |
| chr13 | 28544592 | 28544814 | 0.366068119 | 1.098204357 | 53556 | 223 | 1045 | CDX2 | −1087 | uc001urv.4 | TRUE |
| chr3 | 27772633 | 27772805 | 0.365901525 | 1.097704575 | 129845 | 173 | 8320 | EOMES | −8427 | uc003cdx.4 | FALSE |
| chr7 | 157476887 | 157477308 | 0.364517116 | 1.093551348 | 184519 | 422 | 5799 | PTPRN2 | 857160 | uc011kwb.1 | FALSE |
| chr2 | 119600519 | 119600948 | 0.364509804 | 1.093529413 | 110885 | 430 | 2019 | EN1 | 4811 | uc002tlm.3 | FALSE |
| chr10 | 134901279 | 134901297 | 0.364459738 | 1.093379213 | 30503 | 19 | 84435 | ADGRA1 | 16846 | uc001llx.4 | FALSE |
| chr10 | 102893980 | 102894148 | 0.364386242 | 1.093158725 | 27495 | 169 | 3195 | TLX1 | 2919 | uc021pxd.1 | FALSE |
| chr12 | 114885161 | 114885372 | 0.364348663 | 1.093045988 | 50286 | 212 | 255480 | TBX5-AS1 | 38602 | uc001tvs.2 | FALSE |
| chr19 | 58446312 | 58446600 | 0.364224467 | 1.092673402 | 102629 | 289 | 147686 | ZNF418 | 140 | uc010yho.1 | FALSE |
| chr13 | 100627341 | 100627703 | 0.364216182 | 1.092648547 | 56554 | 363 | 85416 | ZIC5 | −3163 | uc001vom.1 | FALSE |
| chr20 | 21497429 | 21498100 | 0.364046715 | 1.092140146 | 119987 | 672 | 4821 | NKX2-2 | −2765 | uc002wsi.3 | FALSE |
| chr19 | 58446783 | 58446898 | 0.363940568 | 1.091821703 | 102629 | 116 | 147686 | ZNF418 | −43 | uc002qqs.1 | TRUE |
| chr10 | 8078314 | 8078357 | 0.363838239 | 1.091514717 | 21357 | 44 | 399717 | GATA3-AS1 | 17090 | uc010qbg.1 | FALSE |
| chr13 | 108520566 | 108520945 | 0.363421893 | 1.090265678 | 56885 | 380 | 728215 | FAM155A | −1106 | uc001vql.3 | TRUE |
| chr1 | 248366332 | 248366883 | −0.363310978 | 1.089932934 | 20225 | 552 | 127062 | OR2M3 | 0 | uc010pzg.2 | TRUE |
| chr3 | 147125782 | 147125926 | 0.362867682 | 1.088603045 | 137005 | 145 | 84107 | ZIC4 | −1186 | uc003ewd.2 | TRUE |
| chr5 | 180075862 | 180076168 | 0.362820715 | 1.088462144 | 160191 | 307 | 2324 | FLT4 | 456 | uc011dha.1 | FALSE |
| chr7 | 37960317 | 37960873 | 0.362769779 | 1.088309338 | 176104 | 557 | 54749 | EPDR1 | 154 | uc010kxh.3 | FALSE |
| chr13 | 112720171 | 112720244 | 0.362539458 | 1.087618375 | 57391 | 74 | 6656 | SOX1 | −1669 | uc001vsb.1 | TRUE |
| chr7 | 88388646 | 88389282 | 0.362030529 | 1.086091587 | 179131 | 637 | 219578 | ZNF804B | 0 | uc011khi.2 | TRUE |
| chr2 | 19561482 | 19561709 | 0.361972583 | 1.08591775 | 104474 | 228 | 130497 | OSR1 | −3110 | uc002rdc.3 | FALSE |
| chr5 | 140810106 | 140810123 | 0.361343912 | 1.084031737 | 156934 | 18 | 56105 | PCDHGA11 | 9569 | uc011dba.2 | FALSE |
| chr4 | 299022 | 299121 | 0.361245076 | 1.083735229 | 140219 | 100 | 654254 | ZNF732 | 0 | uc021xka.1 | TRUE |
| chr4 | 156129862 | 156130206 | 0.360805508 | 1.082416525 | 147810 | 345 | 4887 | NPY2R | 81 | uc003ior.3 | FALSE |
| chr8 | 55366538 | 55366834 | 0.360506966 | 1.081520899 | 189184 | 297 | 64321 | SOX17 | −3661 | uc003xsb.4 | FALSE |
| chr14 | 59931922 | 59932101 | 0.360441065 | 1.081323195 | 60230 | 180 | 64582 | GPR135 | 0 | uc010apj.3 | TRUE |
| chr11 | 132526947 | 132527438 | −0.360344617 | 1.081033852 | 42175 | 492 | 4978 | OPCML | 285599 | uc010sck.2 | FALSE |
| chr3 | 130235850 | 130236037 | 0.360334793 | 1.081004379 | 136071 | 188 | 256076 | COL6A5 | 171491 | uc010htl.3 | FALSE |
| chr13 | 28367245 | 28368087 | 0.360289536 | 1.080868608 | 53527 | 843 | 219409 | GSX1 | 465 | uc001urr.1 | FALSE |
| chr1 | 91192162 | 91192466 | 0.359444303 | 1.078332909 | 10126 | 305 | 343472 | BARHL2 | −9368 | uc001dns.3 | FALSE |
| chr14 | 96342250 | 96342747 | 0.358502343 | 1.075507028 | 62946 | 498 | 100507043 | TUNAR | −362 | uc001yfe.3 | TRUE |
| chr10 | 105036701 | 105036747 | 0.357906803 | 1.073720409 | 27795 | 47 | 9118 | INA | −173 | uc009xxj.3 | TRUE |
| chr12 | 94543449 | 94543680 | 0.357901827 | 1.07370548 | 48674 | 232 | 10154 | PLXNC1 | 950 | uc001tdc.3 | FALSE |
| chr5 | 140800398 | 140800474 | 0.357782235 | 1.073346705 | 156929 | 77 | 56099 | PCDHGB7 | 3138 | uc003lko.1 | FALSE |
| chr1 | 98511629 | 98511792 | 0.357600431 | 1.072801294 | 10546 | 164 | 406928 | MIR137 | 0 | uc021oqk.1 | TRUE |
| chr14 | 36983518 | 36983694 | 0.357494775 | 1.072484325 | 59157 | 177 | 253970 | SFTA3 | 5123 | uc001wtq.3 | FALSE |
| chr14 | 104552032 | 104552209 | 0.357340657 | 1.072021971 | 64027 | 178 | 374569 | ASPG | 9 | uc001yor.2 | FALSE |
| chr6 | 27259010 | 27259121 | 0.357297994 | 1.071893982 | 162766 | 112 | 94026 | POM121L2 | 20890 | uc011dku.1 | FALSE |
| chr4 | 158141526 | 158141570 | 0.357297705 | 1.071893114 | 147893 | 45 | 2891 | GRIA2 | 231 | uc021xtr.1 | FALSE |
| chr7 | 1980117 | 1980273 | −0.3572513 | 1.0717539 | 173222 | 157 | 8379 | MAD1L1 | 0 | uc003sld.1 | TRUE |
| chr15 | 26108663 | 26108683 | 0.357218915 | 1.071656746 | 65101 | 21 | 57194 | ATP10A | 1634 | uc010ayu.3 | FALSE |
| chr8 | 55367466 | 55367627 | 0.356576393 | 1.069729179 | 189185 | 162 | 64321 | SOX17 | −2868 | uc003xsb.4 | FALSE |
| chr15 | 60285691 | 60285821 | 0.356526546 | 1.069579638 | 67548 | 131 | 27023 | FOXB1 | −10600 | uc002agj.1 | FALSE |
| chr1 | 968558 | 969257 | 0.355669131 | 1.067007392 | 115 | 700 | 375790 | AGRN | 13055 | uc001ack.2 | FALSE |
| chr12 | 54423428 | 54423566 | 0.35533001 | 1.065990029 | 46534 | 139 | 3223 | HOXC6 | 1234 | uc001sev.3 | FALSE |
| chr10 | 23479993 | 23480018 | 0.355222997 | 1.065668991 | 22261 | 26 | 256297 | PTF1A | −1442 | uc001irp.3 | TRUE |
| chr2 | 176983815 | 176983949 | 0.355130886 | 1.065392659 | 114071 | 135 | 3236 | HOXD10 | 2323 | uc002ukj.3 | FALSE |
| chr13 | 37004536 | 37004617 | 0.354995717 | 1.06498715 | 54130 | 82 | 8900 | CCNA1 | −1040 | uc010teo.2 | TRUE |
| chr12 | 85673221 | 85673347 | 0.354982394 | 1.064947182 | 48324 | 127 | 8092 | ALX1 | −689 | uc001tae.4 | TRUE |
| chr13 | 28503060 | 28503373 | 0.354523571 | 1.063570712 | 53543 | 314 | 3651 | PDX1 | 8892 | uc001urt.2 | FALSE |
| chr1 | 240256603 | 240256644 | 0.531599011 | 1.063198021 | 19560 | 42 | 56776 | FMN2 | 1418 | uc010pye.2 | FALSE |
| chr2 | 176964512 | 176964720 | 0.354098968 | 1.062296904 | 114061 | 209 | 3238 | HOXD12 | 0 | uc010zev.1 | TRUE |
| chr7 | 54609953 | 54610022 | 0.353669682 | 1.061009047 | 177223 | 70 | 222008 | VSTM2A | 0 | uc010kzf.3 | TRUE |
| chr19 | 37407257 | 37407374 | 0.353490543 | 1.060471628 | 98823 | 118 | 374900 | ZNF568 | 26 | uc010xtn.2 | FALSE |
| chr17 | 46827458 | 46827626 | 0.353179482 | 1.059538445 | 86681 | 169 | 284076 | TTLL6 | 44060 | uc002ioa.3 | FALSE |
| chr18 | 55095249 | 55095886 | 0.352457286 | 1.057371858 | 92579 | 638 | 9480 | ONECUT2 | −7031 | uc002lgo.3 | FALSE |
| chr14 | 21238111 | 21238436 | −0.351638675 | 1.054916024 | 58218 | 326 | 64184 | EDDM3B | 1525 | uc001vyd.3 | FALSE |
| chr13 | 46425837 | 46425874 | 0.351635057 | 1.054905171 | 54729 | 38 | 283514 | SIAH3 | 0 | uc001vap.3 | TRUE |
| chr19 | 52222446 | 52223099 | 0.351528668 | 1.054586003 | 101527 | 654 | 3036 | HAS1 | 49 | uc010epd.1 | FALSE |
| chr17 | 5019452 | 5019669 | 0.351308993 | 1.053926978 | 81672 | 218 | 7775 | ZNF232 | 6728 | uc002gau.1 | FALSE |
| chr7 | 15725196 | 15725477 | 0.350401652 | 1.051204955 | 174517 | 282 | 4223 | MEOX2 | 831 | uc003stc.3 | FALSE |
| chr10 | 23462117 | 23462616 | 0.350096735 | 1.050290206 | 22258 | 500 | 256297 | PTF1A | −18844 | uc001irp.3 | FALSE |
| chr19 | 21541756 | 21541772 | 0.349263415 | 1.047790245 | 97962 | 17 | 148203 | ZNF738 | 21 | uc002nps.4 | FALSE |
| chr11 | 627152 | 627175 | 0.523450484 | 1.046900967 | 30930 | 24 | 6343 | SCT | 0 | uc001lqo.1 | TRUE |
| chr12 | 114877063 | 114877421 | 0.348724497 | 1.046173491 | 50283 | 359 | 255480 | TBX5-AS1 | 30504 | uc001tvs.2 | FALSE |
| chr1 | 119522188 | 119522637 | 0.348675146 | 1.046025438 | 11731 | 450 | 6913 | TBX15 | 9542 | uc001ehl.1 | FALSE |
| chr13 | 102569538 | 102569876 | 0.348338829 | 1.045016487 | 56678 | 339 | 2259 | FGF14 | 484248 | uc001vpe.2 | FALSE |
| chr13 | 53312966 | 53313529 | 0.348312409 | 1.044937227 | 55233 | 564 | 11061 | CNMD | 418 | uc001vhh.2 | FALSE |
| chrX | 82763219 | 82763247 | 0.348186339 | 1.044559018 | 202780 | 29 | 5456 | POU3F4 | −22 | uc004eeg.2 | TRUE |
| chr7 | 99574383 | 99574466 | −0.348178501 | 1.044535504 | 179897 | 84 | 563 | AZGP1 | −648 | uc003ush.3 | TRUE |
| chr20 | 61808691 | 61809035 | 0.347929978 | 1.043789935 | 122474 | 345 | 406909 | MIR124-3 | −817 | uc002yeg.1 | TRUE |
| chr6 | 105388668 | 105388731 | 0.347901495 | 1.043704486 | 167810 | 64 | 100113403 | LIN28B-AS1 | −266 | uc031spf.1 | TRUE |
| chr6 | 154360344 | 154360483 | 0.347662421 | 1.042987264 | 170587 | 140 | 4988 | OPRM1 | 0 | uc003qpt.1 | TRUE |
| chr2 | 5832882 | 5833169 | 0.347515271 | 1.042545813 | 103479 | 288 | 6664 | SOX11 | 83 | uc002qyj.3 | FALSE |
| chr17 | 46629350 | 46629804 | 0.347220043 | 1.04166013 | 86602 | 455 | 3213 | HOXB3 | 432 | uc002inn.3 | FALSE |
| chr10 | 102501346 | 102501375 | 0.347087507 | 1.04126252 | 27403 | 30 | 5076 | PAX2 | −4093 | uc001krk.4 | FALSE |
| chr11 | 58980665 | 58981095 | −0.347037791 | 1.041113372 | 35427 | 431 | 219972 | MPEG1 | −171 | uc001nnu.4 | TRUE |
| chr1 | 116371187 | 116371475 | 0.346885346 | 1.040656038 | 11521 | 289 | 4808 | NHLH2 | 11858 | uc009wgz.3 | FALSE |
| chr7 | 155259524 | 155259845 | 0.346673018 | 1.040019053 | 184104 | 322 | 2020 | EN2 | 8700 | uc003wmb.3 | FALSE |
| chr11 | 65816463 | 65816521 | 0.519104376 | 1.038208753 | 36659 | 59 | 89792 | GAL3ST3 | 130 | uc001ogw.3 | FALSE |
| chr17 | 56232302 | 56232499 | −0.345746212 | 1.037238636 | 87502 | 198 | 26689 | OR4D1 | −16 | uc010wno.2 | TRUE |
| chr6 | 32036897 | 32036954 | −0.345693947 | 1.037081841 | 163799 | 58 | 7148 | TNXB | 40197 | uc003nzh.1 | FALSE |
| chr7 | 98246001 | 98246006 | 0.517275605 | 1.034551211 | 179677 | 6 | 4885 | NPTX2 | −591 | uc003upl.2 | TRUE |
| chr15 | 53087068 | 53087316 | 0.343747246 | 1.031241737 | 67183 | 249 | 3175 | ONECUT1 | −4859 | uc002aci.2 | FALSE |
| chr19 | 52452317 | 52452447 | 0.343667463 | 1.031002388 | 101558 | 131 | 79898 | ZNF613 | 21629 | uc002pya.2 | FALSE |
| chr3 | 126113658 | 126113682 | 0.343460404 | 1.030381213 | 135523 | 25 | 348807 | CFAP100 | −100 | uc003eiu.1 | TRUE |
| chr1 | 114414312 | 114414408 | −0.343150792 | 1.029452375 | 11354 | 97 | 26191 | PTPN22 | 0 | uc001edu.2 | TRUE |
| chr6 | 78176622 | 78176806 | 0.342966763 | 1.02890029 | 166978 | 185 | 3351 | HTR1B | −3502 | uc003pil.1 | FALSE |
| chr6 | 99292286 | 99292347 | 0.514069282 | 1.028138565 | 167629 | 62 | 5454 | POU3F2 | 9706 | uc003ppe.3 | FALSE |
| chr11 | 2161892 | 2162183 | 0.342658584 | 1.027975751 | 31470 | 292 | 51214 | IGF2-AS | 134 | uc010qxi.2 | FALSE |
| chr2 | 119611101 | 119611362 | 0.342285678 | 1.026857035 | 110888 | 262 | 2019 | EN1 | −5342 | uc002tlm.3 | FALSE |
| chr8 | 4849399 | 4849827 | 0.341012901 | 1.023038702 | 185745 | 429 | 64478 | CSMD1 | 2501 | uc022aqr.1 | FALSE |
| chr6 | 28493312 | 28493601 | −0.341001062 | 1.023003186 | 162969 | 290 | 2880 | GPX5 | −188 | uc003nll.2 | TRUE |
| chr12 | 114847438 | 114847641 | 0.34097672 | 1.022930161 | 50277 | 204 | 255480 | TBX5-AS1 | 879 | uc001tvs.2 | FALSE |
| chr2 | 240169028 | 240169280 | −0.34089031 | 1.022670931 | 118375 | 253 | 9759 | HDAC4 | 51054 | uc010zoa.1 | FALSE |
| chr1 | 207669716 | 207670014 | 0.340520164 | 1.021560491 | 17043 | 299 | 1378 | CR1 | 243 | uc021pij.1 | FALSE |
| chr14 | 48145000 | 48145108 | 0.51025785 | 1.020515699 | 59408 | 109 | 161357 | MDGA2 | −843 | uc001wwj.4 | TRUE |
| chr4 | 188916668 | 188916724 | 0.34010092 | 1.020302759 | 149273 | 57 | 132625 | ZFP42 | −201 | uc003izh.1 | TRUE |
| chr4 | 4864430 | 4864532 | 0.33950906 | 1.018527181 | 141368 | 103 | 4487 | MSX1 | 3038 | uc003gif.3 | FALSE |
| chrX | 145075713 | 145076186 | −0.339278544 | 1.017835632 | 204094 | 474 | 100126303 | MIR890 | 0 | uc022cfo.1 | TRUE |
| chr7 | 153749736 | 153749756 | 0.339042049 | 1.017126146 | 183956 | 21 | 1804 | DPP6 | 165317 | uc003wlj.3 | FALSE |
| chr8 | 9763739 | 9764073 | 0.338330048 | 1.014990143 | 186130 | 335 | 406907 | MIR124-1 | −2757 | uc003wsw.1 | FALSE |
| chr4 | 66535351 | 66535403 | 0.337985264 | 1.013955792 | 144085 | 53 | 2044 | EPHA5 | 250 | uc003hcx.3 | FALSE |
| chr6 | 28510306 | 28510339 | 0.33769053 | 1.01307159 | 162978 | 34 | 2880 | GPX5 | 14198 | uc003nln.2 | FALSE |
| chr17 | 50235393 | 50235965 | 0.337097342 | 1.011292027 | 87173 | 573 | 56934 | CA10 | 0 | uc002itv.4 | TRUE |
| chr12 | 64062498 | 64062526 | 0.336775344 | 1.010326031 | 47335 | 29 | 283417 | DPY19L2 | −144 | uc001srp.1 | TRUE |
| chr10 | 123353822 | 123353956 | 0.336540927 | 1.009622781 | 28969 | 135 | 2263 | FGFR2 | 2203 | uc021pzw.1 | FALSE |
| chr5 | 140778342 | 140778424 | 0.336154147 | 1.008462442 | 156916 | 83 | 56101 | PCDHGB5 | 647 | uc003lkf.2 | FALSE |
| chr1 | 179544765 | 179545091 | 0.335731298 | 1.007193895 | 15161 | 327 | 7827 | NPHS2 | 0 | uc009wxi.3 | TRUE |
| chr9 | 79629843 | 79630305 | 0.335729699 | 1.007189098 | 196096 | 463 | 442425 | FOXB2 | −4266 | uc004ako.1 | FALSE |
| chrX | 139585911 | 139586310 | 0.335594545 | 1.006783635 | 204028 | 400 | 6658 | SOX3 | 915 | uc004fbd.1 | FALSE |
| chr19 | 52873106 | 52873173 | 0.334720753 | 1.00416226 | 101612 | 68 | 400713 | ZNF880 | 0 | uc021uyu.1 | TRUE |
| chr12 | 106979430 | 106979482 | 0.33427916 | 1.00283748 | 49452 | 53 | 5992 | RFX4 | 2745 | uc001tlr.3 | FALSE |
| chr2 | 222435214 | 222435709 | 0.334108529 | 1.002325586 | 116673 | 496 | 2043 | EPHA4 | 1292 | uc002vmr.2 | FALSE |
| chr6 | 30095199 | 30095258 | 0.333832388 | 1.001497164 | 163322 | 60 | 135644 | TRIM40 | −8627 | uc003npk.2 | FALSE |
| chr13 | 100624855 | 100624965 | 0.33340918 | 1.000227541 | 56553 | 111 | 85416 | ZIC5 | −677 | uc001vom.1 | TRUE |
| chr7 | 27169068 | 27169208 | 0.333126225 | 0.999378674 | 175214 | 141 | 3201 | HOXA4 | 1191 | uc003sym.4 | FALSE |
| chr6 | 30175299 | 30175327 | 0.332989503 | 0.99896851 | 163352 | 29 | 7726 | TRIM26 | 5944 | uc003npr.3 | FALSE |
| chr17 | 78735268 | 78735550 | −0.332216843 | 0.996650528 | 90229 | 283 | 57521 | RPTOR | 216643 | uc002jyu.1 | FALSE |
| chr14 | 33402368 | 33402512 | 0.331946328 | 0.995838983 | 58988 | 145 | 64067 | NPAS3 | −5947 | uc001wrs.3 | FALSE |
| chr14 | 59932153 | 59932273 | 0.331877032 | 0.995631095 | 60230 | 121 | 64582 | GPR135 | −94 | uc010apj.3 | TRUE |
| chr12 | 54447349 | 54447584 | 0.331618175 | 0.994854526 | 46543 | 236 | 3221 | HOXC4 | 36707 | uc001sex.3 | FALSE |
| chr4 | 41875470 | 41875784 | 0.331572896 | 0.994718689 | 143323 | 315 | 55161 | TMEM33 | −61353 | uc003gwi.2 | FALSE |
| chr12 | 114846849 | 114847043 | 0.330636001 | 0.991908003 | 50277 | 195 | 255480 | TBX5-AS1 | 290 | uc001tvs.2 | FALSE |
| chr18 | 59000800 | 59001158 | 0.330173229 | 0.990519686 | 92713 | 359 | 28316 | CDH20 | 0 | uc002lif.2 | TRUE |
| chr16 | 23847325 | 23847675 | 0.329595393 | 0.98878618 | 74784 | 351 | 5579 | PRKCB | 25 | uc002dme.3 | FALSE |
| chr11 | 59210634 | 59211431 | −0.32951757 | 0.98855271 | 35437 | 798 | 219982 | OR5A1 | 0 | uc001nnx.1 | TRUE |
| chr6 | 32116653 | 32116780 | 0.493195226 | 0.986390451 | 163826 | 128 | 80863 | PRRT1 | 2920 | uc003nzs.3 | FALSE |
| chr13 | 112724270 | 112724583 | 0.328762123 | 0.98628637 | 57392 | 314 | 6656 | SOX1 | 2357 | uc001vsb.1 | FALSE |
| chr5 | 42953543 | 42953590 | 0.492237785 | 0.984475571 | 151968 | 48 | 648987 | LOC648987 | 65323 | uc021xye.1 | FALSE |
| chr11 | 30037833 | 30038286 | 0.328085904 | 0.984257712 | 33692 | 454 | 3739 | KCNA4 | 291 | uc001msk.3 | FALSE |
| chr3 | 132756986 | 132757087 | 0.32797952 | 0.98393856 | 136193 | 102 | 66000 | TMEM108 | −84 | uc003eph.3 | TRUE |
| chr18 | 56940418 | 56940693 | 0.327848828 | 0.983546484 | 92680 | 276 | 30062 | RAX | 0 | uc010dpp.3 | TRUE |
| chr10 | 8097183 | 8097354 | 0.327525901 | 0.982577702 | 21365 | 172 | 2625 | GATA3 | 516 | uc001ika.3 | FALSE |
| chr22 | 44577211 | 44577240 | −0.327329999 | 0.981989997 | 127309 | 30 | 64098 | PARVG | 0 | uc021wrb.2 | TRUE |
| chr19 | 12175505 | 12175631 | 0.326998132 | 0.980994397 | 96466 | 127 | 284391 | ZNF844 | 0 | uc010dym.1 | TRUE |
| chrX | 122317614 | 122317633 | 0.326945401 | 0.980836204 | 203560 | 20 | 2892 | GRIA3 | −463 | uc031tki.1 | TRUE |
| chr1 | 244213577 | 244213618 | 0.325618608 | 0.976855825 | 19789 | 42 | 10472 | ZBTB18 | 1336 | uc001iad.5 | FALSE |
| chr1 | 67217826 | 67217886 | 0.325062461 | 0.975187384 | 9159 | 61 | 200132 | TCTEX1D1 | −254 | uc001dcv.3 | TRUE |
| chr9 | 140401382 | 140401455 | −0.324268224 | 0.972804672 | 200500 | 74 | 375775 | PNPLA7 | 15854 | uc011mfa.1 | FALSE |
| chr20 | 26190328 | 26190347 | 0.485723195 | 0.97144639 | 120206 | 20 | 284801 | MIR663AHG | −459 | uc002wvk.3 | TRUE |
| chr1 | 44401857 | 44402353 | 0.323814266 | 0.971442799 | 7323 | 497 | 9048 | ARTN | 203 | uc001ckw.3 | FALSE |
| chr20 | 30582838 | 30583001 | 0.323743542 | 0.971230626 | 120329 | 164 | 343702 | XKR7 | 27033 | uc002wxf.2 | FALSE |
| chr1 | 3567214 | 3567303 | 0.485329705 | 0.97065941 | 1346 | 90 | 49856 | WRAP73 | −543 | uc001ako.3 | TRUE |
| chr5 | 2252878 | 2252995 | −0.321250229 | 0.963750686 | 150178 | 118 | 50805 | IRX4 | −365585 | uc031siq.1 | FALSE |
| chr14 | 29235973 | 29236008 | 0.320687659 | 0.962062978 | 58843 | 36 | 2290 | FOXG1 | −270 | uc001wqe.4 | TRUE |
| chr8 | 55382901 | 55383182 | 0.480914618 | 0.961829237 | 189192 | 282 | 64321 | SOX17 | 12406 | uc003xsb.4 | FALSE |
| chr6 | 96463185 | 96463710 | 0.320089204 | 0.960267611 | 167544 | 526 | 10690 | FUT9 | −135 | uc003pop.4 | TRUE |
| chr19 | 58095424 | 58095468 | 0.319383812 | 0.958151435 | 102564 | 45 | 284307 | ZIK1 | −160 | uc002qpg.3 | TRUE |
| chr12 | 130388216 | 130388234 | 0.478018087 | 0.956036175 | 51866 | 19 | 121256 | TMEM132D | −4 | uc009zyl.1 | TRUE |
| chr19 | 37997831 | 37997991 | 0.317689378 | 0.953068133 | 98881 | 161 | 390927 | ZNF793 | 0 | uc010xts.2 | TRUE |
| chr10 | 63212496 | 63212862 | 0.317175802 | 0.951527405 | 24352 | 367 | 219623 | TMEM26 | 346 | uc001jlq.3 | FALSE |
| chr6 | 28304194 | 28304249 | 0.317119227 | 0.951357681 | 162933 | 56 | 64288 | ZSCAN31 | 17723 | uc003nlc.3 | FALSE |
| chr18 | 5543548 | 5543878 | 0.31697427 | 0.950922809 | 91317 | 331 | 23136 | EPB41L3 | 108 | uc002kmu.1 | FALSE |
| chr11 | 71955332 | 71955401 | 0.316234868 | 0.948704605 | 37811 | 70 | 401 | PHOX2A | −112 | uc001osh.4 | TRUE |
| chr7 | 157584081 | 157584183 | −0.474258643 | 0.948517286 | 184582 | 103 | 5799 | PTPRN2 | 750285 | uc003wns.3 | FALSE |
| chr3 | 159756690 | 159756929 | 0.316065853 | 0.94819756 | 137725 | 240 | 3592 | IL12A | 50067 | uc003fcx.3 | FALSE |
| chr19 | 34174345 | 34174665 | −0.316058476 | 0.948175428 | 98373 | 321 | 64377 | CHST8 | 61484 | uc002nuu.3 | FALSE |
| chr9 | 120507228 | 120507431 | 0.472120483 | 0.944240966 | 197787 | 204 | 7099 | TLR4 | 40775 | uc004bkb.4 | FALSE |
| chr2 | 176956573 | 176956678 | 0.471570164 | 0.943140329 | 114059 | 106 | 3239 | HOXD13 | −854 | uc002ukf.1 | TRUE |
| chr15 | 45479642 | 45479755 | 0.471284901 | 0.942569802 | 66703 | 114 | 90525 | SHF | 144 | uc010ueu.2 | FALSE |
| chr6 | 166218512 | 166218519 | 0.471033015 | 0.94206603 | 171520 | 8 | 90632 | LINC00473 | 183008 | uc003qun.3 | FALSE |
| chr7 | 37487613 | 37487633 | 0.470703068 | 0.941406135 | 176082 | 21 | 9844 | ELMO1 | 922 | uc003tfk.2 | FALSE |
| chr8 | 9760880 | 9761141 | 0.313037666 | 0.939112997 | 186130 | 262 | 406907 | MIR124-1 | 0 | uc003wsw.1 | TRUE |
| chr12 | 95942907 | 95942964 | 0.465874517 | 0.931749033 | 48776 | 58 | 84101 | USP44 | 2299 | uc001teg.3 | FALSE |
| chr11 | 59807594 | 59808049 | −0.310401808 | 0.931205423 | 35492 | 456 | 219990 | OOSP2 | 0 | uc001nol.3 | TRUE |
| chr11 | 44332653 | 44332664 | 0.465305524 | 0.930611048 | 34379 | 12 | 60529 | ALX4 | −937 | uc001myb.3 | TRUE |
| chr11 | 5531199 | 5531283 | −0.31010171 | 0.930305129 | 31994 | 85 | 3048 | HBG2 | 135728 | uc001may.1 | FALSE |
| chr14 | 101489415 | 101489528 | −0.309286533 | 0.9278596 | 63521 | 114 | 693121 | MIR411 | −134 | uc021scm.1 | TRUE |
| chr17 | 46711017 | 46711035 | 0.462643949 | 0.925287898 | 86656 | 19 | 406972 | MIR196A1 | −1096 | uc010wln.1 | TRUE |
| chr13 | 77461335 | 77461368 | 0.461674512 | 0.923349023 | 55788 | 34 | 115207 | KCTD12 | −795 | uc010aeu.1 | TRUE |
| chr1 | 190447290 | 190447421 | 0.461588214 | 0.923176428 | 15751 | 132 | 339479 | BRINP3 | −531 | uc001gse.1 | TRUE |
| chrX | 139588759 | 139589424 | 0.307552294 | 0.922656882 | 204029 | 666 | 6658 | SOX3 | −1534 | uc004fbd.1 | TRUE |
| chr16 | 1316081 | 1316439 | −0.305896869 | 0.917690607 | 72260 | 359 | 23430 | TPSD1 | 9808 | uc010brm.1 | FALSE |
| chr2 | 168150602 | 168151004 | 0.30407801 | 0.91223403 | 113388 | 403 | 129446 | XIRP2 | 106809 | uc010fpr.3 | FALSE |
| chr7 | 158799748 | 158799777 | −0.454897701 | 0.909795402 | 184939 | 30 | 154822 | LINC00689 | −1268 | uc003wof.3 | TRUE |
| chr12 | 54384666 | 54384744 | 0.453648982 | 0.907297963 | 46516 | 79 | 3226 | HOXC10 | 5720 | uc001seo.2 | FALSE |
| chr19 | 58446745 | 58446758 | 0.453438996 | 0.906877991 | 102629 | 14 | 147686 | ZNF418 | −5 | uc002qqs.1 | TRUE |
| chr1 | 175568559 | 175568710 | 0.453357834 | 0.906715669 | 14954 | 152 | 7143 | TNR | 144042 | uc010pmz.1 | FALSE |
| chr6 | 166580952 | 166580983 | 0.450660267 | 0.901320533 | 171569 | 32 | 6862 | TBXT | 382 | uc003qut.2 | FALSE |
| chr10 | 118030848 | 118030970 | 0.44910331 | 0.89820662 | 28533 | 123 | 2674 | GFRA1 | 849 | uc001lci.3 | FALSE |
| chr17 | 46711341 | 46711446 | 0.447994911 | 0.895989822 | 86656 | 106 | 406972 | MIR196A1 | −1420 | uc010wln.1 | TRUE |
| chr15 | 89952499 | 89952904 | 0.447793679 | 0.895587358 | 70551 | 406 | 254559 | MIR9-3HG | 31226 | uc002bnw.2 | FALSE |
| chr19 | 57618136 | 57618436 | 0.445991324 | 0.891982648 | 102465 | 301 | 57663 | USP29 | −13073 | uc002qny.3 | FALSE |
| chr7 | 149112318 | 149112402 | 0.445570835 | 0.891141669 | 183334 | 85 | 27153 | ZNF777 | 45651 | uc003wfv.3 | FALSE |
| chr14 | 92790097 | 92790285 | 0.444905794 | 0.889811587 | 62540 | 189 | 123041 | SLC24A4 | 0 | uc001yak.3 | TRUE |
| chr3 | 147077616 | 147077636 | 0.444716032 | 0.889432065 | 136987 | 21 | 84107 | ZIC4 | 32581 | uc021xfe.1 | FALSE |
| chr8 | 132054555 | 132054713 | 0.444208463 | 0.888416925 | 192903 | 159 | 114 | ADCY8 | −1720 | uc003ytd.4 | TRUE |
| chr2 | 208989248 | 208989324 | 0.442499629 | 0.884999257 | 115715 | 77 | 1421 | CRYGD | 0 | uc002vcn.4 | TRUE |
| chr5 | 554886 | 555251 | −0.441522955 | 0.88304591 | 149626 | 366 | 100616381 | MIR4456 | −18889 | uc021xvw.1 | FALSE |
| chr1 | 226288408 | 226288430 | 0.441400655 | 0.882801311 | 18342 | 23 | 440926 | H3P6 | 36730 | uc021pjv.1 | FALSE |
| chr15 | 33011166 | 33011185 | 0.441235552 | 0.882471104 | 65622 | 20 | 26585 | GREM1 | 961 | uc010uby.2 | FALSE |
| chr16 | 6069019 | 6069047 | 0.4403261 | 0.880652201 | 73496 | 29 | 54715 | RBFOX1 | −85 | uc002cyr.1 | TRUE |
| chr5 | 140261653 | 140261663 | 0.440319912 | 0.880639824 | 156787 | 11 | 56137 | PCDHA12 | 6722 | uc003lid.3 | FALSE |
| chr2 | 99004092 | 99004292 | −0.4402428 | 0.880485601 | 109554 | 201 | 1261 | CNGA3 | 10341 | uc010fij.3 | FALSE |
| chr4 | 81106857 | 81107087 | 0.43998444 | 0.87996888 | 144787 | 231 | 56978 | PRDM8 | 433 | uc003hmb.4 | FALSE |
| chr7 | 30722320 | 30722327 | 0.439912712 | 0.879825425 | 175594 | 8 | 1395 | CRHR2 | 17392 | uc003tbn.3 | FALSE |
| chr16 | 49315302 | 49315536 | 0.438491761 | 0.876983521 | 76102 | 235 | 869 | CBLN1 | 206 | uc002efq.3 | FALSE |
| chr6 | 170595920 | 170595947 | 0.438134983 | 0.876269966 | 172415 | 28 | 28514 | DLL1 | 3750 | uc003qxn.3 | FALSE |
| chr1 | 149719461 | 149719536 | 0.437943932 | 0.875887865 | 12323 | 76 | 440689 | H2BC18 | 64392 | uc001esp.4 | FALSE |
| chr12 | 22093960 | 22094009 | 0.437309201 | 0.874618402 | 44379 | 50 | 10060 | ABCC9 | 327 | uc001rfl.1 | FALSE |
| chr13 | 108519318 | 108519554 | 0.435212269 | 0.870424539 | 56885 | 237 | 728215 | FAM155A | 0 | uc001vql.3 | TRUE |
| chr14 | 62583795 | 62583919 | 0.434785206 | 0.869570411 | 60446 | 125 | 646113 | LINC00643 | −156 | uc010apt.2 | TRUE |
| chr1 | 67218135 | 67218165 | 0.434660326 | 0.869320652 | 9159 | 31 | 200132 | TCTEX1D1 | 0 | uc009wav.3 | TRUE |
| chr3 | 125655381 | 125655407 | −0.433775803 | 0.867551605 | 135455 | 27 | 200810 | ALG1L | 480 | uc021xdh.1 | FALSE |
| chr18 | 49866531 | 49866548 | 0.433290757 | 0.866581514 | 92505 | 18 | 1630 | DCC | 0 | uc002lfe.2 | TRUE |
| chr10 | 94834582 | 94834763 | 0.43279043 | 0.86558086 | 26680 | 182 | 1592 | CYP26A1 | 935 | uc001kil.2 | FALSE |
| chr8 | 114444989 | 114445084 | 0.432476648 | 0.864953297 | 191865 | 96 | 114788 | CSMD3 | 4158 | uc003ynx.4 | FALSE |
| chr3 | 147108512 | 147108843 | 0.432075504 | 0.864151008 | 136998 | 332 | 84107 | ZIC4 | 1374 | uc021xfe.1 | FALSE |
| chr6 | 106429316 | 106429443 | 0.431524148 | 0.863048296 | 167878 | 128 | 639 | PRDM1 | −104752 | uc003prd.2 | FALSE |
| chr20 | 26188963 | 26188997 | 0.430822914 | 0.861645827 | 120205 | 35 | 284801 | MIR663AHG | 872 | uc021wbn.1 | FALSE |
| chr7 | 149917170 | 149917263 | 0.430327263 | 0.860654525 | 183447 | 94 | 653857 | ACTR3C | 82806 | uc003wgu.2 | FALSE |
| chr2 | 176985174 | 176985592 | 0.430263807 | 0.860527614 | 114073 | 419 | 3236 | HOXD10 | 3682 | uc010zex.2 | FALSE |
| chr2 | 554363 | 554372 | −0.430174325 | 0.860348649 | 102891 | 10 | 129787 | TMEM18 | 121786 | uc002qwk.3 | FALSE |
| chr12 | 81331660 | 81331718 | 0.430160601 | 0.860321201 | 48255 | 59 | 8825 | LIN7A | 0 | uc001szk.1 | TRUE |
| chr18 | 14748250 | 14748285 | 0.429453582 | 0.858907164 | 91736 | 36 | 374860 | ANKRD30B | 11 | uc021uhy.1 | FALSE |
| chr7 | 121940216 | 121940344 | 0.429345144 | 0.858690288 | 181369 | 129 | 389549 | FEZF1 | 4221 | uc010lko.2 | FALSE |
| chr5 | 170743459 | 170743564 | 0.429239638 | 0.858479277 | 158822 | 106 | 30012 | TLX3 | 7171 | uc003mbf.3 | FALSE |
| chr4 | 172734266 | 172734276 | 0.427854389 | 0.855708778 | 148350 | 11 | 442117 | GALNTL6 | −299 | uc003isv.3 | TRUE |
| chr15 | 42749748 | 42749885 | 0.427333336 | 0.854666672 | 66463 | 138 | 64397 | ZNF106 | 0 | uc010udh.1 | TRUE |
| chr11 | 32461212 | 32461240 | 0.42683061 | 0.853661221 | 33813 | 29 | 51352 | WT1-AS | 3627 | uc010red.2 | FALSE |
| chr6 | 161100242 | 161100376 | 0.426600274 | 0.853200548 | 171236 | 135 | 4018 | LPA | −12835 | uc003qtl.3 | FALSE |
| chr7 | 157346614 | 157347105 | −0.42631498 | 0.85262996 | 184453 | 492 | 5799 | PTPRN2 | 987363 | uc011kwb.1 | FALSE |
| chr7 | 158362729 | 158362809 | 0.425387554 | 0.850775109 | 184842 | 81 | 5799 | PTPRN2 | 17673 | uc003wnr.3 | FALSE |
| chr12 | 113901210 | 113901529 | 0.425030664 | 0.850061328 | 50187 | 320 | 64211 | LHX5 | 8348 | uc001tvj.1 | FALSE |
| chr19 | 4305072 | 4305090 | 0.424683992 | 0.849367983 | 95030 | 19 | 79187 | FSD1 | 481 | uc002lzy.2 | FALSE |
| chrX | 51361516 | 51361547 | 0.424318049 | 0.848636099 | 202048 | 32 | 389857 | CENPVL1 | 63900 | uc022bwn.1 | FALSE |
| chr7 | 27204728 | 27204785 | 0.424189733 | 0.848379466 | 175231 | 58 | 3205 | HOXA9 | 364 | uc003syt.3 | FALSE |
| chr6 | 28176163 | 28176230 | 0.423827554 | 0.847655109 | 162904 | 68 | 222699 | TOB2P1 | 10477 | uc011dla.2 | FALSE |
| chr10 | 118891670 | 118891706 | 0.423470677 | 0.846941355 | 28616 | 37 | 11023 | VAX1 | 6106 | uc010qsq.1 | FALSE |
| chr8 | 35093411 | 35093901 | 0.422610976 | 0.845221952 | 188064 | 491 | 137970 | UNC5D | 436 | uc003xjr.2 | FALSE |
| chr13 | 80911626 | 80911692 | 0.422324524 | 0.844649049 | 55933 | 67 | 10253 | SPRY2 | 2102 | uc001vli.3 | FALSE |
| chr9 | 114557239 | 114557390 | −0.421782803 | 0.843565606 | 197516 | 152 | 158401 | SHOC1 | −16 | uc011lwt.2 | TRUE |
| chr3 | 157813609 | 157813670 | 0.421439714 | 0.842879427 | 137627 | 62 | 6474 | SHOX2 | 10282 | uc010hvw.3 | FALSE |
| chr11 | 32009004 | 32009163 | 0.420670148 | 0.841340296 | 33782 | 160 | 5954 | RCN1 | 170890 | uc010reb.2 | FALSE |
| chr12 | 54396354 | 54396440 | 0.420337744 | 0.840675488 | 46521 | 87 | 3225 | HOXC9 | 2477 | uc001seq.3 | FALSE |
| chr14 | 60973462 | 60973517 | 0.419469485 | 0.83893897 | 60296 | 56 | 4990 | SIX6 | −2421 | uc001xfa.4 | FALSE |
| chr19 | 54466441 | 54466538 | 0.419171457 | 0.838342915 | 101893 | 98 | 59283 | CACNG8 | 151 | uc002qcs.2 | FALSE |
| chr8 | 65281689 | 65282185 | 0.417487232 | 0.834974463 | 189643 | 497 | 100130155 | MIR124-2HG | −3590 | uc022avc.2 | FALSE |
| chr15 | 30517589 | 30517601 | 0.416431508 | 0.832863016 | 65440 | 13 | 26082 | LINC02249 | 29350 | uc001zds.2 | FALSE |
| chr19 | 54926805 | 54927291 | 0.416409701 | 0.832819403 | 101990 | 487 | 57348 | TTYH1 | 200 | uc002qft.3 | FALSE |
| chr1 | 166853578 | 166853789 | 0.416240595 | 0.83248119 | 14394 | 212 | 117143 | TADA1 | −7924 | uc001gdw.3 | FALSE |
| chr6 | 50683213 | 50683593 | 0.416157048 | 0.832314095 | 166043 | 381 | 83741 | TFAP2D | 1956 | uc011dwt.2 | FALSE |
| chr8 | 70983567 | 70983600 | 0.415775152 | 0.831550304 | 189933 | 34 | 63978 | PRDM14 | −5 | uc003xym.3 | TRUE |
| chr5 | 5146320 | 5146343 | −0.415516019 | 0.831032038 | 150399 | 24 | 170690 | ADAMTS16 | 5877 | uc003jdl.3 | FALSE |
| chr19 | 22018746 | 22018791 | 0.41545518 | 0.830910359 | 97995 | 46 | 7594 | ZNF43 | 219 | uc002nqk.4 | FALSE |
| chr3 | 161214690 | 161214813 | −0.415147338 | 0.830294676 | 137808 | 124 | 131149 | OTOL1 | 94 | uc011bpb.2 | FALSE |
| chr7 | 96647882 | 96647888 | 0.414901607 | 0.829803214 | 179561 | 7 | 1749 | DLX5 | 6255 | uc003uol.3 | FALSE |
| chr6 | 6004207 | 6004328 | 0.414487206 | 0.828974412 | 161053 | 122 | 51299 | NRN1 | 0 | uc021ykx.1 | TRUE |
| chr16 | 51187807 | 51188129 | 0.413726652 | 0.827453305 | 76312 | 323 | 6299 | SALL1 | −2624 | uc021tie.1 | FALSE |
| chr1 | 157670328 | 157670710 | −0.413580764 | 0.827161528 | 13558 | 383 | 115352 | FCRL3 | 0 | uc001frb.3 | TRUE |
| chr12 | 54398765 | 54398809 | 0.413400828 | 0.826801656 | 46523 | 45 | 3225 | HOXC9 | 4888 | uc001ser.3 | FALSE |
| chr1 | 91182305 | 91182534 | 0.413289922 | 0.826579843 | 10123 | 230 | 343472 | BARHL2 | 260 | uc001dns.3 | FALSE |
| chr14 | 70653919 | 70653964 | 0.411434022 | 0.822868044 | 61050 | 46 | 6547 | SLC8A3 | 1823 | uc010ara.3 | FALSE |
| chr9 | 122131895 | 122132052 | 0.411300782 | 0.822601563 | 197802 | 158 | 1620 | BRINP1 | −156 | uc004bkc.2 | TRUE |
| chr20 | 24929607 | 24929716 | −0.410033956 | 0.820067913 | 120113 | 110 | 8530 | CST7 | −150 | uc002wtx.2 | TRUE |
| chr1 | 38230845 | 38230887 | 0.40975309 | 0.81950618 | 6511 | 43 | 284656 | EPHA10 | −21 | uc009vvi.3 | TRUE |
| chr2 | 73152672 | 73152809 | 0.409412409 | 0.818824817 | 108184 | 138 | 2016 | EMX1 | 8068 | uc002sin.1 | FALSE |
| chr3 | 147141219 | 147141286 | 0.409272993 | 0.818545985 | 137012 | 68 | 7545 | ZIC1 | 14038 | uc003ewe.3 | FALSE |
| chr2 | 242973936 | 242974096 | −0.408987711 | 0.817975422 | 119042 | 161 | 728323 | LINC01881 | −56748 | uc010zpd.1 | FALSE |
| chr7 | 35298212 | 35298391 | 0.40868873 | 0.81737746 | 175921 | 180 | 57057 | TBX20 | −4501 | uc011kas.2 | FALSE |
| chr10 | 105037503 | 105037661 | 0.408111368 | 0.816222736 | 27795 | 159 | 9118 | INA | 583 | uc001kws.3 | FALSE |
| chr1 | 4714033 | 4714171 | 0.407884694 | 0.815769388 | 1501 | 139 | 55966 | AJAP1 | −934 | uc001alm.1 | TRUE |
| chr6 | 117591684 | 117591827 | 0.407207969 | 0.814415938 | 168613 | 144 | 245806 | VGLL2 | 4963 | uc003pxo.3 | FALSE |
| chr6 | 96463959 | 96464135 | 0.407147788 | 0.814295576 | 167544 | 177 | 10690 | FUT9 | 114 | uc003pop.4 | FALSE |
| chr1 | 173639130 | 173639135 | 0.407067166 | 0.814134331 | 14849 | 6 | 339416 | ANKRD45 | −129 | uc001gja.1 | TRUE |
| chr19 | 13112283 | 13112449 | 0.406965691 | 0.813931381 | 96676 | 167 | 4784 | NFIX | 5699 | uc002mwd.3 | FALSE |
| chr6 | 27569094 | 27569167 | −0.406881747 | 0.813763495 | 162815 | 74 | 100507173 | LINC01012 | −92647 | uc021yrb.1 | FALSE |
| chr3 | 44063541 | 44063593 | 0.406733563 | 0.813467126 | 130854 | 53 | 406929 | MIR138-1 | −92111 | uc011azu.1 | FALSE |
| chr14 | 57284096 | 57284219 | 0.406434376 | 0.812868752 | 60074 | 124 | 100309464 | OTX2-AS1 | 4195 | uc001xcr.3 | FALSE |
| chr3 | 147127579 | 147127662 | 0.406285923 | 0.812571846 | 137005 | 84 | 7545 | ZIC1 | 398 | uc003ewe.3 | FALSE |
| chr4 | 147560770 | 147561203 | 0.405461363 | 0.810922727 | 147360 | 434 | 5458 | POU4F2 | 725 | uc003ikv.3 | FALSE |
| chr18 | 909086 | 909154 | 0.405353949 | 0.810707898 | 91210 | 69 | 116 | ADCYAP1 | 3889 | uc010dkh.4 | FALSE |
| chr10 | 135043492 | 135043506 | 0.405095551 | 0.810191102 | 30576 | 15 | 8433 | UTF1 | −272 | uc001lmc.3 | TRUE |
| chr2 | 45155703 | 45155991 | 0.404652262 | 0.809304523 | 106354 | 289 | 6496 | SIX3 | −13046 | uc002run.2 | FALSE |
| chr18 | 19745316 | 19745458 | 0.403649372 | 0.807298744 | 91797 | 143 | 100128893 | GATA6-AS1 | 3471 | uc031rhq.1 | FALSE |
| chr2 | 182542901 | 182543233 | 0.403519995 | 0.80703999 | 114383 | 333 | 375298 | CERKL | 2159 | uc002uof.4 | FALSE |
| chr2 | 176993017 | 176993089 | 0.403267234 | 0.806534469 | 114075 | 73 | 3234 | HOXD8 | −1333 | uc002ukn.3 | TRUE |
| chr19 | 35395973 | 35396203 | 0.4023945 | 0.804788999 | 98467 | 231 | 100652909 | LINC00904 | −6126 | uc002nxd.2 | FALSE |
| chr11 | 56043040 | 56043514 | −0.401673004 | 0.803346007 | 35125 | 475 | 390155 | OR5T1 | 0 | uc001nio.1 | TRUE |
| chr7 | 107642444 | 107642453 | 0.401495014 | 0.802990027 | 180829 | 10 | 3912 | LAMB1 | 0 | uc003vev.2 | TRUE |
| chr8 | 85097049 | 85097056 | 0.401323073 | 0.802646145 | 190478 | 8 | 138046 | RALYL | 1463 | uc003yct.4 | FALSE |
| chr7 | 960350 | 960411 | −0.401206353 | 0.802412705 | 172778 | 62 | 11033 | ADAP1 | 102 | uc011jvs.2 | FALSE |
| chr1 | 18958924 | 18959268 | 0.400875206 | 0.801750411 | 3911 | 345 | 5081 | PAX7 | 1424 | uc010oct.2 | FALSE |
| chr1 | 242686709 | 242687018 | 0.400788622 | 0.801577244 | 19693 | 310 | 200150 | PLD5 | 980 | uc001hzo.2 | FALSE |
| chr19 | 58545728 | 58545837 | 0.400783844 | 0.801567689 | 102650 | 110 | 284312 | ZSCAN1 | 294 | uc002qrc.1 | FALSE |
| chr12 | 24717058 | 24717178 | 0.400703559 | 0.801407118 | 44466 | 121 | 6660 | SOX5 | −1675 | uc001rfx.4 | TRUE |
| chr5 | 528580 | 528621 | 0.40048731 | 0.800974619 | 149618 | 42 | 6550 | SLC9A3 | −4031 | uc003jbe.2 | FALSE |
| chr2 | 200335558 | 200335874 | 0.400391706 | 0.800783413 | 115028 | 317 | 23314 | SATB2 | 115 | uc002uuz.2 | FALSE |
| chr6 | 38683035 | 38683221 | 0.399941692 | 0.799883383 | 164934 | 187 | 1769 | DNAH8 | 0 | uc021yzh.1 | TRUE |
| chr10 | 85898649 | 85898700 | −0.399873625 | 0.79974725 | 26062 | 52 | 27069 | GHITM | −485 | uc001kcs.1 | TRUE |
| chr18 | 55103734 | 55103785 | 0.399673882 | 0.799347763 | 92583 | 52 | 9480 | ONECUT2 | 817 | uc002lgo.3 | FALSE |
| chr3 | 179755086 | 179755235 | 0.399611994 | 0.799223987 | 138557 | 150 | 51555 | PEX5L | −245 | uc003fki.2 | TRUE |
| chr17 | 46627848 | 46628224 | 0.399446283 | 0.798892566 | 86602 | 377 | 3213 | HOXB3 | 2012 | uc002inn.3 | FALSE |
| chr5 | 87990145 | 87990490 | 0.399434039 | 0.798868078 | 154029 | 346 | 645323 | LINC00461 | −9525 | uc011cua.1 | FALSE |
| chr8 | 145925997 | 145926089 | 0.399381347 | 0.798762693 | 194542 | 93 | 80728 | ARHGAP39 | −14803 | uc011llk.1 | FALSE |
| chr13 | 58208929 | 58209133 | 0.399135531 | 0.798271061 | 55356 | 205 | 27253 | PCDH17 | 3140 | uc010aec.1 | FALSE |
| chr18 | 44777736 | 44777799 | 0.39910431 | 0.798208619 | 92334 | 64 | 652991 | SKOR2 | −2182 | uc031rif.1 | FALSE |
| chr3 | 170302890 | 170303045 | 0.398809648 | 0.797619296 | 138058 | 156 | 57709 | SLC7A14 | 818 | uc003fgz.2 | FALSE |
| chr6 | 28602927 | 28602938 | 0.398151465 | 0.796302929 | 163001 | 12 | 114821 | ZBED9 | −47815 | uc003nlo.3 | FALSE |
| chr13 | 113719136 | 113719172 | −0.397943132 | 0.795886265 | 57692 | 37 | 23263 | MCF2L | 20167 | uc001vst.1 | FALSE |
| chr17 | 58216297 | 58216415 | 0.397881271 | 0.795762542 | 87694 | 119 | 762 | CA4 | −10887 | uc002iym.4 | FALSE |
| chr5 | 63257873 | 63257885 | 0.397519356 | 0.795038712 | 152759 | 13 | 3350 | HTR1A | 234 | uc011cqt.3 | FALSE |
| chr10 | 101287839 | 101287846 | 0.397239586 | 0.794479173 | 27237 | 8 | 159296 | NKX2-3 | −4844 | uc009xwj.3 | FALSE |
| chr14 | 92785924 | 92786270 | −0.397107263 | 0.794214525 | 62537 | 347 | 123041 | SLC24A4 | −2655 | uc001yai.3 | FALSE |
| chr2 | 220299740 | 220299900 | 0.397045816 | 0.794091632 | 116526 | 161 | 10290 | SPEG | 40 | uc010fwg.3 | FALSE |
| chr19 | 36736272 | 36736477 | 0.396608491 | 0.793216982 | 98746 | 206 | 7705 | ZNF146 | 30768 | uc002odo.3 | FALSE |
| chr14 | 101923256 | 101923366 | 0.396270481 | 0.792540961 | 63577 | 111 | 100302145 | MIR1247 | 103393 | uc021sdw.1 | FALSE |
| chr20 | 22559676 | 22559802 | 0.395486422 | 0.790972843 | 120019 | 127 | 140828 | LINC00261 | −396 | uc010zsp.2 | TRUE |
| chr2 | 171573223 | 171573419 | 0.395118244 | 0.790236489 | 113627 | 197 | 389058 | SP5 | 1366 | uc002uge.3 | FALSE |
| chr13 | 100643312 | 100643388 | 0.395069733 | 0.790139466 | 56561 | 77 | 7546 | ZIC2 | 9286 | uc001von.3 | FALSE |
| chr12 | 54441213 | 54441458 | 0.395055194 | 0.790110389 | 46539 | 246 | 3221 | HOXC4 | 30571 | uc001sex.3 | FALSE |
| chr12 | 54357530 | 54357604 | 0.393937319 | 0.787874637 | 46502 | 75 | 100124700 | HOTAIR | 4936 | uc009zne.4 | FALSE |
| chr17 | 37321490 | 37321631 | 0.393177388 | 0.786354776 | 84976 | 142 | 390790 | ARL5C | 783 | uc010wea.2 | FALSE |
| chr6 | 106035490 | 106035528 | −0.393109479 | 0.786218958 | 167857 | 39 | 5550 | PREP | −184491 | uc003prc.3 | FALSE |
| chr12 | 8219020 | 8219358 | −0.393088198 | 0.786176396 | 43493 | 339 | 719 | C3AR1 | −65 | uc001qtv.1 | TRUE |
| chr7 | 155252701 | 155252748 | 0.393007966 | 0.786015932 | 184101 | 48 | 2020 | EN2 | 1877 | uc003wmb.3 | FALSE |
| chr4 | 13545639 | 13545760 | 0.392833492 | 0.785666984 | 142217 | 122 | 579 | NKX3-2 | 354 | uc003gmx.2 | FALSE |
| chr12 | 108169020 | 108169333 | 0.39282654 | 0.78565308 | 49541 | 314 | 121549 | ASCL4 | 858 | uc001tmr.3 | FALSE |
| chr14 | 36992001 | 36992233 | 0.392773562 | 0.785547124 | 59160 | 233 | 7080 | NKX2-1 | −2571 | uc001wtu.3 | FALSE |
| chr18 | 77558846 | 77558916 | 0.392714579 | 0.785429159 | 93436 | 71 | 9150 | CTDP1 | 117416 | uc010xfl.2 | FALSE |
| chrX | 139590383 | 139590479 | 0.392621925 | 0.785243851 | 204029 | 97 | 6658 | SOX3 | −3158 | uc004fbd.1 | FALSE |
| chr5 | 140749735 | 140749745 | 0.392562876 | 0.785125753 | 156904 | 11 | 56110 | PCDHGA5 | 5837 | uc011dat.2 | FALSE |
| chr8 | 26370552 | 26370717 | 0.392356684 | 0.784713368 | 187518 | 166 | 10687 | PNMA2 | 766 | uc003xez.2 | FALSE |
| chr1 | 75602167 | 75602412 | 0.39210165 | 0.7842033 | 9425 | 246 | 431707 | LHX8 | 1600 | uc031pmx.1 | FALSE |
| chr7 | 27291346 | 27291388 | 0.392064803 | 0.784129606 | 175278 | 43 | 2128 | EVX1 | 9027 | uc011jzn.1 | FALSE |
| chr12 | 54339288 | 54339653 | 0.391913182 | 0.783826365 | 46495 | 366 | 3229 | HOXC13 | 6712 | uc031qhm.1 | FALSE |
| chr16 | 86544658 | 86544787 | 0.391653302 | 0.783306603 | 79356 | 130 | 2294 | FOXF1 | 525 | uc002fjl.3 | FALSE |
| chrX | 105066526 | 105066793 | 0.391490864 | 0.782981728 | 203124 | 268 | 203447 | NRK | 0 | uc004emd.3 | TRUE |
| chr10 | 102986973 | 102987257 | 0.391270807 | 0.782541613 | 27516 | 285 | 10660 | LBX1 | 1460 | uc001ksx.3 | FALSE |
| chr19 | 30016147 | 30016478 | 0.390825428 | 0.781650857 | 98139 | 332 | 284395 | LOC284395 | 181 | uc002nse.1 | FALSE |
| chr19 | 1356315 | 1356444 | 0.390781267 | 0.781562533 | 94004 | 130 | 84939 | PWWP3A | 0 | uc010xgm.1 | TRUE |
| chr16 | 89688145 | 89688244 | −0.390659012 | 0.781318024 | 80450 | 100 | 1800 | DPEP1 | 1145 | uc002fnr.4 | FALSE |
| chr7 | 35293753 | 35293759 | 0.390591766 | 0.781183533 | 175919 | 7 | 57057 | TBX20 | −42 | uc011kas.2 | TRUE |
| chr1 | 48190979 | 48191031 | 0.390552284 | 0.781104569 | 7792 | 53 | 388630 | TRABD2B | 271531 | uc021ong.1 | FALSE |
| chr14 | 93706571 | 93706609 | −0.39013664 | 0.78027328 | 62664 | 39 | 55727 | BTBD7 | 54674 | uc010auq.3 | FALSE |
| chr19 | 57323840 | 57323858 | −0.390015641 | 0.780031283 | 102450 | 19 | 100169890 | PEG3-AS1 | 0 | uc010ets.2 | TRUE |
| chr10 | 8085029 | 8085349 | 0.389943578 | 0.779887156 | 21359 | 321 | 399717 | GATA3-AS1 | 10098 | uc010qbg.1 | FALSE |
| chr2 | 177001256 | 177001263 | 0.389872689 | 0.779745379 | 114080 | 8 | 100506783 | HOXD-AS2 | 563 | uc002ukq.3 | FALSE |
| chr11 | 131781246 | 131781257 | 0.389526862 | 0.779053724 | 42115 | 12 | 50863 | NTM | 534 | uc001qgq.3 | FALSE |
| chr13 | 28492265 | 28492425 | 0.38950923 | 0.779018459 | 53540 | 161 | 3651 | PDX1 | −1743 | uc001urt.2 | TRUE |
| chr11 | 128564756 | 128564874 | 0.389035652 | 0.778071304 | 41769 | 119 | 2313 | FLI1 | 945 | uc010sbv.2 | FALSE |
| chr6 | 27513009 | 27513092 | 0.388961853 | 0.777923706 | 162807 | 84 | 7738 | ZNF184 | −72112 | uc003nji.3 | FALSE |
| chr13 | 36729109 | 36729137 | 0.388905744 | 0.777811488 | 54107 | 29 | 54937 | SOHLH2 | 59615 | uc001uvf.3 | FALSE |
| chr13 | 84453665 | 84453836 | 0.388816015 | 0.777632031 | 55975 | 172 | 114798 | SLITRK1 | 2692 | uc001vlk.3 | FALSE |
| chr10 | 131768098 | 131768149 | 0.388788069 | 0.777576138 | 29860 | 52 | 253738 | EBF3 | −6007 | uc001lki.2 | FALSE |
| chr12 | 103351436 | 103351443 | 0.38872002 | 0.77744004 | 49167 | 8 | 429 | ASCL1 | −9 | uc001tjr.4 | TRUE |
| chr12 | 4918391 | 4918848 | 0.388527369 | 0.777054737 | 43043 | 458 | 3742 | KCNA6 | 49 | uc001qng.3 | FALSE |
| chr1 | 146549940 | 146550467 | 0.388507726 | 0.777015452 | 12089 | 528 | 644861 | NBPF13P | 35461 | uc001epd.3 | FALSE |
| chr7 | 27162090 | 27162294 | 0.388456427 | 0.776912854 | 175211 | 205 | 3200 | HOXA3 | 4345 | uc011jzl.2 | FALSE |
| chr5 | 54516779 | 54516805 | 0.388358988 | 0.776717975 | 152318 | 27 | 345643 | MCIDAS | 6338 | uc021xyp.1 | FALSE |
| chr1 | 44883362 | 44883697 | 0.388065637 | 0.776131275 | 7404 | 336 | 55182 | RNF220 | 12312 | uc010okx.1 | FALSE |
| chr3 | 141516638 | 141516705 | 0.38804314 | 0.776086281 | 136815 | 68 | 131890 | GRK7 | 19595 | uc011bnd.2 | FALSE |
| chr12 | 14134486 | 14134816 | 0.387744873 | 0.775489747 | 44049 | 331 | 2904 | GRIN2B | −1464 | uc001rbt.2 | TRUE |
| chr5 | 3596207 | 3596704 | 0.387521123 | 0.775042246 | 150319 | 498 | 79192 | IRX1 | 39 | uc003jde.3 | FALSE |
| chr7 | 151442351 | 151442371 | −0.387455912 | 0.774911824 | 183806 | 21 | 51422 | PRKAG2 | 69586 | uc011kvl.2 | FALSE |
| chr7 | 155258411 | 155258828 | 0.387288055 | 0.774576109 | 184103 | 418 | 2020 | EN2 | 7587 | uc003wmb.3 | FALSE |
| chr1 | 99469819 | 99470129 | 0.386886524 | 0.773773048 | 10567 | 311 | 100129620 | LOC100129620 | 0 | uc001dsd.1 | TRUE |
| chr3 | 147139184 | 147139563 | 0.386698012 | 0.773396024 | 137011 | 380 | 7545 | ZIC1 | 12003 | uc003ewe.3 | FALSE |
| chr7 | 70597599 | 70597687 | 0.38661434 | 0.773228679 | 178081 | 89 | 64409 | GALNT17 | 76 | uc003tvy.4 | FALSE |
| chr11 | 20180666 | 20180700 | 0.386608839 | 0.773217677 | 33398 | 35 | 120237 | DBX1 | 1170 | uc021qez.1 | FALSE |
| chr3 | 122296613 | 122296778 | 0.386557336 | 0.773114671 | 135106 | 166 | 165631 | PARP15 | 164 | uc003efn.2 | FALSE |
| chr3 | 148446998 | 148447161 | −0.386504513 | 0.773009027 | 137035 | 164 | 185 | AGTR1 | 31340 | uc003ewk.4 | FALSE |
| chr14 | 29254530 | 29254680 | 0.385959538 | 0.771919075 | 58851 | 151 | 387978 | LINC01551 | 12620 | uc001wqf.3 | FALSE |
| chr15 | 45409170 | 45409319 | 0.385823153 | 0.771646306 | 66682 | 150 | 405753 | DUOXA2 | 2647 | uc010beb.3 | FALSE |
| chr8 | 136302000 | 136302154 | −0.385738043 | 0.771476087 | 193188 | 155 | 286094 | LINC01591 | 55626 | uc022bbs.1 | FALSE |
| chr10 | 22634432 | 22634439 | 0.385550873 | 0.771101746 | 22195 | 8 | 9576 | SPAG6 | 58 | uc010qct.2 | FALSE |
| chr4 | 1504481 | 1504542 | −0.384943086 | 0.769886172 | 140630 | 62 | 285464 | NA | 119141 | uc003gdf.2 | FALSE |
| chr8 | 126964345 | 126964551 | −0.384827926 | 0.769655851 | 192565 | 207 | 100130231 | LINC00861 | −904 | uc022bbb.1 | TRUE |
| chr8 | 85096015 | 85096037 | 0.384732945 | 0.76946589 | 190477 | 23 | 138046 | RALYL | 429 | uc010lzy.3 | FALSE |
| chr11 | 31841524 | 31841768 | 0.384671449 | 0.769342899 | 33772 | 245 | 440034 | PAX6-AS1 | 3410 | uc009yjr.3 | FALSE |
| chr3 | 125076233 | 125076372 | 0.38460068 | 0.76920136 | 135403 | 140 | 7707 | ZNF148 | 17826 | uc003ehy.3 | FALSE |
| chr7 | 8482325 | 8482614 | 0.384104612 | 0.768209224 | 174366 | 290 | 30010 | NXPH1 | 8740 | uc011jxh.2 | FALSE |
| chrX | 106515943 | 106516174 | 0.383761388 | 0.767522777 | 203168 | 232 | 139212 | PIH1D3 | 66081 | uc004end.3 | FALSE |
| chr10 | 60936383 | 60936451 | 0.383439472 | 0.766878944 | 24228 | 69 | 84457 | PHYHIPL | 35 | uc001jkl.4 | FALSE |
| chr12 | 54448729 | 54448769 | 0.383399291 | 0.766798581 | 46543 | 41 | 3221 | HOXC4 | 1068 | uc001sex.3 | FALSE |
| chr7 | 143582499 | 143582630 | 0.383249802 | 0.766499604 | 183093 | 132 | 9747 | TCAF1 | 16542 | uc011ktu.2 | FALSE |
| chr10 | 126712482 | 126712941 | 0.383023642 | 0.766047284 | 29355 | 460 | 1488 | CTBP2 | 3512 | uc001lie.4 | FALSE |
| chr19 | 48918116 | 48918205 | 0.383013159 | 0.766026319 | 100733 | 90 | 2906 | GRIN2D | 19984 | uc010elx.3 | FALSE |
| chr17 | 75369484 | 75369657 | 0.382957543 | 0.765915086 | 89464 | 174 | 10801 | SEPTIN9 | 212 | uc002jtv.3 | FALSE |
| chr12 | 130333393 | 130333815 | −0.382904921 | 0.765809843 | 51851 | 423 | 121256 | TMEM132D | 54397 | uc009zyl.1 | FALSE |
| chr1 | 36043002 | 36043014 | 0.382830997 | 0.765661993 | 6240 | 13 | 339488 | TFAP2E | 4031 | uc010ohy.2 | FALSE |
| chr19 | 23253834 | 23254131 | 0.382803935 | 0.765607869 | 98064 | 298 | 100129543 | ZNF730 | −45646 | uc031rkc.1 | FALSE |
| chr5 | 134735637 | 134735654 | 0.382800757 | 0.765601513 | 156153 | 18 | 9555 | MACROH2A1 | −60 | uc003lat.1 | TRUE |
| chr12 | 63545288 | 63545523 | 0.382116352 | 0.764232703 | 47331 | 236 | 552 | AVPR1A | 1067 | uc001sro.2 | FALSE |
| chr19 | 22019059 | 22019094 | 0.381994812 | 0.763989624 | 97995 | 36 | 7594 | ZNF43 | 15776 | uc002nqj.4 | FALSE |
| chr5 | 140306377 | 140306458 | 0.381990233 | 0.763980466 | 156791 | 82 | 56135 | PCDHAC1 | 75 | uc003lih.2 | FALSE |
| chr22 | 25678615 | 25678988 | 0.381982457 | 0.763964914 | 125662 | 374 | 91353 | IGLL3P | −35236 | uc021wnj.1 | FALSE |
| chr17 | 59529486 | 59529618 | 0.381898909 | 0.763797818 | 87800 | 133 | 9496 | TBX4 | −161 | uc010ddo.3 | TRUE |
| chr9 | 122132103 | 122132178 | 0.380839282 | 0.761678565 | 197802 | 76 | 1620 | BRINP1 | −364 | uc004bkc.2 | TRUE |
| chr5 | 72595685 | 72595741 | 0.38023867 | 0.760477341 | 153203 | 57 | 134288 | TMEM174 | 126662 | uc010izc.3 | FALSE |
| chr6 | 62996183 | 62996214 | 0.38004689 | 0.760093781 | 166531 | 32 | 202559 | KHDRBS2 | −83 | uc003peg.2 | TRUE |
| chr15 | 35047189 | 35047203 | 0.380014373 | 0.760028745 | 65761 | 15 | 57369 | GJD2 | −407 | uc001zis.2 | TRUE |
| chr8 | 53478454 | 53478624 | 0.3799322 | 0.759864399 | 189080 | 171 | 389658 | ALKAL1 | −433 | uc003xrd.3 | TRUE |
| chr1 | 221067896 | 221068171 | 0.379402814 | 0.758805628 | 17927 | 276 | 3142 | HLX | 15153 | uc001hmv.4 | FALSE |
| chr1 | 63792695 | 63792863 | 0.37930285 | 0.758605701 | 8894 | 169 | 27022 | FOXD3 | 3965 | uc001dax.2 | FALSE |
| chr6 | 29012588 | 29012859 | −0.378958815 | 0.75791763 | 163094 | 272 | 26692 | OR2W1 | 93 | uc003nlw.2 | FALSE |
| chr7 | 4850075 | 4850105 | −0.378940314 | 0.757880629 | 173811 | 31 | 55698 | RADIL | 21735 | uc003snh.1 | FALSE |
| chr13 | 46961008 | 46961115 | 0.37886107 | 0.75772214 | 54767 | 108 | 80183 | RUBCNL | 520 | uc010tfz.2 | FALSE |
| chr10 | 130165379 | 130165501 | −0.378778073 | 0.757556145 | 29669 | 123 | 4288 | MKI67 | −240911 | uc001lke.3 | FALSE |
| chr14 | 101193397 | 101193432 | 0.378671497 | 0.757342993 | 63426 | 36 | 8788 | DLK1 | 195 | uc001yhs.4 | FALSE |
| chr19 | 22805801 | 22806184 | 0.378370543 | 0.756741086 | 98037 | 384 | 57615 | ZNF492 | −10942 | uc002nqw.3 | FALSE |
| chr3 | 147140880 | 147140930 | 0.378209848 | 0.756419697 | 137012 | 51 | 7545 | ZIC1 | 13699 | uc003ewe.3 | FALSE |
| chr19 | 52839924 | 52840042 | 0.378180446 | 0.756360891 | 101602 | 119 | 162963 | ZNF610 | 426 | uc002pyz.4 | FALSE |
| chr14 | 52535964 | 52535973 | 0.377801382 | 0.755602764 | 59679 | 10 | 22795 | NID2 | −18 | uc001wzo.3 | TRUE |
| chr17 | 41832753 | 41832873 | 0.377773897 | 0.755547793 | 85866 | 121 | 50964 | SOST | 3283 | uc002iec.1 | FALSE |
| chr4 | 41747895 | 41748193 | 0.377556667 | 0.755113335 | 143308 | 299 | 8929 | PHOX2B | 2794 | uc003gwf.4 | FALSE |
| chr3 | 172858917 | 172858969 | −0.377326851 | 0.754653703 | 138277 | 53 | 83893 | SPATA16 | 89 | uc003fin.4 | FALSE |
| chr12 | 127211262 | 127211421 | 0.377082005 | 0.754164011 | 51659 | 160 | 387895 | LINC00944 | 45387 | uc001uhl.3 | FALSE |
| chr2 | 177027440 | 177027621 | 0.377018487 | 0.754036975 | 114091 | 182 | 3232 | HOXD3 | −1184 | uc002ukt.1 | TRUE |
| chr10 | 43393728 | 43393846 | 0.376903815 | 0.753807629 | 23285 | 119 | 9790 | BMS1 | 115774 | uc031pup.1 | FALSE |
| chr6 | 138539372 | 138539706 | −0.376851653 | 0.753703306 | 169647 | 335 | 59351 | PBOV1 | 0 | uc003qhv.3 | TRUE |
| chr11 | 86085757 | 86085932 | 0.376719083 | 0.753438166 | 38842 | 176 | 60494 | CCDC81 | 0 | uc001pbx.2 | TRUE |
| chrX | 142723768 | 142723826 | 0.376696642 | 0.753393285 | 204077 | 59 | 139065 | SLITRK4 | 100 | uc004fby.3 | FALSE |
| chr4 | 54970175 | 54970254 | 0.376555703 | 0.753111405 | 143758 | 80 | 5156 | PDGFRA | 726355 | uc010igp.1 | FALSE |
| chr19 | 20844070 | 20844422 | 0.376419117 | 0.752838234 | 97944 | 353 | 199777 | ZNF626 | 0 | uc002npd.1 | TRUE |
| chr3 | 170303190 | 170303540 | 0.376293814 | 0.752587629 | 138058 | 351 | 57709 | SLC7A14 | 323 | uc003fgz.2 | FALSE |
| chr19 | 20162840 | 20162905 | 0.376260187 | 0.752520375 | 97926 | 66 | 91120 | ZNF682 | −12563 | uc002noq.3 | FALSE |
| chr7 | 27157818 | 27157855 | 0.376224056 | 0.752448111 | 175209 | 38 | 3200 | HOXA3 | 1359 | uc011jzl.2 | FALSE |
| chr1 | 159683855 | 159683921 | −0.375751731 | 0.751503462 | 13723 | 67 | 1401 | CRP | 458 | uc001ftx.1 | FALSE |
| chr8 | 136245114 | 136245226 | −0.375619949 | 0.751239898 | 193185 | 113 | 286094 | LINC01591 | −1148 | uc011ljn.2 | TRUE |
| chr2 | 74742786 | 74743009 | 0.375512338 | 0.751024676 | 108409 | 224 | 3196 | TLX2 | 1190 | uc002smb.2 | FALSE |
| chr4 | 41750362 | 41750390 | 0.375304123 | 0.750608247 | 143310 | 29 | 8929 | PHOX2B | 597 | uc003gwf.4 | FALSE |
| chr17 | 46804239 | 46804309 | 0.375111036 | 0.750222071 | 86671 | 71 | 10481 | HOXB13 | 1802 | uc002ioa.3 | FALSE |
| chr19 | 30866202 | 30866365 | 0.374891926 | 0.749783852 | 98203 | 164 | 9745 | ZNF536 | 2874 | uc010edd.1 | FALSE |
| chr19 | 54481620 | 54481859 | 0.374078698 | 0.748157396 | 101897 | 240 | 59283 | CACNG8 | 15330 | uc021vbd.1 | FALSE |
| chr15 | 79381805 | 79381867 | 0.374057062 | 0.748114125 | 69734 | 63 | 5923 | RASGRF1 | 1348 | uc002ber.4 | FALSE |
| chr16 | 12997592 | 12997684 | 0.374042752 | 0.748085505 | 74000 | 93 | 729993 | SHISA9 | 2115 | uc010uyy.2 | FALSE |
| chr8 | 105479248 | 105479318 | 0.373828977 | 0.747657953 | 191630 | 71 | 1807 | DPYS | 0 | uc003yly.4 | TRUE |
| chr19 | 54412885 | 54412993 | 0.373634775 | 0.74726955 | 101884 | 109 | 5582 | PRKCG | 25972 | uc010era.2 | FALSE |
| chr10 | 98129902 | 98130127 | −0.373531008 | 0.747062017 | 26945 | 226 | 7093 | TLL2 | 143556 | uc001kmi.3 | FALSE |
| chr8 | 85097195 | 85097246 | 0.373409554 | 0.746819108 | 190478 | 52 | 138046 | RALYL | 95 | uc003yct.4 | FALSE |
| chr12 | 12867669 | 12867724 | 0.373260433 | 0.746520866 | 43932 | 56 | 1027 | CDKN1B | −2578 | uc001rat.2 | FALSE |
| chr15 | 67326082 | 67326118 | 0.373184802 | 0.746369603 | 68328 | 37 | 4088 | SMAD3 | −32077 | uc002aqj.3 | FALSE |
| chr10 | 34399605 | 34399658 | −0.372879714 | 0.745759428 | 22958 | 54 | 56288 | PARD3 | 704595 | uc001ixs.1 | FALSE |
| chr12 | 71314144 | 71314315 | 0.37287584 | 0.74575168 | 47821 | 172 | 5801 | PTPRR | 269 | uc001swi.2 | FALSE |
| chr14 | 101513644 | 101514051 | −0.372551712 | 0.745103424 | 63533 | 408 | 664612 | MIR539 | 0 | uc021sdg.1 | TRUE |
| chr2 | 131185333 | 131185379 | 0.372514132 | 0.745028263 | 111714 | 47 | 100216479 | FAR2P2 | 740 | uc031roz.1 | FALSE |
| chrX | 150863961 | 150863969 | 0.372431498 | 0.744862995 | 204227 | 9 | 79057 | PRRG3 | 231 | uc022cgt.1 | FALSE |
| chr13 | 102568555 | 102568886 | 0.372417628 | 0.744835256 | 56677 | 332 | 2259 | FGF14 | 109 | uc001vpe.2 | FALSE |
| chrX | 139587695 | 139587868 | 0.372413524 | 0.744827047 | 204028 | 174 | 6658 | SOX3 | −470 | uc004fbd.1 | TRUE |
| chr4 | 4860026 | 4860061 | 0.371688415 | 0.743376829 | 141367 | 36 | 4487 | MSX1 | −1331 | uc003gif.3 | TRUE |
| chr7 | 906726 | 906779 | 0.371682567 | 0.743365135 | 172743 | 54 | 23353 | SUN1 | 14842 | uc003sjl.1 | FALSE |
| chr20 | 5296302 | 5296442 | 0.371380818 | 0.742761635 | 119586 | 141 | 128674 | PROKR2 | 936 | uc010zqy.2 | FALSE |
| chr6 | 32119616 | 32119639 | 0.371380076 | 0.742760151 | 163827 | 24 | 80863 | PRRT1 | 61 | uc003nzs.3 | FALSE |
| chrX | 118407645 | 118407852 | 0.371321076 | 0.742642152 | 203437 | 208 | 10857 | PGRMC1 | 37434 | uc011mts.2 | FALSE |
| chr6 | 27513414 | 27513479 | 0.370829098 | 0.741658195 | 162807 | 66 | 7738 | ZNF184 | −72517 | uc003nji.3 | FALSE |
| chr11 | 14926738 | 14927004 | −0.370770539 | 0.741541079 | 32927 | 267 | 120227 | CYP2R1 | 12987 | uc001mlr.3 | FALSE |
| chr2 | 87048489 | 87048747 | 0.370639539 | 0.741279079 | 109008 | 259 | 926 | CD8B | 40300 | uc002srv.3 | FALSE |
| chr17 | 72322143 | 72322154 | 0.370630452 | 0.741260905 | 88814 | 12 | 124602 | KIF19 | −197 | uc031rei.1 | TRUE |
| chr12 | 54409207 | 54409212 | 0.370502448 | 0.741004897 | 46529 | 6 | 3223 | HOXC6 | −1430 | uc001ses.3 | TRUE |
| chr1 | 77333138 | 77333159 | 0.370298726 | 0.740597452 | 9489 | 22 | 81849 | ST6GALNAC5 | −27 | uc001dhi.3 | TRUE |
| chr8 | 67090250 | 67090581 | 0.370234249 | 0.740468497 | 189713 | 332 | 1392 | CRH | 265 | uc003xvy.2 | FALSE |
| chr12 | 65515290 | 65515470 | 0.370209574 | 0.740419147 | 47454 | 181 | 11197 | WIF1 | 0 | uc001ssk.3 | TRUE |
| chr2 | 177054306 | 177054573 | 0.370142292 | 0.740284584 | 114101 | 268 | 3231 | HOXD1 | 999 | uc021vsq.1 | FALSE |
| chr13 | 96294131 | 96294468 | 0.369986503 | 0.739973007 | 56221 | 338 | 22873 | DZIP1 | 2492 | uc001vml.4 | FALSE |
| chrX | 89177498 | 89177512 | −0.369707434 | 0.739414868 | 202821 | 15 | 90316 | TGIF2LX | 558 | uc004efe.3 | FALSE |
| chr6 | 35286315 | 35286360 | 0.369687072 | 0.739374145 | 164482 | 46 | 50619 | DEF6 | 20720 | uc010jvt.3 | FALSE |
| chr1 | 248549671 | 248549941 | −0.36966621 | 0.73933242 | 20239 | 271 | 254879 | OR2T6 | −969 | uc001iei.1 | TRUE |
| chr2 | 237077920 | 237078026 | 0.369579835 | 0.739159671 | 117855 | 107 | 2637 | GBX2 | −1268 | uc002vvw.1 | TRUE |
| chr1 | 159506621 | 159507162 | −0.369378405 | 0.73875681 | 13719 | 542 | 127385 | OR10J5 | −824 | uc010piw.2 | TRUE |
| chr7 | 19158647 | 19158664 | 0.369226526 | 0.738453052 | 174677 | 18 | 7291 | TWIST1 | −1352 | uc003sum.3 | TRUE |
| chr2 | 238777587 | 238777656 | −0.369147666 | 0.738295332 | 118093 | 70 | 10267 | RAMP1 | 9400 | uc002vxj.3 | FALSE |
| chr1 | 3663339 | 3663435 | 0.36889752 | 0.73779504 | 1384 | 97 | 57212 | TP73-AS1 | 502 | uc001akt.4 | FALSE |
| chr6 | 167275755 | 167275778 | 0.36845189 | 0.73690378 | 171702 | 24 | 6196 | RPS6KA2 | 0 | uc003qvd.1 | TRUE |
| chr11 | 43569076 | 43569269 | 0.36787949 | 0.73575898 | 34304 | 194 | 100313777 | MIR670 | −11937 | uc021qgk.1 | FALSE |
| chr17 | 5820161 | 5820294 | −0.367776399 | 0.735552799 | 81741 | 134 | 339166 | LOC339166 | 144607 | uc002gcm.3 | FALSE |
| chr13 | 79169714 | 79169753 | 0.367665243 | 0.735330485 | 55867 | 40 | 100874222 | OBI1-AS1 | 540724 | uc001vkv.3 | FALSE |
| chr1 | 214153377 | 214153460 | 0.367567449 | 0.735134897 | 17541 | 84 | 5629 | PROX1 | −7818 | uc001hkg.2 | FALSE |
| chr6 | 29795595 | 29795815 | 0.367391836 | 0.734783672 | 163225 | 221 | 3135 | HLA-G | 0 | uc021ytv.1 | TRUE |
| chr2 | 26045244 | 26045287 | 0.367330032 | 0.734660063 | 104944 | 44 | 55252 | ASXL2 | 56025 | uc002rgt.1 | FALSE |
| chr10 | 44162879 | 44163067 | 0.367199307 | 0.734398613 | 23404 | 189 | 414201 | ZNF32-AS3 | 38614 | uc001jbc.3 | FALSE |
| chr12 | 54360460 | 54360511 | 0.367194728 | 0.734389457 | 46503 | 52 | 100124700 | HOTAIR | 2029 | uc009zne.4 | FALSE |
| chr6 | 146755622 | 146755900 | 0.366836053 | 0.733672107 | 170090 | 279 | 2911 | GRM1 | 405288 | uc003qln.1 | FALSE |
| chr13 | 91825341 | 91825667 | −0.366743122 | 0.733486244 | 56052 | 327 | 100874150 | LINC00379 | 38285 | uc031qms.1 | FALSE |
| chr15 | 58357879 | 58357891 | 0.366723798 | 0.733447597 | 67409 | 13 | 8854 | ALDH1A2 | 230 | uc010ugv.2 | FALSE |
| chr12 | 54345212 | 54345679 | 0.366479712 | 0.732959424 | 46497 | 468 | 3228 | HOXC12 | −3035 | uc010soq.2 | FALSE |
| chr7 | 56297564 | 56297579 | 0.366379385 | 0.73275877 | 177440 | 16 | 5723 | PSPH | −113474 | uc003trj.3 | FALSE |
| chr2 | 223164854 | 223164867 | 0.366226129 | 0.732452257 | 116714 | 14 | 151278 | CCDC140 | 1988 | uc002vmt.2 | FALSE |
| chr9 | 19788257 | 19788564 | 0.365920191 | 0.731840383 | 195087 | 308 | 25769 | SLC24A2 | −1240 | uc003zoa.2 | TRUE |
| chr2 | 168103475 | 168103543 | −0.365856488 | 0.731712976 | 113380 | 69 | 129446 | XIRP2 | 59682 | uc010fpr.3 | FALSE |
| chr18 | 55108537 | 55108852 | 0.365680798 | 0.731361597 | 92588 | 316 | 9480 | ONECUT2 | 5620 | uc002lgo.3 | FALSE |
| chr18 | 32847251 | 32847566 | 0.365542055 | 0.731084109 | 92069 | 316 | 100101467 | ZSCAN30 | 22599 | uc002kyn.1 | FALSE |
| chr12 | 103696209 | 103696381 | 0.365540295 | 0.73108059 | 49176 | 173 | 374470 | C12orf42 | 193365 | uc001tjt.2 | FALSE |
| chr19 | 23258096 | 23258484 | 0.365450942 | 0.730901883 | 98066 | 389 | 100129543 | ZNF730 | −41293 | uc031rkc.1 | FALSE |
| chr6 | 35479628 | 35479648 | 0.365331232 | 0.730662463 | 164521 | 21 | 7287 | TULP1 | 999 | uc021yyy.1 | FALSE |
| chr1 | 180203837 | 180204221 | 0.365313258 | 0.730626517 | 15223 | 385 | 89884 | LHX4 | 4404 | uc001goe.2 | FALSE |
| chr14 | 42077327 | 42077674 | 0.365226995 | 0.730453989 | 59314 | 348 | 145581 | LRFN5 | 563 | uc001wvm.3 | FALSE |
| chr1 | 158368889 | 158369112 | −0.365008407 | 0.730016813 | 13638 | 224 | 128360 | OR10T2 | 144 | uc010pih.2 | FALSE |
| chr5 | 16179135 | 16179633 | 0.364839972 | 0.729679944 | 151021 | 499 | 441061 | MARCHF11 | 264 | uc003jfo.2 | FALSE |
| chr1 | 197887714 | 197887955 | 0.364447485 | 0.72889497 | 15925 | 242 | 56956 | LHX9 | 1197 | uc001guk.1 | FALSE |
| chr3 | 62364793 | 62364810 | 0.364384937 | 0.728769873 | 132699 | 18 | 55079 | FEZF2 | −5603 | uc003dli.2 | FALSE |
| chr19 | 1356269 | 1356278 | 0.364313287 | 0.728626574 | 94004 | 10 | 84939 | PWWP3A | 1293 | uc002lsb.2 | FALSE |
| chr7 | 70597058 | 70597065 | 0.364092885 | 0.72818577 | 178081 | 8 | 64409 | GALNT17 | −458 | uc003tvy.4 | TRUE |
| chr6 | 50690647 | 50690975 | 0.364074571 | 0.728149143 | 166045 | 329 | 83741 | TFAP2D | 9390 | uc011dwt.2 | FALSE |
| chr2 | 21229220 | 21229231 | −0.364044805 | 0.728089611 | 104614 | 12 | 338 | APOB | 37714 | uc002red.3 | FALSE |
| chr18 | 67067867 | 67067893 | 0.363992691 | 0.727985382 | 92846 | 27 | 220164 | DOK6 | −391 | uc002lkl.3 | TRUE |
| chr12 | 8025582 | 8025593 | 0.363985696 | 0.727971392 | 43464 | 12 | 144195 | SLC2A14 | 18151 | uc001qtn.3 | FALSE |
| chr5 | 153862394 | 153862450 | 0.363911059 | 0.727822118 | 157999 | 57 | 9421 | HAND1 | −4570 | uc003lvn.3 | FALSE |
| chr7 | 27242005 | 27242044 | 0.363823274 | 0.727646548 | 175254 | 40 | 100316868 | HOTTIP | 1965 | uc022aau.1 | FALSE |
| chr1 | 208040203 | 208040253 | 0.363633941 | 0.727267881 | 17078 | 51 | 947 | CD34 | 23584 | uc001hgv.1 | FALSE |
| chr2 | 54086854 | 54087008 | 0.363561556 | 0.727123112 | 106896 | 155 | 51130 | ASB3 | 162 | uc002rxo.3 | FALSE |
| chr1 | 240160972 | 240161249 | 0.363484367 | 0.726968734 | 19553 | 278 | 645884 | RPS7P5 | −9575 | uc021pld.1 | FALSE |
| chr17 | 5404330 | 5404337 | 0.363269454 | 0.726538907 | 81718 | 8 | 728392 | LOC728392 | −11 | uc010vtc.2 | TRUE |
| chr5 | 35991215 | 35991382 | 0.36319347 | 0.72638694 | 151646 | 168 | 133688 | UGT3A1 | 153 | uc011cor.2 | FALSE |
| chr5 | 87981253 | 87981449 | 0.363178385 | 0.72635677 | 154022 | 197 | 645323 | LINC00461 | −633 | uc011cua.1 | TRUE |
| chr3 | 147129184 | 147129213 | 0.363057963 | 0.726115927 | 137006 | 30 | 7545 | ZIC1 | 2003 | uc003ewe.3 | FALSE |
| chr10 | 63212206 | 63212226 | 0.363039439 | 0.726078878 | 24352 | 21 | 219623 | TMEM26 | 982 | uc001jlq.3 | FALSE |
| chr16 | 1213894 | 1213919 | −0.362863612 | 0.725727225 | 72204 | 26 | 8912 | CACNA1H | 10653 | uc002ckt.3 | FALSE |
| chr1 | 227748712 | 227748719 | 0.362620569 | 0.725241139 | 18496 | 8 | 339500 | ZNF678 | −2501 | uc001hqw.2 | FALSE |
| chr12 | 53612641 | 53612734 | 0.362556913 | 0.725113827 | 46381 | 94 | 5916 | RARG | 1463 | uc010sod.2 | FALSE |
| chr14 | 99681710 | 99681757 | 0.362479206 | 0.724958413 | 63136 | 48 | 64919 | BCL11B | 56065 | uc031qqi.1 | FALSE |
| chr1 | 158258802 | 158259268 | −0.362456494 | 0.724912987 | 13627 | 467 | 911 | CD1C | −295 | uc001fru.3 | TRUE |
| chr12 | 54447220 | 54447243 | 0.362380823 | 0.724761646 | 46543 | 24 | 3221 | HOXC4 | 36578 | uc001sex.3 | FALSE |
| chr20 | 1639250 | 1639816 | −0.362203055 | 0.72440611 | 119246 | 567 | 55423 | SIRPG | −825 | uc002wfm.1 | TRUE |
| chr1 | 20669792 | 20669905 | 0.362199972 | 0.724399945 | 4191 | 114 | 127731 | VWA5B1 | 10533 | uc009vpt.2 | FALSE |
| chr10 | 130085088 | 130085199 | 0.362193776 | 0.724387551 | 29664 | 112 | 4288 | MKI67 | −160620 | uc001lke.3 | FALSE |
| chr8 | 70946891 | 70946946 | 0.362125059 | 0.724250118 | 189927 | 56 | 63978 | PRDM14 | 36616 | uc003xym.3 | FALSE |
| chr1 | 26735606 | 26735630 | 0.361884571 | 0.723769141 | 5094 | 25 | 79727 | LIN28A | −1639 | uc001bmi.1 | TRUE |
| chr12 | 47219737 | 47219793 | 0.361818145 | 0.723636291 | 45461 | 57 | 55089 | SLC38A4 | 0 | uc009zkl.2 | TRUE |
| chr5 | 170738987 | 170739179 | 0.361563904 | 0.723127807 | 158820 | 193 | 30012 | TLX3 | 2699 | uc003mbf.3 | FALSE |
| chr14 | 60952933 | 60952945 | 0.361254578 | 0.722509156 | 60292 | 13 | 317761 | C14orf39 | −169 | uc001xez.4 | TRUE |
| chr13 | 28502461 | 28502559 | 0.36117771 | 0.722355419 | 53543 | 99 | 3651 | PDX1 | 8293 | uc001urt.2 | FALSE |
| chr10 | 16562102 | 16562470 | 0.360970783 | 0.721941566 | 21913 | 369 | 389941 | C1QL3 | 1534 | uc001ioj.1 | FALSE |
| chr14 | 54687160 | 54687314 | 0.360957935 | 0.721915869 | 59827 | 155 | 1033 | CDKN3 | −176359 | uc001xap.3 | FALSE |
| chr5 | 140207498 | 140207609 | 0.3609472 | 0.7218944 | 156760 | 112 | 56143 | PCDHA5 | 6137 | uc011dab.2 | FALSE |
| chr6 | 169051418 | 169051431 | 0.360863866 | 0.721727732 | 172081 | 14 | 64094 | SMOC2 | 33496 | uc011egu.2 | FALSE |
| chr14 | 101459547 | 101459591 | −0.360779395 | 0.72155879 | 63516 | 45 | 767612 | SNORD114-31 | 0 | uc001yjv.3 | TRUE |
| chr12 | 81108011 | 81108034 | 0.360757692 | 0.721515384 | 48238 | 24 | 4617 | MYF5 | −2674 | uc001szg.2 | FALSE |
| chr8 | 132052942 | 132053262 | 0.360631178 | 0.721262356 | 192901 | 321 | 114 | ADCY8 | −107 | uc003ytd.4 | TRUE |
| chr15 | 89346269 | 89346795 | 0.360586549 | 0.721173098 | 70450 | 527 | 176 | ACAN | 0 | uc010upp.1 | TRUE |
| chr1 | 91190891 | 91191001 | 0.360435354 | 0.720870708 | 10126 | 111 | 343472 | BARHL2 | −8097 | uc001dns.3 | FALSE |
| chr3 | 147124417 | 147124523 | 0.360421548 | 0.720843097 | 137005 | 107 | 84107 | ZIC4 | 73 | uc021xfg.1 | FALSE |
| chr19 | 14115611 | 14115967 | 0.360388159 | 0.720776319 | 96835 | 357 | 5989 | RFX1 | 1167 | uc010dzi.2 | FALSE |
| chr7 | 44349389 | 44349704 | 0.360353083 | 0.720706167 | 176611 | 316 | 816 | CAMK2B | 15526 | uc010kyc.2 | FALSE |
| chr19 | 53540902 | 53541186 | 0.360334456 | 0.720668912 | 101705 | 285 | 100271846 | ERVV-2 | −6805 | uc021uzd.1 | FALSE |
| chr8 | 143404187 | 143404298 | −0.360250144 | 0.720500287 | 193754 | 112 | 203062 | TSNARE1 | 31826 | uc011lju.2 | FALSE |
| chr9 | 129387040 | 129387231 | 0.360126371 | 0.720252742 | 198318 | 192 | 4010 | LMX1B | 10318 | uc011maa.2 | FALSE |
| chr11 | 31819444 | 31819464 | 0.360108774 | 0.720217547 | 33758 | 21 | 5080 | PAX6 | 6130 | uc031pzl.1 | FALSE |
| chr13 | 70681804 | 70682019 | 0.359914885 | 0.71982977 | 55572 | 216 | 6315 | ATXN8OS | 459 | uc010aej.1 | FALSE |
| chr12 | 54404893 | 54405379 | 0.359747156 | 0.719494311 | 46527 | 487 | 3224 | HOXC8 | 2003 | uc001ser.3 | FALSE |
| chr12 | 54399468 | 54399494 | 0.359579594 | 0.719159188 | 46523 | 27 | 3225 | HOXC9 | 5591 | uc001ser.3 | FALSE |
| chr14 | 20903555 | 20903611 | 0.359573754 | 0.719147509 | 58148 | 57 | 123103 | KLHL33 | 190 | uc010tli.2 | FALSE |
| chr1 | 67600428 | 67600707 | 0.359388047 | 0.718776095 | 9184 | 280 | 400757 | C1orf141 | 0 | uc031pmv.1 | TRUE |
| chr11 | 5538030 | 5538200 | −0.359095752 | 0.718191504 | 31997 | 171 | 3048 | HBG2 | 128811 | uc001maz.4 | FALSE |
| chr5 | 170738059 | 170738274 | 0.359022955 | 0.718045911 | 158820 | 216 | 30012 | TLX3 | 1771 | uc003mbf.3 | FALSE |
| chr18 | 60263588 | 60263646 | 0.358744915 | 0.717489831 | 92748 | 59 | 54877 | ZCCHC2 | 72930 | uc002liq.3 | FALSE |
| chr1 | 214140156 | 214140498 | −0.358683471 | 0.717366941 | 17539 | 343 | 5629 | PROX1 | −20780 | uc001hkg.2 | FALSE |
| chr1 | 228651908 | 228652067 | 0.358658771 | 0.717317541 | 18681 | 160 | 100616308 | MIR4666A | 2133 | uc021pkh.1 | FALSE |
| chr13 | 58207859 | 58208350 | 0.358531205 | 0.717062411 | 55355 | 492 | 27253 | PCDH17 | 2070 | uc010aec.1 | FALSE |
| chr6 | 100915767 | 100915805 | 0.358133529 | 0.716267058 | 167736 | 39 | 6492 | SIM1 | −2962 | uc010kcu.3 | FALSE |
| chr2 | 228324937 | 228324983 | −0.358016713 | 0.716033426 | 116991 | 47 | 100847081 | MIR5703 | −11865 | uc031rrr.1 | FALSE |
| chr19 | 37825446 | 37825679 | 0.357986823 | 0.715973646 | 98865 | 234 | 284459 | ZNF875 | 0 | uc010xtp.2 | TRUE |
| chr5 | 115299071 | 115299088 | 0.357866344 | 0.715732687 | 155092 | 18 | 206338 | LVRN | 920 | uc003krp.3 | FALSE |
| chr19 | 39694709 | 39694831 | 0.35771741 | 0.71543482 | 99137 | 123 | 342898 | SYCN | 75 | uc002okr.2 | FALSE |
| chr3 | 125875526 | 125875552 | −0.357587788 | 0.715175576 | 135481 | 27 | 10840 | ALDH1L1 | 0 | uc003eio.3 | TRUE |
| chr5 | 1268949 | 1269066 | −0.357507401 | 0.715014803 | 149885 | 118 | 7015 | TERT | 13673 | uc021xvz.1 | FALSE |
| chr13 | 112711941 | 112712009 | 0.357464858 | 0.714929716 | 57385 | 69 | 6656 | SOX1 | −9904 | uc001vsb.1 | FALSE |
| chr3 | 170136489 | 170136499 | 0.357454662 | 0.714909324 | 138039 | 11 | 5010 | CLDN11 | −154 | uc003fgx.3 | TRUE |
| chr8 | 65291523 | 65291542 | 0.357207615 | 0.714415231 | 189646 | 20 | 100130155 | MIR124-2HG | 5748 | uc003xvf.3 | FALSE |
| chr14 | 58064775 | 58064926 | −0.35708817 | 0.714176341 | 60113 | 152 | 341880 | SLC35F4 | 267666 | uc010aoz.1 | FALSE |
| chr1 | 119548825 | 119548852 | 0.357059453 | 0.714118905 | 11743 | 28 | 6913 | TBX15 | −16646 | uc001ehl.1 | FALSE |
| chr22 | 24890794 | 24890809 | 0.357020566 | 0.714041132 | 125589 | 16 | 51733 | UPB1 | 717 | uc002zzz.2 | FALSE |
| chr17 | 46674041 | 46674438 | 0.356982758 | 0.713965516 | 86630 | 398 | 404266 | HOXB-AS3 | 721 | uc021tzk.1 | FALSE |
| chr1 | 158549277 | 158549410 | −0.356930091 | 0.713860182 | 13650 | 134 | 128367 | OR10X1 | 279 | uc010pin.2 | FALSE |
| chr7 | 22233159 | 22233206 | 0.356753866 | 0.713507732 | 174828 | 48 | 9771 | RAPGEF5 | 147 | uc011jym.1 | FALSE |
| chr16 | 1224897 | 1225126 | −0.356283097 | 0.712566194 | 72211 | 230 | 8912 | CACNA1H | 21656 | uc002ckt.3 | FALSE |
| chr5 | 140810920 | 140811102 | 0.356054635 | 0.712109269 | 156934 | 183 | 26025 | PCDHGA12 | 762 | uc003lkt.2 | FALSE |
| chr11 | 71954982 | 71955164 | 0.355916151 | 0.711832301 | 37811 | 183 | 401 | PHOX2A | 56 | uc001osh.4 | FALSE |
| chr5 | 7827115 | 7827133 | −0.355854869 | 0.711709738 | 150556 | 19 | 108 | ADCY2 | 44829 | uc003jea.5 | FALSE |
| chr1 | 177939251 | 177939546 | −0.355651364 | 0.711302728 | 15039 | 296 | 89866 | SEC16B | 0 | uc001gll.4 | TRUE |
| chr15 | 93128681 | 93128729 | −0.355586342 | 0.711172683 | 70909 | 49 | 100144604 | LINC00930 | −13188 | uc002brd.2 | FALSE |
| chr17 | 32484027 | 32484035 | 0.355422464 | 0.710844928 | 84396 | 9 | 40 | ASIC2 | −202 | uc002hhu.3 | TRUE |
| chr2 | 133014805 | 133015268 | 0.355365496 | 0.710730992 | 111922 | 464 | 554226 | ANKRD30BL | 274 | uc021vpu.1 | FALSE |
| chr16 | 89098327 | 89098782 | −0.35509379 | 0.71018758 | 80189 | 456 | 863 | CBFA2T3 | −54823 | uc002fmm.2 | FALSE |
| chr6 | 166422671 | 166422741 | 0.354928552 | 0.709857104 | 171554 | 71 | 441177 | LINC00602 | 21632 | uc011egl.1 | FALSE |
| chr12 | 56040096 | 56040106 | 0.35491825 | 0.7098365 | 46720 | 11 | 121130 | OR10P1 | 9420 | uc010spq.2 | FALSE |
| chr2 | 95663959 | 95663987 | 0.354867346 | 0.709734693 | 109219 | 29 | 4118 | MAL | −27413 | uc002stx.2 | FALSE |
| chr2 | 139537131 | 139537197 | 0.354805238 | 0.709610477 | 112262 | 67 | 11249 | NXPH2 | 614 | uc002tvi.3 | FALSE |
| chr6 | 13274151 | 13274180 | −0.354630071 | 0.709260142 | 161607 | 30 | 51256 | TBC1D7 | 54607 | uc003nak.1 | FALSE |
| chr6 | 27463217 | 27463250 | 0.35461249 | 0.709224981 | 162798 | 34 | 7738 | ZNF184 | −22320 | uc003nji.3 | FALSE |
| chr3 | 14852659 | 14852756 | 0.354589958 | 0.709179915 | 129246 | 98 | 152273 | FGD5 | −7713 | uc003bzc.3 | FALSE |
| chr6 | 5995150 | 5995371 | 0.354513025 | 0.709026049 | 161049 | 222 | 51299 | NRN1 | 8931 | uc021ykx.1 | FALSE |
| chr12 | 54345862 | 54346155 | 0.354502021 | 0.709004042 | 46497 | 294 | 3228 | HOXC12 | −2559 | uc010soq.2 | FALSE |
| chr8 | 57069907 | 57070013 | 0.354488899 | 0.708977798 | 189283 | 107 | 5324 | PLAG1 | 13970 | uc003xsq.4 | FALSE |
| chr11 | 32448293 | 32448678 | 0.354158171 | 0.708316341 | 33809 | 386 | 7490 | WT1 | 3685 | uc001mtm.2 | FALSE |
| chr3 | 147110367 | 147110378 | 0.354134222 | 0.708268444 | 136999 | 12 | 84107 | ZIC4 | 4595 | uc021xfc.1 | FALSE |
| chr1 | 75597304 | 75597887 | 0.353928453 | 0.707856906 | 9423 | 584 | 431707 | LHX8 | 1393 | uc021oou.1 | FALSE |
| chr10 | 22765645 | 22765840 | 0.353911694 | 0.707823387 | 22205 | 196 | 100499489 | LOC100499489 | −38787 | uc021pof.2 | FALSE |
| chr7 | 38331753 | 38331871 | −0.353893769 | 0.707787538 | 176133 | 119 | 445347 | TARP | 24838 | uc010kxi.1 | FALSE |
| chr1 | 147782452 | 147782472 | 0.353843221 | 0.707686442 | 12178 | 21 | 728841 | NBPF8 | 564319 | uc031pom.1 | FALSE |
| chr14 | 106410668 | 106410734 | −0.353784469 | 0.707568937 | 64626 | 67 | 9834 | FAM30A | 26830 | uc001yst.3 | FALSE |
| chr4 | 1398599 | 1398798 | 0.353606255 | 0.70721251 | 140616 | 200 | 285464 | NA | 13259 | uc003gdf.2 | FALSE |
| chr18 | 73167422 | 73167671 | 0.353530782 | 0.707061564 | 92953 | 250 | 284274 | SMIM21 | −27833 | uc002lma.1 | FALSE |
| chr17 | 38084377 | 38084428 | 0.353427315 | 0.706854631 | 85103 | 52 | 94103 | ORMDL3 | −493 | uc002htj.2 | TRUE |
| chr3 | 194208259 | 194208441 | 0.353383925 | 0.70676785 | 139676 | 183 | 401106 | LINC00884 | 390 | uc003fua.2 | FALSE |
| chr9 | 100070007 | 100070142 | 0.353363227 | 0.706726453 | 197014 | 136 | 100499483 | CCDC180 | 97 | uc004axg.2 | FALSE |
| chr17 | 75315081 | 75315108 | 0.353203181 | 0.706406363 | 89454 | 28 | 10801 | SEPTIN9 | 31108 | uc002jtu.4 | FALSE |
| chr17 | 46622516 | 46622522 | 0.353040845 | 0.706081691 | 86599 | 7 | 100874362 | HOXB-AS1 | 803 | uc002inm.3 | FALSE |
| chr16 | 56697088 | 56697229 | 0.352840785 | 0.70568157 | 76657 | 142 | 4495 | MT1G | 4748 | uc002ejv.1 | FALSE |
| chr11 | 104034619 | 104034754 | 0.3528212 | 0.705642401 | 39643 | 136 | 80310 | PDGFD | 273 | uc001phq.3 | FALSE |
| chr12 | 16757954 | 16757985 | 0.352552984 | 0.705105968 | 44191 | 32 | 55885 | LMO3 | 328 | uc001rdj.2 | FALSE |
| chr22 | 17082581 | 17082787 | 0.352434828 | 0.704869656 | 124746 | 207 | 387590 | TPTEP1 | −14 | uc002zlq.4 | TRUE |
| chr14 | 101493252 | 101493361 | −0.352290512 | 0.704581025 | 63523 | 110 | 574408 | MIR329-1 | 130 | uc021scr.1 | FALSE |
| chr19 | 57641963 | 57642143 | −0.352090664 | 0.704181328 | 102471 | 181 | 57663 | USP29 | 10454 | uc002qny.3 | FALSE |
| chr1 | 75596724 | 75596758 | 0.35197263 | 0.70394526 | 9423 | 35 | 431707 | LHX8 | 813 | uc021oou.1 | FALSE |
| chr7 | 8483569 | 8483710 | 0.35196123 | 0.70392246 | 174366 | 142 | 30010 | NXPH1 | 9984 | uc011jxh.2 | FALSE |
| chr2 | 242756029 | 242756362 | −0.351754409 | 0.703508818 | 118955 | 334 | 129807 | NEU4 | 3999 | uc002wcp.2 | FALSE |
| chr4 | 122078167 | 122078348 | 0.35171764 | 0.703435279 | 146391 | 182 | 79931 | TNIP3 | 7147 | uc010ini.3 | FALSE |
| chr11 | 18813544 | 18813556 | 0.35160604 | 0.70321208 | 33272 | 13 | 84867 | PTPN5 | 712 | uc010rdk.3 | FALSE |
| chrX | 102319998 | 102320380 | −0.351462652 | 0.702925304 | 203015 | 383 | 55859 | BEX1 | −830 | uc004ejt.1 | TRUE |
| chr10 | 100996070 | 100996097 | 0.351367018 | 0.702734035 | 27211 | 28 | 60495 | HPSE2 | −438 | uc001kpn.2 | TRUE |
| chr1 | 248020812 | 248021091 | 0.351276449 | 0.702552898 | 20191 | 280 | 25893 | TRIM58 | 311 | uc001ido.3 | FALSE |
| chr2 | 27988785 | 27988858 | 0.351252793 | 0.702505587 | 105230 | 74 | 9553 | MRPL33 | −5726 | uc002rlm.1 | FALSE |
| chr10 | 125425625 | 125425688 | 0.351017043 | 0.702034086 | 29174 | 64 | 2849 | GPR26 | −183 | uc001lhh.3 | TRUE |
| chr7 | 142724374 | 142724597 | −0.350884285 | 0.701768571 | 183009 | 224 | 135924 | OR9A2 | −155 | uc003wcc.1 | TRUE |
| chr11 | 55889797 | 55890274 | −0.350635261 | 0.701270522 | 35117 | 478 | 390152 | OR8H3 | 0 | uc001nii.1 | TRUE |
| chr7 | 27233411 | 27233454 | 0.350511216 | 0.701022432 | 175248 | 44 | 3209 | HOXA13 | 6271 | uc003szb.1 | FALSE |
| chr5 | 76249502 | 76249776 | 0.350452686 | 0.700905372 | 153465 | 275 | 1393 | CRHBP | 822 | uc003ker.3 | FALSE |
| chr15 | 53083518 | 53083532 | 0.350351543 | 0.700703087 | 67181 | 15 | 3175 | ONECUT1 | −1309 | uc002aci.2 | TRUE |
| chr5 | 174159039 | 174159294 | 0.350266373 | 0.700532745 | 159244 | 256 | 4488 | MSX2 | 7464 | uc003mcy.3 | FALSE |
| chr22 | 48884960 | 48885446 | 0.350038024 | 0.700076049 | 127698 | 487 | 25817 | TAFA5 | 0 | uc003bim.4 | TRUE |
| chr1 | 217311151 | 217311172 | 0.349978883 | 0.699957765 | 17728 | 22 | 2104 | ESRRG | −54 | uc001hlc.1 | TRUE |
| chr10 | 102891080 | 102891280 | 0.349743549 | 0.699487097 | 27494 | 201 | 3195 | TLX1 | 19 | uc021pxd.1 | FALSE |
| chrX | 129657974 | 129658061 | 0.349696347 | 0.699392694 | 203715 | 88 | 55855 | DENND10P1 | 29059 | uc010nrh.3 | FALSE |
| chr10 | 134902168 | 134902278 | 0.349597587 | 0.699195174 | 30503 | 111 | 84435 | ADGRA1 | 759 | uc001llx.4 | FALSE |
| chr15 | 101697195 | 101697236 | −0.349596436 | 0.699192872 | 71570 | 42 | 22856 | CHSY1 | 31030 | uc010usd.2 | FALSE |
| chr10 | 23481776 | 23481786 | 0.349470765 | 0.69894153 | 22261 | 11 | 256297 | PTF1A | 316 | uc001irp.3 | FALSE |
| chr4 | 147559579 | 147559648 | 0.349465748 | 0.698931495 | 147360 | 70 | 5458 | POU4F2 | −397 | uc003ikv.3 | TRUE |
| chr10 | 135153937 | 135153961 | −0.349457025 | 0.69891405 | 30633 | 25 | 118471 | PRAP1 | 31043 | uc001lmo.2 | FALSE |
| chr4 | 158143439 | 158143538 | 0.349416729 | 0.698833458 | 147895 | 100 | 2891 | GRIA2 | 1045 | uc010iqh.1 | FALSE |
| chr19 | 9473781 | 9473880 | 0.349373408 | 0.698746816 | 95963 | 100 | 7730 | ZNF177 | 85 | uc002mlk.3 | FALSE |
| chr15 | 88800567 | 88800624 | 0.349330637 | 0.698661275 | 70390 | 58 | 283738 | NTRK3-AS1 | 4606 | uc002bme.2 | FALSE |
| chr3 | 147128123 | 147128157 | 0.34927631 | 0.698552619 | 137005 | 35 | 7545 | ZIC1 | 942 | uc003ewe.3 | FALSE |
| chr11 | 125774406 | 125774447 | 0.349140821 | 0.698281642 | 41529 | 42 | 29118 | DDX25 | 145 | uc001qcz.5 | FALSE |
| chr5 | 1225074 | 1225224 | −0.348772819 | 0.697545638 | 149862 | 151 | 340024 | SLC6A19 | 23364 | uc003jby.2 | FALSE |
| chr4 | 176987174 | 176987313 | 0.348766256 | 0.697532513 | 148496 | 140 | 116966 | WDR17 | 189 | uc003iul.2 | FALSE |
| chr10 | 94180383 | 94180551 | 0.348706658 | 0.697413317 | 26625 | 169 | 100507674 | MARK2P9 | 1965 | uc031pww.1 | FALSE |
| chr5 | 1752840 | 1752864 | −0.34816417 | 0.696328341 | 150055 | 25 | 100422966 | MIR4277 | −43857 | uc021xwg.1 | FALSE |
| chr14 | 24803917 | 24803925 | 0.348080118 | 0.696160236 | 58745 | 9 | 196883 | ADCY4 | 352 | uc001woz.4 | FALSE |
| chr19 | 42571339 | 42571402 | −0.347973731 | 0.695947461 | 99661 | 64 | 64763 | ZNF574 | −1227 | uc002osk.4 | TRUE |
| chr15 | 27212911 | 27213174 | 0.347873212 | 0.695746425 | 65182 | 264 | 2567 | GABRG3 | −3255 | uc001zbf.4 | FALSE |
| chr8 | 25909326 | 25909385 | 0.347873186 | 0.695746372 | 187468 | 60 | 5520 | PPP2R2A | 679251 | uc003xes.2 | FALSE |
| chr1 | 38100826 | 38100837 | 0.347414075 | 0.694828149 | 6484 | 12 | 284654 | RSPO1 | −231 | uc001cbl.2 | TRUE |
| chr2 | 162283189 | 162283434 | 0.347175076 | 0.694350152 | 113205 | 246 | 10716 | TBR1 | 8783 | uc010foy.2 | FALSE |
| chr17 | 46646043 | 46646359 | 0.347148087 | 0.694296173 | 86613 | 317 | 3213 | HOXB3 | 5451 | uc002ino.3 | FALSE |
| chr7 | 63767753 | 63767841 | 0.347100066 | 0.694200132 | 177690 | 89 | 728927 | ZNF736 | −5345 | uc011kdo.2 | FALSE |
| chr2 | 182545771 | 182545934 | 0.347073014 | 0.694146029 | 114385 | 164 | 375298 | CERKL | −379 | uc002uod.3 | TRUE |
| chr4 | 165878136 | 165878219 | 0.346912222 | 0.693824443 | 148103 | 84 | 152756 | FAM218A | 36 | uc003iqx.1 | FALSE |
| chr6 | 28761989 | 28762125 | 0.346858238 | 0.693716476 | 163030 | 137 | 401242 | LINC01623 | 69329 | uc003nlq.2 | FALSE |
| chr14 | 62584278 | 62584513 | 0.346843511 | 0.693687022 | 60446 | 236 | 646113 | LINC00643 | 203 | uc010apt.2 | FALSE |
| chr7 | 136553884 | 136554352 | 0.346809008 | 0.693618016 | 182445 | 469 | 1129 | CHRM2 | 52 | uc003vto.1 | FALSE |
| chr12 | 71314587 | 71314612 | 0.346696054 | 0.693392107 | 47821 | 26 | 5801 | PTPRR | −3 | uc001swi.2 | TRUE |
| chr5 | 76934327 | 76934461 | 0.346665104 | 0.693330208 | 153514 | 135 | 23440 | OTP | 61 | uc003kfg.3 | FALSE |
| chr21 | 36421467 | 36421472 | −0.346564359 | 0.693128717 | 123419 | 6 | 861 | RUNX1 | 123 | uc010gmu.3 | FALSE |
| chr5 | 172659730 | 172660080 | 0.346537148 | 0.693074297 | 159090 | 351 | 1482 | NKX2-5 | 2235 | uc011dfe.2 | FALSE |
| chr7 | 121943990 | 121944173 | 0.346487323 | 0.692974646 | 181372 | 184 | 154860 | FEZF1-AS1 | 278 | uc010lko.2 | FALSE |
| chr14 | 58063727 | 58063811 | −0.346222803 | 0.692445606 | 60112 | 85 | 341880 | SLC35F4 | 268781 | uc010aoz.1 | FALSE |
| chr16 | 899090 | 899108 | −0.346140427 | 0.692280854 | 72030 | 19 | 64788 | LMF1 | 19948 | uc010brg.2 | FALSE |
| chr6 | 27182908 | 27182943 | 0.346064583 | 0.692129166 | 162745 | 36 | 10279 | PRSS16 | −32559 | uc003nja.3 | FALSE |
| chr6 | 100050791 | 100050812 | 0.346057953 | 0.692115907 | 167685 | 22 | 59336 | PRDM13 | −3838 | uc003pqg.1 | FALSE |
| chr6 | 166581538 | 166581929 | 0.345942139 | 0.691884277 | 171569 | 392 | 6862 | TBXT | 228 | uc003qut.2 | FALSE |
| chr10 | 102900130 | 102900365 | 0.345929768 | 0.691859535 | 27499 | 236 | 3195 | TLX1 | 9069 | uc021pxd.1 | FALSE |
| chrX | 113818412 | 113818537 | 0.345923377 | 0.691846755 | 203326 | 126 | 3358 | HTR2C | −14 | uc004epu.1 | TRUE |
| chr16 | 6533163 | 6533187 | 0.345797052 | 0.691594104 | 73511 | 25 | 54715 | RBFOX1 | 463459 | uc010uya.1 | FALSE |
| chrX | 146312598 | 146312617 | −0.34560794 | 0.691215879 | 204108 | 20 | 574512 | MIR507 | −3 | uc022cfv.1 | TRUE |
| chr1 | 101004638 | 101004934 | 0.345529697 | 0.691059393 | 10639 | 297 | 54112 | GPR88 | 910 | uc001dth.3 | FALSE |
| chr17 | 1174148 | 1174473 | 0.345445145 | 0.690890291 | 80945 | 326 | 727857 | BHLHA9 | 290 | uc021tnd.1 | FALSE |
| chr10 | 119000638 | 119000927 | 0.345160057 | 0.690320114 | 28635 | 290 | 6571 | SLC18A2 | 54 | uc009xyy.2 | FALSE |
| chr19 | 11784647 | 11784672 | 0.344967308 | 0.689934615 | 96415 | 26 | 401898 | ZNF833P | 34056 | uc021upi.1 | FALSE |
| chr10 | 109674854 | 109674963 | 0.344899537 | 0.689799075 | 28044 | 110 | 114815 | SORCS1 | −750388 | uc001kyl.3 | FALSE |
| chr1 | 3826425 | 3826643 | −0.344752782 | 0.689505563 | 1444 | 219 | 100133612 | LINC01134 | 9457 | uc001alh.4 | FALSE |
| chr6 | 5026324 | 5026435 | 0.34470161 | 0.68940322 | 160983 | 112 | 10799 | RPP40 | −22053 | uc003mwl.3 | FALSE |
| chr17 | 46699073 | 46699155 | 0.344640974 | 0.689281948 | 86647 | 83 | 3219 | HOXB9 | 4680 | uc002inx.3 | FALSE |
| chr19 | 51111388 | 51111677 | 0.344592285 | 0.68918457 | 101292 | 290 | 100126781 | SNAR-F | 3168 | uc010eoa.1 | FALSE |
| chr8 | 61193715 | 61193970 | 0.344575418 | 0.689150837 | 189463 | 256 | 767 | CA8 | 0 | uc003xub.3 | TRUE |
| chr12 | 2743145 | 2743239 | −0.344504473 | 0.689008945 | 42789 | 95 | 775 | CACNA1C | 513654 | uc010sea.1 | FALSE |
| chr7 | 20818844 | 20818887 | 0.344427332 | 0.688854663 | 174744 | 44 | 221833 | SP8 | 7621 | uc003suz.3 | FALSE |
| chr2 | 123418757 | 123418787 | 0.344360339 | 0.688720677 | 111237 | 31 | 7247 | TSN | 905636 | uc031rox.1 | FALSE |
| chr9 | 79630987 | 79631227 | 0.344328203 | 0.688656406 | 196097 | 241 | 442425 | FOXB2 | −3344 | uc004ako.1 | FALSE |
| chr8 | 140624507 | 140624727 | −0.344054383 | 0.688108766 | 193273 | 221 | 51305 | KCNK9 | 6615 | uc003yve.1 | FALSE |
| chr14 | 21359737 | 21359943 | −0.344024398 | 0.688048797 | 58233 | 207 | 6037 | RNASE3 | 175 | uc001vyj.3 | FALSE |
| chr4 | 206339 | 206562 | 0.344015915 | 0.688031831 | 140213 | 224 | 642280 | ZNF876P | 0 | uc010iba.4 | TRUE |
| chr18 | 44337910 | 44337922 | 0.343916171 | 0.687832342 | 92290 | 13 | 29906 | ST8SIA5 | −871 | uc002lcj.1 | TRUE |
| chr7 | 54271152 | 54271282 | −0.34390644 | 0.68781288 | 177216 | 131 | 3262 | NA | −1038 | uc010kzd.2 | TRUE |
| chr3 | 62861631 | 62861796 | 0.343797965 | 0.68759593 | 132725 | 166 | 8618 | CADPS | −567 | uc003dll.2 | TRUE |
| chr11 | 60282712 | 60282742 | −0.343581941 | 0.687163883 | 35542 | 31 | 503497 | MS4A13 | −144 | uc001nps.3 | TRUE |
| chr4 | 20985927 | 20985984 | 0.343441439 | 0.686882879 | 142522 | 58 | 80333 | KCNIP4 | 319545 | uc003gqe.2 | FALSE |
| chr4 | 3675120 | 3675363 | −0.343354646 | 0.686709292 | 141186 | 244 | 100133461 | LINC02171 | 4219 | uc003ghl.3 | FALSE |
| chr6 | 27559924 | 27559939 | 0.34327082 | 0.686541641 | 162812 | 16 | 100507173 | LINC01012 | −101875 | uc021yrb.1 | FALSE |
| chr2 | 176979345 | 176979788 | 0.342937729 | 0.685875457 | 114069 | 444 | 3236 | HOXD10 | −1704 | uc002ukj.3 | TRUE |
| chr19 | 11998871 | 11999000 | 0.342828372 | 0.685656743 | 96442 | 130 | 7620 | ZNF69 | 201 | uc002mst.4 | FALSE |
| chr14 | 57275166 | 57275282 | 0.342803811 | 0.685607621 | 60072 | 117 | 5015 | OTX2 | 1912 | uc031qot.1 | FALSE |
| chr6 | 29521420 | 29521430 | 0.342728268 | 0.685456536 | 163140 | 11 | 10537 | UBD | 6272 | uc003nmo.3 | FALSE |
| chr19 | 24154607 | 24154730 | 0.342656275 | 0.68531255 | 98089 | 124 | 730087 | ZNF726 | 56923 | uc021urw.1 | FALSE |
| chr7 | 54610192 | 54610320 | 0.342620049 | 0.685240099 | 177223 | 129 | 222008 | VSTM2A | 173 | uc010kzf.3 | FALSE |
| chr19 | 30019529 | 30019647 | 0.342566557 | 0.685133113 | 98141 | 119 | 342865 | VSTM2B | 2038 | uc010xrl.1 | FALSE |
| chr19 | 44952766 | 44952808 | 0.3425523 | 0.6851046 | 99984 | 43 | 7772 | ZNF229 | 0 | uc010ejl.1 | TRUE |
| chr2 | 100824662 | 100824702 | −0.342476312 | 0.684952623 | 109716 | 41 | 164832 | LONRF2 | 100985 | uc002tag.3 | FALSE |
| chr19 | 54668230 | 54668256 | 0.342394312 | 0.684788623 | 101942 | 27 | 147798 | TMC4 | 194 | uc002qdn.3 | FALSE |
| chr14 | 60977856 | 60977961 | 0.342381591 | 0.684763183 | 60299 | 106 | 4990 | SIX6 | 1918 | uc001xfa.4 | FALSE |
| chr10 | 118900324 | 118900422 | 0.34220568 | 0.68441136 | 28619 | 99 | 11023 | VAX1 | −2512 | uc001ldb.1 | FALSE |
| chr12 | 104697526 | 104697532 | 0.342193567 | 0.684387133 | 49262 | 7 | 493861 | EID3 | 16 | uc001tkw.3 | FALSE |
| chr12 | 114841980 | 114842031 | 0.342140584 | 0.684281169 | 50274 | 52 | 6910 | TBX5 | 1937 | uc010syv.2 | FALSE |
| chr4 | 81122687 | 81122726 | 0.342113355 | 0.68422671 | 144795 | 40 | 56978 | PRDM8 | 4030 | uc003hmc.4 | FALSE |
| chr12 | 2944310 | 2944322 | 0.342082119 | 0.684164239 | 42821 | 13 | 83714 | NRIP2 | −89 | uc001qlc.3 | TRUE |
| chr2 | 157177072 | 157177345 | 0.341968234 | 0.683936469 | 112883 | 274 | 4929 | NR4A2 | 9355 | uc010zcg.1 | FALSE |
| chrX | 50213528 | 50213549 | 0.341875168 | 0.683750337 | 202015 | 22 | 139189 | DGKK | 172 | uc010njr.2 | FALSE |
| chr19 | 57337469 | 57337648 | −0.341691032 | 0.683382064 | 102453 | 180 | 23619 | ZIM2 | 9824 | uc002qnu.2 | FALSE |
| chr5 | 156536107 | 156536137 | −0.341617893 | 0.683235785 | 158153 | 31 | 84868 | HAVCR2 | 111 | uc003lwl.3 | FALSE |
| chrX | 91036807 | 91036888 | 0.341515044 | 0.683030088 | 202828 | 82 | 27328 | PCDH11X | 2547 | uc004efh.2 | FALSE |
| chr17 | 46810768 | 46810934 | 0.341466523 | 0.682933046 | 86674 | 167 | 10481 | HOXB13 | −4657 | uc002ioa.3 | FALSE |
| chr12 | 54772688 | 54772804 | 0.341395996 | 0.682791992 | 46622 | 117 | 25946 | ZNF385A | 5544 | uc009zno.1 | FALSE |
| chr7 | 127881269 | 127881280 | 0.341390247 | 0.682780494 | 181678 | 12 | 3952 | LEP | −51 | uc003vml.2 | TRUE |
| chr11 | 60049447 | 60049475 | −0.341278262 | 0.682556525 | 35514 | 29 | 51338 | MS4A4A | 1433 | uc001npc.3 | FALSE |
| chr14 | 101500080 | 101500125 | −0.341242122 | 0.682484245 | 63527 | 46 | 574453 | MIR495 | 0 | uc021scu.1 | TRUE |
| chr1 | 91184770 | 91185028 | 0.341045008 | 0.682090016 | 10123 | 259 | 343472 | BARHL2 | −1976 | uc001dns.3 | TRUE |
| chr4 | 81185022 | 81185228 | 0.340756431 | 0.681512861 | 144802 | 207 | 2250 | FGF5 | −2514 | uc003hmd.3 | FALSE |
| chr4 | 1399938 | 1400189 | 0.340748621 | 0.681497243 | 140616 | 252 | 285464 | NA | 14598 | uc003gdf.2 | FALSE |
| chr19 | 37464417 | 37464508 | 0.340681976 | 0.681363951 | 98827 | 92 | 374900 | ZNF568 | 302 | uc010efi.2 | FALSE |
| chr7 | 27179161 | 27179268 | 0.340582975 | 0.68116595 | 175220 | 108 | 100133311 | HOXA-AS3 | −715 | uc003syr.2 | TRUE |
| chr14 | 103740065 | 103740085 | 0.340275657 | 0.680551314 | 63890 | 21 | 1983 | EIF5 | −60254 | uc001ymq.4 | FALSE |
| chr7 | 136555777 | 136556193 | 0.340252696 | 0.680505392 | 182446 | 417 | 1129 | CHRM2 | 1793 | uc003vto.1 | FALSE |
| chr15 | 89952052 | 89952135 | 0.34024832 | 0.68049664 | 70551 | 84 | 254559 | MIR9-3HG | 30779 | uc002bnw.2 | FALSE |
| chr6 | 28494196 | 28494445 | −0.340208467 | 0.680416934 | 162969 | 250 | 2880 | GPX5 | 407 | uc003nlm.2 | FALSE |
| chr15 | 76634516 | 76634887 | 0.340180361 | 0.680360723 | 69424 | 372 | 64843 | ISL2 | 5369 | uc021sqw.1 | FALSE |
| chr17 | 46824915 | 46825011 | 0.340172225 | 0.680344451 | 86680 | 97 | 284076 | TTLL6 | 46675 | uc002ioa.3 | FALSE |
| chr7 | 143579665 | 143579698 | 0.34003765 | 0.6800753 | 183092 | 34 | 9747 | TCAF1 | 2768 | uc011ktu.2 | FALSE |
| chr17 | 46655818 | 46655829 | 0.339823938 | 0.679647877 | 86619 | 12 | 3213 | HOXB3 | 3987 | uc002inp.3 | FALSE |
| chr2 | 176973690 | 176973835 | 0.339815994 | 0.679631988 | 114065 | 146 | 3237 | HOXD11 | 1606 | uc002uki.3 | FALSE |
| chr7 | 158785291 | 158785469 | 0.339804426 | 0.679608852 | 184932 | 179 | 154822 | LINC00689 | −15576 | uc003wof.3 | FALSE |
| chr3 | 129407365 | 129407530 | 0.339580747 | 0.679161494 | 136015 | 166 | 23023 | TMCC1 | 45 | uc003emy.4 | FALSE |
| chr6 | 97009594 | 97010071 | −0.339529611 | 0.679059222 | 167555 | 478 | 9457 | FHL5 | −353 | uc003pos.2 | TRUE |
| chr7 | 145813915 | 145813946 | 0.339297998 | 0.678595996 | 183163 | 32 | 26047 | CNTNAP2 | 462 | uc003weu.2 | FALSE |
| chr3 | 157813327 | 157813363 | 0.339277466 | 0.678554932 | 137627 | 37 | 6474 | SHOX2 | 10589 | uc010hvw.3 | FALSE |
| chr7 | 39015705 | 39015928 | 0.339275244 | 0.678550488 | 176189 | 224 | 11281 | POU6F2 | −1681 | uc003thb.2 | TRUE |
| chr12 | 113916473 | 113916609 | 0.33920797 | 0.67841594 | 50194 | 137 | 64211 | LHX5 | −6596 | uc001tvj.1 | FALSE |
| chr6 | 127835990 | 127836053 | 0.339136667 | 0.678273333 | 168990 | 64 | 387104 | SOGA3 | 4447 | uc003qbd.3 | FALSE |
| chr7 | 96627700 | 96627821 | 0.338866008 | 0.677732016 | 179551 | 122 | 285987 | DLX6-AS1 | 5789 | uc003uok.3 | FALSE |
| chr7 | 691506 | 691761 | −0.338683483 | 0.677366965 | 172655 | 256 | 5575 | PRKAR1B | 39667 | uc031swi.1 | FALSE |
| chr7 | 27184264 | 27184271 | 0.338495763 | 0.676991526 | 175222 | 8 | 100133311 | HOXA-AS3 | 4281 | uc003syn.2 | FALSE |
| chr5 | 79330929 | 79330943 | 0.338401416 | 0.676802832 | 153685 | 15 | 7060 | THBS4 | −227 | uc021yaw.1 | TRUE |
| chr11 | 124735089 | 124735105 | 0.338322573 | 0.676645145 | 41381 | 17 | 64221 | ROBO3 | −200 | uc001qbc.3 | TRUE |
| chr9 | 1042605 | 1042770 | 0.338294587 | 0.676589173 | 194665 | 166 | 10655 | DMRT2 | −7576 | uc003zgy.4 | FALSE |
| chr17 | 59534998 | 59535253 | 0.338245863 | 0.676491726 | 87804 | 256 | 9496 | TBX4 | 1191 | uc010woy.2 | FALSE |
| chr1 | 207642938 | 207643142 | −0.338177825 | 0.676355649 | 17040 | 205 | 1380 | CR2 | 15293 | uc009xci.1 | FALSE |
| chr19 | 48918712 | 48919198 | 0.338163078 | 0.676326156 | 100734 | 487 | 2906 | GRIN2D | 20580 | uc010elx.3 | FALSE |
| chr1 | 159284082 | 159284298 | −0.338124157 | 0.676248313 | 13710 | 217 | 441911 | OR10J3 | 151 | uc010piu.2 | FALSE |
| chr10 | 54073150 | 54073189 | 0.338085096 | 0.676170191 | 24084 | 40 | 100506939 | PRKG1-AS1 | 699 | uc009xox.2 | FALSE |
| chrX | 78200921 | 78201182 | −0.337924143 | 0.675848286 | 202747 | 262 | 27334 | P2RY10 | 92 | uc004edf.3 | FALSE |
| chr22 | 42679577 | 42679804 | 0.337889929 | 0.675779857 | 127119 | 228 | 388906 | OGFRP1 | 13818 | uc003bcl.3 | FALSE |
| chr12 | 126926911 | 126926925 | −0.337771357 | 0.675542714 | 51648 | 15 | 100128554 | LINC02347 | −102 | uc009zyj.1 | TRUE |
| chr8 | 77585222 | 77585318 | 0.337745522 | 0.675491043 | 190217 | 97 | 100192378 | ZFHX4-AS1 | 10192 | uc003yat.1 | FALSE |
| chr1 | 246728663 | 246728677 | −0.337626772 | 0.675253545 | 20036 | 15 | 64216 | TFB2M | 888 | uc010pys.1 | FALSE |
| chr2 | 220349208 | 220349615 | 0.337588987 | 0.675177973 | 116547 | 408 | 10290 | SPEG | 49508 | uc002vlr.3 | FALSE |
| chr12 | 120241287 | 120241409 | 0.337271443 | 0.674542885 | 50679 | 123 | 11113 | CIT | 873 | uc001txh.2 | FALSE |
| chr13 | 79176272 | 79176572 | 0.337161213 | 0.674322427 | 55869 | 301 | 5457 | POU4F1 | 1123 | uc001vkv.3 | FALSE |
| chr9 | 842914 | 843179 | 0.336929796 | 0.673859592 | 194648 | 266 | 1761 | DMRT1 | 1224 | uc003zgv.3 | FALSE |
| chr1 | 40598772 | 40598779 | 0.336769457 | 0.673538914 | 6819 | 8 | 6018 | RLF | −28262 | uc001cfc.4 | FALSE |
| chr7 | 27265309 | 27265463 | 0.336547479 | 0.673094959 | 175267 | 155 | 2128 | EVX1 | −16701 | uc003szd.1 | FALSE |
| chr5 | 35939847 | 35940037 | −0.336452461 | 0.672904923 | 151642 | 191 | 133690 | CAPSL | −966 | uc003jju.1 | TRUE |
| chr6 | 31651020 | 31651029 | 0.336214183 | 0.672428365 | 163666 | 10 | 80741 | LY6G5C | 788 | uc010jtb.1 | FALSE |
| chr4 | 122686038 | 122686269 | 0.336212799 | 0.672425598 | 146422 | 232 | 100192379 | PP12613 | 298 | uc003idx.1 | FALSE |
| chr7 | 22894898 | 22895283 | −0.336165798 | 0.672331596 | 174888 | 386 | 692210 | SNORD93 | −949 | uc003svl.3 | TRUE |
| chr8 | 144241104 | 144241120 | 0.33594663 | 0.671893261 | 193966 | 17 | 4062 | LY6H | 337 | uc011lka.2 | FALSE |
| chr11 | 32460656 | 32460799 | 0.335800836 | 0.671601672 | 33813 | 144 | 51352 | WT1-AS | 3071 | uc010red.2 | FALSE |
| chr17 | 37560925 | 37561101 | −0.335711293 | 0.671422587 | 85005 | 177 | 5469 | MED1 | 46426 | uc002hrt.3 | FALSE |
| chr13 | 101629969 | 101630201 | −0.335563075 | 0.67112615 | 56639 | 233 | 100885778 | NALCN-AS1 | 269390 | uc031qnb.1 | FALSE |
| chrX | 134305386 | 134305728 | −0.335491979 | 0.670983958 | 203847 | 343 | 54967 | CT55 | 23 | uc004eyl.1 | FALSE |
| chr7 | 6703803 | 6704045 | 0.335433061 | 0.670866122 | 174217 | 243 | 7559 | ZNF12 | 40809 | uc011jxa.1 | FALSE |
| chr12 | 133481390 | 133481466 | 0.335353921 | 0.670707842 | 52685 | 77 | 100289635 | ZNF605 | 51426 | uc001uld.2 | FALSE |
| chr19 | 14016717 | 14016729 | 0.33514019 | 0.670280379 | 96811 | 13 | 79173 | C19orf57 | 180 | uc002mxl.1 | FALSE |
| chr11 | 4928572 | 4928579 | −0.335042735 | 0.670085471 | 31928 | 8 | 119687 | OR51A7 | −21 | uc010qyq.2 | TRUE |
| chr12 | 123754235 | 123754328 | 0.334970266 | 0.669940532 | 51210 | 94 | 8099 | CDK2AP1 | 453 | uc031qjz.1 | FALSE |
| chr7 | 25989735 | 25989763 | 0.334755485 | 0.669510971 | 175117 | 29 | 406940 | MIR148A | −129 | uc011jzf.2 | TRUE |
| chr3 | 185277705 | 185277779 | −0.334630671 | 0.669261343 | 139010 | 75 | 200879 | LIPH | −7336 | uc003fpm.3 | FALSE |
| chr3 | 138821761 | 138821772 | −0.334562781 | 0.669125562 | 136634 | 12 | 60467 | BPESC1 | −1255 | uc003eta.3 | TRUE |
| chr12 | 114883063 | 114883316 | 0.334524804 | 0.669049609 | 50285 | 254 | 255480 | TBX5-AS1 | 36504 | uc001tvs.2 | FALSE |
| chr16 | 1493637 | 1493859 | −0.334469129 | 0.668938257 | 72350 | 223 | 645811 | CCDC154 | 631 | uc010uve.2 | FALSE |
| chr10 | 120355419 | 120355427 | 0.334406239 | 0.668812478 | 28709 | 9 | 2834 | PRLHR | −259 | uc001ldp.1 | TRUE |
| chr3 | 147130477 | 147130536 | 0.334349945 | 0.668699889 | 137007 | 60 | 7545 | ZIC1 | 3296 | uc003ewe.3 | FALSE |
| chr3 | 5137773 | 5137953 | 0.334299462 | 0.668598923 | 128353 | 181 | 55207 | ARL8B | −25977 | uc003bqg.3 | FALSE |
| chr1 | 34329348 | 34329757 | −0.334201247 | 0.668402494 | 6107 | 410 | 127540 | HMGB4 | 0 | uc001bxq.3 | TRUE |
| chr5 | 158524404 | 158524649 | 0.334128754 | 0.668257509 | 158287 | 246 | 1879 | EBF1 | 2139 | uc011ddw.2 | FALSE |
| chr3 | 147136904 | 147136922 | 0.334090959 | 0.668181919 | 137010 | 19 | 7545 | ZIC1 | 9723 | uc003ewe.3 | FALSE |
| chr11 | 55872467 | 55872478 | −0.333785363 | 0.667570727 | 35116 | 12 | 390151 | OR8H2 | −41 | uc010riy.2 | TRUE |
| chr19 | 55043790 | 55043845 | −0.333730502 | 0.667461003 | 102017 | 56 | 90011 | KIR3DX1 | −64 | uc010yfa.1 | TRUE |
| chr19 | 58627859 | 58627987 | −0.333727345 | 0.667454691 | 102671 | 129 | 65982 | ZSCAN18 | 1806 | uc002qrm.2 | FALSE |
| chr22 | 48970993 | 48971051 | 0.333646066 | 0.667292132 | 127718 | 59 | 25817 | TAFA5 | 85705 | uc003bio.4 | FALSE |
| chr2 | 79739940 | 79740185 | 0.333439099 | 0.666878197 | 108620 | 246 | 1496 | CTNNA2 | 0 | uc010ysg.2 | TRUE |
| chr7 | 71801793 | 71802184 | 0.333299731 | 0.666599462 | 178130 | 392 | 83698 | CALN1 | 24 | uc003twa.4 | FALSE |
| chr14 | 97499908 | 97499987 | 0.333114195 | 0.66622839 | 63058 | 80 | 7443 | VRK1 | 236224 | uc001yft.3 | FALSE |
| chr12 | 1943722 | 1943758 | −0.333097412 | 0.666194824 | 42674 | 37 | 654429 | LRTM2 | 14289 | uc010sdx.1 | FALSE |
| chr2 | 239140182 | 239140190 | 0.332804537 | 0.665609075 | 118172 | 9 | 151174 | LINC02610 | 128 | uc002vxy.2 | FALSE |
| chr19 | 23300066 | 23300243 | 0.332713202 | 0.665426404 | 98068 | 178 | 100129543 | ZNF730 | 289 | uc031rkc.1 | FALSE |
| chr14 | 42075406 | 42075554 | 0.332699102 | 0.665398203 | 59313 | 149 | 145581 | LRFN5 | −1210 | uc001wvm.3 | TRUE |
| chr19 | 44324945 | 44324951 | 0.332629558 | 0.665259115 | 99913 | 7 | 284348 | LYPD5 | −137 | uc002oxn.4 | TRUE |
| chr7 | 96622042 | 96622204 | 0.332481492 | 0.664962984 | 179550 | 163 | 285987 | DLX6-AS1 | 11406 | uc003uok.3 | FALSE |
| chr13 | 78550373 | 78550384 | −0.332397531 | 0.664795063 | 55854 | 12 | 100874222 | OBI1-AS1 | 56549 | uc001vkq.1 | FALSE |
| chrX | 151286548 | 151286698 | −0.332118931 | 0.664237862 | 204254 | 151 | 100533997 | MAGEA10-MAGEA5 | 20352 | uc004ffj.3 | FALSE |
| chr19 | 16022523 | 16022934 | 0.3318731 | 0.663746199 | 97144 | 412 | 57834 | CYP4F11 | 22285 | uc002nbs.1 | FALSE |
| chr19 | 54204403 | 54204449 | −0.331863344 | 0.663726687 | 101815 | 47 | 574473 | MIR520B | −32 | uc021uzy.1 | TRUE |
| chr19 | 58400325 | 58400494 | 0.331556614 | 0.663113228 | 102620 | 170 | 730051 | ZNF814 | 0 | uc002qqp.3 | TRUE |
| chr22 | 17083482 | 17083563 | 0.331426082 | 0.662852164 | 124747 | 82 | 387590 | TPTEP1 | 681 | uc002zls.1 | FALSE |
| chr11 | 8190565 | 8190572 | 0.331216917 | 0.662433835 | 32301 | 8 | 79608 | RIC3 | 18 | uc001mgf.4 | FALSE |
| chr4 | 165878055 | 165878085 | 0.331142818 | 0.662285637 | 148103 | 31 | 391712 | TRIM61 | 20733 | uc003iqx.1 | FALSE |
| chr6 | 50692150 | 50692252 | 0.331140631 | 0.662281263 | 166046 | 103 | 83741 | TFAP2D | 10893 | uc011dwt.2 | FALSE |
| chr16 | 54407990 | 54408014 | −0.331042036 | 0.662084072 | 76480 | 25 | 79191 | IRX3 | −87612 | uc002eht.1 | FALSE |
| chr2 | 177025859 | 177025975 | 0.330922273 | 0.661844546 | 114090 | 117 | 3232 | HOXD3 | −2830 | uc002ukt.1 | FALSE |
| chr8 | 87082014 | 87082023 | 0.330905058 | 0.661810115 | 190550 | 10 | 85481 | PSKH2 | −163 | uc011lfy.2 | TRUE |
| chr5 | 140346236 | 140346263 | 0.330888603 | 0.661777207 | 156797 | 28 | 56134 | PCDHAC2 | 489 | uc011dag.2 | FALSE |
| chr4 | 122301740 | 122301816 | 0.330787338 | 0.661574676 | 146405 | 77 | 84109 | QRFPR | 365 | uc010inl.1 | FALSE |
| chr11 | 5009201 | 5009311 | −0.330685808 | 0.661371617 | 31935 | 111 | 56547 | MMP26 | −113 | uc001lzv.3 | TRUE |
| chr17 | 76719635 | 76719640 | 0.330666503 | 0.661333005 | 89739 | 6 | 9267 | CYTH1 | 192 | uc010wtx.1 | FALSE |
| chr3 | 170304760 | 170305197 | −0.330621411 | 0.661242822 | 138059 | 438 | 5010 | CLDN11 | 168107 | uc003fgz.2 | FALSE |
| chr2 | 101086756 | 101086963 | −0.330620156 | 0.661240312 | 109743 | 208 | 129521 | NMS | 0 | uc002tan.1 | TRUE |
| chr15 | 60295294 | 60295312 | 0.330575257 | 0.661150515 | 67555 | 19 | 27023 | FOXB1 | −1109 | uc002agj.1 | TRUE |
| chr11 | 131410868 | 131410879 | −0.330524352 | 0.661048705 | 42074 | 12 | 50863 | NTM | 170497 | uc010sci.2 | FALSE |
| chr6 | 29324300 | 29324414 | −0.330354049 | 0.660708098 | 163113 | 115 | 81696 | OR5V1 | −246 | uc011dlo.2 | TRUE |
| chr18 | 5196874 | 5197066 | 0.330078573 | 0.660157146 | 91302 | 193 | 642597 | AKAIN1 | 189 | uc010wzc.1 | FALSE |
| chr10 | 106400259 | 106400454 | 0.330011088 | 0.660022175 | 27988 | 196 | 22986 | SORCS3 | −405 | uc001kyi.1 | TRUE |
| chr6 | 29274620 | 29274727 | −0.330001097 | 0.660002193 | 163108 | 108 | 442191 | OR14J1 | 153 | uc011dln.2 | FALSE |
| chr12 | 132908454 | 132908739 | −0.329981022 | 0.659962044 | 52439 | 286 | 50614 | GALNT9 | −2549 | uc001ukc.4 | FALSE |
| chr3 | 130063469 | 130063505 | −0.329938637 | 0.659877275 | 136062 | 37 | 256076 | COL6A5 | −854 | uc010hti.2 | TRUE |
| chr14 | 101393226 | 101393397 | −0.329762533 | 0.659525066 | 63479 | 172 | 767562 | SNORD113-2 | −282 | uc001yij.3 | TRUE |
| chr18 | 905450 | 905611 | 0.329545578 | 0.659091156 | 91207 | 162 | 116 | ADCYAP1 | 253 | uc010dkh.4 | FALSE |
| chr17 | 6796941 | 6797034 | 0.329533935 | 0.65906787 | 81837 | 94 | 245 | ALOX12P2 | 40046 | uc002gdw.3 | FALSE |
| chrX | 113865129 | 113865353 | −0.329348085 | 0.658696169 | 203329 | 225 | 677816 | SNORA35 | 0 | uc004epw.1 | TRUE |
| chr7 | 32110650 | 32110988 | 0.329214859 | 0.658429719 | 175704 | 339 | 5137 | PDE1C | 0 | uc003tcn.1 | TRUE |
| chr19 | 30020886 | 30021276 | 0.329164663 | 0.658329326 | 98142 | 391 | 342865 | VSTM2B | 3395 | uc010xrl.1 | FALSE |
| chr3 | 74663689 | 74663796 | 0.328973607 | 0.657947215 | 133491 | 108 | 5067 | CNTN3 | −93346 | uc003dpm.1 | FALSE |
| chr4 | 44449738 | 44450234 | 0.328972293 | 0.657944586 | 143396 | 497 | 386617 | KCTD8 | 590 | uc003gwu.3 | FALSE |
| chr3 | 123813191 | 123813232 | −0.32878502 | 0.657570041 | 135275 | 42 | 8997 | KALRN | 14303 | uc010hru.2 | FALSE |
| chr4 | 154712422 | 154712580 | 0.328761178 | 0.657522356 | 147756 | 159 | 6423 | SFRP2 | −2194 | uc003inv.1 | FALSE |
| chr16 | 31548908 | 31549141 | 0.328665926 | 0.657331852 | 75703 | 234 | 51327 | AHSP | 9705 | uc002ecj.3 | FALSE |
| chr16 | 54689221 | 54689252 | 0.328462727 | 0.656925454 | 76495 | 32 | 643911 | CRNDE | 273438 | uc010vhc.3 | FALSE |
| chr3 | 186079142 | 186079367 | 0.328391194 | 0.656782388 | 139083 | 226 | 1608 | DGKG | 656 | uc011brx.2 | FALSE |
| chr17 | 16570473 | 16570483 | 0.328328911 | 0.656657821 | 82910 | 11 | 57547 | ZNF624 | −13306 | uc010cpi.2 | FALSE |
| chr1 | 1566687 | 1566699 | 0.328287303 | 0.656574605 | 435 | 13 | 142678 | MIB2 | 2201 | uc001agp.3 | FALSE |
| chr3 | 147109784 | 147110229 | 0.328167119 | 0.656334238 | 136999 | 446 | 84107 | ZIC4 | 0 | uc021xfe.1 | TRUE |
| chr5 | 92909069 | 92909429 | 0.328102219 | 0.656204438 | 154181 | 361 | 441094 | NR2F1-AS1 | 7309 | uc003kke.2 | FALSE |
| chr8 | 140714586 | 140714610 | 0.328090424 | 0.656180848 | 193285 | 25 | 51305 | KCNK9 | 689 | uc003yvg.1 | FALSE |
| chr1 | 46951318 | 46951354 | 0.328058562 | 0.656117125 | 7651 | 37 | 127343 | DMBX1 | −21314 | uc001cpw.3 | FALSE |
| chr7 | 37488936 | 37489005 | 0.327985592 | 0.655971185 | 176083 | 70 | 9844 | ELMO1 | −41 | uc010kxg.2 | TRUE |
| chr3 | 147114986 | 147115043 | 0.327934937 | 0.655869874 | 137001 | 58 | 84107 | ZIC4 | 7028 | uc003ewc.2 | FALSE |
| chr4 | 81110205 | 81110459 | 0.327896622 | 0.655793244 | 144789 | 255 | 56978 | PRDM8 | 3781 | uc003hmb.4 | FALSE |
| chr13 | 93879670 | 93879769 | 0.3276186 | 0.655237201 | 56086 | 100 | 10082 | GPC6 | 592 | uc001vlt.3 | FALSE |
| chr20 | 29994173 | 29994192 | −0.32756187 | 0.65512374 | 120237 | 20 | 245934 | DEFB121 | 6449 | uc002wvv.2 | FALSE |
| chr20 | 47934802 | 47934987 | 0.327536324 | 0.655072648 | 121511 | 186 | 441951 | ZFAS1 | 39623 | uc002xup.1 | FALSE |
| chr4 | 111539291 | 111539352 | 0.327438468 | 0.654876936 | 146004 | 62 | 5308 | PITX2 | 4902 | uc003iag.1 | FALSE |
| chr8 | 70984199 | 70984270 | 0.327392389 | 0.654784779 | 189933 | 72 | 63978 | PRDM14 | −637 | uc003xym.3 | TRUE |
| chr2 | 131721099 | 131721477 | 0.327342773 | 0.654685546 | 111780 | 379 | 50649 | ARHGEF4 | 32574 | uc010fmx.1 | FALSE |
| chr14 | 105658685 | 105658949 | −0.327293198 | 0.654586397 | 64386 | 265 | 256281 | NUDT14 | −11025 | uc001yqi.4 | FALSE |
| chr2 | 176977458 | 176977773 | 0.32722591 | 0.654451821 | 114068 | 316 | 3237 | HOXD11 | 5374 | uc002ukj.3 | FALSE |
| chr22 | 50623687 | 50623692 | 0.326981515 | 0.653963031 | 127986 | 6 | 80305 | TRABD | −667 | uc003bjr.2 | TRUE |
| chr13 | 92051400 | 92051576 | 0.326970211 | 0.653940423 | 56063 | 177 | 2262 | GPC5 | 465 | uc010tif.2 | FALSE |
| chr7 | 1275252 | 1275258 | 0.326831621 | 0.653663242 | 172933 | 7 | 340260 | UNCX | 2598 | uc011jvw.2 | FALSE |
| chr10 | 71332804 | 71333037 | 0.326758989 | 0.653517977 | 24769 | 234 | 50674 | NEUROG3 | 173 | uc001jpp.3 | FALSE |
| chr10 | 86958767 | 86959046 | −0.326649218 | 0.653298435 | 26123 | 280 | 100507470 | GRID1-AS1 | −378442 | uc001kdk.2 | FALSE |
| chr17 | 17626790 | 17627194 | 0.326617863 | 0.653235726 | 83062 | 405 | 10743 | RAI1 | 42003 | uc002grm.3 | FALSE |
| chr3 | 6904134 | 6904261 | 0.326598346 | 0.653196693 | 128390 | 128 | 2917 | GRM7 | 1332 | uc003bqm.2 | FALSE |
| chr12 | 114878570 | 114878877 | 0.326462148 | 0.652924296 | 50283 | 308 | 255480 | TBX5-AS1 | 32011 | uc001tvs.2 | FALSE |
| chr7 | 53286921 | 53287126 | 0.326370277 | 0.652740554 | 177201 | 206 | 285877 | POM121L12 | 183572 | uc003tpz.3 | FALSE |
| chr5 | 101632321 | 101632327 | 0.326300433 | 0.652600866 | 154543 | 7 | 353189 | SLCO4C1 | −68 | uc003knm.3 | TRUE |
| chr2 | 162275690 | 162275746 | 0.326139181 | 0.652278362 | 113201 | 57 | 10716 | TBR1 | 1284 | uc010foy.2 | FALSE |
| chr4 | 104641319 | 104641548 | 0.326106267 | 0.652212534 | 145725 | 230 | 6870 | TACR3 | −346 | uc003hxe.1 | TRUE |
| chr16 | 56227690 | 56228114 | 0.32597741 | 0.651954819 | 76578 | 425 | 26077 | DKFZP434H168 | 323 | uc002eiv.2 | FALSE |
| chr4 | 111537570 | 111537907 | 0.325925065 | 0.65185013 | 146003 | 338 | 5308 | PITX2 | 6347 | uc003iag.1 | FALSE |
| chrX | 144903661 | 144903691 | 0.325796061 | 0.651592122 | 204090 | 31 | 84631 | SLITRK2 | 795 | uc011mwt.2 | FALSE |
| chr19 | 37825320 | 37825388 | 0.32575788 | 0.651515759 | 98865 | 69 | 284459 | ZNF875 | 16507 | uc002ofz.3 | FALSE |
| chr12 | 114878144 | 114878163 | 0.32563535 | 0.6512707 | 50283 | 20 | 255480 | TBX5-AS1 | 31585 | uc001tvs.2 | FALSE |
| chr10 | 130298983 | 130299087 | −0.325599418 | 0.651198837 | 29680 | 105 | 4288 | MKI67 | −374515 | uc001lke.3 | FALSE |
| chr7 | 1460836 | 1460880 | −0.325187224 | 0.650374447 | 173004 | 45 | 79778 | MICALL2 | 21240 | uc003ski.4 | FALSE |
| chr10 | 102590168 | 102590225 | 0.325051445 | 0.65010289 | 27417 | 58 | 5076 | PAX2 | 84700 | uc001krp.1 | FALSE |
| chr3 | 27770995 | 27771053 | 0.325023017 | 0.650046034 | 129845 | 59 | 8320 | EOMES | −6789 | uc003cdx.4 | FALSE |
| chrX | 100333950 | 100334126 | 0.324885799 | 0.649771599 | 202904 | 177 | 59353 | TMEM35A | 114 | uc004egw.3 | FALSE |
| chr5 | 113699194 | 113699342 | 0.32481688 | 0.64963376 | 154993 | 149 | 3781 | KCNN2 | 1178 | uc031skt.1 | FALSE |
| chr8 | 143055450 | 143055526 | −0.324734335 | 0.649468671 | 193682 | 77 | 100616268 | MIR4472-1 | −202174 | uc022bbz.1 | FALSE |
| chr1 | 2984275 | 2984445 | 0.324731756 | 0.649463513 | 1043 | 171 | 440556 | PRDM16-DT | 0 | uc010nzg.1 | TRUE |
| chr1 | 209848901 | 209849185 | 0.324672741 | 0.649345482 | 17208 | 285 | 50486 | G0S2 | 231 | uc001hhi.4 | FALSE |
| chr9 | 35757158 | 35757314 | 0.32466883 | 0.64933766 | 195492 | 157 | 692094 | MSMP | −2884 | uc003zyb.2 | FALSE |
| chr11 | 31846414 | 31846533 | 0.324616872 | 0.649233744 | 33775 | 120 | 440034 | PAX6-AS1 | 8300 | uc009yjr.3 | FALSE |
| chr17 | 8869213 | 8869285 | 0.324567993 | 0.649135986 | 82317 | 73 | 23533 | PIK3R5 | −184 | uc002glu.4 | TRUE |
| chr4 | 46391929 | 46392253 | 0.32453743 | 0.64907486 | 143415 | 325 | 2555 | GABRA2 | 0 | uc011bzc.1 | TRUE |
| chr22 | 30877800 | 30877907 | −0.324258392 | 0.648516785 | 125991 | 108 | 376844 | SDC4P | −57 | uc003aic.1 | TRUE |
| chr2 | 200468832 | 200469026 | 0.324252913 | 0.648505826 | 115032 | 195 | 150538 | SATB2-AS1 | 136011 | uc002uuz.2 | FALSE |
| chr4 | 41746947 | 41747092 | 0.324123361 | 0.648246723 | 143308 | 146 | 8929 | PHOX2B | 3895 | uc003gwf.4 | FALSE |
| chr1 | 50886949 | 50886969 | 0.324121071 | 0.648242143 | 7883 | 21 | 63950 | DMRTA2 | 335 | uc010ona.2 | FALSE |
| chrX | 46937658 | 46937829 | 0.324076091 | 0.648152182 | 201675 | 172 | 9104 | RGN | 0 | uc010nhp.1 | TRUE |
| chr1 | 181452039 | 181452044 | 0.323999798 | 0.647999596 | 15331 | 6 | 777 | CACNA1E | −642 | uc001gow.3 | TRUE |
| chr17 | 3117953 | 3117981 | −0.323972504 | 0.647945009 | 81311 | 29 | 8383 | OR1A1 | −934 | uc010vrc.2 | TRUE |
| chr17 | 43339223 | 43339328 | 0.323904159 | 0.647808317 | 86188 | 106 | 124783 | SPATA32 | 151 | uc010wjk.1 | FALSE |
| chr3 | 22413680 | 22413960 | 0.323898808 | 0.647797615 | 129621 | 281 | 79750 | ZNF385D | 163 | uc010hfb.1 | FALSE |
| chr6 | 163730283 | 163730368 | −0.323675513 | 0.647351026 | 171372 | 86 | 135138 | PACRG | 581281 | uc011egh.2 | FALSE |
| chr17 | 36718198 | 36718549 | 0.323669151 | 0.647338302 | 84860 | 352 | 80725 | SRCIN1 | 1683 | uc002hqf.1 | FALSE |
| chr3 | 158390632 | 158390671 | 0.323578577 | 0.647157155 | 137647 | 40 | 85476 | GFM1 | 26678 | uc003fch.3 | FALSE |
| chr15 | 87667171 | 87667390 | −0.323386967 | 0.646773934 | 70351 | 220 | 123624 | AGBL1 | 981929 | uc002bmc.1 | FALSE |
| chr7 | 30721888 | 30722114 | 0.323349671 | 0.646699342 | 175594 | 227 | 1395 | CRHR2 | 27 | uc022abg.1 | FALSE |
| chr2 | 87017953 | 87018054 | 0.323318782 | 0.646637564 | 109001 | 102 | 925 | CD8A | 783 | uc010ytn.2 | FALSE |
| chr15 | 95870409 | 95870440 | 0.323228689 | 0.646457377 | 71090 | 32 | 400456 | LINC01197 | −80 | uc002btm.3 | TRUE |
| chr7 | 70597921 | 70598282 | 0.323203112 | 0.646406223 | 178081 | 362 | 64409 | GALNT17 | 398 | uc003tvy.4 | FALSE |
| chr8 | 1338463 | 1338706 | −0.323201084 | 0.646402168 | 185363 | 244 | 286083 | LOC286083 | −87636 | uc022aqn.1 | FALSE |
| chr8 | 69245801 | 69246056 | −0.323096058 | 0.646192117 | 189851 | 256 | 116328 | C8orf34 | 2844 | uc003xxx.4 | FALSE |
| chr2 | 172952415 | 172952883 | 0.323041225 | 0.646082451 | 113730 | 469 | 1745 | DLX1 | 2207 | uc002uhm.3 | FALSE |
| chr14 | 22917797 | 22917809 | −0.322989802 | 0.645979605 | 58387 | 13 | 1603 | DAD1 | 140334 | uc001wgl.2 | FALSE |
| chr15 | 78632670 | 78632677 | 0.322968019 | 0.645936038 | 69625 | 8 | 1381 | CRABP1 | 4 | uc002bdp.2 | FALSE |
| chr4 | 13530923 | 13530948 | 0.322926304 | 0.645852607 | 142211 | 26 | 285547 | LINC01097 | 2093 | uc021xmh.1 | FALSE |
| chr1 | 63790044 | 63790202 | 0.322868428 | 0.645736856 | 8893 | 159 | 27022 | FOXD3 | 1314 | uc001dax.2 | FALSE |
| chr19 | 49700006 | 49700021 | 0.322803409 | 0.645606817 | 100976 | 16 | 54795 | TRPM4 | 14223 | uc002pmy.3 | FALSE |
| chr11 | 111385450 | 111385461 | 0.322607912 | 0.645215824 | 39968 | 12 | 399949 | C11orf88 | −49 | uc001plo.1 | TRUE |
| chr13 | 65532408 | 65532581 | −0.322521265 | 0.64504253 | 55499 | 174 | 283491 | OR7E156P | 1220840 | uc001vig.1 | FALSE |
| chr6 | 28367544 | 28367571 | 0.322440824 | 0.644881649 | 162948 | 28 | 9753 | ZSCAN12 | 0 | uc011dlh.2 | TRUE |
| chr5 | 87967728 | 87967845 | 0.322100118 | 0.644200237 | 154016 | 118 | 645323 | LINC00461 | 1301 | uc021ybe.1 | FALSE |
| chr6 | 43211208 | 43211213 | 0.32203178 | 0.64406356 | 165469 | 6 | 84630 | TTBK1 | −9 | uc003ouq.1 | TRUE |
| chr12 | 72667150 | 72667236 | 0.321749317 | 0.643498634 | 47888 | 87 | 283392 | TRHDE-AS1 | 53 | uc010stv.2 | FALSE |
| chr7 | 31725748 | 31725973 | −0.321724373 | 0.643448747 | 175690 | 226 | 10842 | PPP1R17 | −658 | uc003tcl.3 | TRUE |
| chr12 | 115102713 | 115102726 | 0.321717681 | 0.643435361 | 50301 | 14 | 6926 | TBX3 | 19243 | uc010syw.1 | FALSE |
| chrX | 82765593 | 82765734 | 0.321649434 | 0.643298868 | 202782 | 142 | 5456 | POU3F4 | 2324 | uc004eeg.2 | FALSE |
| chr7 | 156812806 | 156812941 | 0.32162518 | 0.64325036 | 184330 | 136 | 645249 | MNX1-AS1 | 9255 | uc003wne.2 | FALSE |
| chr2 | 182321786 | 182321855 | 0.321593887 | 0.643187774 | 114372 | 70 | 3676 | ITGA4 | 167 | uc002unu.3 | FALSE |
| chr10 | 23982387 | 23982480 | 0.321517172 | 0.643034343 | 22277 | 94 | 56243 | KIAA1217 | −1195 | uc001irs.3 | TRUE |
| chr11 | 69832045 | 69832232 | 0.32151376 | 0.64302752 | 37459 | 188 | 55107 | ANO1 | −92176 | uc001opj.3 | FALSE |
| chr3 | 133464971 | 133465180 | 0.321225722 | 0.642451444 | 136250 | 210 | 7018 | TF | 171 | uc011blt.2 | FALSE |
| chr12 | 119418676 | 119418804 | 0.321126963 | 0.642253926 | 50618 | 129 | 84530 | SRRM4 | −496 | uc001txa.2 | TRUE |
| chr1 | 166039754 | 166039858 | −0.321026469 | 0.642052939 | 14365 | 105 | 149297 | FAM78B | 96100 | uc021peg.1 | FALSE |
| chr16 | 88236664 | 88236800 | −0.32097633 | 0.641952661 | 79820 | 137 | 54971 | BANP | 233040 | uc002fkr.3 | FALSE |
| chr6 | 41254433 | 41254471 | −0.320964814 | 0.641929629 | 165140 | 39 | 54210 | TREM1 | 0 | uc021yzj.1 | TRUE |
| chr10 | 134599151 | 134599372 | 0.320801631 | 0.641603263 | 30355 | 222 | 84504 | NKX6-2 | 165 | uc001llr.2 | FALSE |
| chr1 | 209405050 | 209405064 | 0.32071888 | 0.641437759 | 17162 | 15 | 642587 | MIR205HG | −197104 | uc009xcn.3 | FALSE |
| chr5 | 23304089 | 23304101 | −0.320711113 | 0.641422227 | 151226 | 13 | 56979 | PRDM9 | −203623 | uc003jgo.3 | FALSE |
| chrX | 144899592 | 144899995 | 0.320700623 | 0.641401245 | 204088 | 404 | 84631 | SLITRK2 | 245 | uc011mwq.2 | FALSE |
| chr2 | 176933286 | 176933407 | 0.320497519 | 0.640995039 | 114049 | 122 | 344191 | EVX2 | 15283 | uc010zeu.2 | FALSE |
| chr11 | 4566169 | 4566853 | −0.320355399 | 0.640710798 | 31886 | 685 | 119772 | OR52M1 | 0 | uc010qyf.2 | TRUE |
| chr7 | 569979 | 570041 | −0.320263022 | 0.640526045 | 172598 | 63 | 441307 | HRAT92 | 9951 | uc021zyh.1 | FALSE |
| chr5 | 2757221 | 2757244 | 0.320242791 | 0.640485583 | 150234 | 24 | 153571 | C5orf38 | 4959 | uc011cmj.3 | FALSE |
| chr19 | 53661755 | 53662261 | 0.32019489 | 0.64038978 | 101724 | 507 | 84671 | ZNF347 | 52 | uc002qbc.2 | FALSE |
| chr8 | 49292486 | 49292685 | 0.320088054 | 0.640176107 | 188878 | 200 | 7336 | UBE2V2 | 371491 | uc011ldk.1 | FALSE |
| chr2 | 68546467 | 68546507 | 0.319968185 | 0.63993637 | 107745 | 41 | 25927 | CNRIP1 | 512 | uc010fdd.1 | FALSE |
| chr10 | 124901908 | 124901928 | 0.319907731 | 0.639815461 | 29135 | 21 | 340784 | HMX3 | 6341 | uc001lhc.1 | FALSE |
| chr14 | 52734286 | 52734325 | 0.319857956 | 0.639715911 | 59690 | 40 | 5729 | PTGDR | −106 | uc001wzq.3 | TRUE |
| chr7 | 156813574 | 156813602 | 0.319812399 | 0.639624799 | 184330 | 29 | 645249 | MNX1-AS1 | 10023 | uc003wne.2 | FALSE |
| chr4 | 156129424 | 156129574 | 0.319760217 | 0.639520435 | 147810 | 151 | 4887 | NPY2R | −207 | uc003ioq.3 | TRUE |
| chr12 | 119212248 | 119212313 | 0.319753509 | 0.639507018 | 50612 | 66 | 84530 | SRRM4 | −206987 | uc001txa.2 | FALSE |
| chr10 | 60936794 | 60936801 | 0.31967634 | 0.63935268 | 24228 | 8 | 84457 | PHYHIPL | 446 | uc001jkm.4 | FALSE |
| chr21 | 31972533 | 31972672 | −0.319583746 | 0.639167491 | 123164 | 140 | 337979 | KRTAP22-1 | −768 | uc011add.2 | TRUE |
| chr5 | 140181058 | 140181074 | 0.319523785 | 0.63904757 | 156748 | 17 | 56145 | PCDHA3 | 275 | uc003lhf.2 | FALSE |
| chr11 | 57005876 | 57005981 | −0.319486238 | 0.638972476 | 35183 | 106 | 187 | APLNR | −949 | uc001njn.4 | TRUE |
| chr1 | 41119940 | 41119988 | 0.319275175 | 0.63855035 | 6872 | 49 | 9783 | RIMS3 | 11336 | uc001cfv.1 | FALSE |
| chr2 | 207507001 | 207507096 | 0.318948537 | 0.637897074 | 115581 | 96 | 200726 | FAM237A | −46 | uc010fuh.1 | TRUE |
| chr13 | 78493066 | 78493100 | 0.31867095 | 0.6373419 | 55852 | 35 | 1910 | EDNRB | 803 | uc001vko.2 | FALSE |
| chr7 | 1451205 | 1451285 | −0.318560771 | 0.637121542 | 172999 | 81 | 79778 | MICALL2 | 30835 | uc003ski.4 | FALSE |
| chr12 | 129338534 | 129338841 | 0.318512718 | 0.637025437 | 51788 | 308 | 144423 | GLT1D1 | 453 | uc001uhy.1 | FALSE |
| chr11 | 89224506 | 89224559 | 0.318344569 | 0.636689138 | 38968 | 54 | 50507 | NOX4 | 94 | uc001pcy.3 | FALSE |
| chr5 | 27038782 | 27038802 | 0.318313856 | 0.636627712 | 151264 | 21 | 1007 | CDH9 | −93 | uc003jgs.1 | TRUE |
| chr13 | 93879303 | 93879361 | 0.318270687 | 0.636541373 | 56086 | 59 | 10082 | GPC6 | 225 | uc001vlt.3 | FALSE |
| chr6 | 33154938 | 33155135 | −0.318198485 | 0.63639697 | 164100 | 198 | 1302 | COL11A2 | 5110 | uc003oda.3 | FALSE |
| chr12 | 127212416 | 127212528 | 0.318057238 | 0.636114475 | 51660 | 113 | 387895 | LINC00944 | 44280 | uc001uhl.3 | FALSE |
| chr6 | 1393336 | 1393675 | 0.317916769 | 0.635833539 | 160485 | 340 | 2295 | FOXF2 | 3267 | uc003mtm.3 | FALSE |
| chr13 | 108518419 | 108518445 | 0.317806426 | 0.635612852 | 56885 | 27 | 728215 | FAM155A | 1015 | uc001vql.3 | FALSE |
| chr10 | 102497298 | 102497354 | 0.31749315 | 0.634986301 | 27401 | 57 | 5076 | PAX2 | −8114 | uc001krk.4 | FALSE |
| chr10 | 118031864 | 118031870 | 0.317387388 | 0.634774776 | 28533 | 7 | 2674 | GFRA1 | 1256 | uc001lci.3 | FALSE |
| chr8 | 139507782 | 139508113 | −0.317269855 | 0.63453971 | 193237 | 332 | 51059 | FAM135B | 952 | uc003yuz.3 | FALSE |
| chr3 | 62861245 | 62861311 | 0.317169309 | 0.634338618 | 132725 | 67 | 8618 | CADPS | −181 | uc003dll.2 | TRUE |
| chr14 | 103655712 | 103655773 | 0.317105173 | 0.634210345 | 63878 | 62 | 100131366 | LINC00605 | −347 | uc001ymn.3 | TRUE |
| chr1 | 241587609 | 241587783 | 0.316966173 | 0.633932346 | 19636 | 175 | 6000 | RGS7 | −67131 | uc001hyv.2 | FALSE |
| chr2 | 237082393 | 237082534 | 0.316636744 | 0.633273488 | 117856 | 142 | 2637 | GBX2 | −5741 | uc002vvw.1 | FALSE |
| chr5 | 115297853 | 115297989 | 0.316205242 | 0.632410483 | 155092 | 137 | 206338 | LVRN | −162 | uc003kro.3 | TRUE |
| chr3 | 108896930 | 108897005 | −0.31569897 | 0.63139794 | 134353 | 76 | 677779 | LINC00488 | −7 | uc003dxn.4 | TRUE |
| chr5 | 2223624 | 2223732 | −0.315260398 | 0.630520797 | 150174 | 109 | 50805 | IRX4 | −336331 | uc031siq.1 | FALSE |
| chr19 | 2046085 | 2046193 | 0.315231367 | 0.630462735 | 94310 | 109 | 2872 | MKNK2 | 497 | uc010xgu.1 | FALSE |
| chr5 | 2755158 | 2755376 | 0.315138808 | 0.630277615 | 150233 | 219 | 153571 | C5orf38 | 2896 | uc011cmj.3 | FALSE |
| chr5 | 134363973 | 134364387 | 0.315099381 | 0.630198762 | 156096 | 415 | 5307 | PITX1 | 5577 | uc003laj.2 | FALSE |
| chr22 | 48607064 | 48607198 | −0.314852115 | 0.629704229 | 127688 | 135 | 100422916 | MIR3201 | −62978 | uc021wrs.1 | FALSE |
| chrX | 51637971 | 51638041 | 0.314828628 | 0.629657256 | 202059 | 71 | 9500 | MAGED1 | 1273 | uc004dpo.3 | FALSE |
| chr19 | 12203066 | 12203198 | 0.314708559 | 0.629417118 | 96471 | 133 | 388507 | ZNF788P | 0 | uc002mtd.3 | TRUE |
| chr12 | 58021295 | 58021309 | 0.314573765 | 0.62914753 | 47104 | 15 | 2583 | B4GALNT1 | 5155 | uc009zpz.2 | FALSE |
| chr12 | 54422447 | 54422488 | 0.314490391 | 0.628980782 | 46533 | 42 | 3223 | HOXC6 | 253 | uc001sev.3 | FALSE |
| chr15 | 81426347 | 81426360 | 0.314297778 | 0.628595555 | 69939 | 14 | 161502 | CFAP161 | 34598 | uc002bgb.3 | FALSE |
| chr2 | 97136508 | 97136556 | 0.31428532 | 0.628570641 | 109366 | 49 | 93082 | NEURL3 | 34538 | uc010yup.1 | FALSE |
| chr7 | 38468944 | 38468985 | −0.314217587 | 0.628435174 | 176155 | 42 | 273 | AMPH | 192 | uc003tgw.1 | FALSE |
| chr1 | 228652478 | 228652528 | 0.31415519 | 0.62831038 | 18681 | 51 | 100616308 | MIR4666A | 2703 | uc021pkh.1 | FALSE |
| chr10 | 26505127 | 26505156 | 0.313985074 | 0.627970149 | 22391 | 30 | 2572 | GAD2 | −80 | uc009xkr.3 | TRUE |
| chr1 | 229568252 | 229568360 | 0.313820731 | 0.627641463 | 18783 | 109 | 58 | ACTA1 | 1483 | uc001htm.3 | FALSE |
| chr5 | 42995369 | 42995527 | 0.313809181 | 0.627618363 | 151977 | 159 | 648987 | LOC648987 | 23386 | uc021xye.1 | FALSE |
| chr1 | 66998806 | 66998812 | 0.313713653 | 0.627427306 | 9138 | 7 | 84251 | SGIP1 | −1013 | uc001dcr.3 | TRUE |
| chr7 | 27531429 | 27531652 | −0.313686375 | 0.627372751 | 175290 | 224 | 11112 | HIBADH | 170968 | uc003szi.3 | FALSE |
| chr14 | 88620324 | 88620361 | −0.313676123 | 0.627352246 | 62124 | 38 | 54207 | KCNK10 | 116894 | uc001xwm.3 | FALSE |
| chr19 | 51830634 | 51830960 | 0.313628896 | 0.627257792 | 101448 | 327 | 402665 | IGLON5 | 15532 | uc002pwd.3 | FALSE |
| chr11 | 14995322 | 14995536 | 0.313420258 | 0.626840515 | 32931 | 215 | 796 | CALCA | −1490 | uc001mlt.2 | TRUE |
| chr13 | 25085301 | 25085405 | −0.313415321 | 0.626830641 | 53266 | 105 | 143 | PARP4 | 1543 | uc010tdc.2 | FALSE |
| chr2 | 176945526 | 176945899 | 0.313328268 | 0.626656535 | 114054 | 374 | 344191 | EVX2 | 2791 | uc010zeu.2 | FALSE |
| chr18 | 55107755 | 55108068 | 0.313102682 | 0.626205365 | 92588 | 314 | 9480 | ONECUT2 | 4838 | uc002lgo.3 | FALSE |
| chr4 | 96470286 | 96470293 | 0.312879495 | 0.625758991 | 145433 | 8 | 8633 | UNC5C | 68 | uc003htq.3 | FALSE |
| chr2 | 88428522 | 88428542 | −0.312855123 | 0.625710247 | 109060 | 21 | 2168 | FABP1 | −872 | uc002sst.2 | TRUE |
| chr3 | 133118839 | 133118940 | −0.312741053 | 0.625482106 | 136216 | 102 | 8419 | BFSP2 | 49 | uc003epn.1 | FALSE |
| chr5 | 87980976 | 87981068 | 0.312423437 | 0.624846873 | 154022 | 93 | 645323 | LINC00461 | −356 | uc011cua.1 | TRUE |
| chr6 | 28478515 | 28478579 | −0.312384629 | 0.624769259 | 162965 | 65 | 257202 | GPX6 | 4991 | uc021yrx.1 | FALSE |
| chr13 | 78493297 | 78493305 | 0.31233153 | 0.624663059 | 55852 | 9 | 1910 | EDNRB | 598 | uc001vko.2 | FALSE |
| chr7 | 152618527 | 152618877 | −0.312173873 | 0.624347746 | 183917 | 351 | 57180 | ACTR3B | 161693 | uc011kvp.2 | FALSE |
| chr16 | 54317042 | 54317413 | 0.312161119 | 0.624322238 | 76469 | 372 | 79191 | IRX3 | 2965 | uc002eht.1 | FALSE |
| chr10 | 102483843 | 102484048 | 0.311814198 | 0.623628396 | 27394 | 206 | 5076 | PAX2 | −21420 | uc001krk.4 | FALSE |
| chr10 | 3679062 | 3679085 | −0.31167102 | 0.62334204 | 20942 | 24 | 1316 | KLF6 | 148388 | uc001ihb.2 | FALSE |
| chr6 | 130686592 | 130686697 | 0.311636023 | 0.623272046 | 169102 | 106 | 154075 | SAMD3 | −22 | uc003qbx.4 | TRUE |
| chr15 | 48470754 | 48470794 | 0.311544446 | 0.623088892 | 66811 | 41 | 50804 | MYEF2 | −196 | uc001zwi.4 | TRUE |
| chr3 | 192445533 | 192445538 | 0.311480976 | 0.622961953 | 139497 | 6 | 2257 | FGF12 | −145 | uc003fsy.3 | TRUE |
| chr2 | 45160445 | 45160490 | 0.311173852 | 0.622347704 | 106356 | 46 | 6496 | SIX3 | −8547 | uc002run.2 | FALSE |
| chr5 | 156607836 | 156607853 | −0.31110203 | 0.62220406 | 158163 | 18 | 3702 | ITK | −54 | uc003lwo.1 | TRUE |
| chr19 | 13113668 | 13113893 | 0.310659461 | 0.621318921 | 96677 | 226 | 4784 | NFIX | 7084 | uc002mwd.3 | FALSE |
| chr1 | 98514282 | 98514388 | 0.310659395 | 0.62131879 | 10547 | 107 | 400765 | MIR137HG | 861 | uc001drx.2 | FALSE |
| chr17 | 47091339 | 47091521 | 0.310374776 | 0.620749551 | 86728 | 183 | 10642 | IGF2BP1 | 16565 | uc010dbj.3 | FALSE |
| chr11 | 107799979 | 107800031 | 0.310363695 | 0.620727391 | 39775 | 53 | 54734 | RAB39A | 702 | uc001pjt.3 | FALSE |
| chr7 | 27163331 | 27163820 | 0.310353615 | 0.62070723 | 175211 | 490 | 3200 | HOXA3 | 2819 | uc003syk.3 | FALSE |
| chr6 | 30135113 | 30135162 | −0.310154069 | 0.620308139 | 163338 | 50 | 89870 | TRIM15 | 4130 | uc010jrx.3 | FALSE |
| chr1 | 17086061 | 17086071 | 0.310056755 | 0.62011351 | 3620 | 11 | 11223 | MST1L | 481 | uc001azp.5 | FALSE |
| chr1 | 157738450 | 157738458 | −0.3100292 | 0.6200584 | 13562 | 9 | 79368 | FCRL2 | 8464 | uc001frd.2 | FALSE |
| chr7 | 27282431 | 27282444 | 0.310020643 | 0.620041286 | 175273 | 14 | 2128 | EVX1 | 112 | uc011jzn.1 | FALSE |
| chr4 | 175752696 | 175752781 | −0.30960332 | 0.619206639 | 148460 | 86 | 8001 | GLRA3 | −2231 | uc003ity.1 | FALSE |
| chr1 | 70035392 | 70035479 | 0.309485494 | 0.618970988 | 9314 | 88 | 57554 | LRRC7 | 2524 | uc001deo.1 | FALSE |
| chr21 | 38076869 | 38077042 | 0.309283823 | 0.618567646 | 123497 | 174 | 6493 | SIM2 | 4878 | uc002yvr.2 | FALSE |
| chr17 | 46692534 | 46692553 | 0.309197811 | 0.618395621 | 86644 | 20 | 3218 | HOXB8 | −233 | uc002inw.3 | TRUE |
| chr11 | 121970694 | 121970725 | 0.309165677 | 0.618331355 | 41068 | 32 | 399959 | MIR100HG | 434 | uc010rzr.1 | FALSE |
| chr13 | 37004721 | 37004737 | 0.309025355 | 0.618050709 | 54130 | 17 | 8900 | CCNA1 | −920 | uc010teo.2 | TRUE |
| chr3 | 156009151 | 156009319 | 0.308668764 | 0.617337529 | 137507 | 169 | 7881 | KCNAB1 | 375 | uc010hvt.1 | FALSE |
| chr18 | 31803633 | 31803674 | 0.308522398 | 0.617044795 | 92037 | 42 | 8715 | NOL4 | −118 | uc002kxt.4 | TRUE |
| chr10 | 22634199 | 22634218 | 0.308441449 | 0.616882899 | 22195 | 20 | 9576 | SPAG6 | −156 | uc001iri.3 | TRUE |
| chr17 | 48546297 | 48546321 | 0.307998824 | 0.615997649 | 87010 | 25 | 80221 | ACSF2 | 607 | uc010dbr.3 | FALSE |
| chr19 | 52956674 | 52956683 | 0.3078837 | 0.615767401 | 101624 | 10 | 147660 | ZNF578 | −146 | uc002pzm.3 | TRUE |
| chr5 | 140420142 | 140420475 | 0.307746907 | 0.615493814 | 156806 | 334 | 29930 | PCDHB1 | −10504 | uc003lik.1 | FALSE |
| chr1 | 119529219 | 119529266 | 0.307717869 | 0.615435738 | 11733 | 48 | 6913 | TBX15 | 2913 | uc001ehl.1 | FALSE |
| chr10 | 118304534 | 118304610 | −0.30736468 | 0.61472936 | 28544 | 77 | 5406 | PNLIP | −818 | uc001lcm.3 | TRUE |
| chr13 | 112786612 | 112786727 | −0.307364551 | 0.614729102 | 57411 | 116 | 6656 | SOX1 | 64699 | uc001vsb.1 | FALSE |
| chr7 | 27204052 | 27204349 | 0.307247529 | 0.614495058 | 175231 | 298 | 3205 | HOXA9 | 800 | uc003syt.3 | FALSE |
| chr17 | 37783488 | 37783689 | 0.307194823 | 0.614389645 | 85035 | 202 | 84152 | PPP1R1B | 311 | uc010cvx.3 | FALSE |
| chr7 | 63667212 | 63667446 | −0.307114399 | 0.614228798 | 177682 | 235 | 730291 | ZNF735 | −135 | uc011kdn.2 | TRUE |
| chr6 | 32064246 | 32064258 | 0.306946136 | 0.613892272 | 163807 | 13 | 7148 | TNXB | 12893 | uc003nzl.2 | FALSE |
| chr7 | 98990837 | 98991138 | 0.306721473 | 0.613442946 | 179791 | 302 | 10095 | ARPC1B | 18539 | uc003uqe.3 | FALSE |
| chr5 | 57878295 | 57878390 | 0.305772539 | 0.611545077 | 152558 | 96 | 115827 | RAB3C | −549 | uc003jrp.3 | TRUE |
| chr5 | 140345655 | 140345966 | 0.30547846 | 0.61095692 | 156797 | 312 | 56134 | PCDHAC2 | 0 | uc003lii.3 | TRUE |
| chrX | 39949510 | 39949685 | 0.305443543 | 0.610887086 | 201472 | 176 | 54880 | BCOR | 7034 | uc004deq.4 | FALSE |
| chr17 | 46656543 | 46656572 | 0.305393816 | 0.610787632 | 86619 | 30 | 3213 | HOXB3 | 3244 | uc002inq.3 | FALSE |
| chr1 | 75595919 | 75595970 | 0.305249292 | 0.610498584 | 9423 | 52 | 431707 | LHX8 | 8 | uc021oou.1 | FALSE |
| chr5 | 35946095 | 35946185 | −0.304913979 | 0.609827958 | 151643 | 91 | 133688 | UGT3A1 | 45350 | uc003jju.1 | FALSE |
| chr10 | 123922863 | 123922970 | 0.30480521 | 0.60961042 | 29018 | 108 | 10579 | TACC2 | 36634 | uc001lfz.3 | FALSE |
| chr19 | 54411668 | 54411773 | 0.303690177 | 0.607380354 | 101883 | 106 | 5582 | PRKCG | 24755 | uc010era.2 | FALSE |
| chr3 | 192444985 | 192445040 | 0.303622262 | 0.607244524 | 139497 | 56 | 2257 | FGF12 | 348 | uc003fsy.3 | FALSE |
| chr19 | 3178413 | 3178512 | −0.303059851 | 0.606119702 | 94673 | 100 | 8698 | S1PR4 | −224 | uc002lxg.3 | TRUE |
| chr2 | 108602860 | 108602906 | 0.302908064 | 0.605816128 | 110253 | 47 | 60482 | SLC5A7 | −89 | uc002tdv.3 | TRUE |
| chr7 | 78368660 | 78368756 | −0.302800677 | 0.605601354 | 178802 | 97 | 9863 | MAGI2 | 31882 | uc011kgs.1 | FALSE |
| chr9 | 139640053 | 139640325 | −0.302633796 | 0.605267593 | 200177 | 273 | 158062 | LCN6 | 149 | uc031tfv.1 | FALSE |
| chr6 | 45905407 | 45905524 | −0.30208575 | 0.604171501 | 165817 | 118 | 53405 | CLIC5 | 78102 | uc031sos.1 | FALSE |
| chr10 | 124459311 | 124459378 | −0.300851847 | 0.601703694 | 29082 | 68 | 399814 | C10orf120 | 0 | uc001lgn.3 | TRUE |

(2) Targeted DMR Validation on Plasma cfDNA Methylomes

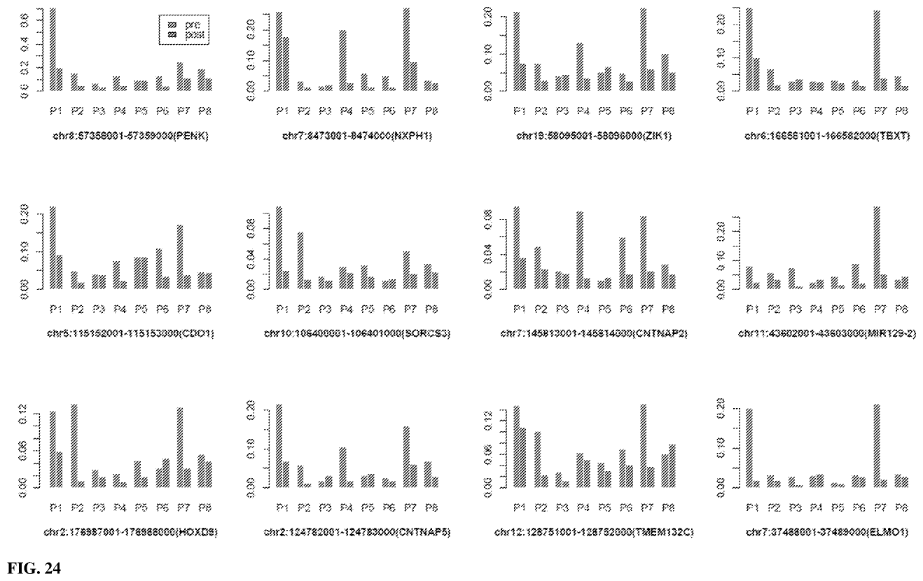

In this section, we focus on validating the significant DMRs detected between tumor and normal tissues. We first performed a paired DMR screening test by comparing the matched pre- and post-treatment cfMBD-seq data, using a fixed window size at 1 kb. In the top 200 DMRs detected in the TCGA dataset, we found a total of 23 overlapping gene regions reached significance (p<0.05) in the plasma DMR test (Table 9), including the two regions in ZNF154 and ELMO1 (top five candidate DMRs from the TCGA analysis). Another two regions ADCYAP1 and PIEZO2 from the top five candidate DMRs also reached marginal significance (p-value ˜0.06). The normalized methylation levels at these top regions across the 16 plasma samples are depicted in FIG. 24 (ranked by plasma DMR p-values). The top five validated regions are located in the promoter regions of genes PENK, NXPH1, ZIK1, TBXT and CDO1. A clear pattern revealed by FIG. 24 is that the TCGA-based DMRs showed the best discriminating power between pre- and post-treatment samples for patients P1 and P7, followed by P4 and P8. Overall, patients P1 and P7 showed the most drastic methylation changes between the pre- and post-treatment samples across most targeted DMRs, potentially due to the fact that both patients (both are male) and had T4 tumors. The pattern of methylation is heterogenous between different patients as evidenced by results in FIG. 24. For example, changes in methylation in patient P4 are more pronounced in DMRs in NXPH1, ZIK1 and CNTNAP2, while changes in P2 (also with a T4 tumor) are more pronounced in HOXD9, TMEMI32C and SORCS3.

TABLE 9
Significant regions (top DMRs from the
TCGA data) from the plasma DMR test

| gene | p-value | region |
|---|---|---|
| PENK | 2.07E−06 | chr8: 57358240-57358713 |
| NXPH1 | 0.000378458 | chr7: 8473279-8473462 |
| ZIK1 | 0.001505289 | chr19: 58095581-58095659 |
| TBXT | 0.001608036 | chr6: 166582188-166582393 |
| CDO1 | 0.001669653 | chr5: 115152420-115152785 |
| SORCS3 | 0.002271258 | chr10: 106400667-106401479 |
| CNTNAP2 | 0.002857663 | chr7: 145813417-145813439 |
| MIR129-2 | 0.004182202 | chr11: 43602845-43602965 |
| HOXD9 | 0.004993118 | chr2: 176987383-176987465 |
| CNTNAP5 | 0.005298808 | chr2: 124782253-124782713 |
| TMEM132C | 0.007690648 | chr12: 128751460-128752058 |
| ELMO1 | 0.01064238 | chr7: 37488162-37488671 |
| KCNK12 | 0.01145063 | chr2: 47797133-47797590 |
| NKX2 | 0.01145462 | chr8: 23564025-23564591 |
| NETO1 | 0.0122865 | chr18: 70534298-70535406 |
| KCNA3 | 0.01328834 | chr1: 111217194-111217785 |
| ZNF154 | 0.01601584 | chr19: 58220295-58220837 |
| SHISA3 | 0.01624042 | chr4: 42399792-42399858 |
| MARCHF11 | 0.023 | chr5: 16179873-16180266 |
| CLVS2 | 0.02808693 | chr6: 123317408-123317875 |
| ZNF415 | 0.03157706 | chr19: 53635967-53636229 |
| TAC1 | 0.04545757 | chr7: 97361241-97361408 |
| GALR1 | 0.04715619 | chr18: 74961966-74962794 |
| PIEZO2 | 0.06011878 | chr18: 11148769-11149763 |
| ADCYAP1 | 0.06878985 | chr18: 904523-905156 |

(3) Genome-Wide cfDNA DMR Analysis on Patient Subgroups

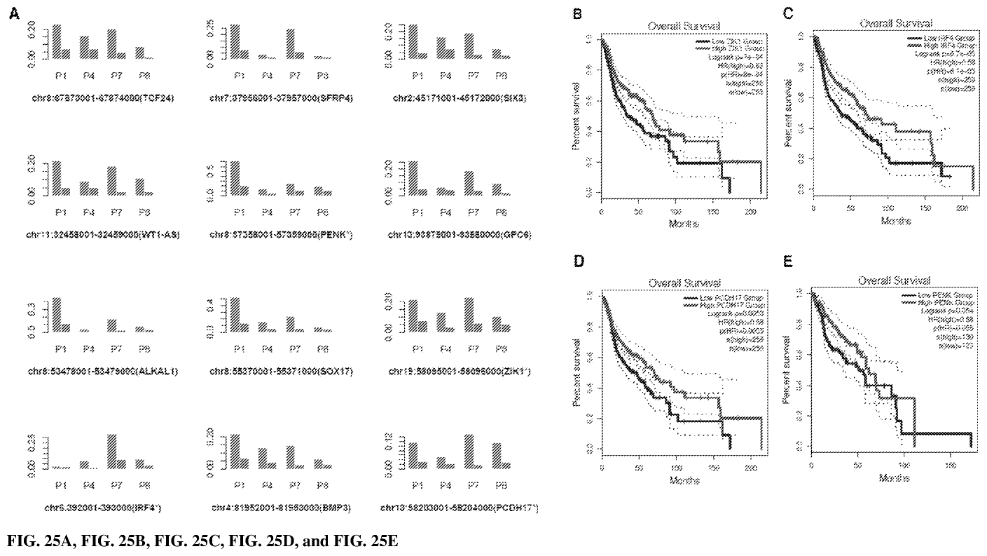

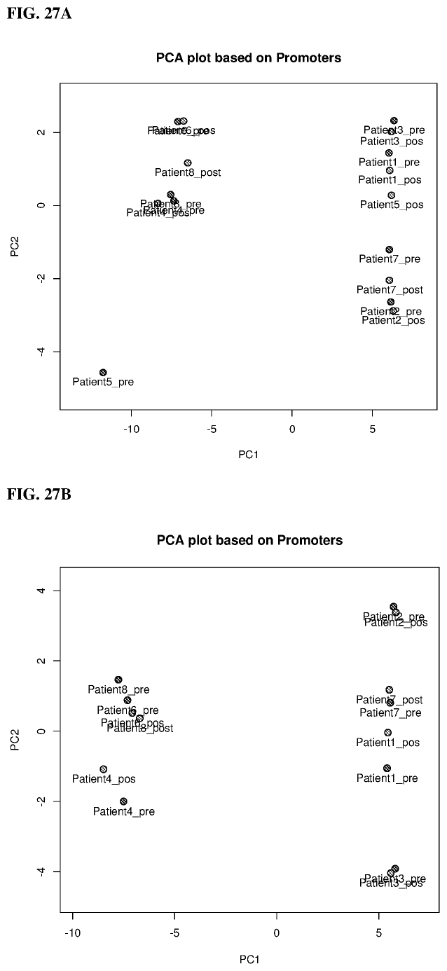

Given the heterogeneity of head and neck cancer at the molecular level and the limited sample size, we reason that it is more powerful to perform DMR analyses based on subsets of patients based on their clinical characteristics. The patient subset information and the resulted top significant or suggestive DMRs (defined as a p-value <0.1 level for adjusted p-value or at 10⁻⁷ level for unadjusted p-value) are listed in Table 7 Table 10. It is interesting to note that the DMR test based on the four patients (P1, P4, P7 and P8) that had the most concordant patterns with TCGA data resulted in the highest number of significant DMRs, followed by the two patient subsets containing T4 tumors (P1, P2, P3 and P7). We excluded patient P5 in most tests (except for the tongue-site subgroup) because the genome-wide PCA analysis (FIG. 27) indicated that the pre-treatment cfDNA methylation profiles could be a potential outlier, which may explain why there was no significant DMR when all patients are included in the test. The multiple-contrast DMR tests can also be considered as sensitivity analyses that provide further confidence for overlapped findings. It was observed that the genomic regions in gene PENK, SFRP4 and SOX17 were selected in at least three tests, indicating they can be further prioritized for biomarker validation. FIG. 25A shows the detailed methylation levels in top regions that were identified based on the TCGA-concordant subgroup, including two top-ranked validated DMRs listed in FIG. 24 (PENK and ZIK1). Similar to the pattern observed in FIG. 24, patients P1 and P7 showed the most drastic changes between the pre- and post-treatment samples. Through assessing the gene expression levels of these top genes in TCGA-HNSC data, we observed that four genes (ZIK1 IRF4, PCDH17 and PENK) demonstrated significant or suggestive association with patient overall survival, as shown in FIG. 25B-E. Collectively, these findings indicate that these four genes may have tumor suppressor functions and are often hypermethylated in HNSC tumor samples or pre-treatment plasma samples.

TABLE 7
Table of top cfDNA DMRs identified based on comparing pre- and post-treatment
cfDNA methylation profiles by considering different patient subgroups.

| Patient group/subgroup | Subset rationale | Top cfDNA DMRs (hg19) ^{†} |
|---|---|---|
| Paired samples from P1, P4, | Patterns most | chr7: 37956001-37957000 (SFRP4) |
| P7, and P8 | consistent with | chr8: 67873001-67874000 (TCF24) |
| | TCGA results | chr2: 45171001-45172000 (SIX3*) |
| | | chr11: 32458001-32459000 (WT1-AS) |
| | | chr8: 53478001-53479000 (ALKAL1) |
| | | chr8: 57358001-57359000 (PENK) |
| | | chr13: 93879001-93880000 (GPC6) |
| | | chr8: 55370001-55371000 (SOX17) |
| | | chr4: 183062001-183063000 (TENM3-AS1*) |
| | | chr1: 119535001-119536000 (TBX15*) |
| | | chr19: 58095001-58096000 (ZIK1) |
| Paired samples from P1, P2, | Patients with T4 | chr7: 37956001-37957000 (SFRP4) |
| P3, and P7 | tumors (male | chr1: 47696001-47697000 (TAL1) |
| | patients) | chr5: 1594001-1595000(SDHAP3) |
| | | chr7: 87257001-87258000 |
| | | (ABCB1/RUNDC3B) |
| | | chr8: 57358001-57359000(PENK) |
| Paired samples from P3, P5, | Tongue site only | chr11: 43602001-43603000 (MIR129-2) |
| P6, and P7 | | |
| P4, P6, and P8 | Female patients | No region significant at 10⁻⁷ level |
| | only | |
| Paired samples from P1, P2, | All T4 patients + | chr7: 37956001-37957000 (SFRP4) |
| P3, P4 and P7 | P4 (signature | chr8: 57358001-57359000 (PENK) |
| | patient) | chr8: 55370001-55371000 (SOX17) |
| | | chr3: 129693001-129694000 (TRH) |
| | | chr14: 48143001-48144000 (MDGA2*) |
| | | chr14: 48145001-48146000 (MDGA2) |
| Paired samples from P1, P2, | All patients | chr8: 57358001-57359000 (PENK) |
| P3, P4, P6, P7, and P8 | except | chr7: 37956001-37957000 (SFRP4) |
| | P5 (potential | chr8: 55370001-55371000 (SOX17) |
| | outlier) | |

^{†} Unless otherwise specific, DMRs are significant at 0.1 level for adjusted p-value or at 10−7 496 level for unadjusted p-value. DMRs not in the promoter region of the gene are indicated by “*”.

(4) Clustering Analysis of Targeted Plasma cfDNA Methylation Regions

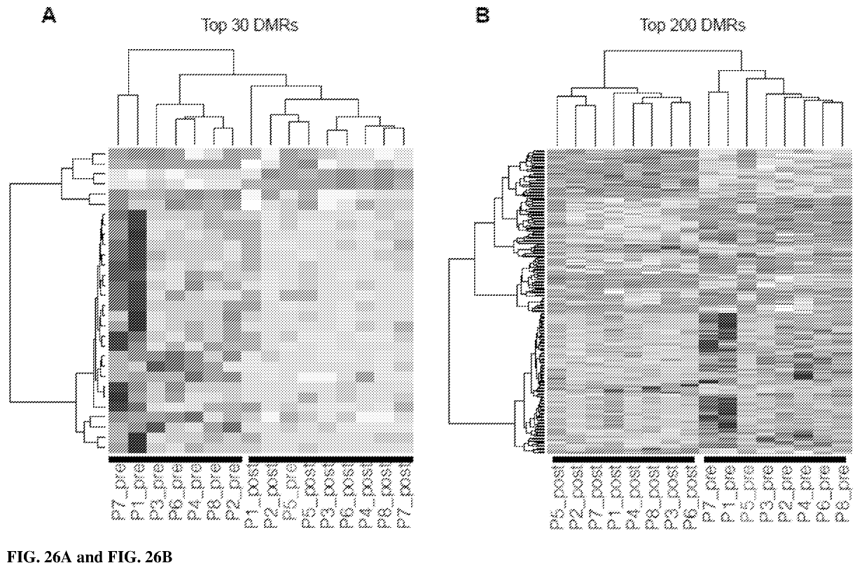

Finally, we tested the performance of top DMRs (as well as the model saturation in terms of the number of biomarkers included) in discriminating pre- and post-treatment plasma samples. The two heatmaps in FIG. 26 illustrate the unsupervised clustering results generated based on top 30 DMRs and top 200 DMRs (from the DMR test using all samples but P5), respectively. It shows that the top 30 regions (most of them are hypermethylated in pre-treatment samples) are already sufficient to separate pre- and post-treatment plasma samples except for the P5 pre-treatment sample. This was expected, because the global PCA analysis also indicated that this sample could be a potential outlier. But when top 200 regions were included, this sample, together with all other samples, can be correctly separated.

### c) Discussion

While the current study does not have sufficient sample size to comment on prognostic value of cfDNA methylation, in this study, we successfully demonstrate the feasibility of isolating cfDNA from plasma and a two-pronged approach in identifying top candidate biomarkers by first identifying DMRs from the TCGA dataset and then validating them in our cfDNA samples.

A biomarker for minimal residual disease is advantageous, especially in the setting of oral cavity squamous cell carcinoma patients, a population where 5-year survival rates are estimated between 40-60% for patients with advanced-stage disease, and the majority of the recurrence occurs in the first 2 years. Moreover, when the biomarkers can predict tumor immune response it is even more preferable. DNAme has the potential to incorporate both of the above features in addition to it being highly tissue specific, and more responsive to genetic variations and environmental exposure. HNSCC is a heterogenous disease and thus focusing on cfDNA DNAme may be advantageous to increase specificity. Previous studies have focused on targeted panels of methylation in either serum/plasma of HNSCC patients. Recently, a study by Burgener et al. did demonstrate tumor-naïve detection of ctDNA by simultaneously profiling mutations and methylation.

In our study, we identified hypermethylation of zinc finger genes, such as ZNF154, in the TCGA tumor samples compared to normal tissue. This is consistent with previous studies where their expression has been shows to correlate with tumor suppressor activity. ZNF154 has been reported as diagnostic marker in liquid biopsies in multiple cancers.

Our study also indicates that top 30 DMRs are sufficient to differentiate between pre-treatment and post-treatment samples indicating that a signature based on these 30 DMRs may be sufficient to determine minimal residual disease. Many genes in the top DMR list have also been indicated as liquid biopsy methylation biomarkers in other cancer types, such as ZNF154 fir multiple cancers, ELMO1 for gastric cancer, indicating that they are reliable cancer-relevant epigenetic biomarkers. The promoter methylation level of IRF4 and PCDH117 are potential liquid biopsy biomarkers for colorectal cancer and bladder cancer.

The top five validated regions were located in the promoter regions of genes PENK, NXPH1, ZIK1, TBXT and CDO1. Through our analysis 4 candidate genes were identified that may have prognostic value in addition to their role in determining minimal residual disease—ZIK1, IRF4, PCDH17 and PENK. ZIK1 (ZNF762) is part of the Zinc Finger protein group with a KRAB-A domain and found on chromosome 19. KRAB box-A is a transcription repressor module and it is plausible that ZIK1 is epigenetically regulated tumor suppressor gene. Interferon regulatory factor 4 (IRF4) is a member of the Interferon family and is specifically expressed in lymphocytes regulating immune responses, immune cell proliferation and differentiation. While its role in hematologic malignancies, IRF4 expression in lung adenocarcinoma has been associated with favorable prognosis. Protocadherin 17 (PCDH17) is part of the cadherin superfamily responsible for cell adhesion and possible tumor growth, migration and invasion. PCDH17 methylation has been noted in urological cancers including esophageal, gastric, colon, and bladder cancers. Proenkephalin (PENK) is expressed in nervous and neuroendocrine systems as part of the opioid pathway, but is also involved in cell cycle regulation and implicated in head and neck, gastric, colon, breast, pancreatic, osteosarcoma, and bladder cancers. NXPH1 is primarily expressed in nervous system and is a secreted glycoprotein that forms complexes with alpha neurexins—a group of protein that promote adhesion between dendrites and axons. In breast cancer samples, NXPH1 methylation levels were lower compared to normal tissues and was more likely to be methylated in low-grade dysplasia than in high-grade dysplasia. In prostate cancers with Gleason score ≥7, NXPH1 expression level was upregulated and was incorporated in a 10 gene signature that predicted biochemical recurrence. However, a negative correlation was noted in patients with pancreatic cancer with regards to lymph node metastasis. NXPH1 methylation has also been implicated in neuroblastoma and was incorporated in a 5 gene prognostic signature where it was down regulated indicative of playing a tumor suppressive role. This indicates that tissue specific changes maybe at play. T-box transcription factor T (TBXT) expression is implicated in mesodermal specification during vertebrate development and is epigenetically silenced in human fetus development at 12 weeks. TBXT expression has been reported in a number of solid malignancies including head and neck, lung, breast, colon, prostate and chordoma—with hypothesis that it promotes epithelial-mesenchymal transition and targeting it may help in cancer control. Promoter methylation CDO1 has also been identified as diagnostic biomarkers in lung cancer.

Limitations of this study include limited samples size to draw definitive prognostic conclusion. cfDNA has been correlated with overall stage and subsite. Our study included primarily advanced stage disease and mainly oral cavity squamous cell carcinomas. It is well established that advanced stage cancers and different subsites can have different methylation pattern and lymphatic drainage, thus it is plausible that similar results may not be evident in lower stage disease. In summary, we identified multiple candidate DMRs that allowed distinction between pre-treatment and post-treatment samples indicating its utility for minimal residual disease and potential as prognostic biomarker.

### 4. Example 4

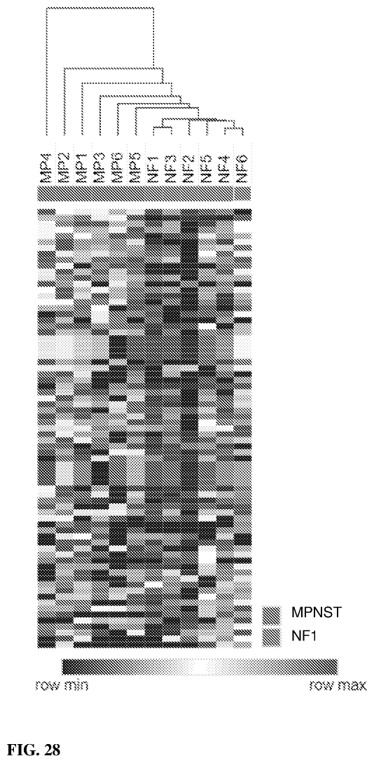

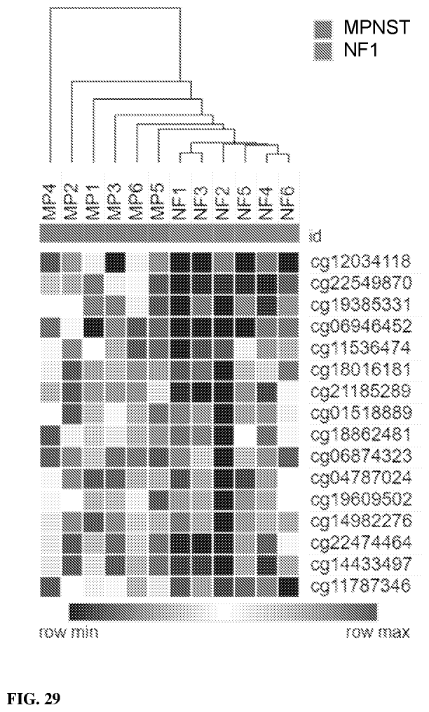

To test if hypermethylation at selected CpG sites in tumor tissues can also be detectable in plasma samples from patients with MPNST (Malignant Peripheral Nerve Sheath Tumor), we collected blood samples from 6 patients with MPNST and 6 patients with NF1. Since the cfMBD-seq can detect genomic regions that are hypermethylated in plasma samples, we first converted the 73 CpG sites into genomic regions, which resulted in 68 sites mapping to CpG islands. For the five CpG sites without CpG island location, 300 bp was added to either site to generate a 601 bp interval for final data analysis. We first performed hierarchical clustering analysis using all 73 selected regions and observed perfect separation between MPNST and NF1 groups with clear trend of hypermethylation in MPNST group (FIG. 28). Statistical analysis showed 70 of 73 selected genomic regions with greater than 1.2-fold higher methylation in MPNST patients than NF1 patients. Further analysis identified statistical significance in 30 CpG islands hypermethylated in MPNST compared to NF1 (P<0.05 and FC>1.5), of which 16 CpG islands with FDR<0.1 (FIG. 29). Except cg01518889 mapping to N_shelf, the remaining 15 CpG sites were located within CpG islands.

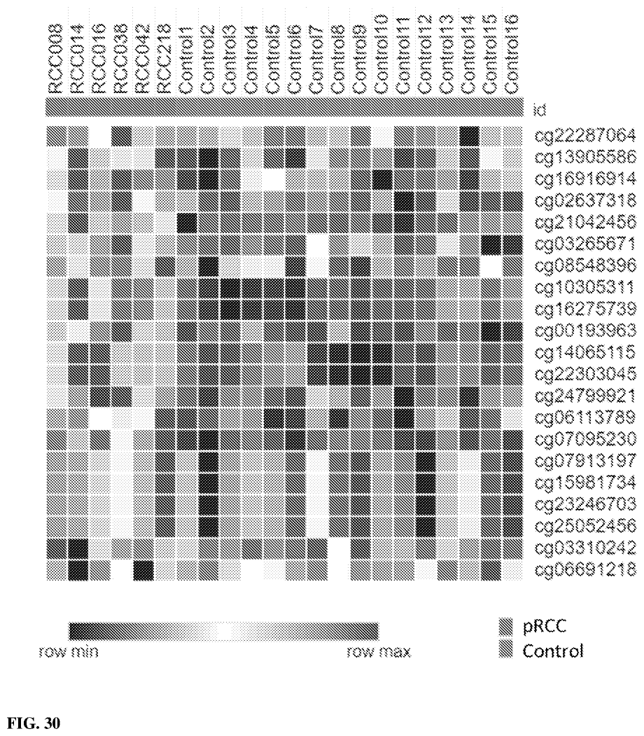

To further assess potential clinical use of hypermethylated CpG sites, we applied the cfMBD-seq to the plasma samples from 6 pRCC patients and 16 healthy controls. Among 79 probe sets with hypermethylation in tumor tissues, we confirmed 21 probe sets showing cfDNA methylation difference with FDR<0.2 and fold changes >1.5. 20 of the 21 significant probe sets showed hypermethylation in patients with pRCC when compared to healthy controls (FIG. 30). This data support feasibility of using cfDNA to detect tumor-specific methylation papillary renal cell carcinoma (pRCC).

### E. References

Arechederra, M.; Daian, F.; Yim, A.; Bazai, S. K.; Richelme, S.; Dono, R.; Saurin, A. J.; Habermann, B. H.; Maina, F. Hypermethylation of gene body CpG islands predicts high dosage of functional oncogenes in liver cancer. Nat Commun 2018, 9, 3164, doi:10.1038/s41467-018-05550-5.
Aryee, M. J.; Jaffe, A. E.; Corrada-Bravo, H.; Ladd-Acosta, C.; Feinberg, A. P.; Hansen, K. D.; Irizarry, R. A. Minfi: a flexible and comprehensive Bioconductor package for the analysis of Infinium DNA methylation microarrays. Bioinformatics 2014, 30, 1363-1369, doi:10.1093/bioinformatics/btu049.
Bhatlekar, S.; Fields, J. Z.; Boman, B. M. Role of HOX Genes in Stem Cell Differentiation and Cancer. Stem Cells Int 2018, 2018, 3569493, doi:10.1155/2018/3569493.
Blighe K, R. S., Lewis M. EnhancedVolcano: Publication-ready volcano plots with enhanced colouring and labeling. 2021.
Chan, K. C.; Jiang, P.; Chan, C. W.; Sun, K.; Wong, J.; Hui, E. P.; Chan, S. L.; Chan, W. C.; Hui, D. S.; Ng, S. S., et al. Noninvasive detection of cancer-associated genome-wide hypomethylation and copy number aberrations by plasma DNA bisulfite sequencing. Proc Natl Acad Sci USA 2013, 110, 18761-18768, doi:10.1073/pnas.1313995110.
Chan, R. F.; Shabalin, A. A.; Xie, L. Y.; Adkins, D. E.; Zhao, M.; Turecki, G.; Clark, S. L.; Aberg, K. A.; van den Oord, E. Enrichment methods provide a feasible approach to comprehensive and adequately powered investigations of the brain methylome. Nucleic Acids Res 2017, 45, e97, doi:10.1093/nar/gkx143.
Chen, S.; Zhou, Y.; Chen, Y.; Gu, J. fastp: an ultra-fast all-in-one FASTQ preprocessor. Bioinformatics 2018, 34, i884-i890, doi:10.1093/bioinformatics/bty560.
Esteller, M. Cancer epigenomics: DNA methylomes and histone-modification maps. Nat Rev Genet 2007, 8, 286-298, doi:10.1038/nrg2005.
Esteller, M. Epigenetics in cancer. The New England journal of medicine 2008, 358, 1148-1159, doi:10.1056/NEJMra072067.
Friedman, J.; Hastie, T.; Tibshirani, R. Regularization Paths for Generalized Linear Models via Coordinate Descent. J Stat Softw 2010, 33, 1-22.
Gai, W.; Sun, K. Epigenetic Biomarkers in Cell-Free DNA and Applications in Liquid Biopsy. Genes (Basel) 2019, 10, doi:10.3390/genes10010032.
Heitzer, E.; Haque, I. S.; Roberts, C. E. S.; Speicher, M. R. Current and future perspectives of liquid biopsies in genomics-driven oncology. Nat Rev Genet 2019, 20, 71-88, doi:10.1038/s41576-018-0071-5.
Hiam-Galvez, K. J.; Allen, B. M.; Spitzer, M. H. Systemic immunity in cancer. Nat Rev Cancer 2021, 21, 345-359, doi:10.1038/s41568-021-00347-z.
Hu, Y.; Ulrich, B. C.; Supplee, J.; Kuang, Y.; Lizotte, P. H.; Feeney, N. B.; Guibert, N. M.; Awad, M. M.; Wong, K. K.; Janne, P. A., et al. False-Positive Plasma Genotyping Due to Clonal Hematopoiesis. Clin Cancer Res 2018, 24, 4437-4443, doi:10.1158/1078-0432.CCR-18-0143.
Huang, J.; Soupir, A. C.; Wang, L. Cell-free DNA methylome profiling by MBD-seq with ultra-low input. Epigenetics 2021, 10.1080/15592294.2021.1896984, 1-14, doi:10.1080/15592294.2021.1896984.
Huang, J.; Wang, L. Cell-Free DNA Methylation Profiling Analysis-Technologies and Bioinformatics. Cancers (Basel) 2019, 11, doi:10.3390/cancers11111741.
Irizarry, R. A.; Ladd-Acosta, C.; Wen, B.; Wu, Z.; Montano, C.; Onyango, P.; Cui, H.; Gabo, K.; Rongione, M.; Webster, M., et al. The human colon cancer methylome shows similar hypo- and hypermethylation at conserved tissue-specific CpG island shores. Nat Genet 2009, 41, 178-186, doi:10.1038/ng.298.
Jones, P. A. Functions of DNA methylation: islands, start sites, gene bodies and beyond. Nat Rev Genet 2012, 13, 484-492, doi:10.1038/nrg3230.
Kalinski, P. Regulation of immune responses by prostaglandin E2. J Immunol 2012, 188, 21-28, doi:10.4049/jimmunol.1101029.
Krijthe, J. H. Rtsne: T-Distributed Stochastic Neighbor Embedding using a Barnes-Hut Implementation. 2015.
Kuhn, M. Building Predictive Models in R Using the caret Package. Journal of Statistical Software 2008, dx.doi.org/10.18637/jss.v028.i05, doi:dx.doi.org/10.18637/jss.v028.i05.
Langmead, B.; Salzberg, S. L. Fast gapped-read alignment with Bowtie 2. Nat Methods 2012, 9, 357-359, doi:10.1038/nmeth.1923.
Lasseter, K.; Nassar, A. H.; Hamieh, L.; Berchuck, J. E.; Nuzzo, P. V.; Korthauer, K.; Shinagare, A. B.; Ogorek, B.; McKay, R.; Thorner, A. R., et al. Plasma cell-free DNA variant analysis compared with methylated DNA analysis in renal cell carcinoma. Genet Med 2020, 10.1038/s41436-020-0801-x, doi:10.1038/s41436-020-0801-x.
Leary, R. J.; Sausen, M.; Kinde, I.; Papadopoulos, N.; Carpten, J. D.; Craig, D.; O'Shaughnessy, J.; Kinzler, K. W.; Parmigiani, G.; Vogelstein, B., et al. Detection of chromosomal alterations in the circulation of cancer patients with whole-genome sequencing. Sci Transl Med 2012, 4, 162ra154, doi:10.1126/scitranslmed.3004742.
Li, H.; Handsaker, B.; Wysoker, A.; Fennell, T.; Ruan, J.; Homer, N.; Marth, G.; Abecasis, G.; Durbin, R.; Genome Project Data Processing, S. The Sequence Alignment/Map format and SAMtools. Bioinformatics 2009, 25, 2078-2079, doi:10.1093/bioinformatics/btp352.
Liu, M. C.; Oxnard, G. R.; Klein, E. A.; Swanton, C.; Seiden, M. V.; Consortium, C. Sensitive and specific multi-cancer detection and localization using methylation signatures in cell-free DNA. Ann Oncol 2020, 31, 745-759, doi:10.1016/j.annonc.2020.02.011.
Love, M. I.; Huber, W.; Anders, S. Moderated estimation of fold change and dispersion for RNA-seq data with DESeq2. Genome Biol 2014, 15, 550, doi:10.1186/s13059-014-0550-8.
Luo, H.; Wei, W.; Ye, Z.; Zheng, J.; Xu, R. H. Liquid Biopsy of Methylation Biomarkers in Cell-Free DNA. Trends Mol Med 2021, 27, 482-500, doi:10.1016/j.molmed.2020.12.011.
Luo, H.; Zhao, Q.; Wei, W.; Zheng, L.; Yi, S.; Li, G.; Wang, W.; Sheng, H.; Pu, H.; Mo, H., et al. Circulating tumor DNA methylation profiles enable early diagnosis, prognosis prediction, and screening for colorectal cancer. Sci Transl Med 2020, 12, doi:10.1126/scitranslmed.aax7533.
Marini, F.; Binder, H. pcaExplorer: an R/Bioconductor package for interacting with RNA-seq principal components. Bmc Bioinformatics 2019, 20, 331, doi:10.1186/s12859-019-2879-1.
Moss, J.; Magenheim, J.; Neiman, D.; Zemmour, H.; Loyfer, N.; Korach, A.; Samet, Y.; Maoz, M.; Druid, H.; Arner, P., et al. Comprehensive human cell-type methylation atlas reveals origins of circulating cell-free DNA in health and disease. Nat Commun 2018, 9, 5068, doi:10.1038/s41467-018-07466-6.
Nair, S. S.; Coolen, M. W.; Stirzaker, C.; Song, J. Z.; Statham, A. L.; Strbenac, D.; Robinson, M. D.; Clark, S. J. Comparison of methyl-DNA immunoprecipitation (MeDIP) and methyl-CpG binding domain (MBD) protein capture for genome-wide DNA methylation analysis reveal CpG sequence coverage bias. Epigenetics 2011, 6, 34-44, doi:10.4161/epi.6.1.13313.
Nian, J.; Sun, X.; Ming, S.; Yan, C.; Ma, Y.; Feng, Y.; Yang, L.; Yu, M.; Zhang, G.; Wang, X. Diagnostic Accuracy of Methylated SEPT9 for Blood-based Colorectal Cancer Detection: A Systematic Review and Meta-Analysis. Clin Transl Gastroenterol 2017, 8, e216, doi:10.1038/ctg.2016.66.
Nuzzo, P. V.; Berchuck, J. E.; Korthauer, K.; Spisak, S.; Nassar, A. H.; Abou Alaiwi, S.; Chakravarthy, A.; Shen, S. Y.; Bakouny, Z.; Boccardo, F., et al. Detection of renal cell carcinoma using plasma and urine cell-free DNA methylomes. Nat Med 2020, 26, 1041-1043, doi:10.1038/s41591-020-0933-1.
Quinlan, A. R. BEDTools: The Swiss-Army Tool for Genome Feature Analysis. Curr Protoc Bioinformatics 2014, 47, 11 12 11-34, doi:10.1002/0471250953.bi1112s47.
Roadmap Epigenomics, C.; Kundaje, A.; Meuleman, W.; Ernst, J.; Bilenky, M.; Yen, A.; Heravi-Moussavi, A.; Kheradpour, P.; Zhang, Z.; Wang, J., et al. Integrative analysis of 111 reference human epigenomes. Nature 2015, 518, 317-330, doi:10.1038/nature14248.
Robertson, K. D. DNA methylation, methyltransferases, and cancer. Oncogene 2001, 20, 3139-3155, doi:10.1038/sj.onc.1204341.
Rolfo, C.; Russo, A. Liquid biopsy for early stage lung cancer moves ever closer. Nat Rev Clin Oncol 2020, 17, 523-524, doi:10.1038/s41571-020-0393-z.
Shabalin, A. A.; Hattab, M. W.; Clark, S. L.; Chan, R. F.; Kumar, G.; Aberg, K. A.; van den Oord, E. RaMWAS: fast methylome-wide association study pipeline for enrichment platforms. Bioinformatics 2018, 34, 2283-2285, doi:10.1093/bioinformatics/bty069.
Shen, S. Y.; Burgener, J. M.; Bratman, S. V.; De Carvalho, D. D. Preparation of cfMeDIP-seq libraries for methylome profiling of plasma cell-free DNA. Nat Protoc 2019, 14, 2749-2780, doi:10.1038/s41596-019-0202-2.
Shen, S. Y.; Singhania, R.; Fehringer, G.; Chakravarthy, A.; Roehrl, M. H. A.; Chadwick, D.; Zuzarte, P. C.; Borgida, A.; Wang, T. T.; Li, T. T., et al. Sensitive tumour detection and classification using plasma cell-free DNA methylomes. Nature 2018, 563, 579-+, doi:10.1038/s41586-018-0703-0.
Siravegna, G.; Marsoni, S.; Siena, S.; Bardelli, A. Integrating liquid biopsies into the management of cancer. Nature Reviews Clinical Oncology 2017, 14, 531-548, doi:10.1038/nrclinonc.2017.14.
Sprang, M.; Paret, C.; Faber, J. CpG-Islands as Markers for Liquid Biopsies of Cancer Patients. Cells 2020, 9, doi:10.3390/cells9081820.
Su, Y.; Fang, H. B.; Jiang, F. An epigenetic classifier for early stage lung cancer. Clin Epigenetics 2018, 10, 68, doi:10.1186/s13148-018-0502-3.
Sun, K.; Jiang, P.; Chan, K. C.; Wong, J.; Cheng, Y. K.; Liang, R. H.; Chan, W. K.; Ma, E. S.; Chan, S. L.; Cheng, S. H., et al. Plasma DNA tissue mapping by genome-wide methylation sequencing for noninvasive prenatal, cancer, and transplantation assessments. Proc Natl Acad Sci USA 2015, 112, E5503-5512, doi:10.1073/pnas.1508736112.
Sung, H.; Ferlay, J.; Siegel, R. L.; Laversanne, M.; Soerjomataram, I.; Jemal, A.; Bray, F. Global Cancer Statistics 2020: GLOBOCAN Estimates of Incidence and Mortality Worldwide for 36 Cancers in 185 Countries. CA Cancer J Clin 2021, 71, 209-249, doi:10.3322/caac.21660.
Suzuki, M. M.; Bird, A. DNA methylation landscapes: provocative insights from epigenomics. Nat Rev Genet 2008, 9, 465-476, doi:10.1038/nrg2341.
Vrba, L.; Futscher, B. W. A suite of DNA methylation markers that can detect most common human cancers. Epigenetics 2018, 13, 61-72, doi:10.1080/15592294.2017.1412907.
Wan, J. C. M.; Massie, C.; Garcia-Corbacho, J.; Mouliere, F.; Brenton, J. D.; Caldas, C.; Pacey, S.; Baird, R.; Rosenfeld, N. Liquid biopsies come of age: towards implementation of circulating tumour DNA. Nat Rev Cancer 2017, 17, 223-238, doi:10.1038/nrc.2017.7.
Warren, J. D.; Xiong, W.; Bunker, A. M.; Vaughn, C. P.; Furtado, L. V.; Roberts, W. L.; Fang, J. C.; Samowitz, W. S.; Heichman, K. A. Septin 9 methylated DNA is a sensitive and specific blood test for colorectal cancer. BMC Med 2011, 9, 133, doi:10.1186/1741-7015-9-133.
Wei, M.; Zhang, C.; Tian, Y.; Du, X.; Wang, Q.; Zhao, H. Expression and Function of WNT6: From Development to Disease. Front Cell Dev Biol 2020, 8, 558155, doi:10.3389/fcell.2020.558155.
Xu, R. H.; Wei, W.; Krawczyk, M.; Wang, W.; Luo, H.; Flagg, K.; Yi, S.; Shi, W.; Quan, Q.; Li, K., et al. Circulating tumour DNA methylation markers for diagnosis and prognosis of hepatocellular carcinoma. Nat Mater 2017, 16, 1155-1161, doi:10.1038/nmat4997.

1. A method of treating a cancer, said method comprising
a) obtaining a fluid biological sample;
b) extracting cfDNA;
c) generating methylated filler DNA;
d) ligating an adapter to the cfDNA and combining with filler DNA thereby creating methylation cfDNA library;
e) enriching for methylated cfDNA;
f) amplifying and sequencing enriched methylated cfDNA library;
g) assaying CpG islands for hypermethylation relative to a normal control; wherein the presence of CpG hypermethylation at a CpG islands chr4:174427892-174428192, chr7:27265159-27265493, chr7:65037625-65037864, chr8:124172801-124173541, chr12:54408427-54408713, chr13:28549840-28550246, chr1:50798668-50799536, chr5:92939796-92940216, or chr12:114881650-114881937, and/or at CpG island associated with CLIP4 (chr2:29337984-29338909), LONRF2 (chr2:100937780-100939059), RNF217 (chr6:125283125-125284389), MEIS1 (chr2:66672432-66673636), ZNF638 (chr2:71503548-71504233), WNT6 (chr2:219736133-219736592), MGST2 (chr4:140655963-140657135), PTGER4 (chr5:40679503-40682081), C9orf129 (chr9:96108467-96108992), B4GALNT1 (chr12:58021295-58022037), HOXB8 (chr17:46691521-46692097), TBX4 (chr17:59539363-59539834), SOX9 (chr17:70112825-70114271), RNF220 (chr1:44883137-44884272), CELF2 (chr10:11059443-11060524), and/or DBX1 (chr11:20177609-20178824) indicates the presence of a cancer; and
h) treating the cancer with an effective amount of a therapeutic agent.

2. A method of detecting a cancer, said method comprising
a) obtaining a fluid biological sample;
b) extracting cfDNA;
c) generating methylated filler DNA;
d) ligating an adapter to the cfDNA and combining with filler DNA thereby creating methylation cfDNA library;
e) enriching for methylated cfDNA;
f) amplifying and sequencing enriched methylated cfDNA library; and
g) assaying CpG islands for hypermethylation relative to a normal control; wherein the presence of CpG hypermethylation at a CpG islands chr4:174427892-174428192, chr7:27265159-27265493, chr7:65037625-65037864, chr8:124172801-124173541, chr12:54408427-54408713, chr13:28549840-28550246, chr1:50798668-50799536, chr5:92939796-92940216, or chr12:114881650-114881937, and/or at CpG island associated with CLIP4 (chr2:29337984-29338909), LONRF2 (chr2:100937780-100939059), RNF217 (chr6:125283125-125284389), MEIS1 (chr2:66672432-66673636), ZNF638 (chr2:71503548-71504233), WNT6 (chr2:219736133-219736592), MGST2 (chr4:140655963-140657135), PTGER4 (chr5:40679503-40682081), C9orf129 (chr9:96108467-96108992), B4GALNT1 (chr12:58021295-58022037), HOXB8 (chr17:46691521-46692097), TBX4 (chr17:59539363-59539834), SOX9 (chr17:70112825-70114271), RNF220 (chr1:44883137-44884272), CELF2 (chr10:11059443-11060524), and/or DBX1 (chr11:20177609-20178824) indicates the presence of a cancer.

3. A method of grading a cancer, said method comprising
a) obtaining a fluid biological sample;
b) extracting cfDNA;
c) generating methylated filler DNA;
d) ligating an adapter to the cfDNA and combining with filler DNA thereby creating methylation cfDNA library;
e) enriching for methylated cfDNA;
f) amplifying and sequencing enriched methylated cfDNA library; and
g) assaying CpG islands for hypermethylation relative to a normal control; wherein the presence of CpG hypermethylation at a CpG islands chr4:174427892-174428192, chr7:27265159-27265493, chr7:65037625-65037864, chr8:124172801-124173541, chr12:54408427-54408713, chr13:28549840-28550246, chr1:50798668-50799536, chr5:92939796-92940216, or chr12:114881650-114881937, and/or at CpG island associated with CLIP4 (chr2:29337984-29338909), LONRF2 (chr2:100937780-100939059), RNF217 (chr6:125283125-125284389), MEIS1 (chr2:66672432-66673636), ZNF638 (chr2:71503548-71504233), WNT6 (chr2:219736133-219736592), MGST2 (chr4:140655963-140657135), PTGER4 (chr5:40679503-40682081), C9orf129 (chr9:96108467-96108992), B4GALNT1 (chr12:58021295-58022037), HOXB8 (chr17:46691521-46692097), TBX4 (chr17:59539363-59539834), SOX9 (chr17:70112825-70114271), RNF220 (chr1:44883137-44884272), CELF2 (chr10:11059443-11060524), and/or DBX1 (chr11:20177609-20178824) indicates presence of hypermethylation.

4. The method of claim 1, wherein the cancer is pancreatic, colorectal, or lung cancer.

5. A method of typing a cancer, said method comprising
a) obtaining a fluid biological sample;
b) extracting cfDNA;
c) generating methylated filler DNA;
d) ligating an adapter to the cfDNA and combining with filler DNA thereby creating methylation cfDNA library;
e) enriching for methylated cfDNA;
f) amplifying and sequencing enriched methylated cfDNA library; and
g) assaying CpG islands for hypermethylation relative to a normal control; wherein the presence of CpG hypermethylation at a CpG islands associated with CLIP4 (chr2:29337984-29338909), LONRF2 (chr2:100937780-100939059), and/or RNF217 (chr6:125283125-125284389) indicate colorectal cancer; wherein the presence of CpG hypermethylation at a CpG islands at chr4:174427892-174428192, chr7:27265159-27265493, chr7:65037625-65037864, chr8:124172801-124173541, and/or chr12:54408427-54408713, and/or at CpG islands associated with MEIS1 (chr2:66672432-66673636), ZNF638 (chr2:71503548-71504233), WNT6 (chr2:219736133-219736592), MGST2 (chr4:140655963-140657135), PTGER4 (chr5:40679503-40682081), C9orf129 (chr9:96108467-96108992), B4GALNT1 (chr12:58021295-58022037), HOXB8 (chr17:46691521-46692097), TBX4 (chr17:59539363-59539834), and/or SOX9 (chr17:70112825-70114271) indicate lung cancer; and wherein the presence of CpG hypermethylation at a CpG islands at chr13:28549840-28550246, chr1:50798668-50799536, chr5:92939796-92940216, and/or chr12:114881650-114881937, and/or at CpG island associated with RNF220 (chr1:44883137-44884272), CELF2 (chr10:11059443-11060524), and/or DBX1 (chr11:20177609-20178824) indicates the presence of a pancreatic cancer.

6. The method of claim 1, wherein the fluid biological sample comprises blood, serum, plasma, or cerebral spinal fluid.

7. The method of claim 1, wherein the methylated filler DNA is generated by treating amplicons of Enterobacteria phage λ DNA with CpG methyltransferase.

8. The method of claim 1, wherein the normal control comprise autologous noncancerous tissue from the subject or a control standard.

9. The method of claim 2, wherein the cancer is pancreatic, colorectal, or lung cancer.

10. The method of claim 2, wherein the fluid biological sample comprises blood, serum, plasma, or cerebral spinal fluid.

11. The method of claim 2, wherein the methylated filler DNA is generated by treating amplicons of Enterobacteria phage λ DNA with CpG methyltransferase.

12. The method of claim 2, wherein the normal control comprise autologous noncancerous tissue from the subject or a control standard.
cancer.

13. The method of claim 3, wherein the cancer is pancreatic, colorectal, or lung cancer.

14. The method of claim 3, wherein the fluid biological sample comprises blood, serum, plasma, or cerebral spinal fluid.

15. The method of claim 3, wherein the methylated filler DNA is generated by treating amplicons of Enterobacteria phage, DNA with CpG methyltransferase.

16. The method of claim 3, wherein the normal control comprise autologous noncancerous tissue from the subject or a control standard.

17. The method of claim 5, wherein the cancer is pancreatic, colorectal, or lung cancer.

18. The method of claim 5, wherein the fluid biological sample comprises blood, serum, plasma, or cerebral spinal fluid.

19. The method of claim 5, wherein the methylated filler DNA is generated by treating amplicons of Enterobacteria phage λ DNA with CpG methyltransferase.

20. The method of claim 5, wherein the normal control comprise autologous noncancerous tissue from the subject or a control standard.


SEQUENCE LISTING (2 sequences)

SEQ ID NO: 1 - DNA, 25 bp, synthetic construct
  source 1..25: mol_type = other DNA
cgatgggttaattcgctcgttgtgg

SEQ ID NO: 2 - DNA, 20 bp, synthetic construct
  source 1..20: mol_type = other DNA
gcacaacggaaagagcactg